(19) United States
(12) Patent Application Publication
(10) Pub. No.: US 2024/0197843 A1
(43) Pub. Date: Jun. 20, 2024

(54) BACTERIA ENGINEERED TO TREAT DISORDERS IN WHICH OXALATE IS DETRIMENTAL

(71) Applicant: Synlogic Operating Company, Inc., Cambridge, MA (US)

(72) Inventors: Vincent M. Isabella, Medford, MA (US); David Lubkowicz, Somerville, MA (US); Michael James, Melrose, MA (US); Caroline Kurtz, Sudbury, MA (US)

(73) Assignee: Synlogic Operating Company, Inc., Cambridge, MA (US)

(21) Appl. No.: 18/283,268

(22) PCT Filed: Mar. 24, 2022
(86) PCT No.: PCT/US2022/021748

§ 371 Date: Sep. 21, 2023

Related U.S. Application Data
(60) Provisional application No. 63/285,158, filed on Dec. 2, 2021.
(60) Provisional application No. 63/275,046, filed on Nov. 3, 2021.
(60) Provisional application No. 63/209,737, filed on Jun. 11, 2021.
(60) Provisional application No. 63/165,613, filed on Mar. 24, 2021.

(51) Int. Cl. A61K 38/53; A61K 31/4439; A61K 35/00; A61K 35/741; A61K 38/45; A61K 38/51; A61P 13/04; C12N 9/00; C12N 9/10; C12N 9/88; C12N 15/52; C12N 15/70

(52) U.S. Cl. CPC A61K 38/53; A61K 31/4439; A61K 35/741; A61K 38/45; A61K 38/51; A61P 13/04; C12N 9/13; C12N 9/88; C12N 9/93; C12N 15/52; C12N 15/70; C12Y 208/03016; C12Y 401/01008; C12Y 602/01008; A61K 2035/115; C12N 2800/101; C12N 2830/003

(57) ABSTRACT

The present invention provides recombinant bacterial cells comprising at least one heterologous gene encoding at least one oxalate catabolism enzyme. In another aspect, the recombinant bacterial cells further comprise at least one heterologous gene encoding an importer of oxalate. The invention further provides pharmaceutical compositions comprising the recombinant bacteria, and methods for treating disorders in which oxalate is detrimental, such as hyperoxaluria, using the pharmaceutical compositions of the invention.

### RELATED APPLICATIONS

The instant application claims priority to U.S. Provisional Application No. 63/285,158, filed on Dec. 2, 2021; U.S. Provisional Application No. 63/275,046, filed Nov. 3, 2021; U.S. Provisional Application No. 63/209,737, filed Jun. 11, 2021; and U.S. Provisional Application No. 63/165,613, filed Mar. 24, 2021, entire contents of each of which are expressly incorporated by reference herein in their entireties.

### SEQUENCE LISTING

The instant application contains a Sequence Listing which has been submitted electronically in ASCII format and is hereby incorporated by reference in its entirety. Said ASCII copy, created on Mar. 23, 2022, is named 126046-06120_SL.txt and is 421,375 bytes in size.

### BACKGROUND

Oxalate, the ionic form of oxalic acid, arises in the human body from dietary intake or from endogenous synthesis. Oxalate is ubiquitous in plants and plant-derived foods, and as such, is inevitably part of the human diet. Endogenously-synthesized oxalate is primarily derived from glyoxylate in the liver where excess glyoxylate is converted to oxalate by glycolate oxidase or lactate dehydrogenase (Robijn et al., Kidney Int. 80: 1146-58 (2011)). Healthy individuals normally excrete urinary oxalate in ranges between 20-40 mg of oxalate per 24 hours. However, urinary oxalate excretion at concentrations exceeding 40-45 mg per 24 hours is clinically considered hyperoxaluria (Robijn et al. (2011)). Hyperoxaluria is characterized by increased urinary excretion of and elevated systemic levels of oxalate, and urinary oxalate levels are typically about 90-500 mg per 24 hours in primary hyperoxaluria and about 45-130 mg per 24 hours in enteric hyperoxaluria. If left untreated, hyperoxaluria can cause significant morbidity and mortality, including the development of renal stones (kidney stones), nephrocalcinosis (increased calcium in the kidney), crystallopathy and most significantly, End Stage Renal Disease (Tasian et al., J. Am. Soc. Nephrol., 2018; 29(6):1731-1740 and Siener et al., Kidney International, 2013:83:1144-1149).

Hyperoxalurias can generally be divided into two clinical categories: primary and secondary hyperoxalurias. Primary hyperoxalurias are autosomal-recessive inherited diseases resulting from mutations in one of several genes involved in oxalate metabolism (Hoppe et al., Nephr. Dial. Transplant. 26: 3609-15 (2011)). The primary hyperoxalurias are characterized by elevated urinary oxalate excretion which ultimately may result in recurrent urolithiasis, crystallopathy, progressive nephrocalcinosis and early end-stage renal disease. In addition, when chronic renal insufficiency occurs in patients with primary hyperoxalurias, systemic deposition of calcium oxalate (also known as oxalosis) may occur in various organ systems which can lead to bone disease, erythropoietin refractory anemia, skin ulcers, digital gangrene, cardiac arrhythmias, and cardiomyopathy (Hoppe et al. (2011)).

Primary hyperoxaluria type I (PHI) is the most common and severe form of hyperoxaluria, and is caused by a defect in the vitamin B6-dependent hepatic peroxisomal enzyme, alanine glyoxalate aminotransferase (AGT, encoded by the AGXT gene), which catalyzes the transamination of glyoxalate to glycine (Purdue et al., J. Cell Biol. 111: 2341-51 (1990); Hoppe et al., Kidney Int. 75: 1264-71 (2009)). AGT deficiency allows glyoxylate to be reduced to glycolate which is then oxidized to produce oxalate. Over 140 mutations of the human AGXT gene have been identified (Williams et al., Hum. Mut. 30: 910-7 (2009)). Primary hyperoxaluria type II (PHII) is caused by mutations of the enzyme glyoxylate/hydroxypyruvate reductase (GRHPR), an enzyme having glyoxylate reductase (GR), hydroxypyruvate reductase (HPR), and D-glycerate dehydrogenase (DGDH) activities (see, e.g., Cramer et al., Hum. Mol. Gen. 8:2063-9 (1999)). More than a dozen mutation of the human GRHPR gene have been identified (Cregeen et al., Hum. Mut. 22: 497 (2003)). Both PHI and PHII result in severe hyperoxaluria (Robijn et al. (2011)). Primary hyperoxaluria type III (PHIII) is caused by a mutation in the HOGA1 gene, which encodes a 4-hydroxy 2-oxoglutarate aldolase, a mitochondrial enzyme that breaks down 4-hydroxy 2-oxoglutarate into pyruvate and glyoxalate (Pitt et al., JIMD Reports 15: 1-6 (2015)). 15 mutations in the human HOGA1 gene have been identified (Bhasin et al., World J. Nephrol. 4: 235-44 (2015)).

Secondary hyperoxaluria typically results from conditions underlying increased absorption of oxalate, including increased dietary intake of oxalate, increased intestinal absorption of oxalate, excessive intake of oxalate precursors, gut microflora imbalances, and genetic variations of intestinal oxalate transporters (Bhasin et al., 2015; Robijn et al. (2011)). Increased oxalate absorption with consequent hyperoxaluria, often referred to as enteric hyperoxaluria, is observed in patients with a variety of intestinal disorders, including the syndrome of bacterial overgrowth, Crohn's disease, inflammatory bowel disease, as well as other malabsorptive states, such as, after jejunoileal bypass for obesity, after gastric ulcer surgery, and chronic mesenteric ischemia (Pardi et al., Am. J. Gastroenterol. 93: 500-14 (1998); Hylander et al., Scand. J. Gastroent. 15: 349-52 (1980); Canos et al., Can. Med. Assoc. J. 124: 729-33 (1981); Drenick et al., Ann. Intern. Med. 89: 594-9 (1978)). In addition, hyperoxaluria may also occur following renal transplantation (Robijn et al. (2011)). Patients with secondary hyperoxalurias and enteric hyperoxalurias are predisposed to developing calcium oxalate stones, which may lead to significant renal damage and ultimately result in End Stage Renal Disease.

Currently available treatments for hyperoxalurias are inadequate. Strategies for the treatment of primary hyperoxalurias include reducing urinary oxalate with pyridoxine, which is only effective in less than half of patients with PHI, and ineffective in patients with PHII and PHIII (Hoppe et al. (2011)). Further, treatments with citrate, orthophosphate, and magnesium to increase the urinary solubility of calcium oxalate, and thus preserve renal function, are not well characterized (Hoppe et al. (2011)). Other strategies for the treatment of secondary and enteric hyperoxalurias, which are quite arduous and often ineffective, include reducing the dietary intake of oxalate, oral calcium supplementation, and the use of bile acid sequestrants (Parivar et al, J. Urol. 155: 432-40 (1996); Hylander et al. (1980); McLeod and Churchhill, J. Urol. 148: 974-8 (1992)). Generally, dietary restrictions are not entirely effective because patients cannot readily identify the food products to avoid (Parivar et al. (1996)). Therefore, there is significant unmet need for effective, reliable, and/or long-term treatment of hyperoxalurias.

### SUMMARY

The present disclosure provides engineered bacterial cells, pharmaceutical compositions thereof, and methods of modulating and treating disorders in which oxalate is detrimental. Specifically, the engineered bacteria disclosed herein have been constructed to comprise genetic circuits composed of, for example, one or more oxalate catabolism genes to treat the disease, as well as other optional circuitry designed to ensure the safety and non-colonization of a subject that is administered the engineered bacteria, such as, for example, auxotrophies. These engineered bacteria are safe and well tolerated and augment the innate activities of the subject's microbiome to achieve a therapeutic effect.

In one embodiment, disclosed herein is a method for reducing the levels of oxalate in a subject, the method comprising administering to the subject a pharmaceutical composition comprising a recombinant bacterium comprising one or more gene sequences encoding one or more oxalate catabolism enzymes operably linked to a directly or indirectly first promoter that is not associated with the oxalate catabolism enzyme gene in nature, thereby reducing the levels of oxalate in the subject. In one embodiment, the one or more gene sequences is operably linked directly to the first promoter. In another embodiment, the one or more gene sequences is operably linked indirectly to the first promoter. In one embodiment, the first promoter is an inducible promoter. In another embodiment, the first promoter is a constitutive promoter.

In one embodiment, the recombinant bacterium has an oxalate consumption activity of 1 μmol/1×10⁹ cell. In one embodiment, the recombinant bacterium has an oxalate consumption activity of about 50-600 mg/day, about 100-550 mg/day, about 100-500 mg/day, about 100-400 mg/day, about 100-300 mg/day. In one embodiment, the recombinant bacterium has an oxalate consumption activity of about 150-300 mg/day. In one embodiment, the recombinant bacterium has an oxalate consumption activity of about 50 mg/day, about 100 mg/day, about 150 mg/day, about 200 mg/day, about 210 mg/day, about 211 mg/day, about 225 mg/day, about 250 mg/day, about 275 mg/day, about 300 mg/day, about 325 mg/day, about 350 mg/day, about 350 mg/day, about 375 mg/day, about 400 mg/day, about 425 mg/day, about 450 mg/day, about 475 mg/day, about 500 mg/day, about 525 mg/day, about 550 mg/day, about 575 mg/day, or about 600 mg/day. In one embodiment, the recombinant bacterium has an oxalate consumption activity of about 50 mg/day, about 100 mg/day, about 150 mg/day, about 200 mg/day, about 211 mg/day, about 225 mg/day, about 250 mg/day, about 275 mg/day, about 300 mg/day, about 325 mg/day, about 350 mg/day, about 350 mg/day, about 375 mg/day, about 400 mg/day, about 425 mg/day, about 450 mg/day, about 475 mg/day, about 500 mg/day, about 525 mg/day, about 550 mg/day, about 575 mg/day, or about 600 mg/day under anaerobic conditions. In one embodiment, the recombinant bacterium has an oxalate consumption activity of about 50 mg/day, about 100 mg/day, about 150 mg/day, about 200 mg/day, about 211 mg/day, about 225 mg/day, about 250 mg/day, about 275 mg/day, about 300 mg/day, about 325 mg/day, about 350 mg/day, about 350 mg/day, about 375 mg/day, about 400 mg/day, about 425 mg/day, about 450 mg/day, about 475 mg/day, about 500 mg/day, about 525 mg/day, about 550 mg/day, about 575 mg/day, or about 600 mg/day under anaerobic conditions when administered to the subject three times per day. In one embodiment, the anaerobic conditions are conditions in the intestine and/or colon of the subject.

In one embodiment, the recombinant bacterium has an oxalate consumption activity of about 0.2 μmole/hr, about 0.5 μmole/hr, about 0.8 μmole/hr, about 1.0 μmole/hr, about 1.2 μmole/hr, about 1.5 μmole/hr, or about 1.6 μmole/hr under anaerobic conditions. In one embodiment, the recombinant bacterium has an oxalate consumption activity of at least 0.2 μmole/hr, at least 0.5 μmole/hr, at least 0.8 μmole/hr, at least 1.0 μmole/hr, at least 1.2 μmole/hr, at least 1.5 μmole/hr, or at least 1.6 mole/hr under anaerobic conditions. In one embodiment, the recombinant bacterium has an oxalate consumption activity of about 0.2 μmole/hr to about 1.6 μmole/hr, about 0.5 μmole/hr to about 1.5 mole/hr, or about 1.0 μmole/hr to about 1.5 μmole/hr under anaerobic conditions. In one embodiment, the recombinant bacterium has an oxalate consumption activity of about 0.5 μmole/hr to about 1.5 μmole/hr under anaerobic conditions. In one embodiment, the anaerobic conditions are conditions in the intestine and/or colon of the subject.

In one embodiment, the method reduces acute levels of oxalate in the subject by about two fold. In one embodiment, the method reduces acute levels of oxalate in the subject by about three fold. In one embodiment, the method reduces chronic levels of oxalate in the subject by about two fold. In one embodiment, the method reduces chronic levels of oxalate in the subject by about three fold.

In one embodiment, the method reduces acute levels of oxalate in the subject to about 25 mg/day, about 30 mg/day, about 40 mg/day, about 50 mg/day, about 60 mg/day, about 70 mg/day, about 80 mg/day, about 90 mg/day, or about 100 mg/day. In one embodiment, the method reduces chronic levels of oxalate in the subject to about 25 mg/day, about 30 mg/day, about 40 mg/day, about 50 mg/day, about 60 mg/day, about 70 mg/day, about 80 mg/day, about 90 mg/day, or about 100 mg/day.

In one embodiment, UOx in the subject is reduced by at least about 14%, at least about 15%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 45%, or at least about 50% in the subject after administration as compared to a control level of UOx. In one embodiment, UOx in the subject is reduced by at least about 14% to about 50%, at least about 14% to about 45%, at least about 15% to about 50%, at least about 15% to about 45%, at least about 15% to about 40%, at least about 20% to about 50%, at least about 25% to about 50%, at least about 30% to about 50%, at least about 20% to about 40%, at least about 20% to about 45%, at least about 25% to about 45, or at least about 25% to about 40% in the subject after administration as compared to a control level of UOx. In one embodiment, the control level of UOx is a level of UOx in the subject prior to administration. In another embodiment, the control level of UOx is a level of UOx in a subject, or in a population of subjects, having an oxalate disease or disorder who did not receive treatment, wherein the disease or disorder is hyperoxaluria, primary hyperoxaluria, dietary hyperoxaluria, enteric hyperoxaluri, short bowel syndrome, chronic pancreatitis, inflammatory bowel disease (IBD), cystic fibrosis, kidney disease, and/or Roux-en-Y gastric bypass. In one embodiment, the disease or disorder is short bowel syndrome or Roux-en-Y gastric bypass.

In one embodiment, UOx:creatinine ratio in the subject is reduced by at least about 14%, at least about 15%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 45%, or at least about 50% in the subject after administration as compared to a control UOx:creatinine ratio. In one embodiment, UOx:creatinine ratio in the subject is reduced by at least about 14% to about 50%, at least about 14% to about 45%, at least about 15% to about 50%, at least about 15% to about 45%, at least about 15% to about 40%, at least about 20% to about 50%, at least about 25% to about 50%, at least about 30% to about 50%, at least about 20% to about 40%, at least about 20% to about 45%, at least about 25% to about 45, or at least about 25% to about 40% in the subject after administration as compared to a control UOx:creatinine ratio. In one embodiment, the control UOx:creatinine ratio is a level of UOx in the subject prior to administration. In another embodiment, the control UOx:creatinine ratio is a UOx:creatinine ratio in a subject, or in a population of subjects, having an oxalate disease or disorder who did not receive treatment, wherein the disease or disorder is hyperoxaluria, primary hyperoxaluria, dietary hyperoxaluria, enteric hyperoxaluri, short bowel syndrome, chronic pancreatitis, inflammatory bowel disease (IBD), cystic fibrosis, kidney disease, and/or Roux-en-Y gastric bypass. In one embodiment, the disease or disorder is short bowel syndrome or Roux-en-Y gastric bypass.

In one embodiment, the method reduces acute levels of oxalate in the subject by at least about 40% by day 5 after administration. In one embodiment, the method reduces acute levels of oxalate in the subject by at least about 50% by day 5 after administration. In one embodiment, the method reduces acute levels of oxalate in the subject by at least about 60% by day 5 after administration. In one embodiment, the method reduces acute levels of oxalate in the subject by at least about 70% by day 5 after administration. In one embodiment, the method reduces acute levels of oxalate in the subject by at least about 80% by day 5 after administration.

In one embodiment, the method reduces acute levels of oxalate in the subject by at least about 10% by about 24 hours after administration. In one embodiment, the method reduces acute levels of oxalate in the subject by at least about 15% by about 24 hours after administration. In one embodiment, the method reduces acute levels of oxalate in the subject by at least about 20% by about 24 hours after administration.

In one embodiment, the level of oxalate, or the acute level of oxalate, or the chronic level of oxalate, is a level of urinary oxalate (UOx). In one embodiment, the level of UOx in the subject is less than 44 mg/24 h after administration. In one embodiment, the mean 24-hour urinary oxalate level in the subject after administration is less than 44 mg, less than 43 mg, less than 42 mg, less than 41 mg, less than 40 mg, less than 39 mg, less than 38 mg, about 45 mg to about 35 mg, about 44 mg to about 36 mg, about 43 mg to about 37 mg, about 42 mg to about 38 mg, about 41 mg to about 39 mg, or about 40 mg.

In one embodiment, the recombinant bacterium is of the genus Escherichia. In one embodiment, the recombinant bacterium is of the species Escherichia coli strain Nissle.

In one embodiment, the pharmaceutical composition is administered orally. In one embodiment, the subject is fed a meal within one hour of administering the pharmaceutical composition. In one embodiment, the subject is fed a meal concurrently with administering the pharmaceutical composition. In one embodiment, the subject is a human subject.

In one embodiment, disclosed herein is a recombinant bacterium comprising one or more gene sequences encoding one or more oxalate catabolism enzymes operably linked directly or indirectly to a first promoter that is not associated with the oxalate catabolism enzyme gene in nature. In one embodiment, the one or more gene sequences is operably linked directly to the first promoter. In another embodiment, the one or more gene sequences is operably linked indirectly to the first promoter. In one embodiment, the first promoter is an inducible promoter. In another embodiment, the first promoter is a constitutive promoter.

In one embodiment, the recombinant bacterium has an oxalate consumption activity of 1 μmol/1×10⁹ cell. In one embodiment, the recombinant bacterium has an oxalate consumption activity of about 150-300 mg/day under anaerobic conditions. In one embodiment, the recombinant bacterium has an oxalate consumption activity of about 200 mg/day under anaerobic conditions. In one embodiment, the recombinant bacterium has an oxalate consumption activity of about 200 mg/day under anaerobic conditions when administered to the subject three times per day. In one embodiment, the anaerobic conditions are conditions in the intestine and/or colon of the subject.

In one embodiment, the one or more gene sequences comprise a scaaE3 gene, an frc gene, and an oxdC gene. In one embodiment, the scaaE3 gene comprises a sequence having at least 90%, 91%, 92%, 93%, 94%, 95%, 96%, 97%, 98%, or 99% identity to, comprises, or consists of SEQ ID NO: 3. In one embodiment, the frc gene comprises a sequence having at least 90%, 91%, 92%, 93%, 94%, 95%, 96%, 97%, 98%, or 99% identity to, comprises, or consists of SEQ ID NO: 1. In one embodiment, the scaaE3 gene comprises SEQ ID NO: 3. In one embodiment, the oxdC gene comprises a sequence having at least 90%, 91%, 92%, 93%, 94%, 95%, 96%, 97%, 98%, or 99% identity to, comprises, or consists of SEQ ID NO: 2. In one embodiment, the frc gene comprises SEQ ID NO: 2.

In one embodiment, the recombinant bacterium further comprises a gene encoding an oxalate importer. In one embodiment, the gene encoding the oxalate importer is an oxlT gene. In one embodiment, the oxlT gene comprises a sequence having at least 90%, 91%, 92%, 93%, 94%, 95%, 96%, 97%, 98%, or 99% identity to, comprises, or consists of SEQ ID NO: 11. In one embodiment, the oxlT gene comprises SEQ ID NO: 11.

In one embodiment, the recombinant bacterium further comprises an auxotrophy. In one embodiment, the auxotrophy is a thyA auxotrophy. In one embodiment, thyA has at least 90%, 91%, 92%, 93%, 94%, 95%, 96%, 97%, 98%, or 99% identity to, comprises, or consists of SEQ ID NO: 62.

In one embodiment, the recombinant bacterium further comprises a deletion in an endogenous phage. In one embodiment, the endogenous phage comprises a sequence having at least 90%, 91%, 92%, 93%, 94%, 95%, 96%, 97%, 98%, or 99% identity to, comprises, or consists of SEQ ID NO: 63. In one embodiment, the endogenous phase comprises a sequence of SEQ ID NO: 63.

In one embodiment, the recombinant bacterium does not comprise a gene encoding for antibiotic resistance. In one embodiment, the first promoter is an inducible promoter. In one embodiment, the inducible promoter is induced by low oxygen or anaerobic conditions, temperature, or the hypoxic environment of a tumor. In one embodiment, the inducible promoter is an FNR promoter. In one embodiment, the FNR promoter is a promoter selected from the group consisting of any one of SEQ ID NOs: 13-29.

In one embodiment, the recombinant bacterium comprises an oxlT gene under the control of an inducible promoter, optionally an FNR promoter; an scaaE3 gene, an oxcd gene, and an frc gene under the control of an inducible promoter, optionally an FNR promoter, a thyA deletion (or auxotrophy) and a deletion of endogenous phage 3. In one embodiment, the recombinant bacterium comprises HA910::FNR_oxlT, HA12::FNR_scaaE3-oxcd-frc, ΔthyA, Δphage 3.

In some embodiments, the recombinant bacterial cell further comprises a modified endogenous colibactin island.

In some embodiments, the modified endogenous colibactin island comprises one or more modified clb sequences selected from the group consisting of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), clbR (SEQ ID NO: 1082), and clbS (SEQ ID NO: 1803).

In some embodiments, the modified endogenous colibactin island comprises a deletion of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), and clbR (SEQ ID NO: 1082).

In one embodiment, the recombinant bacterium is SYN5752, SYN7169, or SYNB8802. In one embodiment, the recombinant bacterium is SYNB8802.

In one embodiment, the subject has hyperoxaluria. In one embodiment, the hyperoxaluria is primary hyperoxaluria, dietary hyperoxaluria, or enteric hyperoxaluria. In one embodiment, the subject has short bowel syndrome, chronic pancreatitis, inflammatory bowel disease (IBD), cystic fibrosis, kidney disease, and/or Roux-en-Y gastric bypass.

In one embodiment, the subject has urinary oxalate (Uox) levels of at least 70 mg/day prior to the administering. In one embodiment, the subject exhibits a decrease in Uox levels of at least 10%, 15%, 20%, 25%, 30%, 35%, 40%, 45%, 50%, 55%, 60%, 65%, 70%, 75%, or 80% after the administering. In one embodiment, the subject has eGFR<30 mL/min/1.73 m², requires hemodialysis, or has systemic oxalosis prior to the administering.

In one embodiment, the recombinant bacteria are administered at a dose of about 1×10¹¹ live recombinant bacteria, about 2×10¹¹ live recombinant bacteria, about 3×10¹¹ live recombinant bacteria, about 4×10¹¹ live recombinant bacteria, about 4.5×10¹¹ live recombinant bacteria, about 5×10¹¹ live recombinant bacteria, about 6×10¹¹ live recombinant bacteria, about 1×10¹² live recombinant bacteria, or about 2×10¹² live recombinant bacteria. In one embodiment, the recombinant bacteria are administered at a dose of about 6×10¹¹ live recombinant bacteria. In one embodiment, the recombinant bacteria are administered at a dose of about 3×10¹¹ live recombinant bacteria. In one embodiment, the recombinant bacteria are administered at a dose of about 1×10¹¹ live recombinant bacteria. In one embodiment, the administering is about 4.5×10¹¹ live recombinant bacteria. In one embodiment, the administering is about 5×10¹¹ live recombinant bacteria. In one embodiment, the recombinant bacteria are administered at a dose of about 1×10¹² live recombinant bacteria. In one embodiment, the recombinant bacteria are administered at a dose of about 2×10¹² live recombinant bacteria. In one embodiment, the administering are about 5×10¹¹ live recombinant bacteria with meals three times per day. In one embodiment, the recombinant bacteria are administered at a dose of about 1×10¹¹ live recombinant bacteria to about 2×10¹² live recombinant bacteria. In one embodiment, the recombinant bacteria are administered at a dose of about 1×10¹² live recombinant bacteria to about 2×10¹² live recombinant bacteria. In one embodiment, the recombinant bacteria are administered at a dose of about 5×10¹¹ live recombinant bacteria to about 2×10¹² live recombinant bacteria.

In one embodiment, the administering is once per day. In another embodiment, the administering is twice per day. In another embodiment, the administering is oral, with meals, once per day. In another embodiment, the administering is oral, with meals, twice per day. In another embodiment, the administering is oral, with meals, three times per day.

In another embodiment, a proton pump inhibitor (PPI) is administered to the subject. In another embodiment, the PPI is esomeprazole. In another embodiment, esomeprazole is administered at 40 mg once daily. In another embodiment, the administering of the PPI is once a day. In another embodiment, galactose is administered to the subject in combination with, e.g., at the same time as, or in the same composition or formulation as, the recombinant bacteria described herein. In another embodiment, the administering of galactose is once a day, twice per day, three times per day, or with meals. In a specific embodiment, the galactose is administered to the subject in the same composition or formulation as the recombinant bacteria described herein. In one embodiment, the galactose is D-galactose. In another embodiment, a proton pump inhibitor (PPI) and galactose, e.g., D-galactose, are administered to the subject in combination with the recombinant bacteria described herein. In another embodiment, the PPI is esomeprazole. In another embodiment, esomeprazole is administered at 40 mg once daily. In another embodiment, the administering of the PPI and galactose is once a day, twice per day, three times per day, or with meals.

In another embodiment, galactose is administered at about 0.1 g to about 3 g, about 0.1 g to about 2.5 g, about 0.1 g to about 2.0 g, about 0.1 g to about 1.5 g, about 0.1 g to about 1.0 g, about 0.1 g to about 0.5 g, about 0.5 g to about 3 g, about 0.5 g to about 2.5 g, about 0.5 g to about 2.0 g, about 0.5 g to about 1.5 g, about 0.5 g to about 1.0 g, about 1.0 g to about 3 g, about 1.0 g to about 2.5 g, about 1.0 g to about 2.0 g, about 1.0 g to about 1.5 g, about 1.5 g to about 3 g, about 1.5 g to about 2.5 g, about 1.5 g to about 2.0 g, about 2.0 g to about 3 g, about 2.0 g to about 2.5 g, or about 2.5 g to about 3 g. In some embodiments, galactose is administered at about 1.0 g. In some embodiments, galactose is administered at about 0.5 g. In some embodiments, galactose is administered at about 2.0 g. In some embodiments, the disclosure provides a bacterial cell that has been genetically engineered to comprise one or more genes, gene cassettes, and/or synthetic circuits encoding one or more oxalate catabolism enzyme(s) or oxalate catabolism pathway, and is capable of metabolizing oxalate and/or other metabolites, such as oxalyl-CoA. Thus, the genetically engineered bacterial cells and pharmaceutical compositions comprising the bacterial cells may be used to treat and/or prevent diseases associated with disorders in which oxalate is detrimental, such as primary hyperoxalurias and secondary hyperoxalurias.

In some embodiments, the disclosure provides a bacterial cell that has been engineered to comprise gene sequence(s) encoding one or more oxalate catabolism enzyme(s). In some embodiments, the disclosure provides a bacterial cell has been engineered to comprise gene sequence(s) encoding one or more oxalate catabolism enzyme(s) and is capable of reducing the level of oxalate and/or other metabolites, for example, oxalyl-CoA. In some embodiments, the bacterial cell has been engineered to comprise gene sequence(s) encoding one or more transporter(s) (importer(s)) of oxalate. In some embodiments, the bacterial cell has been engineered to comprise gene sequence(s) encoding one or more exporter(s) of formate. In some embodiments, the engineered bacteria comprise gene sequence(s) encoding one or more polypeptide(s) which mediate both the transport (import) of oxalate and the export of formate (e.g., oxalate:formate antiporter(s)). In some embodiments, the engineered bacteria comprise gene sequence(s) encoding one or more of the following: (i) one or more transporter(s) of oxalate; (ii) one or more exporter(s) of formate; (iii) one or more polypeptide(s) which mediate both the transport (import) of oxalate and the export of formate (e.g., oxalate:formate antiporter(s)); and (iv) any combination thereof. In some embodiments, the bacterial cell has been engineered to comprise gene sequence(s) encoding one or more oxalate catabolism enzyme(s) and one or more transporter(s) (importer(s)) of oxalate. In some embodiments, the bacterial cell of the disclosure has been genetically engineered to comprise gene sequence(s) encoding one or more oxalate catabolism enzyme(s) and one or more exporter(s) of formate. In some embodiments, genetically engineered bacteria comprise gene sequence(s) encoding one or more oxalate catabolism enzyme(s) and one or more polypeptide(s), which mediate both the transport (import) of oxalate and the export of formate (e.g., oxalate:formate antiporter(s)). In some embodiments, the bacterial cell has been engineered to comprise gene sequence(s) encoding one or more oxalate catabolism enzyme(s) and gene sequence(s) encoding one or more of the following: (i) one or more transporter(s) of oxalate; (ii) one or more exporter(s) of formate; (iii) one or more polypeptide(s) which mediate both the transport (import) of oxalate and the export of formate (e.g., oxalate:formate antiporter(s)); and (iv) any combination thereof.

In some embodiments, the gene sequence(s) encoding one or more oxalate catabolism enzyme(s) is operably linked to an inducible promoter. In some embodiments, the gene sequence(s) encoding one or more oxalate transporter(s) (importer(s)) is operably linked to an inducible promoter. In some embodiments, the gene sequence(s) encoding one or more exporter(s) of formate is operably linked to an inducible promoter. In some embodiments, the gene sequence(s) encoding one or more polypeptide(s) which mediate both the transport (import) of oxalate and the export of formate (e.g., oxalate:formate antiporter(s)) is operably linked to an inducible promoter. In some embodiments, the gene sequence(s) encoding one or more oxalate catabolism enzyme(s) and the gene sequence(s) encoding one or more oxalate transporter(s) (importer(s)) are operably linked to an inducible promoter. In some embodiments, the gene sequence(s) encoding one or more oxalate catabolism enzyme(s) and the gene sequence(s) encoding one or more exporter(s) of formate are operably linked to an inducible promoter. In some embodiments, the gene sequence(s) encoding one or more oxalate catabolism enzyme(s) and the gene sequence(s) encoding one or more polypeptide(s) which mediate both the transport (import) of oxalate and the export of formate (e.g., oxalate:formate antiporter(s)) are operably linked to an inducible promoter. In some embodiments, any one or more of the following gene sequences, if present in the bacterial cell, are operably linked to an inducible promoter: (i) gene sequence(s) encoding one or more oxalate catabolism enzyme(s); (ii) gene sequence(s) encoding one or more oxalate transporter(s); (iii) gene sequence(s) encoding one or more exporter(s) of formate; and (iv) gene sequence(s) encoding one or more polypeptide(s) which mediate both the transport (import) of oxalate and the export of formate (e.g., oxalate:formate antiporter(s)).

In some embodiments, the disclosure provides a bacterial cell which has been engineered to comprise gene sequence(s) encoding one or more oxalate catabolism enzyme(s) operably linked to an inducible promoter that is induced under low oxygen and/or anaerobic conditions, e.g., such as those conditions found in the mammalian gut. In some embodiments, the disclosure provides a bacterial cell which has been engineered to comprise gene sequence(s) encoding one or more oxalate transporter(s) (importer(s)) operably linked to an inducible promoter that is induced under low oxygen and/or anaerobic conditions, e.g., such as those conditions found in the mammalian gut. In some embodiments, the disclosure provides a bacterial cell which has been engineered to comprise gene sequence(s) encoding one or more exporter(s) of formate operably linked to an inducible promoter that is induced under low oxygen and/or anaerobic conditions, e.g., such as those conditions found in the mammalian gut. In some embodiments, the disclosure provides a bacterial cell which has been engineered to comprise gene sequence(s) encoding one or more polypeptide(s) which mediate both the transport (import) of oxalate and the export of formate (e.g., oxalate:formate antiporter(s)) operably linked to an inducible promoter that is induced under low oxygen and/or anaerobic conditions, e.g., such as those conditions found in the mammalian gut. In some embodiments, the gene sequence(s) encoding one or more oxalate catabolism enzyme(s) and the gene sequence(s) encoding one or more oxalate transporter(s) (importer(s)) are operably linked to an inducible promoter that is induced under low oxygen and/or anaerobic conditions. In some embodiments, the gene sequence(s) encoding one or more oxalate catabolism enzyme(s) and the gene sequence(s) encoding one or more exporter(s) of formate are operably linked to an inducible promoter that is induced under low oxygen and/or anaerobic conditions. In some embodiments, the gene sequence(s) encoding one or more oxalate catabolism enzyme(s) and the gene sequence(s) encoding one or more polypeptide(s) which mediate both the transport (import) of oxalate and the export of formate (e.g., oxalate:formate antiporter(s)) are operably linked to an inducible promoter that is induced under low oxygen and/or anaerobic conditions. In some embodiments, any one or more of the following gene sequences, if present in the bacterial cell, are operably linked to an inducible promoter that is induced under low oxygen and/or anaerobic conditions: (i) gene sequence(s) encoding one or more oxalate catabolism enzyme(s); (ii) gene sequence(s) encoding one or more oxalate transporter(s); (iii) gene sequence(s) encoding one or more exporter(s) of formate; and (iv) gene sequence(s) encoding one or more polypeptide(s) which mediate both the transport (import) of oxalate and the export of formate (e.g., oxalate:formate antiporter(s)). In one embodiment, the inducible promoter is a lacI promoter which can be induced with IPTG. In one embodiment, one or more of the above gene sequences are operably linked to an IPTG inducible promoter, e.g., the P_{tac} promoter, having lacI operator. In one embodiment, the lac repressor gene, lacI, is placed upstream of the gene P_{tac}—construct in reverse orientation to allow for divergent transcription.

In some embodiments, the inducible promoter is a IPTG inducible promoter. In some embodiments, the IPTG inducible promoter comprises a sequence having at least 90%, 91%, 92%, 93%, 94%, 95%, 96%, 97%, 98%, or 99% identity to, comprises, or consists of SEQ ID NO: 1107. In some embodiments, the recombinant bacterium further comprises a gene sequence encoding a repressor of the Lac promoter. In some embodiments, the gene sequence encoding a repressor comprises a sequence having at least 90%, 91%, 92%, 93%, 94%, 95%, 96%, 97%, 98%, or 99% identity to, comprises, or consists of SEQ ID NO: 1105. In some embodiments, the repressor comprises a sequence having at least 90%, 91%, 92%, 93%, 94%, 95%, 96%, 97%, 98%, or 99% identity to, comprises, or consists of SEQ ID NO: 1106.

In another embodiment, the inducible promoter is a pBAD promoter which can be induced with arabinose. In one embodiment, one or more of the above gene sequences are operably linked to an temperature inducible promoter, having a operators for cI38 or cI857 repressor binding. In one embodiment, the cI38 or cI857 repressor gene, is placed upstream of the gene operably linked to the temperature sensitive promoter in reverse orientation to allow for divergent transcription.

In some embodiments, the disclosure provides a bacterial cell that has been engineered to comprise gene sequence(s) encoding one or more oxalate catabolism enzyme(s) that is operably linked to an inducible promoter that is induced by environmental signals and/or conditions found in the mammalian gut (e.g., induced by metabolites (e.g., oxalate metabolites) or other biomolecules found in the mammalian gut, and/or induced by inflammatory conditions (e.g., reactive nitrogen species and/or reactive oxygen species)). The environmental signals and/or conditions found in the mammalian gut may be signals and conditions found in a healthy mammalian gut or signals and conditions found in a diseased mammalian gut, such as the gut of a subject having hyperoxaluria or other condition in which the level of oxalate and/or an oxalate metabolite is elevated, and/or the gut of a subject having an inflammatory condition, such as irritable bowel disease, an autoimmune disease, and any other condition that results in inflammation in the gut. In some embodiments, the disclosure provides a bacterial cell which has been engineered to comprise gene sequence(s) encoding one or more oxalate catabolism enzyme(s) operably linked to an inducible promoter that is induced under inflammatory conditions, e.g., such as inflammatory conditions found in a mammalian gut. In some embodiments, the disclosure provides a bacterial cell which has been engineered to comprise gene sequence(s) encoding one or more oxalate transporter(s) (importer(s)) operably linked to an inducible promoter that is induced under inflammatory conditions, e.g., such as inflammatory conditions found in a mammalian gut. In some embodiments, the disclosure provides a bacterial cell which has been engineered to comprise gene sequence(s) encoding one or more exporter(s) of formate operably linked to an inducible promoter that is induced under inflammatory conditions, e.g., such as inflammatory conditions found in a mammalian gut. In some embodiments, the disclosure provides a bacterial cell which has been engineered to comprise gene sequence(s) encoding one or more polypeptide(s) which mediate both the transport (import) of oxalate and the export of formate (e.g., oxalate:formate antiporter(s)) operably linked to an inducible promoter that is induced under inflammatory conditions, e.g., such as inflammatory conditions found in a mammalian gut. In some embodiments, the gene sequence(s) encoding one or more oxalate catabolism enzyme(s) and the gene sequence(s) encoding one or more oxalate transporter(s) (importer(s)) are operably linked to an inducible promoter that is induced under inflammatory conditions. In some embodiments, the gene sequence(s) encoding one or more oxalate catabolism enzyme(s) and the gene sequence(s) encoding one or more exporter(s) of formate are operably linked to an inducible promoter that is induced under inflammatory conditions. In some embodiments, the gene sequence(s) encoding one or more oxalate catabolism enzyme(s) and the gene sequence(s) encoding one or more polypeptide(s) which mediate both the transport (import) of oxalate and the export of formate (e.g., oxalate:formate antiporter(s)) are operably linked to an inducible promoter that is induced under inflammatory conditions. In some embodiments, any one or more of the following gene sequences, if present in the bacterial cell, are operably linked to an inducible promoter that is induced under inflammatory conditions: (i) gene sequence(s) encoding one or more oxalate catabolism enzyme(s); (ii) gene sequence(s) encoding one or more oxalate transporter(s); (iii) gene sequence(s) encoding one or more exporter(s) of formate; and (iv) gene sequence(s) encoding one or more polypeptide(s) which mediate both the transport (import) of oxalate and the export of formate (e.g., oxalate:formate antiporter(s)).

In some embodiments, the disclosure provides a bacterial cell that has been engineered to comprise gene sequence(s) encoding one or more polypeptide(s) capable of reducing the level of oxalate and/or other metabolites, for example, oxalyl-CoA, in low-oxygen environments, e.g., the gut. In some embodiments, the bacterial cell that has been engineered to comprise gene sequence(s) encoding one or more of the following: (i) one or more oxalate catabolism enzyme(s); (ii) one or more oxalate transporter(s); (ii) one or more formate exporter(s); and (iv) one or more oxalate:formate antiporter(s). In some embodiments, the bacterial cell has been genetically engineered to comprise one or more circuits encoding one or more oxalate catabolism enzyme(s) and is capable of processing and reducing levels of oxalate, and/or oxalyl-CoA e.g., in low-oxygen environments, e.g., the gut. Thus, in some embodiments, the genetically engineered bacterial cells and pharmaceutical compositions comprising the bacterial cells of the disclosure may be used to import excess oxalate and/or oxalyl-CoA into the bacterial cell in order to treat and/or prevent conditions associated with disorders in which oxalate is detrimental, such as primary hyperoxalurias and secondary hyperoxalurias. In some embodiments, the genetically engineered bacterial cells and pharmaceutical compositions comprising the bacterial cells of the disclosure may be used to convert excess oxalate and/or oxalyl-CoA into non-toxic molecules in order to treat and/or prevent conditions associated with disorders in which oxalate is detrimental, such as primary hyperoxalurias and secondary hyperoxalurias.

In some embodiments, the one or more gene sequences comprise a scaaE3 gene, an frc gene, and an oxdC gene.

In some embodiments, the scaaE3 gene comprises a sequence having at least 90%, 91%, 92%, 93%, 94%, 95%, 96%, 97%, 98%, or 99% identity to, comprises, or consists of SEQ ID NO: 3.

In some embodiments, the frc gene comprises a sequence having at least 90%, 91%, 92%, 93%, 94%, 95%, 96%, 97%, 98%, or 99% identity to, comprises, or consists of SEQ ID NO: 1.

In some embodiments, the oxdC gene comprises a sequence having at least 90%, 91%, 92%, 93%, 94%, 95%, 96%, 97%, 98%, or 99% identity to, comprises, or consists of SEQ ID NO: 2.

In some embodiments, the recombinant bacterium further comprises a gene encoding an oxalate importer.

In some embodiments, the gene encoding the oxalate importer is an oxlT gene.

In some embodiments, the oxlT gene comprises a sequence having at least 90%, 91%, 92%, 93%, 94%, 95%, 96%, 97%, 98%, or 99% identity to, comprises, or consists of SEQ ID NO: 11.

In some embodiments, the recombinant bacterium further comprises an auxotrophy.

In some embodiments, the auxotrophy is a thyA auxotrophy.

In some embodiments, the recombinant bacterium further comprises a deletion in an endogenous phage.

In some embodiments, the endogenous phage comprises a sequence having at least 90%, 91%, 92%, 93%, 94%, 95%, 96%, 97%, 98%, or 99% identity to, comprises, or consists of SEQ ID NO: 63.

In some embodiments, the recombinant bacterial cell further comprises a modified endogenous colibactin island.

In some embodiments, the modified endogenous colibactin island comprises one or more modified clb sequences selected from the group consisting of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), clbR (SEQ ID NO: 1082), and clbS (SEQ ID NO: 1803).

In some embodiments, the modified endogenous colibactin island comprises a deletion of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), and clbR (SEQ ID NO: 1082).

In some embodiments, the recombinant bacterium does not comprise a gene encoding for antibiotic resistance.

In some embodiments, the first promoter is an inducible promoter, optionally when the inducible promoter is a FNR promoter, optionally wherein the FNR promoter comprises a sequence having at least 90%, 91%, 92%, 93%, 94%, 95%, 96%, 97%, 98%, or 99% identity to, comprises, or consists of any one of SEQ ID NOs: 13-29.

In some embodiments, the inducible promoter is a Pr/Pl promoter. In one embodiment, the Pr/Pl promoter comprises a sequence having at least 90%, 91%, 92%, 93%, 94%, 95%, 96%, 97%, 98%, or 99% identity to, comprises, or consists of any one of SEQ ID NOs: 206, 213, and 219. In some embodiments, the recombinant bacterium further comprises a gene sequence encoding a mutant repressor of the Pr/Pl promoter. In some embodiments, the gene sequence encoding a mutant repressor comprises a sequence having at least 90%, 91%, 92%, 93%, 94%, 95%, 96%, 97%, 98%, or 99% identity to, comprises, or consists of any one of SEQ ID NOs: 210 and 214.

In some embodiments, the inducible promoter is a IPTG inducible promoter. In one embodiment, wherein the IPTG inducible promoter comprises a sequence having at least 90%, 91%, 92%, 93%, 94%, 95%, 96%, 97%, 98%, or 99% identity to, comprises, or consists of SEQ ID NO: 1107. In some embodiments, the recombinant bacterium further comprises a gene sequence encoding a repressor of the Lac promoter. In some embodiments, the gene sequence encoding a repressor comprises a sequence having at least 90%, 91%, 92%, 93%, 94%, 95%, 96%, 97%, 98%, or 99% identity to, comprises, or consists of SEQ ID NO: 1105. In some embodiments, the repressor comprises a sequence having at least 90%, 91%, 92%, 93%, 94%, 95%, 96%, 97%, 98%, or 99% identity to, comprises, or consists of SEQ ID NO: 1106.

The present invention provides recombinant bacterial cells, pharmaceutical compositions thereof, and methods of modulating and treating disorders in which oxalate is detrimental. The genetically engineered bacterial cells and pharmaceutical compositions comprising the bacterial cells of the invention may be used to convert excess oxalate and/or oxalic acid into non-toxic molecules in order to treat and/or prevent conditions associated with disorders in which oxalate is detrimental, such as primary hyperoxalurias and secondary hyperoxalurias. In some embodiments, a bacterial cell has been engineered to comprise at least one heterologous gene encoding at least one oxalate catabolism enzyme and is capable of processing and reducing levels of oxalate, in low-oxygen environments, e.g., the gut. In some embodiments, a bacterial cell has been engineered to comprise at least one heterologous gene encoding an importer of oxalate and is capable of reducing levels of oxalate, in low-oxygen environments, e.g., the gut. In some embodiments, a bacterial cell has been engineered to comprise at least one heterologous gene encoding an exporter of formate and is capable of reducing levels of oxalate, in low-oxygen environments, e.g., the gut. In some embodiments, a bacterial cell has been engineered to comprise at least one heterologous gene encoding an oxalate:formate antiporter and is capable of reducing levels of oxalate, in low-oxygen environments, e.g., the gut. In some embodiments, a bacterial cell has been engineered to comprise at least one heterologous gene encoding at least one oxalate catabolism enzyme and is capable of processing and reducing levels of oxalate, in inflammatory environments, such as may be present in the gut. In some embodiments, a bacterial cell has been engineered to comprise at least one heterologous gene encoding an importer of oxalate and is capable of reducing levels of oxalate, in inflammatory environments, e.g., such as may be present in the gut. In some embodiments, a bacterial cell has been engineered to comprise at least one heterologous gene encoding an exporter of formate and is capable of reducing levels of oxalate, in inflammatory environments, e.g., such as may be present in the gut. In some embodiments, a bacterial cell has been engineered to comprise at least one heterologous gene encoding an oxalate:formate antiporter and is capable of reducing levels of oxalate, in inflammatory environments, e.g., such as may be present in the gut.

In some embodiments, the at least one oxalate catabolism enzyme converts oxalate to formate or formyl CoA. In some embodiments, the at least one oxalate catabolism enzyme is selected from an oxalate-CoA ligase, (e.g., ScAAE3 from S. cerevisiae), an oxalyl-CoA decarboxylase (Oxc, e.g., from O. formigenes), and a formyl-CoA transferase (e.g., Frc, e.g., from O. formigenes). In some embodiments, the at least one heterologous gene encoding at least one oxalate catabolism enzyme is selected from a frc gene and an oxc gene In one embodiment, the at least one heterologous gene encoding an oxalate transporter is an oxlT gene. In some embodiments, the at least one heterologous gene encoding at least one oxalate catabolism enzyme is located on a plasmid in the bacterial cell. In some embodiments, the at least one heterologous gene encoding at least one oxalate catabolism enzyme is located on a chromosome in the bacterial cell. In some embodiments, the at least one heterologous gene encoding an oxalate transporter is located on a plasmid in the bacterial cell. In some embodiments, the at least one heterologous gene encoding the oxalate transporter is located on a chromosome in the bacterial cell. In some embodiments, the at least one heterologous gene encoding a formate exporter is located on a plasmid in the bacterial cell. In some embodiments, the at least one heterologous gene encoding a formate exporter is located on a chromosome in the bacterial cell. In some embodiments, the at least one heterologous gene encoding an oxalate:formate antiporter is located on a plasmid in the bacterial cell. In some embodiments, the at least one heterologous gene encoding an oxalate:formate antiporter is located on a chromosome in the bacterial cell.

In some embodiments, the engineered bacterial cell is a probiotic bacterial cell. In some embodiments, the engineered bacterial cell is a member of a genus selected from the group consisting of Bacteroides, Bifidobacterium, Clostridium, Escherichia, Lactobacillus and Lactococcus. In some embodiments, the engineered bacterial cell is of the genus Escherichia. In some embodiments, the recombinant bacterial cell is of the species Escherichia coli strain Nissle.

In some embodiments, the engineered bacterial cell is an auxotroph in a gene that is complemented when the engineered bacterial cell is present in a mammalian gut. In some embodiments, the mammalian gut is a human gut. In some embodiments, the engineered bacterial cell is an auxotroph in diaminopimelic acid or an enzyme in the thymine biosynthetic pathway. In some embodiments, the engineered bacterial cell further comprises a heterologous gene encoding a substance that is toxic to the bacterial cell that is operably linked to an inducible promoter, wherein the inducible promoter is directly or indirectly induced by an environmental condition not naturally present in the mammalian gut.

In another aspect, the invention provides a pharmaceutical composition comprising a recombinant bacterial cell comprising at least one heterologous gene encoding at least one oxalate catabolism enzyme operably linked to a first inducible promoter and a pharmaceutically acceptable carrier. In another aspect, the invention provides a pharmaceutical composition comprising a recombinant bacterial cell comprising at least one heterologous gene encoding at least one oxalate catabolism enzyme operably linked to a first inducible promoter, at least one heterologous gene encoding an oxalate transporter operably linked to a second inducible promoter, which may be the same or different promoter from the first inducible promoter, and a pharmaceutically acceptable carrier. In another aspect, the invention provides a pharmaceutical composition comprising a recombinant bacterial cell comprising at least one heterologous gene encoding at least one oxalate catabolism enzyme operably linked to a first inducible promoter, at least one heterologous gene encoding a formate exporter operably linked to a second inducible promoter, which may be the same or different promoter from the first inducible promoter, and a pharmaceutically acceptable carrier. In another aspect, the invention provides a pharmaceutical composition comprising a recombinant bacterial cell comprising at least one heterologous gene encoding at least one oxalate catabolism enzyme operably linked to a first inducible promoter, at least one heterologous gene encoding an oxalate:formate antiporter operably linked to a second inducible promoter, which may be the same or different promoter from the first inducible promoter, and a pharmaceutically acceptable carrier. In any of these embodiments, the first promoter and the second promoter may be separate copies of the same promoter. In some embodiments, the first inducible promoter, the second inducible promoter, or the first inducible promoter and the second inducible promoter, are each directly induced by environmental conditions. In some embodiments, the first inducible promoter, the second inducible promoter, or the first inducible promoter and the second inducible promoter, are each indirectly induced by environmental conditions. In some embodiments, the first inducible promoter, the second inducible promoter, or the first inducible promoter and the second inducible promoter, are each directly or indirectly induced by environmental conditions found in the gut of a mammal. In some embodiments, the first inducible promoter, the second inducible promoter, or the first inducible promoter and the second inducible promoter, are each directly or indirectly induced by low-oxygen or anaerobic conditions. In some embodiments, the first inducible promoter, the second inducible promoter, or the first inducible promoter and the second inducible promoter, are each directly or indirectly induced by inflammatory conditions. In some embodiments, the first inducible promoter, the second inducible promoter, or the first inducible promoter and the second inducible promoter, are each an FNR responsive promoter. In some embodiments, the first inducible promoter, the second inducible promoter, or the first inducible promoter and the second inducible promoter, are each an RNS responsive promoter. In some embodiments, the first inducible promoter, the second inducible promoter, or the first inducible promoter and the second inducible promoter, are each an ROS responsive promoter. In another aspect, the invention provides a method for treating a disease or disorder in which oxalate is detrimental in a subject, the method comprising administering a an engineered bacterial cell or a pharmaceutical composition comprising an engineered bacterial cell to the subject, wherein the engineered bacterial cell comprises gene sequence encoding one or more oxalate catabolism enzyme(s). In another aspect, the invention provides a method for treating a disease or disorder in which oxalate is detrimental in a subject, the method comprising administering an engineered bacterial cell or a pharmaceutical composition comprising an engineered bacterial cell to the subject, wherein the engineered bacterial cell comprises gene sequence encoding one or more oxalate transporter(s). In another aspect, the invention provides a method for treating a disease or disorder in which oxalate is detrimental in a subject, the method comprising administering an engineered bacterial cell or a pharmaceutical composition comprising an engineered bacterial cell to the subject, wherein the engineered bacterial cell comprises gene sequence encoding one or more formate exporter(s). In another aspect, the invention provides a method for treating a disease or disorder in which oxalate is detrimental in a subject, the method comprising administering an engineered bacterial cell or a pharmaceutical composition comprising an engineered bacterial cell to the subject, wherein the engineered bacterial cell comprises gene sequence encoding one or more oxalate:formate antiporter(s). In another aspect, the invention provides a method for treating a disease or disorder in which oxalate is detrimental in a subject, the method comprising administering an engineered bacterial cell or a pharmaceutical composition comprising an engineered bacterial cell to the subject, wherein the engineered bacterial cell comprises gene sequence encoding one or more of the following: (i) oxalate catabolism enzyme(s); (ii) one or more oxalate transporter(s); (iii) one or more formate exporter(s); and (iv) one or more oxalate:formate antiporter(s).

In another aspect, the invention provides a method for treating a disease or disorder in which oxalate is detrimental in a subject, the method comprising administering an engineered bacterial cell or a pharmaceutical composition comprising an engineered bacterial cell to the subject, wherein the engineered bacterial cell expresses at least one heterologous gene encoding at least one oxalate catabolism enzyme in response to an exogenous environmental condition in the subject, thereby treating the disease or disorder in which oxalate is detrimental in the subject. In some embodiments, the engineered bacterial cell further expresses one or more of the following: (i) at least one heterologous gene encoding an importer of oxalate; (ii) at least one heterologous gene encoding an exporter of formate; and/or (iii) at least one heterologous gene encoding an oxalate:formate antiporter. In one aspect, the invention provides a method for treating a disorder in which oxalate is detrimental in a subject, the method comprising administering an engineered bacterial cell or a pharmaceutical composition of the invention to the subject, thereby treating the disorder in which oxalate is detrimental in the subject. In another aspect, the invention provides a method for decreasing a level of oxalate in plasma of a subject, the method comprising administering an engineered bacterial cell or a pharmaceutical composition of the invention to the subject, thereby decreasing the level of oxalate in the plasma of the subject. In another aspect, the invention provides a method for decreasing a level of oxalate in urine of a subject, the method comprising administering an engineered bacterial cell or a pharmaceutical composition of the invention to the subject, thereby decreasing the level of oxalate in the urine of the subject. In one embodiment, the level of oxalate is decreased in plasma of the subject after administering the engineered bacterial cell or pharmaceutical composition to the subject. In another embodiment, the level of oxalate is reduced in urine of the subject after administering the engineered bacterial cell or pharmaceutical composition to the subject. In one embodiment, the engineered bacterial cell or pharmaceutical composition is administered orally. In another embodiment, the method further comprises isolating a plasma sample from the subject or a urine sample from the subject after administering the engineered bacterial cell or pharmaceutical composition to the subject, and determining the level of oxalate in the plasma sample from the subject or the urine sample from the subject. In another embodiment, the method further comprises comparing the level of oxalate in the plasma sample from the subject or the urine sample from the subject to a control level of oxalate. In one embodiment, the control level of oxalate is the level of oxalate in the plasma of the subject or in the urine of the subject before administration of the engineered bacterial cell or pharmaceutical composition.

In one embodiment, the disorder in which oxalate is detrimental is a hyperoxaluria. In one embodiment, the hyperoxaluria is primary hyperoxaluria type I. In another embodiment, the hyperoxaluria is primary hyperoxaluria type II. In another embodiment, the hyperoxaluria is primary hyperoxaluria type III. In one embodiment, the hyperoxaluria is enteric hyperoxaluria. In another embodiment, the hyperoxaluria is dietary hyperoxaluria. In another embodiment, the hyperoxaluria is idiopathic hyperoxaluria.

In one embodiment, the subject is fed a meal within one hour of administering the pharmaceutical composition. In another embodiment, the subject is fed a meal concurrently with administering the pharmaceutical composition. In one embodiment, the recombinant bacterium is capable of decreasing urinary oxalate in the subject after administration by at least 20%, at least 25%, at least 30%, at least 35%, at least 40%, or at least 45%.

In one embodiment, the decrease is a decrease as compared to a level of urinary oxalate in the subject prior to administration. In another embodiment, the decrease is a decrease as compared to a level of urinary oxalate in a subject, or a population of subjects, having hyperoxaluria that has not been treated with the recombinant bacterium. In one embodiment, the method further comprises measuring the level of urinary oxalate in the subject prior to administration. In another embodiment, the method further comprises measuring the level of urinary oxalate in the subject after administration. In one embodiment, the method comprises measuring the level of urinary oxalate in the subject prior to administration and after administration.

In one embodiment, the recombinant bacterium is capable of decreasing fecal oxalate in the subject after administration by at least 20%, at least 30%, at least 40%, at least 50%, at least 60%, at least 70%, at least 80%, or at least 85%. In one embodiment, the decrease is a decrease as compared to a level of fecal oxlate in the subject prior to administration. In another embodiment, the decrease is a decrease as compared to a level of fecal oxalate in a subject, or a population of subjects, having hyperoxaluria that has not been treated with the recombinant bacterium. In one embodiment, the method further comprises measuring the level of fecal oxalate in the subject prior to administration. In another embodiment, the method further comprises measuring the level of fecal oxalate in the subject after administration. In one embodiment, the method comprises measuring the level of fecal oxalate in the subject prior to administration and after administration.

In one embodiment, disclosed herein is a method for reducing the levels of oxalate in a subject, the method comprising administering to the subject a pharmaceutical composition comprising a recombinant bacterium comprising: one or more gene sequences encoding one or more oxalate catabolism enzymes operably linked to a directly or indirectly first promoter that is not associated with the oxalate catabolism enzyme gene in nature, wherein the one or more gene sequences comprise a scaaE3 gene, an frc gene, and an oxdC gene, wherein the scaaE3 gene comprises a sequence having at least 90%, 91%, 92%, 93%, 94%, 95%, 96%, 97%, 98%, or 99% identity to, comprises, or consists of SEQ ID NO: 3, wherein the frc gene comprises a sequence having at least 90%, 91%, 92%, 93%, 94%, 95%, 96%, 97%, 98%, or 99% identity to, comprises, or consists of SEQ ID NO: 1, and wherein the oxdC gene comprises a sequence having at least 90%, 91%, 92%, 93%, 94%, 95%, 96%, 97%, 98%, or 99% identity to, comprises, or consists of SEQ ID NO: 2, a gene encoding an oxalate importer, wherein the gene encoding the oxalate importer is an oxlT gene, and wherein the oxlT gene comprises a sequence having at least 90%, 91%, 92%, 93%, 94%, 95%, 96%, 97%, 98%, or 99% identity to, comprises, or consists of SEQ ID NO: 11, a Δ thyA auxotrophy, a deletion in an endogenous phage, a modified endogenous colibactin island, thereby reducing the levels of oxalate in the subject.

In one embodiment, the endogenous phage comprises a sequence having at least 90%, 91%, 92%, 93%, 94%, 95%, 96%, 97%, 98%, or 99% identity to, comprises, or consists of SEQ ID NO: 63.

In one embodiment, the modified endogenous colibactin island comprises one or more modified clb sequences selected from the group consisting of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), clbR (SEQ ID NO: 1082), and clbS (SEQ ID NO: 1803).

In one embodiment, the modified endogenous colibactin island comprises a deletion of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), and clbR (SEQ ID NO: 1082).

In one embodiment, the recombinant bacterium does not comprise a gene encoding for antibiotic resistance.

In one embodiment, the recombinant bacterium has an oxalate consumption activity of at least about 1 μmol/1×10⁹ cell.

In one embodiment, the recombinant bacterium has an oxalate consumption activity of about 50 to about 600 mg/day under anaerobic conditions. In one embodiment, the recombinant bacterium has an oxalate consumption activity of about 211 mg/day under anaerobic conditions. In one embodiment, the recombinant bacterium has an oxalate consumption activity of about 211 mg/day under anaerobic conditions when administered to the subject three times per day.

In one embodiment, the anaerobic conditions are conditions in the intestine and/or colon of the subject.

In one embodiment, the method reduces acute levels of oxalate in the subject by about two fold. In one embodiment, the method reduces acute levels of oxalate in the subject by about three fold. In one embodiment, the method reduces chronic levels of oxalate in the subject by about two fold. In one embodiment, the method reduces chronic levels of oxalate in the subject by about three fold.

In one embodiment, the method reduces acute levels of oxalate in the subject to about 25 mg/day, about 30 mg/day, about 40 mg/day, about 50 mg/day, about 60 mg/day, about 70 mg/day, about 80 mg/day, about 90 mg/day, or about 100 mg/day. In one embodiment, the method reduces chronic levels of oxalate in the subject to about 25 mg/day, about 30 mg/day, about 40 mg/day, about 50 mg/day, about 60 mg/day, about 70 mg/day, about 80 mg/day, about 90 mg/day, or about 100 mg/day.

In one embodiment, the recombinant bacterium is of the genus Escherichia. In one embodiment, the recombinant bacterium is of the species Escherichia coli strain Nissle.

In one embodiment, the pharmaceutical composition is administered orally. In one embodiment, the subject is fed a meal within one hour of administering the pharmaceutical composition. In one embodiment, the subject is fed a meal concurrently with administering the pharmaceutical composition.

In one embodiment, the subject is a human subject.

In one embodiment, the first promoter is an inducible promoter, optionally when the inducible promoter is a FNR promoter, optionally wherein the FNR promoter comprises a sequence having at least 90%, 91%, 92%, 93%, 94%, 95%, 96%, 97%, 98%, or 99% identity to, comprises, or consists of any one of SEQ ID NOs: 13-29.

In some embodiments, the inducible promoter is a Pr/Pl promoter. In one embodiment, the Pr/Pl promoter comprises a sequence having at least 90%, 91%, 92%, 93%, 94%, 95%, 96%, 97%, 98%, or 99% identity to, comprises, or consists of any one of SEQ ID NOs: 206, 213, and 219. In some embodiments, the recombinant bacterium further comprises a gene sequence encoding a mutant repressor of the Pr/Pl promoter. In some embodiments, the gene sequence encoding a mutant repressor comprises a sequence having at least 90%, 91%, 92%, 93%, 94%, 95%, 96%, 97%, 98%, or 99% identity to, comprises, or consists of any one of SEQ ID NOs: 210 and 214.

In some embodiments, the inducible promoter is a IPTG inducible promoter. In one embodiment, the IPTG inducible promoter comprises a sequence having at least 90%, 91%, 92%, 93%, 94%, 95%, 96%, 97%, 98%, or 99% identity to, comprises, or consists of SEQ ID NO: 1107. In some embodiments, the recombinant bacterium further comprises a gene sequence encoding a repressor of the Lac promoter. In some embodiments, the gene sequence encoding a repressor comprises a sequence having at least 90%, 91%, 92%, 93%, 94%, 95%, 96%, 97%, 98%, or 99% identity to, comprises, or consists of SEQ ID NO: 1105. In some embodiments, the repressor comprises a sequence having at least 90%, 91%, 92%, 93%, 94%, 95%, 96%, 97%, 98%, or 99% identity to, comprises, or consists of SEQ ID NO: 1106.

In one embodiment, the recombinant bacterium is SYNB8802v1.

In one embodiment, the subject has hyperoxaluria. In one embodiment, the hyperoxaluria is primary hyperoxaluria, dietary hyperoxaluria, or enteric hyperoxaluria.

In one embodiment, the subject has short bowel syndrome, chronic pancreatitis, inflammatory bowel disease (IBD), cystic fibrosis, kidney disease, and/or Roux-en-Y gastric bypass. In one embodiment, the subject has short bowel syndrome and/or Roux-en-Y gastric bypass.

In one embodiment, the subject has urinary oxalate (Uox) levels of at least 70 mg/day prior to the administering.

In one embodiment, the subject exhibits a decrease in Uox levels of at least 10%, 15%, 20%, 25%, 30%, 35%, 40%, 45%, 50%, 55%, 60%, 65%, 70%, 75%, or 80% after the administering.

In one embodiment, the subject has eGFR<30 mL/min/1.73 m², requires hemodialysis, or has systemic oxalosis prior to the administering.

In one embodiment, the recombinant bacteria is administered at a dose of about 1×10¹¹ live recombinant bacteria, about 3×10¹¹ live recombinant bacteria, about 4.5×10¹¹ live bacteria, about 5×10¹¹ live recombinant bacteria, about 6×10¹¹ live recombinant bacteria, about 1×10¹² live recombinant bacteria, or about 2×10¹² live recombinant bacteria. In one embodiment, the administering is about 4.5×10¹¹ live recombinant bacteria.

In one embodiment, the recombinant bacteria is administered once daily, twice daily, or three times daily. In one embodiment, the administering is about 5×10¹¹ live recombinant bacteria with meals three times per day.

In one embodiment, the method further administering a proton pump inhibitor (PPI) to the subject. In one embodiment, the PPI is esomeprazole. In another embodiment, esomeprazole is administered at 40 mg once daily. In one embodiment, the administering of the PPI is once a day.

In one embodiment, the pharmaceutical composition further comprises galactose. In one embodiment, galactose is D-galactose. In another embodiment, galactose is present in the composition at about 0.1 g to about 3 g, about 0.1 g to about 2.5 g, about 0.1 g to about 2.0 g, about 0.1 g to about 1.5 g, about 0.1 g to about 1.0 g, about 0.1 g to about 0.5 g, about 0.5 g to about 3 g, about 0.5 g to about 2.5 g, about 0.5 g to about 2.0 g, about 0.5 g to about 1.5 g, about 0.5 g to about 1.0 g, about 1.0 g to about 3 g, about 1.0 g to about 2.5 g, about 1.0 g to about 2.0 g, about 1.0 g to about 1.5 g, about 1.5 g to about 3 g, about 1.5 g to about 2.5 g, about 1.5 g to about 2.0 g, about 2.0 g to about 3 g, about 2.0 g to about 2.5 g, or about 2.5 g to about 3 g. In some embodiments, galactose is present in the composition at about 1.0 g. In some embodiments, galactose is present in the composition at about 0.5 g. In some embodiments, galactose is present in the composition at about 2.0 g.

In one embodiment, disclosed herein is a recombinant bacterium comprising: one or more gene sequences encoding one or more oxalate catabolism enzymes operably linked to a directly or indirectly first promoter that is not associated with the oxalate catabolism enzyme gene in nature, wherein the one or more gene sequences comprise a scaaE3 gene, an frc gene, and an oxdC gene, wherein the scaaE3 gene comprises a sequence having at least 90%, 91%, 92%, 93%, 94%, 95%, 96%, 97%, 98%, or 99% identity to, comprises, or consists of SEQ ID NO: 3, wherein the frc gene comprises a sequence having at least 90%, 91%, 92%, 93%, 94%, 95%, 96%, 97%, 98%, or 99% identity to, comprises, or consists of SEQ ID NO: 1, and wherein the oxdC gene comprises a sequence having at least 90%, 91%, 92%, 93%, 94%, 95%, 96%, 97%, 98%, or 99% identity to, comprises, or consists of SEQ ID NO: 2, a gene encoding an oxalate importer, wherein the gene encoding the oxalate importer is an oxlT gene, and wherein the oxlT gene comprises a sequence having at least 90%, 91%, 92%, 93%, 94%, 95%, 96%, 97%, 98%, or 99% identity to, comprises, or consists of SEQ ID NO: 11, a Δ thyA auxotrophy, a deletion in an endogenous phage, a modified endogenous colibactin island.

In one embodiment, the endogenous phage comprises a sequence having at least 90%, 91%, 92%, 93%, 94%, 95%, 96%, 97%, 98%, or 99% identity to, comprises, or consists of SEQ ID NO: 63.

In one embodiment, the modified endogenous colibactin island comprises one or more modified clb sequences selected from the group consisting of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), clbR (SEQ ID NO: 1082), and clbS (SEQ ID NO: 1803).

In one embodiment, the modified endogenous colibactin island comprises a deletion of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), and clbR (SEQ ID NO: 1082).

In one embodiment, the recombinant bacterium does not comprise a gene encoding for antibiotic resistance.

In one embodiment, the first promoter is an inducible promoter, optionally when the inducible promoter is a FNR promoter, optionally wherein the FNR promoter comprises a sequence having at least 90%, 91%, 92%, 93%, 94%, 95%, 96%, 97%, 98%, or 99% identity to, comprises, or consists of any one of SEQ ID NOs: 13-29.

In one embodiment, the recombinant bacterium has an oxalate consumption activity of at least about 1 μmol/1×10⁹ cell. In one embodiment, the recombinant bacterium has an oxalate consumption activity of about 50-600 mg/day under anaerobic conditions.

In one embodiment, the recombinant bacterium is SYNB8802v1. In one embodiment, the recombinant bacterium is SYNB8802.

In one embodiment, the recombinant bacterium has an oxalate consumption activity of about 0.2 μmole/hr to about 1.6 μmole/hr, about 0.5 μmole/hr to about 1.5 μmole/hr, or about 1.0 μmole/hr to about 1.5 μmole/hr under anaerobic conditions. In one embodiment, the recombinant bacterium has an oxalate consumption activity of about 0.5 μmole/hr to about 1.5 μmole/hr under anaerobic conditions. In one embodiment, the recombinant bacterium has an oxalate consumption activity of about 0.2 μmole/hr to about 1.6 μmole/hr, about 0.5 μmole/hr to about 1.5 μmole/hr, or about 1.0 mole/hr to about 1.5 μmole/hr under anaerobic conditions. In one embodiment, the recombinant bacterium has an oxalate consumption activity of about 0.5 μmole/hr to about 1.5 μmole/hr under anaerobic conditions.

In one embodiment, the recombinant bacterium is capable of decreasing urinary oxalate in the subject after administration by at least 20%, at least 25%, at least 30%, at least 35%, at least 40%, or at least 45%. In one embodiment, the recombinant bacterium is capable of decreasing fecal oxalate in the subject after administration by at least 20%, at least 30%, at least 40%, at least 50%, at least 60%, at least 70%, at least 80%, or at least 85%.

### BRIEF DESCRIPTION OF THE DRAWINGS

FIG. 1 depicts a graph showing the results of an in vitro oxalate degradation assay using an engineered E. coli. Nissle strain as compared to a wild type E. coli Nissle strain.

FIG. 2A depicts a bar graph showing the results of an in vivo oxalate consumption experiment by measuring acute ¹³C-oxalate urinary recovery when using an engineered E. coli Nissle strain (Engineered EcN) as compared to a wild type E. coli Nissle strain (EcN). FIG. 2B depicts a bar graph showing the results of an in vivo oxalate consumption experiment by measuring chronic urinary oxalate recovery when using an engineered E. coli Nissle strain (Engineered EcN) as compared to a wild type E. coli Nissle strain (EcN).

FIG. 3 is a figure summarizing the disease pathogenesis of enteric hyperoxaluria.

FIG. 4A depicts the components of strain SYNB8802, and FIG. 4B depicts a graph showing the results of an in vitro oxalate degradation assay using SYNB8802 as compared to a wild type E. coli Nissle strain. FIG. 4C depicts as graph showing the results of an in vitro oxalate degradation and formate production assay using SYNB8802 as compared to a wild type E. coli Nissle strain.

FIG. 5A depicts oxalate consumption with SYN-HOX (SYN5752) when SYN-HOX was activated in simulated stomach and colon fluid. FIG. 5B depicts that an engineered E. coli Nissle strain (Engineered EcN), SYN5752 consumed oxalate in mice in the gut. SYN5752 is an integrated strain with antibiotic resistance. SYN7169 is an integrated strain with antibiotic resistance, auxotrophy, and phage 3 deletion. ¹³C-oxalate consumption was measured in multiple acute mouse studies, and the efficacy of the strain ranged between 50-75%. SYN7169 behaved similarly to SYN5752 in this mouse model. FIG. 5C depicts oxalate consumption with SYNB8802 in the gastrointestinal (GI) tract of healthy mice. Data presented as mean urinary ¹³C-oxalate recovery normalized by creatinine±standard error of the mean. Statistical analysis was performed using one-way analysis of variance followed by Dunnett's multiple comparison test. ****p<0.0001.

FIG. 6 depicts an attenuation of urinary oxalate increase in healthy monkeys.

FIG. 7A depicts a bar graph showing dose-dependent recovery of urinary oxalate in healthy monkeys (NHP) after treatment with SYN7169. FIG. 7B depicts a bar graph showing dose-dependent recovery of urinary ¹³C-oxalate in healthy monkeys after treatment with SYN7169. FIG. 7C depicts oxalate and ¹³C-oxalate consumption in the GI tract of cynomolgus monkeys with acute hyperoxaluria. Data presented as mean urinary oxalate or ¹³C-oxalate recovery normalized by creatinine±standard error of the mean. Statistical analysis was performed using paired t-test. **p<0.01.

FIG. 8A depicts that SYNB8802 is viable in vivo and cleared from feces of mice by 24 hours.

FIG. 8B depicts recovered SYN-HOX (SYNB8802) from feces of cynomolgus monkeys over 6 and 24 hours.

FIG. 9 depicts oxalate consumption with SYNB8802 lyophilized (Lyo) and frozen liquid (FL) in non-human primates (NHP).

FIG. 10 depicts oxalate consumption in mice with SYN-HOX (SYN7169) lyophilized (Lyo) and frozen liquid based on CFU and live cell.

FIG. 11A depicts a graph modeling dose-dependent recovery of urinary oxalate in human patients after treatment with SYNB8802. FIG. 11B depicts a schematic of enteric hyperoxaluria in silico simulation (ISS) model. FIG. 11C depicts in silico simulation (ISS), urinary oxalate percent change from baseline after dosing with SYNB8802. This modeling suggests that SYNB8802 has the potential to achieve >20% urinary oxalate lowering at target dose range.

FIG. 12 is a schematic summarizing the organization of the clinical trial.

FIG. 13 depicts a graph of baseline urinary oxalate after high oxalate/low calcium diet in healthy volunteers.

FIG. 14A depicts a graph of dose-responsive and reproducible urinary oxalate lowering after SYNB8802 administration and 600 mg daily oxalate. Lower percent change urinary oxalate is better.

FIG. 14B depicts a graph of as in FIG. 14A after SYNB8802 administration and 400 mg daily oxalate. Lower percent change urinary oxalate is better. LS mean change over Placebo, +/−90% CI, all days baseline and treated.

FIG. 15A depicts a graph of the change of urinary oxalate after administration of SYNB8802 at a dose of 3×10¹¹ live cells. FIG. 15B depicts a graph of urinary oxalate levels in healthy volunteers administered the placebo or SYNB8802 at a dose of 3×10¹¹ live cells. LS mean change over Placebo, +/−90% std error of measurement, all days; and 24 hr UOx after 5 days of dosing, +/−90% std error of measurement. 600 mg daily oxalate.

FIG. 16 depicts a graph of the change of urinary oxalate in healthy volunteers. LS Mean % change over pbo+/−SEM.

FIG. 17 depicts a graph of in vitro simulation (IVS) Left y-axis: Rate of oxalate degradation in μmol/h/10⁹ cells. X-axis=time in hours. Left X-axis 0-4 hours, Right X-axis 6-48 h. Dots represent an average of a triplicate with error bars representing standard deviation. Data points in the box on the left represent incubation in simulated gastric fluid (SGF). Data points in the middle box represent incubation in simulated intestinal fluid (SIF). Data points in the box on the right represent incubation in simulated colonic fluid (SCF).

FIG. 18A depicts an in silico simulation (ISS) enzyme activity and pH inhibition model. Michaelis-Menten model of enzyme kinetics. Vmax defines the maximal enzyme velocity (consumption rate of oxalate by SYNB8802). Km defines the oxalate concentration at which half-maximal enzyme velocity occurs. Vmax and Km were determined through in vitro simulation.

FIG. 18B depicts Simulated gastric pH as a function of time following a solid meal (dark blue). This function is a power exponential decay with a half-life of 110 minutes and a shape parameter equal to 1.81. Likelihood of time spent in the stomach based on gastric residence time distribution (light blue). The distribution is truncated to a maximum of 4 hours, and the median gastric residence time is 110 minutes.

FIG. 18C depicts simulated normalized SYNB8802 activity in the stomach as a function of time.

FIG. 18D depicts Simulated normalized SYNB8802 activity in the small intestine as a function of time previously spent in the stomach. Function is equivalent to gastric function with an upper limit imposed based on intestinal pH.

FIG. 19A depicts in silico simulation (ISS) model validation and simulated urinary oxalate lowering subsequent dietary oxalate removal by SYNB8802. Validation of simulated urinary oxalate excretion against clinical data. Simulated urinary oxalate on a free-living diet and on three days of a high-oxalate diet (dark blue); points and error bars represent mean and standard deviation, respectively, across 30 simulated healthy subjects. Observed urinary oxalate on a free-living diet and on three days of a high-oxalate, low-calcium (HOLC) diet (light blue); points and error bars represent mean and standard deviation, respectively, across 30 healthy subjects.

FIG. 19B depicts simulated urinary oxalate and urinary oxalate reduction for healthy subjects consuming 200 mg/day dietary oxalate without SYNB8802 and with 1×10¹¹, 2×10¹¹, and 5×10¹¹ SYNB8802 cells TID over ten days. Points represent simulations under a baseline assumption of dietary oxalate absorption in healthy subjects (Holmes et al., 2001). Error bars represent a simulated range of dietary oxalate absorption (0.75×-1.25× baseline).

FIG. 20 depicts SYNB8802 pH inhibition in vitro simulation. SYNB8802 activity as a function of exposure time to medium at pH ranging from 2.0 to 7.0. Points and error bars in black represent in vitro measurements (n=3 replicate cultures per group; mean±SD). Blue curves represent exponential decay models fit to in vitro measurements for each pH level.

FIG. 21A depicts separation of UOx in active and placebo groups started from the BID (twice a day) day and maintained throughout the dosing period, when subjects were given 400 mg oxalate daily in their diets. The active group was administered 3e11 live cells of SYNB8802.

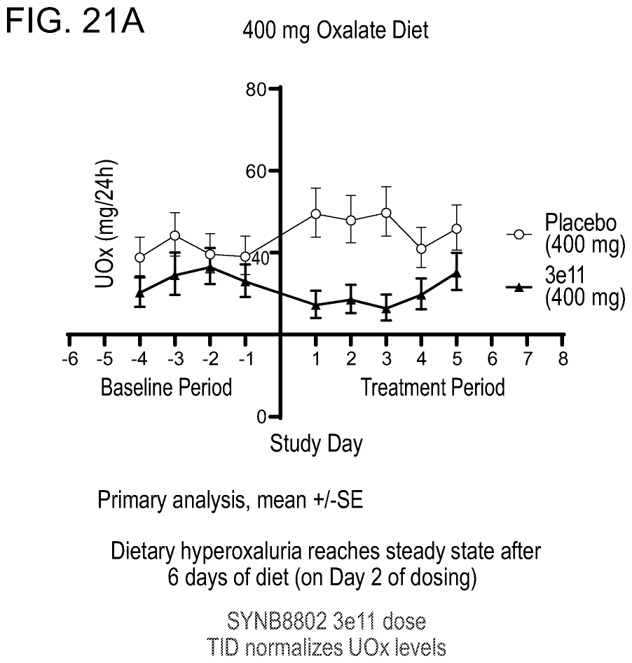

FIG. 21B depicts separation of UOx in active and placebo groups started from the BID (twice a day) day and maintained throughout the dosing period, when subjects were given 600 mg oxalate daily in their diets. The active group was administered 3e11 live cells of SYNB8802.

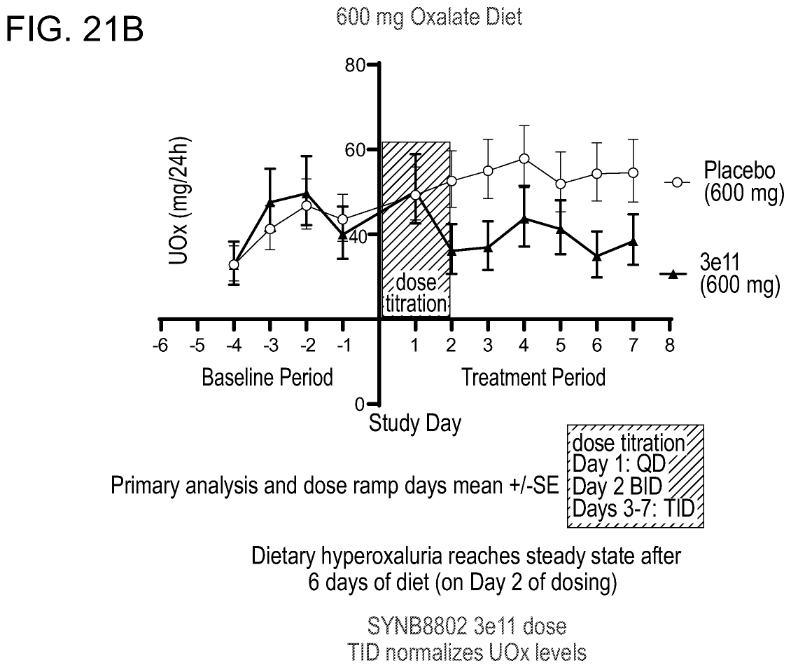

FIG. 22 depicts dose-related reduction of fecal oxalate when the subjects were administered 600 mg oxalate daily in their diets.

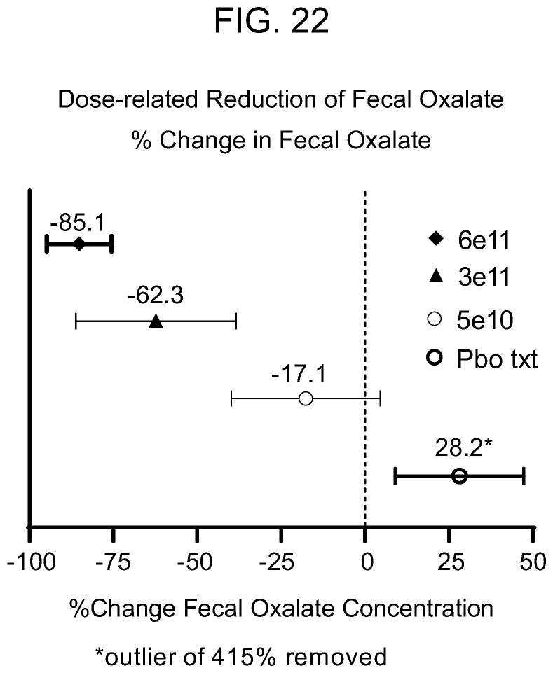

FIG. 23 is a schematic summarizing the organization of the Part 1a-study design.

FIG. 24 depicts reduction of oxalate with SYNB8802 (HOX+pks) and SYNB8802v1 (HOX−pks).

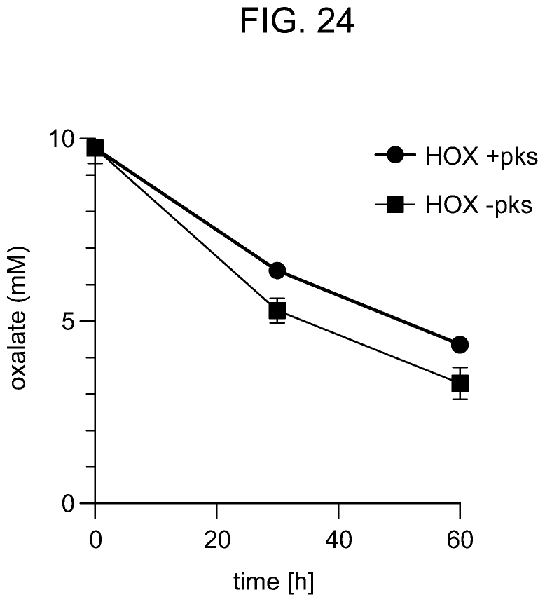

FIG. 25 depicts ISS model validation against Phase 1 data.

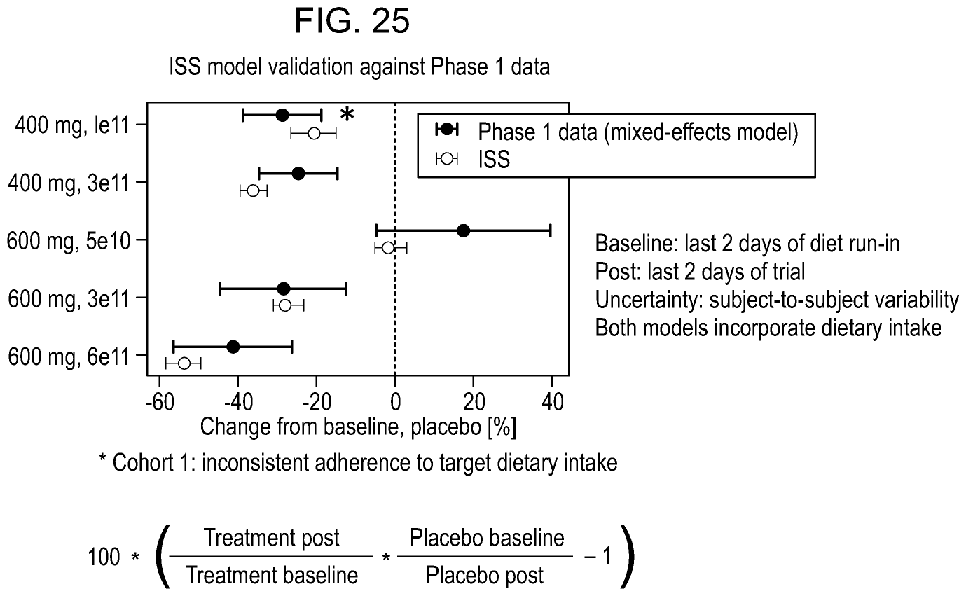

FIG. 26 depicts a schematic of an exemplary oxalate consuming strain, SYNB8802.

### DETAILED DESCRIPTION

Oxalate arises from a variety of dietary and endogenous sources and is considered an end-product of human metabolism. Under physiological conditions, the absorbed dietary and endogenously produced oxalate is excreted by the kidneys as urinary oxalate (UOx). (Mitchell T, et al. Dietary oxalate and kidney stone formation Am J Physiol Renal Physiol. 2019; 316:F409-13). In a healthy person, only a small fraction of ingested oxalate is absorbed. The contribution of endogenous oxalate production and dietary oxalate absorption to UOx is approximately equal in a healthy person (Holmes R, Goodman H, Assimos D. Contribution of dietary oxalate to urinary oxalate excretion. Kidney Int. 2001; 59:270-76). An increase in either gastrointestinal (GI) oxalate absorption or hepatic oxalate production increases plasma oxalate (POx) and thus UOx, and contributes to the risk of stone formation and other adverse renal outcomes (Curhan G, Taylor E. 24-h uric acid excretion and the risk of kidney stones. Kidney Int. 2008; 73:489-96). Approximately 85% of kidney stones are due to calcium oxalate supersaturation in the urine.

Enteric hyperoxaluria (EH) can result from increased gut oxalate absorption, increased oxalate bioavailability from food, decreased intestinal oxalate degradation, decreased intestinal secretion of oxalate, or increased endogenous production of oxalate. Under normal conditions, dietary calcium forms a complex with oxalate in the gut lumen and renders it insoluble and therefore unavailable for absorption. Diseases of the GI tract leading to fat malabsorption and increased free fatty acids in the gut can lead to increased soluble oxalate in the colon and increased colonic absorption of oxalate by preventing the formation of the calcium-oxalate complex.

Consequently, EH is commonly observed in patients with underlying digestive diseases affecting fat absorption such as patients with a history of weight loss surgery, inflammatory bowel disease (IBD), cystic fibrosis, short-bowel syndrome, and chronic biliary or pancreatic pathologies. The prevalence of EH patients with kidney stones in the United States has been estimated to be <250,000, with the most frequent underlying malabsorptive enteric conditions being Roux-n-Y (RnY) gastric bypass (>60%) and IBD (20%) (Tasian G, Wade B, Gaebler J, Kausz A, Medicis J, Wyatt C. Prevalence of kidney stones in patients with enteric disorders. Paper presented at American Society of Nephrology Washington, D C, 2019).

The gut microbiota and certain genetic anomalies can also have an impact on oxalate homeostasis. Certain commensal bacterial strains in the human gut microbiome, including Oxalobacter spp., Bifidobacterium spp., and Lactobacillus spp., can degrade oxalate and may be capable of modulating intestinal oxalate secretion. Furthermore, inherited defects in the SLC26 family of anion exchangers may predispose individuals to EH (Freel R, et al., Ieal oxalate absorption and urinary oxalate excretion are enhanced in Slc26a6-null mice. Am J Physiol. 2006; 290:G719-28.)

Enteric hyperoxaluria can lead to the formation of kidney stones. Increased UOx is also a risk factor for acute kidney injury and chronic kidney disease (CKD) (Lumlertgul N, Siribamrungwong M, Jaber B, Susantitaphong P. Secondary oxalate nephropathy: A systematic review. Kidney Int Rep. 2018; 3:1363-72). Oxalate nephropathy in EH can lead to progressive renal deterioration and eventually to end-stage kidney disease, requiring dialysis. When glomerular filtration rate falls below 30 to 40 m/min per 1.73 m2, POx levels can increase markedly, predisposing to the formation of calcium oxalate crystal deposits in extrarenal tissues, a process called systemic oxalosis. Although this is a rare manifestation of EH, CKD patients can present with involvement of the retina, joints, skin, and cardiovascular system, with severe consequences.

There are no approved pharmacological therapies for treating hyperoxaluria. The management of hyperoxaluria is aimed at decreasing the risk of recurrent kidney stones and involves controlling and lowering the intake of dietary oxalate and fat, increasing dietary calcium intake, and ensuring adequate fluid intake (Pearle M S, Goldfarb D S, Assimos D G, Curhan G, Denu-Ciocca C J, Matlaga B R, et al. Medical management of kidney stones: AUA guideline. J Urol. 2014; 92(2):316-24). However, the efficacy of dietary treatment, especially in those with severe hyperoxaluria, is limited. Adherence to a low oxalate diet for a prolonged time is challenging, due to the presence of oxalate in many foods (e.g., green vegetables, nuts, grains, fruits, chocolate). The absorption of oxalate is also increased with the typical Western diet with a high salt, high fat, and low-calcium content. Thus, there is an unmet medical need for a well-tolerated, chronic therapy for patients with secondary hyperoxaluria and its associated complications, such as kidney stones and oxalate nephropathy.

The disclosure includes engineered and programmed microorganisms, e.g., bacteria, yeast, viruses etc., pharmaceutical compositions thereof, and methods of modulating and treating disorders in which oxalate is detrimental. In some embodiments, the microorganism, e.g., bacterium, yeast, or virus, has been genetically engineered to comprise heterologous gene sequence(s) encoding one or more oxalate catabolism enzyme(s). In some embodiments, the microorganism, e.g., bacterium, yeast, or virus, has been genetically engineered to comprise heterologous gene sequence(s) encoding one or more oxalate catabolism enzyme(s) and is capable of processing and reducing oxalate and/or oxalic acid in low-oxygen environments, e.g., the gut. In some embodiments, the engineered microorganism comprises heterologous gene sequence(s) encoding one or more oxalate catabolism enzyme(s) and is capable of transporting oxalic acid and/or oxalate and/or another related metabolite(s) into the bacterium. Thus, the recombinant microorganism and pharmaceutical compositions comprising the microorganism of the invention may be used to catabolize oxalate or oxalic acid to treat and/or prevent conditions associated with disorders in which oxalate is detrimental. In one embodiment, the disorder in which oxalate is detrimental is a disorder involving the abnormal levels of oxalate, such as primary hyperoxalurias (i.e., PHI, PHII, and PHIII), secondary hyperoxaluria, enteric hyperoxaluria, dietary hyperoxaluria, or idiopathic hyperoxaluria.

In some embodiments, the engineered microorganism comprise gene sequence(s) encoding one or more of the following: (i) one or more transporter(s) of oxalate; (ii) one or more exporter(s) of formate; (iii) one or more polypeptide(s) which mediate both the transport (import) of oxalate and the export of formate (e.g., oxalate:formate antiporter(s)); and (iv) any combination thereof. In some embodiments, the microorganism has been engineered to comprise gene sequence(s) encoding one or more oxalate catabolism enzyme(s) and one or more of the following: (i) one or more transporter(s) of oxalate; (ii) one or more exporter(s) of formate; (iii) one or more polypeptide(s) which mediate both the transport (import) of oxalate and the export of formate (e.g., oxalate:formate antiporter(s)); and (iv) any combination thereof.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “microorganism” or “recombinant microorganism” refers to a microorganism, e.g., bacterial or viral cell, or bacteria or virus, that has been genetically modified from its native state. Thus, a “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells disclosed herein may comprise exogenous nucleotide sequences on plasmids. Alternatively, recombinant bacterial cells may comprise exogenous nucleotide sequences stably incorporated into their chromosome.

A “programmed or engineered microorganism” refers to a microorganism, e.g., bacterial or viral cell, or bacteria or virus, that has been genetically modified from its native state to perform a specific function. Thus, a “programmed or engineered bacterial cell” or “programmed or engineered bacteria” or “genetically engineered bacterial cell or bacteria” refers to a bacterial cell or bacteria that has been genetically modified from its native state to perform a specific function, e.g., to metabolize a metabolite, e.g., oxalate. In certain embodiments, the programmed or engineered bacterial cell has been modified to express one or more proteins, for example, one or more proteins that have a therapeutic activity or serve a therapeutic purpose. The programmed or engineered bacterial cell may additionally have the ability to stop growing or to destroy itself once the protein(s) of interest have been expressed.

As used herein, the term “gene” refers to a nucleic acid fragment that encodes a protein or fragment thereof, optionally including regulatory sequences preceding (5′ non-coding sequences) and following (3′ non-coding sequences) the coding sequence. In one embodiment, a “gene” does not include regulatory sequences preceding and following the coding sequence. A “native gene” refers to a gene as found in nature, optionally with its own regulatory sequences preceding and following the coding sequence. A “chimeric gene” refers to any gene that is not a native gene, optionally comprising regulatory sequences preceding and following the coding sequence, wherein the coding sequences and/or the regulatory sequences, in whole or in part, are not found together in nature. Thus, a chimeric gene may comprise regulatory sequences and coding sequences that are derived from different sources, or regulatory and coding sequences that are derived from the same source, but arranged differently than is found in nature.

As used herein, the term “gene sequence” is meant to refer to a genetic sequence, e.g., a nucleic acid sequence. The gene sequence or genetic sequence is meant to include a complete gene sequence or a partial gene sequence. The gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence.

As used herein, a “heterologous” gene or “heterologous sequence” refers to a nucleotide sequence that is not normally found in a given cell in nature. As used herein, a heterologous sequence encompasses a nucleic acid sequence that is exogenously introduced into a given cell and can be a native sequence (naturally found or expressed in the cell) or non-native sequence (not naturally found or expressed in the cell) and can be a natural or wild-type sequence or a variant, non-natural, or synthetic sequence. “Heterologous gene” includes a native gene, or fragment thereof, that has been introduced into the host cell in a form that is different from the corresponding native gene. For example, a heterologous gene may include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. As used herein, the term “endogenous gene” refers to a native gene in its natural location in the genome of an organism. As used herein, the term “transgene” refers to a gene that has been introduced into the host organism, e.g., host bacterial cell, genome.

As used herein, a “non-native” nucleic acid sequence refers to a nucleic acid sequence not normally present in a microorganism, e.g., an extra copy of an endogenous sequence, or a heterologous sequence such as a sequence from a different species, strain, or substrain of bacteria or virus, or a sequence that is modified and/or mutated as compared to the unmodified sequence from bacteria or virus of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In some embodiments, “non-native” refers to two or more nucleic acid sequences that are not found in the same relationship to each other in nature. The non-native nucleic acid sequence may be present on a plasmid or chromosome. In some embodiments, the genetically engineered microorganism of the disclosure comprises a gene and/or gene cassette that is operably linked to a promoter that is not associated with said gene in nature. For example, in some embodiments, the genetically engineered bacteria disclosed herein comprise a gene or gene cassette encoding one or more oxalate-metabolizing enzyme(s) described herein and/or one or more oxalate transporter(s), one or more exporter(s) (e.g., of formate) and/or one or more antiporter(s)(e.g., oxalate:formate antiporter(s)) that is operably linked to a directly or indirectly inducible promoter that is not associated with said gene in nature, e.g., an FNR responsive one or more oxalate-metabolizing enzyme(s) described herein and/or one or more oxalate transporter(s), one or more exporter(s) (e.g., of formate) and/or one or more antiporter(s)(e.g., oxalate:formate antiporter(s)) (or other promoter disclosed herein) operably linked to a gene encoding a one or more oxalate-metabolizing enzyme(s) described herein and/or one or more oxalate transporter(s), one or more exporter(s) (e.g., of formate) and/or one or more antiporter(s)(e.g., oxalate:formate antiporter(s)). In some embodiments, the genetically engineered virus of the disclosure comprises a gene or gene cassette that is operably linked to a directly or indirectly inducible promoter that is not associated with said gene or gene cassette in nature, e.g., a promoter operably linked to a gene and/or gene cassette encoding one or more oxalate-metabolizing enzyme(s) and/or one or more oxalate transporter(s) and/or one or more exporter(s) (e.g., of formate) and/or one or more antiporter(s)(e.g., oxalate:formate antiporter(s)).

As used herein, the term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, signal sequences, and stem-loop structures. In one embodiment, the regulatory sequence comprises a promoter, e.g., an FNR responsive promoter or other promoter disclosed herein.

“Operably linked” refers to the association of nucleic acid sequences on a single nucleic acid fragment so that the function of one is affected by the other. A regulatory element is operably linked with a coding sequence when it is capable of affecting the expression of the gene coding sequence, regardless of the distance between the regulatory element and the coding sequence. More specifically, operably linked refers to a nucleic acid sequence, e.g., a gene or gene cassette encoding one or more an oxalate catabolism enzyme, that is joined to a regulatory sequence in a manner which allows expression of the nucleic acid sequence, e.g., the gene(s) or gene cassettes encoding one or more oxalate catabolism enzyme(s) and/or one or more oxalate transporter(s), one or more exporter(s) (e.g., of formate) and/or one or more antiporter(s)(e.g., oxalate:formate antiporter(s)). In other words, the regulatory sequence acts in cis. In one embodiment, a gene may be “directly linked” to a regulatory sequence in a manner which allows expression of the gene. In another embodiment, a gene may be “indirectly linked” to a regulatory sequence in a manner which allows expression of the gene. In one embodiment, two or more genes may be directly or indirectly linked to a regulatory sequence in a manner which allows expression of the two or more genes. A regulatory region or sequence is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive. A “constitutive promoter” refers to a promoter that allows for continual transcription of the coding sequence or gene under its control.

“Constitutive promoter” refers to a promoter that is capable of facilitating continuous transcription of a coding sequence or gene under its control and/or to which it is operably linked. Constitutive promoters and variants are well known in the art and include, but are not limited to, Ptac promoter, BBa_J23100, a constitutive Escherichia coli σ^{S} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), P_{liaG} (BBa_K823000), P_{lepA} (BBa_K823002), P_{veg}(BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), and a bacteriophage SP6 promoter (e.g., SP6 promoter (BBa_J64998)).

An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition. A “directly inducible promoter” refers to a regulatory region, wherein the regulatory region is operably linked to a gene and/or gene cassette encoding one or more oxalate-metabolizing enzyme(s) and/or one or more oxalate transporter(s) and/or one or more exporter(s) (e.g., of formate) and/or one or more antiporter(s) (e.g., oxalate:formate antiporter(s)), where, in the presence of an inducer of said regulatory region, the protein or polypeptide is expressed. An “indirectly inducible promoter” refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by “inducible promoter.” Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, a P_{TetR} promoter, and a P_{LacI} promoter, each of which are described in more detail herein. Examples of other inducible promoters are provided herein below.

As used herein, “stably maintained” or “stable” bacterium is used to refer to a bacterial host cell carrying non-native genetic material, e.g., a gene and/or gene cassette encoding one or more oxalate-metabolizing enzyme(s) and/or one or more oxalate transporter(s) and/or one or more exporter(s) (e.g., of formate) and/or one or more antiporter(s)(e.g., oxalate:formate antiporter(s)), which is incorporated into the host genome or propagated on a self-replicating extra-chromosomal plasmid, such that the non-native genetic material is retained, expressed, and propagated. The stable bacterium is capable of survival and/or growth in vitro, e.g., in medium, and/or in vivo, e.g., in the gut. For example, the stable bacterium may be a genetically engineered bacterium comprising a gene and/or gene cassette encoding one or more oxalate-metabolizing enzyme(s) and/or one or more oxalate transporter(s) and/or one or more exporter(s) (e.g., of formate) and/or one or more antiporter(s)(e.g., oxalate:formate antiporter(s)), in which the plasmid or chromosome carrying the gene is stably maintained in the bacterium, such that the one or more oxalate-metabolizing enzyme(s) and/or one or more oxalate transporter(s) and/or one or more exporter(s) (e.g., of formate) and/or one or more antiporter(s)(e.g., oxalate:formate antiporter(s)) can be expressed in the bacterium, and the bacterium is capable of survival and/or growth in vitro and/or in vivo. In some embodiments, copy number affects the stability of expression of the non-native genetic material. In some embodiments, copy number affects the level of expression of the non-native genetic material.

As used herein, the term “expression” refers to the transcription and stable accumulation of sense (mRNA) or anti-sense RNA derived from a nucleic acid, and/or to translation of an mRNA into a polypeptide.

As used herein, the term “plasmid” or “vector” refers to an extrachromosomal nucleic acid, e.g., DNA, construct that is not integrated into a bacterial cell's genome. Plasmids are usually circular and capable of autonomous replication. Plasmids may be low-copy, medium-copy, or high-copy, as is well known in the art. Plasmids may optionally comprise a selectable marker, such as an antibiotic resistance gene, which helps select for bacterial cells containing the plasmid and which ensures that the plasmid is retained in the bacterial cell. A plasmid disclosed herein may comprise a nucleic acid sequence encoding a heterologous gene, e.g., a gene and/or gene cassette encoding one or more oxalate-metabolizing enzyme(s) and/or one or more oxalate transporter(s) and/or one or more exporter(s) (e.g., of formate) and/or one or more antiporter(s) (e.g., oxalate:formate antiporter(s)).

As used herein, the term “transform” or “transformation” refers to the transfer of a nucleic acid fragment into a host bacterial cell, resulting in genetically-stable inheritance. Host bacterial cells comprising the transformed nucleic acid fragment are referred to as “recombinant” or “transgenic” or “transformed” organisms.

The term “genetic modification,” as used herein, refers to any genetic change. Exemplary genetic modifications include those that increase, decrease, or abolish the expression of a gene, including, for example, modifications of native chromosomal or extrachromosomal genetic material. Exemplary genetic modifications also include the introduction of at least one plasmid, modification, mutation, base deletion, base addition, base substitution, and/or codon modification of chromosomal or extrachromosomal genetic sequence(s), gene over-expression, gene amplification, gene suppression, promoter modification or substitution, gene addition (either single or multi-copy), antisense expression or suppression, or any other change to the genetic elements of a host cell, whether the change produces a change in phenotype or not. Genetic modification can include the introduction of a plasmid, e.g., a plasmid comprising a gene and/or gene cassette encoding one or more oxalate-metabolizing enzyme(s) and/or one or more oxalate transporter(s) and/or one or more exporter(s) (e.g., of formate) and/or one or more antiporter(s) (e.g., oxalate:formate antiporter(s)) operably linked to a promoter, into a bacterial cell. Genetic modification can also involve a targeted replacement in the chromosome, e.g., to replace a native gene promoter with an inducible promoter, regulated promoter, strong promoter, or constitutive promoter. Genetic modification can also involve gene amplification, e.g., introduction of at least one additional copy of a native gene into the chromosome of the cell. Alternatively, chromosomal genetic modification can involve a genetic mutation.

As used herein, the term “genetic mutation” refers to a change or changes in a nucleotide sequence of a gene or related regulatory region that alters the nucleotide sequence as compared to its native or wild-type sequence. Mutations include, for example, substitutions, additions, and deletions, in whole or in part, within the wild-type sequence. Such substitutions, additions, or deletions can be single nucleotide changes (e.g., one or more point mutations), or can be two or more nucleotide changes, which may result in substantial changes to the sequence. Mutations can occur within the coding region of the gene as well as within the non-coding and regulatory sequence of the gene. The term “genetic mutation” is intended to include silent and conservative mutations within a coding region as well as changes which alter the amino acid sequence of the polypeptide encoded by the gene. A genetic mutation in a gene coding sequence may, for example, increase, decrease, or otherwise alter the activity (e.g., enzymatic activity) of the gene's polypeptide product. A genetic mutation in a regulatory sequence may increase, decrease, or otherwise alter the expression of sequences operably linked to the altered regulatory sequence.

Specifically, the term “genetic modification that reduces export of oxalate from the bacterial cell” refers to a genetic modification that reduces the rate of export or quantity of export of an oxalate from the bacterial cell, as compared to the rate of export or quantity of export of oxalate from a bacterial cell not having said modification, e.g., a wild-type bacterial cell. In one embodiment, a recombinant bacterial cell having a genetic modification that reduces export of oxalate from the bacterial cell comprises a genetic mutation in a native gene. In another embodiment, a recombinant bacterial cell having a genetic modification that reduces export of oxalate from the bacterial cell comprises a genetic mutation in a native promoter, which reduces or inhibits transcription of a gene encoding an oxalate exporter. In another embodiment, a recombinant bacterial cell having a genetic modification that reduces export of oxalate from the bacterial cell comprises a genetic mutation leading to overexpression of a repressor of an exporter of oxalate. In another embodiment, a recombinant bacterial cell having a genetic modification that reduces export of oxalate from the bacterial cell comprises a genetic mutation which reduces or inhibits translation of the gene encoding the oxalate exporter.

Moreover, the term “genetic modification that increases import of oxalate into the bacterial cell” refers to a genetic modification that increases the uptake rate or increases the uptake quantity of oxalate into the cytosol of the bacterial cell, as compared to the uptake rate or uptake quantity of the oxalate into the cytosol of a bacterial cell not having said modification, e.g., a wild-type bacterial cell. In some embodiments, an engineered bacterial cell having a genetic modification that increases import of oxalate into the bacterial cell refers to a bacterial cell comprising a heterologous gene sequence (native or non-native) encoding one or more importer/transporter(s) of oxalate. In some embodiments, the genetically engineered bacteria comprising genetic modification that increases import of oxalate into the bacterial cell comprise gene sequence(s) encoding an oxalate transporter or other metabolite transporter or an antiporter, e.g. an oxalate:formate antiporter, that transports oxalate into the bacterial cell. The transporter can be any transporter that assists or allows import of oxalate into the cell. In certain embodiments, the oxalate transporter is antiporter, e.g. an oxalate:formate antiporter, e.g., OxlT, e.g. from O. formigenes. In certain embodiments, the engineered bacterial cell contains gene sequence encoding OxlT, e.g. from O. formigenes. In some embodiments, the engineered bacteria comprise more than one copy of gene sequence encoding an oxalate transporter, e.g., an oxalate:formate antiporter, e.g., OxlT, e.g. from O. formigenes. In some embodiments, the engineered bacteria comprise gene sequence(s) encoding more than one oxalate transporter, e.g., two or more different oxalate transporters.

As used herein, the term “transporter” is meant to refer to a mechanism, e.g., protein, proteins, or protein complex, for importing a molecule, e.g., amino acid, peptide (di-peptide, tri-peptide, polypeptide, etc.), toxin, metabolite, substrate, as well as other biomolecules into the microorganism from the extracellular milieu. As used herein, the term “transporter” also includes antiporters, which can import and export metabolites, e.g. such as oxalate:formate antiporters described herein. As used herein, the terms “transporter” and “importer” are used equivalently.

The term “oxalate” as used herein, refers to the dianion of the formula C₂O₄²⁻. Oxalate is the conjugate base of oxalic acid. The term “oxalic acid,” as used herein, refers to a dicarboxylic acid with the chemical formula H₂C₂O₄.

As used herein, the phrase “exogenous environmental condition” or “exogenous environment signal” refers to settings, circumstances, stimuli, or biological molecules under which a promoter described herein is directly or indirectly induced. The phrase “exogenous environmental conditions” is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, the exogenous environmental conditions are low-oxygen, microaerobic, or anaerobic conditions, such as the environment of the mammalian gut. In some embodiments, exogenous environmental conditions are molecules or metabolites that are specific to the mammalian gut, e.g., propionate. In some embodiments, the exogenous environmental condition is a tissue-specific or disease-specific metabolite or molecule(s). In some embodiments, the exogenous environmental condition is specific to a disease, e.g., hyperoxaluria. In some embodiments, the exogenous environmental condition is a low-pH environment. In some embodiments, the genetically engineered microorganism of the disclosure comprises a pH-dependent promoter. In some embodiments, the genetically engineered microorganism of the disclosure comprise an oxygen level-dependent promoter. In some aspects, bacteria have evolved transcription factors that are capable of sensing oxygen levels. Different signaling pathways may be triggered by different oxygen levels and occur with different kinetics. An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR (fumarate and nitrate reductase), ANR, and DNR. Corresponding FNR-responsive promoters, ANR (anaerobic nitrate respiration)-responsive promoters, and DNR (dissimilatory nitrate respiration regulator)-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 1.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic conditions by the global transcriptional regulator FNR that is naturally found in Nissle. Under anaerobic conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrs, FNR, P-FNRS promoter and other such related designations to indicate the promoter PfnrS.

TABLE 1
Examples of transcription factors and
responsive genes and regulatory regions

| | Examples of responsive genes, promoters, |
|---|---|
| Transcription Factor | and/or regulatory regions: |
| FNR | nirB, ydfZ, pdhR, focA, ndH, hlyE, narK, |
| | narX, narG, yfiD, tdcD |
| ANR | arcDABC |
| DNR | norb, norC |

In some embodiments, the exogenous environmental conditions are in the presence or absence of reactive oxygen species (ROS). In other embodiments, the exogenous environmental conditions are the presence or absence of reactive nitrogen species (RNS). In some embodiments, exogenous environmental conditions are biological molecules that are involved in the inflammatory response, for example, molecules present in an inflammatory disorder of the gut. In some embodiments, the exogenous environmental conditions or signals exist naturally or are naturally absent in the environment in which the recombinant bacterial cell resides. In some embodiments, the exogenous environmental conditions or signals are artificially created, for example, by the creation or removal of biological conditions and/or the administration or removal of biological molecules.

In some embodiments, the exogenous environmental condition(s) and/or signal(s) stimulates the activity of an inducible promoter. In some embodiments, the exogenous environmental condition(s) and/or signal(s) that serves to activate the inducible promoter is not naturally present within the gut of a mammal. In some embodiments, the inducible promoter is stimulated by a molecule or metabolite that is administered in combination with the pharmaceutical composition of the disclosure, for example, tetracycline, arabinose, or any biological molecule that serves to activate an inducible promoter. In some embodiments, the exogenous environmental condition(s) and/or signal(s) is added to culture media comprising a recombinant bacterial cell of the disclosure. In some embodiments, the exogenous environmental condition that serves to activate the inducible promoter is naturally present within the gut of a mammal (for example, low oxygen or anaerobic conditions, or biological molecules involved in an inflammatory response). In some embodiments, the loss of exposure to an exogenous environmental condition (for example, in vivo) inhibits the activity of an inducible promoter, as the exogenous environmental condition is not present to induce the promoter (for example, an aerobic environment outside the gut). “Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

“Microorganism” refers to an organism or microbe of microscopic, submicroscopic, or ultramicroscopic size that typically consists of a single cell. Examples of microorganisms include bacteria, viruses, parasites, fungi, certain algae, and protozoa. In some aspects, the microorganism is engineered (“engineered microorganism”) to produce one or more therapeutic molecules, e.g., oxalate catabolism enzyme(s). In certain embodiments, the engineered microorganism is an engineered bacterium. In certain embodiments, the engineered microorganism is an engineered virus.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria do not contain lipopolysaccharides (LPS). In some embodiments, non-pathogenic bacteria are commensal bacteria. Examples of non-pathogenic bacteria include, but are not limited to certain strains belonging to the genus Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Escherichia coli Nissle, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis and Saccharomyces boulardii (Sonnenborn et al., 2009; Dinleyici et al., 2014; U.S. Pat. Nos. 6,835,376; 6,203,797; 5,589,168; 7,731,976). Non-pathogenic bacteria also include commensal bacteria, which are present in the indigenous microbiota of the gut. In one embodiment, the disclosure further includes non-pathogenic Saccharomyces, such as Saccharomyces boulardii. Naturally pathogenic bacteria may be genetically engineered to reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. In some embodiments, the probiotic bacteria are Gram-negative bacteria. In some embodiments, the probiotic bacteria are Gram-positive bacteria. Some species, strains, and/or subtypes of non-pathogenic bacteria are currently recognized as probiotic bacteria. Examples of probiotic bacteria include, but are not limited to, certain strains belonging to the genus Bifidobacteria, Escherichia coli, Lactobacillus, and Saccharomyces e.g., Bifidobacterium bifidum, Enterococcus faecium, Escherichia coli strain Nissle, Lactobacillus acidophilus, Lactobacillus bulgaricus, Lactobacillus paracasei, and Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, the term “auxotroph” or “auxotrophic” refers to an organism that requires a specific factor, e.g., an amino acid, a sugar, or other nutrient) to support its growth. An “auxotrophic modification” is a genetic modification that causes the organism to die in the absence of an exogenously added nutrient essential for survival or growth because it is unable to produce said nutrient. As used herein, the term “essential gene” refers to a gene which is necessary to for cell growth and/or survival. Essential genes are described in more detail infra and include, but are not limited to, DNA synthesis genes (such as thyA), cell wall synthesis genes (such as dapA), and amino acid genes (such as serA and metA).

As used herein, the terms “modulate” and “treat” and their cognates refer to an amelioration of a disease, disorder, and/or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease, disorder, and/or condition, either physically (e.g., stabilization of a discernible symptom), physiologically (e.g., stabilization of a physical parameter), or both. In another embodiment, “modulate” and “treat” refer to slowing the progression or reversing the progression of a disease, disorder, and/or condition. As used herein, “prevent” and its cognates refer to delaying the onset or reducing the risk of acquiring a given disease, disorder and/or condition or a symptom associated with such disease, disorder, and/or condition.

Those in need of treatment may include individuals already having a particular medical disease, as well as those at risk of having, or who may ultimately acquire the disease. The need for treatment is assessed, for example, by the presence of one or more risk factors associated with the development of a disease, the presence or progression of a disease, or likely receptiveness to treatment of a subject having the disease. Disorders in which oxalate is detrimental, e.g., a hyperoxaluria, may be caused by inborn genetic mutations for which there are no known cures. Diseases can also be secondary to other conditions, e.g., an intestinal disorder. Treating diseases in which oxalate is detrimental, such as a primary hyperoxaluria or secondary hyperoxaluria, may encompass reducing normal levels of oxalate and/or oxalic acid, reducing excess levels of oxalate and/or oxalic acid, or eliminating oxalate, and/or oxalic acid, and does not necessarily encompass the elimination of the underlying disease.

As used herein, the term “catabolism” refers to the cellular uptake of oxalate, and/or degradation of oxalate into its corresponding oxalyl CoA, and/or the degradation of oxalyl CoA formate and carbon dioxide. In one embodiment, the cellular uptake of oxalate occurs in the kidney. In one embodiment, the cellular uptake occurs in the liver. In one embodiment, the cellular uptake of oxalate occurs in the intestinal tract. In one embodiment, the cellular uptake of oxalate occurs in the stomach. In one embodiment, the cellular uptake is mediated by a SLC26 transporting protein (see Robijn et al. (2011)). In one embodiment, the cellular uptake is mediated by the transport protein SLC26A1. In one embodiment, the cellular uptake is mediated by the transport protein SLC26A6. In one embodiment, the cellular uptake of oxalate is mediated by a paracellular transport system. In one embodiment, the cellular uptake of oxalate is mediated by a transcellular transport system.

In one embodiment, “abnormal catabolism” refers to a decrease in the rate of cellular uptake of oxalate. In one embodiment, “abnormal catabolism” refers to any condition(s), disorder(s), disease(s), predisposition(s), and/or genetic mutations(s) that result in daily urinary oxalate excretion over 40 mg per 24 hours. In one embodiment, “abnormal catabolism” refers to an inability and/or decreased capacity of an organ and/or system to process and/or mediate the cellular uptake of oxalate. In one embodiment, said inability or decreased capacity of an organ and/or system to process and/or mediate the cellular uptake of oxalate is caused by the increased endogenous production of oxalate. In one embodiment, increased endogenous production of oxalate results from the absence of, or a deficiency in, the peroxisomal liver enzyme AGT. In one embodiment, increased endogenous production of oxalate results from the absence of, or a deficiency in the enzyme GRHPR. In one embodiment, increased endogenous production of oxalate results from the absence of, or a deficiency in the enzyme 4-hydroxy-2-oxoglutarate aldolase. In one embodiment, said inability or decreased capacity of an organ and/or system to process and/or mediate the cellular uptake of oxalate is caused by increased absorption of oxalate. In one embodiment, said increased absorption of oxalate results from an increased dietary intake of oxalate. In one embodiment, said increased absorption of oxalate results from increased intestinal absorption of oxalate. In one embodiment, said increased absorption of oxalate results from excessive intake of oxalate precursors. In one embodiment, said increased absorption of oxalate results from a decrease in intestinal oxalate-degrading microorganisms. In one embodiment, said increased absorption of oxalate results from genetic variations of intestinal oxalate transporters.

In one embodiment, a “disorder in which oxalate is detrimental” is a disease or disorder involving the abnormal, e.g., increased, levels of oxalate and/or oxalic acid or molecules directly upstream, such as glyoxylate. In one embodiment, the disorder in which oxalate is detrimental is a disorder or disease in which hyperoxaluria is observed in the subject. In one embodiment the disorder in which oxalate is detrimental refers to any condition(s), disorder(s), disease(s), predisposition(s), and/or genetic mutations(s) that result in daily urinary oxalate excretion over 40 mg per 24 hours. In one embodiment the disorder in which oxalate is detrimental is a disorder or disease selected from the group consisting of: PHI, PHII, PHII, secondary hyperoxaluria, enteric hyperoxaluria, syndrome of bacterial overgrowth, Crohn's disease, inflammatory bowel disease, hyperoxaluria following renal transplantation, hyperoxaluria after a jejunoileal bypass for obesity, hyperoxaluria after gastric ulcer surgery, chronic mesenteric ischemia, gastric bypass, cystic fibrosis, short bowel syndrome, biliary/pancreatic diseases (e.g., chronic pancreatitis).

As used herein a “pharmaceutical composition” refers to a preparation of genetically engineered microorganism of the disclosure, e.g., genetically engineered bacteria or virus, with other components such as a physiologically suitable carrier and/or excipient. In one embodiment, the pharmaceutical composition is a frozen liquid composition. In another embodiment, the pharmaceutical composition is a lyophilized composition.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial or viral compound. An adjuvant is included under these phrases.

The term “excipient” refers to an inert substance added to a pharmaceutical composition to further facilitate administration of an active ingredient. Examples include, but are not limited to, calcium bicarbonate, sodium bicarbonate calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition, e.g., a disorder in which oxalate is detrimental. A therapeutically effective amount may, for example, be sufficient to treat, prevent, reduce the severity, delay the onset, and/or reduce the risk of occurrence of one or more symptoms of a disease or condition associated with daily urinary oxalate excretion over 40 mg per 24 hours. A therapeutically effective amount, as well as a therapeutically effective frequency of administration, can be determined by methods known in the art and discussed below.

As used herein, the term “bacteriostatic” or “cytostatic” refers to a molecule or protein which is capable of arresting, retarding, or inhibiting the growth, division, multiplication or replication of recombinant bacterial cell of the disclosure.

As used herein, the term “bactericidal” refers to a molecule or protein which is capable of killing the recombinant bacterial cell of the disclosure.

As used herein, the term “toxin” refers to a protein, enzyme, or polypeptide fragment thereof, or other molecule which is capable of arresting, retarding, or inhibiting the growth, division, multiplication or replication of the recombinant bacterial cell of the disclosure, or which is capable of killing the recombinant bacterial cell of the disclosure. The term “toxin” is intended to include bacteriostatic proteins and bactericidal proteins. The term “toxin” is intended to include, but not limited to, lytic proteins, bacteriocins (e.g., microcins and colicins), gyrase inhibitors, polymerase inhibitors, transcription inhibitors, translation inhibitors, DNases, and RNases. The term “anti-toxin” or “antitoxin,” as used herein, refers to a protein or enzyme which is capable of inhibiting the activity of a toxin. The term anti-toxin is intended to include, but not limited to, immunity modulators, and inhibitors of toxin expression. Examples of toxins and antitoxins are known in the art and described in more detail infra.

As used herein, the term “oxalate catabolic or catabolism enzyme” or “oxalate catabolic or catabolism enzyme” or “oxalate metabolic enzyme” refers to any enzyme that is capable of metabolizing oxalate or capable of reducing accumulated oxalate or that can lessen, ameliorate, or prevent one or more diseases, or disease symptoms in which oxalate is detrimental. Examples of oxalate enzymes include, but are not limited to, formyl-CoA:oxalate CoA-transferase (also called formyl-CoA transferase), e.g., Frc from O. formigenes, oxalyl-CoA synthetase (also called oxalate-CoA ligase), e.g., Saccharomyces cerevisiae acyl-activating enzyme 3 (ScAAE3) from Saccharomyces cerevisiae, Oxalyl-CoA Decarboxylase, e.g., Oxc from O. formigenes (also referred to herein is oxdC or oxalate decarboxylase), acetyl-CoA:oxalate CoA-transferase (ACOCT), e.g., YfdE from E. coli and any other enzymes that catabolizes oxalate, oxalyl-CoA or any other metabolite thereof. Catabolism enzymes also include alanine glyoxalate aminotransferase (AGT, encoded by the AGXT gene, e.g. the human form), glyoxylate/hydroxypyruvate reductase (GRHPR; an enzyme having glyoxylate reductase (GR), hydroxypyruvate reductase (HPR), and D-glycerate dehydrogenase (DGDH) activities, e.g., the human form), and 4-hydroxy 2-oxoglutarate aldolase (encoded by the HOGA1 gene, e.g. in humans, and which breaks down 4-hydroxy 2-oxoglutarate into pyruvate and glyoxalate). Functional deficiencies in these proteins result in the accumulation of oxalate or its corresponding α-keto acid in cells and tissues. Oxalate metabolic enzymes of the present disclosure include both wild-type or modified oxalate metabolic enzymes and can be produced using recombinant and synthetic methods or purified from nature sources. Oxalate metabolic enzymes include full-length polypeptides and functional fragments thereof, as well as homologs and variants thereof. oxalate metabolic enzymes include polypeptides that have been modified from the wild-type sequence, including, for example, polypeptides having one or more amino acid deletions, insertions, and/or substitutions and may include, for example, fusion polypeptides and polypeptides having additional sequence, e.g., regulatory peptide sequence, linker peptide sequence, and other peptide sequence.

As used herein, the term “conventional hyperoxaluria treatment” or “conventional hyperoxaluria therapy” refers to treatment or therapy that is currently accepted, considered current standard of care, and/or used by most healthcare professionals for treating a disease or disorders in which oxalate is detrimental. It is different from alternative or complementary therapies, which are not as widely used.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the genetically engineered bacteria or virus of the current invention. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: -Ala, Pro, Gly, Gln, Asn, Ser, Thr; -Cys, Ser, Tyr, Thr; -Val, Ile, Leu, Met, Ala, Phe; -Lys, Arg, His; -Phe, Tyr, Trp, His; and -Asp, Glu.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the invention. Such variants generally retain the functional activity of the peptides of the present invention. Variants include peptides that differ in amino acid sequence from the native and wt peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety.

As used herein the term “codon-optimized” refers to the modification of codons in the gene or coding regions of a nucleic acid molecule to reflect the typical codon usage of the host organism without altering the polypeptide encoded by the nucleic acid molecule. Such optimization includes replacing at least one, or more than one, or a significant number, of codons with one or more codons that are more frequently used in the genes of the host organism. A “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Bacteria

The genetically engineered microorganisms, or programmed microorganisms, such as genetically engineered bacteria of the disclosure are capable of producing one or more enzymes for metabolizing an oxalate and/or a metabolite thereof. In some aspects, the disclosure provides a bacterial cell that comprises one or more heterologous gene sequence(s) encoding an oxalate catabolism enzyme or other protein that results in a decrease in oxalate levels.

In certain embodiments, the genetically engineered bacteria are obligate anaerobic bacteria. In certain embodiments, the genetically engineered bacteria are facultative anaerobic bacteria. In certain embodiments, the genetically engineered bacteria are aerobic bacteria. In some embodiments, the genetically engineered bacteria are Gram-positive bacteria. In some embodiments, the genetically engineered bacteria are Gram-positive bacteria and lack LPS. In some embodiments, the genetically engineered bacteria are Gram-negative bacteria. In some embodiments, the genetically engineered bacteria are Gram-positive and obligate anaerobic bacteria. In some embodiments, the genetically engineered bacteria are Gram-positive and facultative anaerobic bacteria. In some embodiments, the genetically engineered bacteria are non-pathogenic bacteria. In some embodiments, the genetically engineered bacteria are commensal bacteria. In some embodiments, the genetically engineered bacteria are probiotic bacteria. In some embodiments, the genetically engineered bacteria are naturally pathogenic bacteria that are modified or mutated to reduce or eliminate pathogenicity. Exemplary bacteria include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium cochlearum, Clostridiumfelsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera. In certain embodiments, the genetically engineered bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Oxalobacter formigenes bacterial cell. Saccharomyces boulardii. In certain embodiments, the genetically engineered bacteria are selected from Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coli, Escherichia coli Nissle, Lactobacillus acidophilus, Lactobacillus plantarum, Lactobacillus reuteri, and Lactococcus lactis bacterial cell. In one embodiment, the bacterial cell is a Bacteroides fragilis bacterial cell. In one embodiment, the bacterial cell is a Bacteroides thetaiotaomicron bacterial cell. In one embodiment, the bacterial cell is a Bacteroides subtilis bacterial cell. In one embodiment, the bacterial cell is a Bifidobacterium bifidum bacterial cell. In one embodiment, the bacterial cell is a Bifidobacterium infantis bacterial cell. In one embodiment, the bacterial cell is a Bifidobacterium lactis or B. infantis bacterial cell. In one embodiment, the bacterial cell is a Clostridium butyricum bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment the bacterial cell is a Bifidobacterium lactis bacterial cell. In one embodiment, the bacterial cell is a Clostridium butyricum bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment, the bacterial cell is a Lactobacillus reuteri bacterial cell. In one embodiment, the bacterial cell is a Lactococcus lactis bacterial cell. In one embodiment, the bacterial cell is a Oxalobacter formigenes bacterial cell. In another embodiment, the bacterial cell does not include Oxalobacter formigenes.

In some embodiments, the genetically engineered bacteria are Escherichia coli strain Nissle 1917 (E. coli Nissle), a Gram-negative bacterium of the Enterobacteriaceae family that has evolved into one of the best characterized probiotics (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli α-hemolysin, P-fimbrial adhesins) (Schultz, 2008). In addition, it has been shown that E. coli Nissle does not carry pathogenic adhesion factors, does not produce any enterotoxins or cytotoxins, is not invasive, and not uropathogenic (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's therapeutic efficacy and safety have convincingly been proven (Ukena et al., 2007).

In one embodiment, the recombinant bacterial cell of the invention does not colonize the subject having the disorder in which oxalate is detrimental.

One of ordinary skill in the art would appreciate that the genetic modifications disclosed herein may be adapted for other species, strains, and subtypes of bacteria. Furthermore, genes from one or more different species can be introduced into one another, e.g., an oxalate catabolism gene from Lactococcus lactis can be expressed in Escherichia coli. Unmodified E. coli Nissle and the genetically engineered bacteria of the invention may be destroyed, e.g., by defense factors in the gut or blood serum (Sonnenborn et al., 2009). In some embodiments, the residence time is calculated for a human subject. In some embodiments, residence time in vivo is calculated for the genetically engineered bacteria disclosed herein.

In some embodiments, the bacterial cell is a genetically engineered bacterial cell. In another embodiment, the bacterial cell is a recombinant bacterial cell. In some embodiments, the disclosure comprises a colony of bacterial cells disclosed herein.

In another aspect, the disclosure provides a recombinant bacterial culture which comprises bacterial cells disclosed herein. In one aspect, the disclosure provides a recombinant bacterial culture which reduces levels of oxalate or oxalic acid in the media of the culture. In one embodiment, the levels of the oxalate or oxalic acid are reduced by about 50%, about 75%, or about 100% in the media of the cell culture after a period of time, e.g., 1 hour, e.g., under inducing conditions. In another embodiment, the levels of the oxalate or oxalic acid are reduced by about two-fold, three-fold, four-fold, five-fold, six-fold, seven-fold, eight-fold, nine-fold, or ten-fold in the media of the cell culture after a period of time, e.g., 1 hour, e.g., under inducing conditions. In one embodiment, the levels of the oxalate or oxalic acid are reduced below the limit of detection in the media of the cell culture.

In some embodiments of the above described genetically engineered bacteria, the gene and/or gene cassette encoding one or more oxalate catabolism enzyme(s) is present on a plasmid in the bacterium. In some embodiments of the above described genetically engineered bacteria, the gene and/or gene cassette encoding one or more oxalate catabolism enzyme(s) is present on a plasmid in the bacterium and operatively linked on the plasmid to a promoter that is induced under low-oxygen or anaerobic conditions, such as any of the promoters disclosed herein. In other embodiments, the gene and/or gene cassette encoding one or more oxalate catabolism enzyme(s) is present in the bacterial chromosome. In other embodiments, the gene and/or gene cassette encoding one or more oxalate catabolism enzyme(s) is present in the bacterial chromosome and is operatively linked in the chromosome to the promoter that is induced under low-oxygen or anaerobic conditions, such as any of the promoters disclosed herein. In some embodiments of the above described genetically engineered bacteria, the gene and/or gene cassette encoding one or more oxalate catabolism enzyme(s) is present on a plasmid in the bacterium and operatively linked on the plasmid to the promoter that is induced under inflammatory conditions, such as any of the promoters disclosed herein. In other embodiments, the gene and/or gene cassette encoding one or more oxalate catabolism enzyme(s) is present in the bacterial chromosome and is operatively linked in the chromosome to the promoter that is induced under inflammatory conditions, such as any of the promoters disclosed herein.

In some embodiments, the genetically engineered bacteria comprising the gene and/or gene cassette encoding one or more oxalate catabolism enzyme(s) further comprise gene sequence(s) encoding an oxalate transporter. In some embodiments, the genetically engineered bacteria comprising the gene and/or gene cassette encoding one or more oxalate catabolism enzyme(s) further comprise gene sequence(s) encoding a formate exporter. In some embodiments, the genetically engineered bacteria comprising the gene and/or gene cassette encoding one or more oxalate catabolism enzyme(s) further comprise gene sequence(s) encoding an oxalate:formate antiporter. In some embodiments, the genetically engineered bacteria comprising the gene and/or gene cassette encoding one or more oxalate catabolism enzyme(s) further comprise gene sequence(s) encoding one or more of the following: an oxalate transporter, a formate exporter, and/or an oxalate:formate antiporter.

In some embodiments, the genetically engineered bacteria comprising the gene and/or gene cassette encoding one or more oxalate catabolism enzyme(s) and/or oxalate transporter, and/or formate exporter, and/or oxalate:formate antiporter is an auxotroph. In one embodiment, the genetically engineered bacteria is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapf, flhD, metB, metC, proAB, and thi1 auxotroph. In some embodiments, the engineered bacteria have more than one auxotrophy, for example, they may be a ΔthyA and ΔdapA auxotroph.

In some embodiments, the genetically engineered bacteria comprising the gene and/or gene cassette encoding one or more oxalate catabolism enzyme(s) and/or oxalate transporter, and/or formate exporter, and/or oxalate:formate antiporter further comprise gene sequence(s) encoding a secretion protein or protein complex for secreting a biomolecule, such as any of the secretion systems disclosed herein.

In some embodiments, the genetically engineered bacteria comprising the gene and/or gene cassette encoding one or more oxalate catabolism enzyme(s) and/or oxalate transporter, and/or formate exporter, and/or oxalate:formate antiporter further comprise gene sequence(s) encoding one or more antibiotic gene(s), such as any of the antibiotic genes disclosed herein.

In some embodiments, the genetically engineered bacteria comprising a gene and/or gene cassette encoding one or more oxalate catabolism enzyme(s) and/or oxalate transporter, and/or formate exporter, and/or oxalate:formate antiporter further comprise a kill-switch circuit, such as any of the kill-switch circuits provided herein. For example, in some embodiments, the genetically engineered bacteria further comprise one or more genes encoding one or more recombinase(s) under the control of an inducible promoter, and an inverted toxin sequence. In some embodiments, the genetically engineered bacteria further comprise one or more genes encoding an antitoxin. In some embodiments, the engineered bacteria further comprise one or more genes encoding one or more recombinase(s) under the control of an inducible promoter and one or more inverted excision genes, wherein the excision gene(s) encode an enzyme that deletes an essential gene. In some embodiments, the genetically engineered bacteria further comprise one or more genes encoding an antitoxin. In some embodiments, the engineered bacteria further comprise one or more genes encoding a toxin under the control of a promoter having a TetR repressor binding site and a gene encoding the TetR under the control of an inducible promoter that is induced by arabinose, such as P_{araBAD}. In some embodiments, the genetically engineered bacteria further comprise one or more genes encoding an antitoxin.

In some embodiments, the genetically engineered bacteria is an auxotroph comprising the gene and/or gene cassette encoding one or more oxalate catabolism enzyme(s) and further comprises a kill-switch circuit, such as any of the kill-switch circuits described herein.

In some embodiments of the above described genetically engineered bacteria, the gene and/or gene cassette encoding one or more oxalate catabolism enzyme(s) is present on a plasmid in the bacterium. In some embodiments, the gene and/or gene cassette encoding one or more oxalate catabolism enzyme(s) is present in the bacterial chromosome. In some embodiments, the genetically engineered bacteria comprise one or more gene and/or gene cassette(s) encoding one or more oxalate transporter(s) that transports oxalate into the bacterial cell. In some embodiments, the gene sequence(s) encoding an oxalate transporter is present on a plasmid in the bacterium. In some embodiments, the gene sequence(s) encoding an oxalate transporter is present in the bacterial chromosome. In some embodiments, the gene sequence encoding a secretion protein or protein complex for secreting a biomolecule, such as any of the secretion systems disclosed herein, is present on a plasmid in the bacterium. In some embodiments, the gene sequence encoding a secretion protein or protein complex for secreting a biomolecule, such as any of the secretion systems disclosed herein, is present in the bacterial chromosome. In some embodiments, the gene sequence(s) encoding an antibiotic resistance gene is present on a plasmid in the bacterium. In some embodiments, the gene sequence(s) encoding an antibiotic resistance gene is present in the bacterial chromosome.

Oxalate Catabolism Enzymes

O. formigenes was the first oxalate-degrading obligate anaerobe to be described in humans and has served as the paradigm organism in which anaerobic oxalate degradation has been studied. O. formigenes has three enzymes involved in the catabolism of oxalic acid. First extracellular oxalate is taken up by the membrane-associated oxalate-formate antiporter, OxlT, encoded by the oxlT gene. The frc gene encodes formyl-CoA transferase, Frc, which activates the intracellular oxalate to form oxalyl-CoA. This is decarboxylated in a thiamine PPi-dependent reaction by the oxalyl-CoA decarboxylase, Oxc, enzyme, expressed from the oxc gene. Formate and carbon dioxide are the end products, and the oxalate-formate antiporter, OxlT, catalyzes the export of the intracellular formate out of the cells. In O. formigenes, the generation of energy is coupled to oxalate transport, mediated by the oxalate transport membrane protein OxlT, (as described in Abratt and Reid Oxalate-Degrading Bacteria of the Human Gut as Probiotics in the Management of Kidney Stone Disease, the contents of which is herein incorporated by reference in its entirety, and references therein).

As used herein, the term “oxalate catabolism enzyme” refers to an enzyme involved in the catabolism of oxalate to its corresponding oxalyl-CoA molecule, the catabolism of oxalyl-CoA to formate and carbon dioxide, or the catabolism of oxalate to another metabolite. Enzymes involved in the catabolism of oxalate are well known to those of skill in the art. For example, in the obligate anaerobe Oxalobacter formigenes, the formyl coenzyme A transferase FRC (encoded by the frc gene) transfers a coenzyme A moiety to oxalic acid, forming oxalyl-CoA (see, e.g., Sidhu et al., J. Bacteriol. 179: 3378-81 (1997), the entire contents of which are expressly incorporated herein by reference). Subsequently, the oxalyl-CoA is subject to a reaction mediated by the oxalyl-CoA decarboxylase OXC (encoded by the oxc gene), which leads to the formation of formate and carbon dioxide (see, e.g., Lung et al., J. Bacteriol. 176: 2468-72 (1994), the entire contents of which are expressly incorporated herein by reference). Further, the E. coli protein YfdW (Protein Data Bank Accession No. 1pt5) and YfdU (Protein Data Bank Accession No. E0SNC8) are a formyl-CoA transferase and an oxalyl-CoA decarboxylase that have been shown to be functional homologs of the O. formigenes FRC and OXC enzymes (see, e.g., Toyota et al., J. Bact. 190: 2256-64 (2008); Werther et al., FEBS J. 277: 2628-40 (2010); Fontenot et al., J. Bact. 195: 1446-55 (2013)).

Another oxalate catabolism enzyme, acetyl-CoA:oxalate CoA-transferase, converts acetyl-CoA and oxalate to oxalyl-CoA and acetate. In a non-limiting example, the acetyl-CoA:oxalate CoA-transferase is YfdE from E. coli (e.g., described in Function and X-ray crystal structure of Escherichia coli YfdE; PLoS One. 2013 Jul. 23; 8(7):e67901). Acetyl-CoA substrate a very ubiquitous metabolite in bacteria, such as E. coli, and acetate produced can for example diffuse into the extracellular space without the need of a transporter. In one example, acetyl-CoA:oxalate CoA-transferase reaction can be followed by oxalyl-CoA decarboxylase OXC (encoded by the oxc gene), which leads to the formation of formate and carbon dioxide. Formate can exit the cell, for example through a formate exporter, including but not limited to, OxlT from O. formigenes.

Another exemplary oxalate catabolism enzyme oxalyl-CoA synthetase (OCL; also called oxalate-CoA ligase), which converts oxalate and CoA and ATP to oxalyl-CoA and AMP and diphosphate. In a non-limiting example, the oxalate-CoA ligase is Saccharomyces cerevisiae acyl-activating enzyme 3 (ScAAE3) (e.g., described in Foster and Nakata, An oxalyl-CoA synthetase is important for oxalate metabolism in Saccharomyces cerevisiae. FEBS Lett. 2014 Jan. 3; 588(1):160-6). In one example, oxalate-CoA ligase can be followed by oxalyl-CoA decarboxylase OXC (encoded by the oxc gene), which leads to the formation of formate and carbon dioxide. Formate can exit the cell, for example through a formate exporter, including but not limited to, OxlT from O. formigenes.

In some embodiments, the genetically engineered bacteria of the disclosure comprise one or more gene(s) and/or gene cassette(s) encoding at least one oxalate catabolism enzyme. In some embodiments, the engineered bacteria comprise one or more gene(s) and/or gene cassette(s) encoding at least one oxalate catabolism enzyme and are capable of converting oxalate into oxalyl-CoA. In some embodiments, the engineered bacteria comprise one or more gene(s) and/or gene cassette(s) encoding at least one oxalate catabolism enzyme and are capable of converting oxalyl-CoA into formate and carbon dioxide. In some embodiments, the engineered bacteria comprise one or more gene(s) and/or gene cassette(s) encoding at least one oxalate catabolism enzyme and are capable of converting oxalate into oxalyl-CoA, and oxalyl-CoA into formate and carbon dioxide. In some embodiments, the engineered bacteria of the disclosure comprise one or more gene(s) and or gene cassette encoding one or more oxalate catabolism enzyme(s) which convert oxalate and formyl CoA into oxalyl-CoA and formate. In some embodiments, the engineered bacteria of the disclosure comprise one or more gene(s) and/or gene cassette(s) encoding one or more oxalate catabolism enzyme(s) which convert oxalate and acetyl-coA into oxalyl-CoA and acetate. In some embodiments, the engineered bacteria of the disclosure comprise one or more gene(s) and/or gene cassette(s) encoding one or more oxalate catabolism enzyme(s) which convert oxalate and CoA into oxalyl-CoA (e.g., by converting one ATP to AMP plus diphosphate). In some embodiments, the engineered bacteria of the disclosure comprise one or more gene(s) and/or gene cassette(s) encoding one or more oxalate catabolism enzyme(s) which convert oxalyl-CoA to carbon dioxide and formyl-CoA. In some embodiments, the engineered bacteria produce formate as a result of oxalate catabolism. In some embodiments, the engineered bacteria produce formate and carbon dioxide as a result of oxalate catabolism. In some embodiments, the engineered bacteria produce acetate as a result of oxalate catabolism. In some embodiments, the engineered bacteria produce acetate and carbon dioxide as a result of oxalate catabolism. In some embodiments, the engineered bacteria produce formate, acetate, and carbon dioxide as a result of oxalate catabolism.

In some embodiments, the genetically engineered bacteria comprise gene sequence(s) encoding one or more oxalate catabolism enzyme (s). In some embodiments, the one or more oxalate catabolism enzyme(s) increases the rate of oxalate and/or oxalyl-CoA catabolism in the cell. In some embodiments, the one or more oxalate catabolism enzyme(s) decreases the level of oxalate in the cell. In some embodiments, the one or more oxalate catabolism enzyme(s) decreases the level of oxalyl-CoA in the cell. In some embodiments, the one or more oxalate catabolism enzyme(s) decreases the level of oxalic acid in the cell.

In some embodiments, the one or more oxalate catabolism enzyme(s) increases the level of oxalyl-CoA in the cell as compared to the level of its corresponding oxalate in the cell. In some embodiments, the one or more oxalate catabolism enzyme(s) increases the level of formate and carbon dioxide in the cell as compared to the level of its corresponding oxalyl-CoA in the cell. In some embodiments, the one or more oxalate catabolism enzyme(s) decreases the level of the oxalate and/or oxalyl CoA as compared to the level of oxalate in the cell.

Enzymes involved in the catabolism of oxalate may be expressed or modified in the bacteria of the invention in order to enhance catabolism of oxalate. Specifically, when at least one oxalate catabolism enzyme is expressed in the engineered bacterial cells of the invention, the engineered bacterial cells convert more oxalate into oxalyl-CoA, or convert more oxalyl-CoA into formate and carbon dioxide when the catabolism enzyme is expressed than unmodified bacteria of the same bacterial subtype under the same conditions. Thus, the genetically engineered bacteria comprising a heterologous gene encoding an oxalate catabolism enzyme can catabolize oxalate and/or oxalyl-CoA to treat disorders in which oxalate is detrimental, such as PHI, PHII, PHIII, and secondary hyperoxaluria, enteric hyperoxaluria, and idiopathic hyperoxaluria.

In one embodiment, the bacterial cell of the invention comprises at least one heterologous gene encoding at least one oxalate catabolism enzyme. In one embodiment, the bacterial cell of the invention comprises at least one heterologous gene encoding an importer of oxalate and at least one heterologous gene encoding at least one oxalate catabolism enzyme. In one embodiment, the bacterial cell of the invention comprises at least one heterologous gene encoding an exporter of formate and at least one heterologous gene encoding at least one oxalate catabolism enzyme. In one embodiment, the bacterial cell of the invention comprises at least one heterologous gene encoding an oxalate:formate antiporter and at least one heterologous gene encoding at least one oxalate catabolism enzyme.

In some embodiments, the invention provides a bacterial cell that comprises at least one heterologous gene encoding at least one oxalate catabolism enzyme operably linked to a first promoter. In one embodiment, the bacterial cell comprises at least one gene encoding at least one oxalate catabolism enzyme from a different organism, e.g., a different species of bacteria. In another embodiment, the bacterial cell comprises more than one copy of a native gene encoding an oxalate catabolism enzyme. In yet another embodiment, the bacterial cell comprises at least one native gene encoding at least one oxalate catabolism enzyme, as well as at least one copy of at least one gene encoding an oxalate catabolism enzyme from a different organism, e.g., a different species of bacteria. In one embodiment, the bacterial cell comprises at least one, two, three, four, five, or six copies of a gene encoding an oxalate catabolism enzyme. In one embodiment, the bacterial cell comprises multiple copies of a gene encoding an oxalate catabolism enzyme.

Oxalate catabolism enzymes are known in the art. In some embodiments, AN oxalate catabolism enzyme is encoded by at least one gene encoding at least one oxalate catabolism enzyme derived from a bacterial species. In some embodiments, an oxalate catabolism enzyme is encoded by a gene encoding an oxalate catabolism enzyme derived from a non-bacterial species. In some embodiments, an oxalate catabolism enzyme is encoded by a gene derived from a eukaryotic species, e.g., a yeast species or a plant species. In one embodiment, an oxalate catabolism enzyme is encoded by a gene derived from a human. In one embodiment, the gene sequence(s) encoding the one or more oxalate catabolism enzyme(s) is derived from an organism of the genus or species that includes, but is not limited to, Bifidobacterium, Bordetella, Bradyrhizobium, Burkholderia, Clostridium, Enterococcus, Escherichia, Eubacterium, Lactobacillus, Magnetospirillium, Mycobacterium, Neurospora, Oxalobacter, e.g., Oxalobacter formigenes, Ralstonia, Rhodopseudomonas, Shigella, Thermoplasma, and Thauera, e.g., Bifidobacterium animalis, Bifidobacterium bifidum, Bifidobacterium infantis, Bifidobacterium lactis, Bifidobacterium longum, Bordatella bronchiseptica, Bordatella parapertussis, Burkholderia fungorum, Burkholderia xenovorans, Bradyrhizobium japonicum, Clostridium acetobutylicum, Clostridium difficile, Clostridium scindens, Clostridium sporogenes, Clostridium tentani, Enterococcus faecalis, Escherichia coli, Eubacterium lentum, Lactobacillus acidophilus, Lactobacillus bulgaricus, Lactobacillus casei, Lactobacillus gasseri, Lactobacillus plantarum, Lactobacillus rhamnosus, Lactococcus lactis, Magnetospirillium magentotaticum, Mycobacterium avium, Mycobacterium intracellulare, Mycobacterium kansasii, Mycobacterium leprae, Mycobacterium smegmatis, Mycobacterium tuberculosis, Mycobacterium ulcerans, Neurospora crassa, Oxalobacter formigenes, Providencia rettgeri, Eubacterium lentum, Ralstonia eutropha, Ralstonia metallidurans, Rhodopseudomonas palustris, Shigella flexneri, Thermoplasma volcanium, and Thauera aromatica.

In one embodiment, one or more oxalate catabolism enzyme(s) encoded by the genetically engineered bacteria are derived from O. formigenes, e.g., oxc and frc described above.

In one embodiment, one or more oxalate catabolism enzyme(s) encoded by the engineered bacteria are derived from Enterococcus faecalis. An inducible oxalate catabolism system has been described in Enterococcus faecalis, which comprised homologs to O. formigenes Frc and Oxc (Hokama et al., Oxalate-degrading Enterococcus faecalis. Microbiol. Immunol. 44, 235-240).

In one embodiment, one or more oxalate catabolism enzyme(s) encoded by the engineered bacteria are derived from are from Eubacterium lentum. The oxalate-degrading proteins oxalyl-CoA decarboxylase and formyl-CoA transferase were reportedly isolated from this strain (Ito, H., Kotake, T., and Masai, M. (1996). In vitro degradation of oxalic acid by human feces. Int. J. Urol. 3, 207-211.).

In one embodiment, one or more oxalate catabolism enzyme(s) encoded by the engineered bacteria are derived from Providencia rettgeri, which have shown to have homologs to O. formigenes Frc and Oxc (e.g., as described in Abratt and Reid, Oxalate-degrading bacteria of the human gut as probiotics in the management of kidney stone disease; Adv Appl Microbiol. 2010; 72:63-87, and references therein).

In one embodiment, one or more oxalate catabolism enzyme(s) encoded by the engineered bacteria are derived from E. coli, e.g. from the yfdXWUVE operon. For example, the ydfU is thought to be a oxc homolog. In one embodiment, one or more oxalate catabolism enzyme(s) encoded by the engineered bacteria are derived from Lactobacillus and/or Bifidobacterium species. In a non-limiting example one or more oxalate catabolism enzyme(s) are derived from oxc and frc homologs Lactobacillus and/or Bifidobacterium species. Non-limiting examples of such Lactobacillus species include Lactobacillus, plantarum, Lactobacillus brevis, Lactobacillus acidophilus, Lactobacillus casei, Lactobacillus gasseri, Lactobacillus rhamnosus, and Lactobacillus salivarius. Non-limiting examples of such Bifidobacterium species include Bifidobacterium infantis, Bifidobacterium animalis, Bifidobacterium breve, Bifidobacterium longum, Bifidobacterium lactis, and Bifidobacterium adolescentis.

In one embodiment, the gene sequence(s) encoding the one or more oxalate catabolism enzyme(s) has been codon-optimized for use in the recombinant bacterial cell of the invention. In one embodiment, the gene sequence(s) encoding the one or more oxalate catabolism enzyme(s) has been codon-optimized for use in Escherichia coli. In one embodiment, the gene sequence(s) encoding the one or more oxalate catabolism enzyme(s) has not been codon-optimized for use in Escherichia coli. In another embodiment, the gene sequence(s) encoding the one or more oxalate catabolism enzyme(s) has been codon-optimized for use in Lactococcus. When the gene sequence(s) encoding the one or more oxalate catabolism enzyme(s) is expressed in the recombinant bacterial cells of the invention, the bacterial cells catabolize more oxalate or oxalyl-CoA than unmodified bacteria of the same bacterial subtype under the same conditions (e.g., culture or environmental conditions). Thus, the genetically engineered bacteria comprising at least one heterologous gene encoding at least one oxalate catabolism enzyme may be used to catabolize excess oxalate, oxalic acid, and/or oxalyl-CoA to treat a disorder in which oxalate is detrimental, such as PHI, PHII, PHIII, and secondary hyperoxaluria, enteric hyperoxaluria, and idiopathic hyperoxaluria.

The present invention further comprises genes encoding functional fragments of an oxalate catabolism enzyme or functional variants of an oxalate catabolism enzyme. As used herein, the term “functional fragment thereof” or “functional variant thereof” of an oxalate catabolism enzyme relates to an element having qualitative biological activity in common with the wild-type oxalate catabolism enzyme from which the fragment or variant was derived. For example, a functional fragment or a functional variant of a mutated oxalate catabolism enzyme is one which retains essentially the same ability to catabolize oxalyl-CoA as the oxalate catabolism enzyme from which the functional fragment or functional variant was derived. For example, a polypeptide having oxalate catabolism enzyme activity may be truncated at the N-terminus or C-terminus and the retention of oxalate catabolism enzyme activity assessed using assays known to those of skill in the art, including the exemplary assays provided herein. In one embodiment, the recombinant bacterial cell of the invention comprises a heterologous gene encoding an oxalate catabolism enzyme functional variant. In another embodiment, the recombinant bacterial cell of the invention comprises a heterologous gene encoding an oxalate catabolism enzyme functional fragment.

Assays for testing the activity of an oxalate catabolism enzyme, an oxalate catabolism enzyme functional variant, or an oxalate catabolism enzyme functional fragment are well known to one of ordinary skill in the art. For example, oxalate catabolism can be assessed by expressing the protein, functional variant, or fragment thereof, in a recombinant bacterial cell that lacks endogenous oxalate catabolism enzyme activity. Oxalate catabolism activity can be assessed by quantifying oxalate degradation in the culture media as described by Federici et al., Appl. Environ. Microbiol. 70: 5066-73 (2004), the entire contents of which are expressly incorporated herein by reference. Formyl-CoA transferase and oxalyl-CoA decarboxylase activities can be measured by capillary electrophoresis as described in Turroni et al., J. Appl. Microbiol. 103: 1600-9 (2007).

As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.). In one embodiment, the gene or protein is at least 90%, 91%, 92%, 93%, 94$, 95%, 96%, 97%, 98%, 99% or 100% identical to a gene or protein disclosed herein.

The present invention encompasses genes encoding an oxalate catabolism enzyme comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T. Similarly, contemplated is replacing a basic amino acid with another basic amino acid (e.g., replacement among Lys, Arg, His), replacing an acidic amino acid with another acidic amino acid (e.g., replacement among Asp and Glu), replacing a neutral amino acid with another neutral amino acid (e.g., replacement among Ala, Gly, Ser, Met, Thr, Leu, Ile, Asn, Gln, Phe, Cys, Pro, Trp, Tyr, Val).

In some embodiments, the gene encoding an oxalate catabolism enzyme is mutagenized; mutants exhibiting increased activity are selected; and the mutagenized gene encoding the oxalate catabolism enzyme is isolated and inserted into the bacterial cell of the invention. In one embodiment, spontaneous mutants that arise that allow bacteria to grow on oxalate as the sole carbon source can be screened for and selected. The gene comprising the modifications described herein may be present on a plasmid or chromosome. Non-limiting examples of oxalate catabolism enzymes of the disclosure are listed in Table 2.

TABLE 2
Oxalate Catabolismenzyme Polynucleotide Sequences

| Description | SEQ ID NO |
|---|---|
| frc (formyl-CoA transferase from O. formigenes) | SEQ ID NO: 1 |
| oxc (oxalylCoA decarboxylase from O. formigenes) | SEQ ID NO: 2 |
| also referred to as oxdc (oxalate decarboxylase) | |
| ScAAE3 (oxalate-CoA ligase from S. cerevisiae) | SEQ ID NO: 3 |
| yfdE (Acetyl-CoA:oxalate CoA-transferase from E. coli) | SEQ ID NO: 4 |

In one embodiment, the gene sequence(s) encoding the one or more oxalate catabolism enzyme(s) comprises a formyl-CoA:oxalate CoA-transferase sequence. In one embodiment, the formyl-CoA:oxalate CoA-transferase is frc, e.g., from O. formigenes. Accordingly, in one embodiment, the frc gene has at least about 80% identity with the entire sequence of SEQ ID NO: 1. Accordingly, in one embodiment, the frc gene has at least about 90% identity with the entire sequence of SEQ ID NO: 1. Accordingly, in one embodiment, the frc gene has at least about 95% identity with the entire sequence of SEQ ID NO: 1. Accordingly, in one embodiment, the frc gene has at least about 85%, 86%, 87%, 88%, 89%, 90%, 91%, 92%, 93%, 94%, 95%, 96%, 97%, 98%, or 99% identity with the entire sequence of SEQ ID NO: 1. In another embodiment, the frc gene comprises the sequence of SEQ ID NO: 1. In yet another embodiment the frc gene consists of the sequence of SEQ ID NO:1.

In one embodiment, the gene sequence(s) encoding the one or more oxalate catabolism enzyme(s) comprises a oxalyl-CoA decarboxylase sequence. In one embodiment, the oxalyl-CoA decarboxylase is oxc, e.g., from O. formigenes. Accordingly, in one embodiment, the oxc gene has at least about 80% identity with the entire sequence of SEQ ID NO: 2. Accordingly, in one embodiment, the oxc gene has at least about 90% identity with the entire sequence of SEQ ID NO: 2. Accordingly, in one embodiment, the oxc gene has at least about 95% identity with the entire sequence of SEQ ID NO: 2. Accordingly, in one embodiment, the oxc gene has at least about 85%, 86%, 87%, 88%, 89%, 90%, 91%, 92%, 93%, 94%, 95%, 96%, 97%, 98%, or 99% identity with the entire sequence of SEQ ID NO: 2. In another embodiment, the oxc gene comprises the sequence of SEQ ID NO: 2. In yet another embodiment the oxc gene consists of the sequence of SEQ ID NO: 2. In another embodiment, the oxc gene consists of the sequence of SEQ ID NO: 2.

In one embodiment, the at least one gene encoding the at least one oxalate catabolism enzyme comprises an oxalate-CoA ligase sequence. In one embodiment, the oxalate-CoA ligase is ScAAE3 from S. cerevisiae. Accordingly, in one embodiment, the ScAAE3 gene has at least about 80% identity with the entire sequence of SEQ ID NO: 3. Accordingly, in one embodiment, the ScAAE3 gene has at least about 90% identity with the entire sequence of SEQ ID NO: 3. Accordingly, in one embodiment, the ScAAE3 gene has at least about 95% identity with the entire sequence of SEQ ID NO: 3. Accordingly, in one embodiment, the ScAAE3 gene has at least about 85%, 86%, 87%, 88%, 89%, 90%, 91%, 92%, 93%, 94%, 95%, 96%, 97%, 98%, or 99% identity with the entire sequence of SEQ ID NO: 3. In another embodiment, the ScAAE3 gene comprises the sequence of SEQ ID NO: 3. In yet another embodiment the ScAAE3 gene consists of the sequence of SEQ ID NO: 3.

In one embodiment, the at least one gene encoding the at least one oxalate catabolism enzyme comprises an acetyl-CoA:oxalate CoA-transferase sequence. In one embodiment, the acetyl-CoA:oxalate CoA-transferase is YfdE from E. coli from S. cerevisiae. Accordingly, in one embodiment, the YfdE gene has at least about 80% identity with the entire sequence of SEQ ID NO: 4. Accordingly, in one embodiment, the YfdE gene has at least about 90% identity with the entire sequence of SEQ ID NO: 4. Accordingly, in one embodiment, the YfdE gene has at least about 95% identity with the entire sequence of SEQ ID NO: 4. Accordingly, in one embodiment, the YfdE gene has at least about 85%, 86%, 87%, 88%, 89%, 90%, 91%, 92%, 93%, 94%, 95%, 96%, 97%, 98%, or 99% identity with the entire sequence of SEQ ID NO: 4. In another embodiment, the YfdE gene comprises the sequence of SEQ ID NO: 4. In yet another embodiment the YfdE gene consists of the sequence of SEQ ID NO: 4.

Table 3 lists non-limiting examples of oxalate catabolism enzyme polypeptide sequences.

TABLE 3
Polypeptide Sequences of Oxalate Catabolism Enzymes

| Description | SEQ ID NO |
|---|---|
| Frc (Formyl-CoA transferase from O. formigenes) | SEQ ID NO: 5 |
| Oxc (oxalylCoA decarboxylase from O. formigenes); | SEQ ID NO: 6 |
| also referred to as oxcd herein | |
| ScAAE3 (Oxalate-CoA ligase from S. cerevisiae) | SEQ ID NO: 7 |
| yfdE (Acetyl-CoA:oxalate CoA-transferase from | SEQ ID NO: 8 |
| E. coli) | |
| yfdW (formyl CoA transferase from E. coli) | SEQ ID NO: 9 |
| yfdU (oxalyl-CoA decarboxylase E. coli) | SEQ ID NO: 10 |

In one embodiment, one or more polypeptide(s) encoded by the oxalate catabolism cassette(s) and expressed by the genetically engineered bacteria comprises a formyl-CoA transferase, e.g. frc from O. formigenes. In one embodiment the polypeptide(s) have at least about 80% identity with SEQ ID NO: 5. In another embodiment, one or more polypeptide(s) encoded by the oxalate catabolism gene(s) or gene cassette(s) and expressed by the genetically engineered bacteria have at least about 85% identity with SEQ ID NO: 5. In one embodiment, one or more polypeptide(s) encoded by the oxalate catabolism gene(s) or gene cassette(s) and expressed by the genetically engineered bacteria have at least about 90% identity with SEQ ID NO: 5. In one embodiment, one or more polypeptide(s) encoded by the oxalate catabolism gene(s) or gene cassette(s) and expressed by the genetically engineered bacteria have at least about 95% identity with SEQ ID NO: 5. In another embodiment, one or more polypeptide(s) encoded by the oxalate catabolism gene(s) or gene cassette(s) and expressed by the genetically engineered bacteria have at least about 96%, 97%, 98%, or 99% identity with SEQ ID NO: 5. Accordingly, in one embodiment, one or more polypeptide(s) encoded by the oxalate catabolism gene(s) or gene cassette(s) and expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 5. In another embodiment, one or more polypeptide(s) encoded by the oxalate catabolism gene(s) or gene cassette(s) and expressed by the genetically engineered bacteria comprise the sequence of SEQ ID NO: 5. In yet another embodiment one or more polypeptide(s) encoded by the oxalate catabolism gene(s) or gene cassette(s) and expressed by the genetically engineered bacteria consist of the sequence of SEQ ID NO: 5.

In one embodiment, one or more polypeptide(s) encoded by the oxalate catabolism cassette(s) and expressed by the engineered bacteria comprises a oxalyl-CoA decarboxylase, e.g. oxc from O. formigenes. In one embodiment the polypeptide(s) have at least about 80% identity with SEQ ID NO: 6. In another embodiment, one or more polypeptide(s) encoded by the oxalate catabolism gene(s) or gene cassette(s and expressed by the engineered bacteria have at least about 85% identity with SEQ ID NO: 6. In one embodiment, one or more polypeptide(s) encoded by the oxalate catabolism gene(s) or gene cassette(s) and expressed by the engineered bacteria have at least about 90% identity with SEQ ID NO: 6. In one embodiment, one or more polypeptide(s) encoded by the oxalate catabolism gene(s) or gene cassette(s) and expressed by the engineered bacteria have at least about 95% identity with SEQ ID NO: 6. In another embodiment, one or more polypeptide(s) encoded by the oxalate catabolism gene(s) or gene cassette(s) and expressed by the engineered bacteria have at least about 96%, 97%, 98%, or 99% identity with SEQ ID NO: 6. Accordingly, in one embodiment, one or more polypeptide(s) encoded by the oxalate catabolism gene(s) or gene cassette(s) and expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 6. In another embodiment, one or more polypeptide(s) encoded by the oxalate catabolism gene(s) or gene cassette(s) and expressed by the engineered bacteria comprise the sequence of SEQ ID NO: 6. In yet another embodiment one or more polypeptide(s) encoded by the oxalate catabolism gene(s) or gene cassette(s) and expressed by the genetically engineered bacteria consist of the sequence of SEQ ID NO: 6.

In one embodiment, one or more polypeptide(s) encoded by the oxalate catabolism gene(s) or gene cassette(s) and expressed by the engineered bacteria comprises an oxalate-CoA ligase, e.g. ScAAE3 from S. cerevisiae. In one embodiment the polypeptide(s) have at least about 80% identity with SEQ ID NO: 7. In another embodiment, one or more polypeptide(s) encoded by the oxalate catabolism gene(s) or gene cassette(s and expressed by the engineered bacteria have at least about 85% identity with SEQ ID NO: 7. In one embodiment, one or more polypeptide(s) encoded by the oxalate catabolism gene(s) or gene cassette(s) and expressed by the engineered bacteria have at least about 90% identity with SEQ ID NO: 7. In one embodiment, one or more polypeptide(s) encoded by the oxalate catabolism gene(s) or gene cassette(s) and expressed by the engineered bacteria have at least about 95% identity with SEQ ID NO: 7. In another embodiment, one or more polypeptide(s) encoded by the oxalate catabolism gene(s) or gene cassette(s) and expressed by the engineered bacteria have at least about 96%, 97%, 98%, or 99% identity with SEQ ID NO: 7. Accordingly, in one embodiment, one or more polypeptide(s) encoded by the oxalate catabolism gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 7. In another embodiment, one or more polypeptide(s) encoded by the oxalate catabolism gene(s) or gene cassette(s) and expressed by the engineered bacteria comprise the sequence of SEQ ID NO: 7. In yet another embodiment one or more polypeptide(s) encoded by the oxalate catabolism cassette(s) and expressed by the genetically engineered bacteria consist of the sequence of SEQ ID NO: 7.

In one embodiment, one or more polypeptide(s) encoded by the oxalate catabolism cassette(s) and expressed by the engineered bacteria comprises an Acetyl-CoA:oxalate CoA-transferase from, e.g. YfdE from E. coli. In one embodiment the polypeptide(s) have at least about 80% identity with SEQ ID NO: 8. In another embodiment, one or more polypeptide(s) encoded by the oxalate catabolism gene(s) or gene cassette(s and expressed by the engineered bacteria have at least about 85% identity with SEQ ID NO: 8. In one embodiment, one or more polypeptide(s) encoded by the oxalate catabolism gene(s) or gene cassette(s) and expressed by the engineered bacteria have at least about 90% identity with SEQ ID NO: 8. In one embodiment, one or more polypeptide(s) encoded by the oxalate catabolism gene(s) or gene cassette(s) and expressed by the engineered bacteria have at least about 95% identity with SEQ ID NO: 8. In another embodiment, one or more polypeptide(s) encoded by the oxalate catabolism gene(s) or gene cassette(s) and expressed by the engineered bacteria have at least about 96%, 97%, 98%, or 99% identity with SEQ ID NO: 8. Accordingly, in one embodiment, one or more polypeptide(s) encoded by the oxalate catabolism gene(s) or gene cassette(s) and expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 8. In another embodiment, one or more polypeptide(s) encoded by the oxalate catabolism gene(s) or gene cassette(s) and expressed by the engineered bacteria comprise the sequence of SEQ ID NO: 8. In yet another embodiment one or more polypeptide(s) encoded by the oxalate catabolism gene(s) or gene cassette(s) and expressed by the engineered bacteria consist of the sequence of with SEQ ID NO: 8.

In one embodiment, one or more polypeptide(s) encoded by the oxalate catabolism gene(s) or gene cassette(s) and expressed by the genetically engineered bacteria comprises a formyl CoA transferase, e.g., yfdW from E. coli. In one embodiment the polypeptide(s) have at least about 80% identity with SEQ ID NO: 9. In another embodiment, one or more polypeptide(s) encoded by the oxalate catabolism gene(s) or gene cassette(s and expressed by the engineered bacteria have at least about 85% identity with SEQ ID NO: 9. In one embodiment, one or more polypeptide(s) encoded by the oxalate catabolism gene(s) or gene cassette(s) and expressed by the engineered bacteria have at least about 90% identity with SEQ ID NO: 9. In one embodiment, one or more polypeptide(s) encoded by the oxalate catabolism gene(s) or gene cassette(s) and expressed by the engineered bacteria have at least about 95% identity with SEQ ID NO: 9. In another embodiment, one or more polypeptide(s) encoded by the oxalate catabolism gene(s) or gene cassette(s) and expressed by the engineered bacteria have at least about 96%, 97%, 98%, or 99% identity with SEQ ID NO: 9. Accordingly, in one embodiment, one or more polypeptide(s) encoded by the oxalate catabolism gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 9. In another embodiment, one or more polypeptide(s) encoded by the oxalate catabolism gene(s) or gene cassette(s) and expressed by the engineered comprise the sequence of SEQ ID NO: 9. In yet another embodiment one or more polypeptide(s) encoded by the oxalate catabolism gene(s) or gene cassette(s) and expressed by the engineered bacteria consist of the sequence of SEQ ID NO: 9.

In one embodiment, one or more polypeptide(s) encoded by the oxalate catabolism gene(s) or gene cassette(s) and expressed by the engineered bacteria comprises a oxalyl-CoA decarboxylase, e.g., yfdU from E. coli. In one embodiment the polypeptide(s) have at least about 80% identity with SEQ ID NO: 10. In another embodiment, one or more polypeptide(s) encoded by the oxalate catabolism gene(s) or gene cassette(s and expressed by the engineered bacteria have at least about 85% identity with SEQ ID NO: 10. In one embodiment, one or more polypeptide(s) encoded by the oxalate catabolism gene(s) or gene cassette(s) and expressed by the engineered bacteria have at least about 90% identity with SEQ ID NO: 10. In one embodiment, one or more polypeptide(s) encoded by the oxalate catabolism gene(s) or gene cassette(s) and expressed by the engineered bacteria have at least about 95% identity with SEQ ID NO: 10. In another embodiment, one or more polypeptide(s) encoded by the oxalate catabolism gene(s) or gene cassette(s) and expressed by the engineered bacteria have at least about 96%, 97%, 98%, or 99% identity with SEQ ID NO: 10. Accordingly, in one embodiment, one or more polypeptide(s) encoded by the oxalate catabolism gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 10. In another embodiment, one or more polypeptide(s) encoded by the oxalate catabolism gene(s) or gene cassette(s) and expressed by the engineered bacteria comprise the sequence of SEQ ID NO: 10. In yet another embodiment one or more polypeptide(s) encoded by the oxalate catabolism gene(s) or gene cassette(s) and expressed by the engineered bacteria consist of the sequence of SEQ ID NO: 10.

In one embodiment, the recombinant bacteria comprise a nucleotide sequence having at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 1103. In yet another embodiment, the recombinant bacteria comprise the sequence of SEQ ID NO: 1103. In yet another embodiment, the recombinant bacteria consists of the sequence of SEQ ID NO: 1103.

In one embodiment, the recombinant bacteria comprise a nucleotide sequence having at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 1104. In yet another embodiment, the recombinant bacteria comprise the sequence of SEQ ID NO: 1104. In yet another embodiment, the recombinant bacteria consists of the sequence of SEQ ID NO: 1104.

In one embodiment, the recombinant bacteria comprise a nucleotide sequence having at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 1103 and SEQ ID NO: 1104. In yet another embodiment, the recombinant bacteria comprise the sequence of SEQ ID NO: 1103 and SEQ ID NO: 1104. In yet another embodiment, the recombinant bacteria consists of the sequence of SEQ ID NO: 1103 and SEQ ID NO: 1104.

In one embodiment, the gene sequence(s) encoding the one or more oxalate catabolism enzyme(s) is directly operably linked to a first promoter. In another embodiment, the gene sequence(s) encoding the one or more oxalate catabolism enzyme(s) is indirectly operably linked to a first promoter. In one embodiment, the promoter is not operably linked with the at least one gene encoding the oxalate catabolism enzyme in nature.

In some embodiments, the gene sequence(s) encoding the one or more oxalate catabolism enzyme(s) is expressed under the control of a constitutive promoter. In another embodiment, the gene sequence(s) encoding the one or more oxalate catabolism enzyme(s) is expressed under the control of an inducible promoter. In some embodiments, the gene sequence(s) encoding the one or more oxalate catabolism enzyme(s) is expressed under the control of a promoter that is directly or indirectly induced by exogenous environmental conditions. In one embodiment, the gene sequence(s) encoding the one or more oxalate catabolism enzyme(s) is expressed under the control of a promoter that is directly or indirectly induced by low-oxygen or anaerobic conditions, such as the environmental conditions of a mammalian gut, wherein expression of the gene sequence(s) encoding the one or more oxalate catabolism enzyme(s) is activated under low-oxygen or anaerobic environments, such as the environment of a mammalian gut. In some embodiments, the gene sequence(s) encoding the one or more oxalate catabolism enzyme(s) is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, a P_{TetR} promoter, and a P_{LacI} promoter, each of which are described in more detail herein. Inducible promoters are described in more detail infra.

The at least one gene encoding the at least one oxalate catabolism enzyme may be present on a plasmid or chromosome in the bacterial cell. In one embodiment, the gene sequence(s) encoding the one or more oxalate catabolism enzyme(s) is located on a plasmid in the bacterial cell. In another embodiment, the gene sequence(s) encoding the one or more oxalate catabolism enzyme(s) is located in the chromosome of the bacterial cell. In yet another embodiment, a native copy of the gene sequence(s) encoding the one or more oxalate catabolism enzyme(s) is located in the chromosome of the bacterial cell, and at least one gene encoding at least one oxalate catabolism enzyme from a different species of bacteria is located on a plasmid in the bacterial cell. In yet another embodiment, a native copy of the gene sequence(s) encoding the one or more oxalate catabolism enzyme(s) is located on a plasmid in the bacterial cell, and at least one gene encoding the at least one oxalate catabolism enzyme from a different species of bacteria is located on a plasmid in the bacterial cell. In yet another embodiment, a native copy of the gene sequence(s) encoding the one or more oxalate catabolism enzyme(s) is located in the chromosome of the bacterial cell, and at least one gene encoding the at least one oxalate catabolism enzyme from a different species of bacteria is located in the chromosome of the bacterial cell.

In some embodiments, the gene sequence(s) encoding the one or more oxalate catabolism enzyme(s) is expressed on a low-copy plasmid. In some embodiments, the gene sequence(s) encoding the one or more oxalate catabolism enzyme(s) is expressed on a high-copy plasmid. In some embodiments, the high-copy plasmid may be useful for increasing expression of the at least one oxalate catabolism enzyme, thereby increasing the catabolism of oxalate, oxalic acid, and/or oxalyl-CoA.

In some embodiments, a recombinant bacterial cell of the invention comprising at least one gene encoding at least one oxalate catabolism enzyme expressed on a high-copy plasmid does not increase oxalate catabolism or decrease oxalate and/or oxalic acid levels as compared to a recombinant bacterial cell comprising the same gene expressed on a low-copy plasmid in the absence of a heterologous importer of oxalate and additional copies of a native importer of oxalate. Furthermore, in some embodiments that incorporate an importer of oxalate into the recombinant bacterial cell, there may be additional advantages to using a low-copy plasmid comprising the gene sequence(s) encoding the one or more oxalate catabolism enzyme(s) in conjunction in order to enhance the stability of expression of the oxalate catabolism enzyme, while maintaining high oxalate catabolism and to reduce negative selection pressure on the transformed bacterium. In alternate embodiments, the importer of oxalate is used in conjunction with a high-copy plasmid.

Transporter (Importer) of Oxalate

The uptake of oxalate into the anaerobic bacterium, Oxalobacter formigenes, has been found to occur via the oxalate transporter OxlT (see, e.g., Ruan et al., J. Biol. Chem. 267: 10537-43 (1992), the entire contents of which are expressly incorporated herein by reference). OxlT catalyzes the exchange of extracellular oxalate, a divalent anion, for intracellular formate, a monovalent cation that is derived from the decarboxylation of oxalate, thus generating a proton-motive force. Other proteins that mediate the import of oxalate are well known to those of skill in the art.

Oxalate transporters, e.g., oxalate importers, may be expressed or modified in the bacteria of the invention in order to enhance oxalate transport into the cell. Specifically, when the importer of oxalate is expressed in the recombinant bacterial cells of the invention, the bacterial cells import more oxalate into the cell when the importer is expressed than unmodified bacteria of the same bacterial subtype under the same conditions. Thus, the genetically engineered bacteria comprising one or more heterologous gene sequence(s) encoding an importer of oxalate may be used to import oxalate into the bacteria so that any gene sequence(s) encoding an oxalate catabolism enzyme(s) expressed in the organism can be used to treat disorders in which oxalate is detrimental, such as PHI, PHII, PHIII, and secondary hyperoxaluria, enteric hyperoxaluria, and idiopathic hyperoxaluria. In one embodiment, the bacterial cell of the invention comprises a heterologous gene sequence(s) encoding a transporter (importer) of oxalate. In one embodiment, the bacterial cell of the invention comprises a heterologous gene sequence(s) encoding transporter of oxalate and one or more heterologous gene sequence(s) encoding one or more oxalate catabolism enzyme(s). In one embodiment, the bacterial cell of the invention comprises a heterologous gene sequence(s) encoding transporter of oxalate and one or more heterologous gene sequence(s) encoding one or more polypeptides selected from a formate exporter, an oxalate:formate antiporter, and combinations thereof. In one embodiment, the bacterial cell of the invention comprises a heterologous gene sequence(s) encoding a transporter of oxalate, one or more heterologous gene sequence(s) encoding one or more oxalate catabolism enzyme(s), and one or more heterologous gene sequence(s) encoding one or more polypeptides selected from a formate exporter, an oxalate:formate antiporter, and combinations thereof.

Thus, in some embodiments, the invention provides a bacterial cell that comprises one or more heterologous gene sequence(s) encoding an oxalate catabolism enzyme operably linked to a first promoter and one or more heterologous gene sequence(s) encoding an transporter (importer) of oxalate. In some embodiments, the invention provides a bacterial cell that comprises one or more heterologous gene sequence(s) encoding a transporter (importer) of oxalate operably linked to the first promoter. In another embodiment, the invention provides a bacterial cell that comprises one or more heterologous gene sequence(s) encoding one or more oxalate catabolism enzyme(s) operably linked to a first promoter and one or more heterologous gene sequence(s) encoding a transporter (importer) of oxalate operably linked to a second promoter. In one embodiment, the first promoter and the second promoter are separate copies of the same promoter. In another embodiment, the first promoter and the second promoter are different promoters.

In one embodiment, the bacterial cell comprises one or more gene sequence(s) encoding an transporter (importer) of oxalate from a different organism, e.g., a different species of bacteria. In one embodiment, the bacterial cell comprises one or more native gene sequence(s) encoding an transporter (importer) of oxalate. In some embodiments, the one or more native gene sequence(s) encoding an transporter (importer) of oxalate is not modified. In another embodiment, the bacterial cell comprises more than one copy of one or more native gene sequence(s) encoding a transporter (importer) of oxalate. In yet another embodiment, the bacterial cell comprises a copy of one or more gene sequence(s) encoding a native transporter (importer) of oxalate, as well as one or more copy of one or more heterologous gene sequence(s) encoding a transporter of oxalate from a different bacterial species. In one embodiment, the bacterial cell comprises one or more, two, three, four, five, or six copies of the one or more heterologous gene sequence(s) encoding a transporter of oxalate. In one embodiment, the bacterial cell comprises multiple copies of the one or more heterologous gene sequence(s) encoding a transporter of oxalate.

In some embodiments, the transporter of oxalate is encoded by an transporter of oxalate gene derived from a bacterial genus or species, including but not limited to, Oxalobacter. In some embodiments, the transporter of oxalate gene is derived from a bacteria of the species Oxalobacter formigenes. In some embodiments, the transporter is the OxlT Oxalate:Formate Antiporter from Oxalobacter formigenes

In other embodiments, transporter of oxalate is encoded by a gene selected from the oxalate:formate antiporter (OFA) family. The OFA family members belong to the major facilitator superfamily and are widely distributed in nature, being present in the bacterial, archaeal, and eukaryotic kingdoms (see., e.g., Pao et al., Major Facilitator Superfamily Microbiol. Mol Bio. Rev. March 1998 vol. 62 no. 1 1-34). In a non-limiting example, the transporter is a homolog and/or ortholog of the Oxalobacter formigenes oxalate:formate antiporter. In another non-limiting example, the transporter is a bacterially derived homolog and/or ortholog of the Oxalobacter formigenes oxalate:formate antiporter (OxlT). The present invention further comprises genes encoding functional fragments of an transporter of oxalate or functional variants of an transporter of oxalate. As used herein, the term “functional fragment thereof” or “functional variant thereof” of an transporter of oxalate relates to an element having qualitative biological activity in common with the wild-type transporter of oxalate from which the fragment or variant was derived. For example, a functional fragment or a functional variant of a mutated transporter of oxalate protein is one which retains essentially the same ability to import oxalate into the bacterial cell as does the transporter protein from which the functional fragment or functional variant was derived. In one embodiment, the recombinant bacterial cell of the invention comprises one or more heterologous gene sequence(s) encoding a functional fragment of a transporter of oxalate. In another embodiment, the recombinant bacterial cell of the invention comprises one or more heterologous gene sequence(s) encoding a functional variant of a transporter of oxalate.

Assays for testing the activity of a transporter of oxalate, an transporter of oxalate functional variant, or an transporter of oxalate functional fragment are well known to one of ordinary skill in the art. For example, oxalate import can be assessed by preparing detergent-extracted proteoliposomes from recombinant bacterial cells expressing the protein, functional variant, or fragment thereof, and determining [¹⁴C]oxalate uptake as described in Abe et al., J. Biol. Chem. 271: 6789-93 (1996), the entire contents of which are expressly incorporated herein by reference.

In one embodiment the genes encoding the transporter of oxalate have been codon-optimized for use in the host organism. In one embodiment, the genes encoding the transporter of oxalate have been codon-optimized for use in Escherichia coli.

The present invention also encompasses genes encoding a transporter of oxalate comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the one or more gene sequence(s) encoding a transporter of oxalate is mutagenized; mutants exhibiting increased oxalate transport are selected; and the mutagenized one or more gene sequence(s) encoding an transporter of oxalate is isolated and inserted into the bacterial cell of the invention. In some embodiments, the one or more gene sequence(s) encoding an transporter of oxalate is mutagenized; mutants exhibiting decreased oxalate transport are selected; and the mutagenized one or more gene sequence(s) encoding an transporter of oxalate is isolated and inserted into the bacterial cell of the invention. The transporter modifications described herein may be present on a plasmid or chromosome.

Table 4 lists polypeptide and polynucleotide sequences for a non-limiting example of an Oxalate:formate antiporter.

TABLE 4
OxlT sequences

| Description | SEQ ID NO |
|---|---|
| OxlT coding region (oxalate:formate antiporter from | SEQ ID NO: 11 |
| O. formigenes) | |
| OxlT (oxalate:formate antiporter from O. formigenes) | SEQ ID NO: 12 |

In one embodiment, the oxalate importer is the oxalate:formate antiporter OxlT. In one embodiment, the OxlT gene has at least about 80% identity to SEQ ID NO: 11. Accordingly, in one embodiment, the OxlT gene has at least about 90% identity to SEQ ID NO: 11. Accordingly, in one embodiment, the OxlT gene has at least about 95% identity to SEQ ID NO: 11. Accordingly, in one embodiment, the OxlT gene has at least about 85%, 86%, 87%, 88%, 89%, 90%, 91%, 92%, 93%, 94%, 95%, 96%, 97%, 98%, or 99% identity to SEQ ID NO: 11. In another embodiment, the OxlT gene comprises the sequence of SEQ ID NO: 11. In yet another embodiment the OxlT gene consists of the sequence of SEQ ID NO: 11.

In one embodiment, one or more polypeptide(s) encoded by one or more gene(s) or gene cassette(s) and expressed by the genetically engineered bacteria is the oxalate:formate antiporter OxlT. In one embodiment the polypeptide(s) have at least about 80% identity with SEQ ID NO: 12. In another embodiment, one or more polypeptide(s) encoded by one or more gene(s) or gene cassette(s) and expressed by the engineered bacteria have at least about 85% identity with SEQ ID NO: 12. In one embodiment, one or more polypeptide(s) encoded by one or more gene(s) or gene cassette(s) and expressed by the engineered bacteria have at least about 90% identity with SEQ ID NO: 12. In one embodiment, one or more polypeptide(s) encoded by one or more gene(s) or gene cassette(s) and expressed by the engineered bacteria have at least about 95% identity with SEQ ID NO: 12. In another embodiment, one or more polypeptide(s) encoded by one or more gene(s) or gene cassette(s) and expressed by the engineered bacteria have at least about 96%, 97%, 98%, or 99% identity with SEQ ID NO: 12. Accordingly, in one embodiment, one or more polypeptide(s) encoded by one or more gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 12. In another embodiment, one or more polypeptide(s) encoded by one or more gene(s) or gene cassette(s) and expressed by the engineered bacteria comprise the sequence of SEQ ID NO: 12. In yet another embodiment one or more polypeptide(s) encoded by one or more gene(s) or gene cassette(s) and expressed by the engineered bacteria consist of the sequence of SEQ ID NO: 12.

In some embodiments, the bacterial cell comprises one or more heterologous gene sequence(s) encoding at least one oxalate catabolism enzyme(s) operably linked to a first promoter and one or more heterologous gene sequence(s) encoding an importer of oxalate. In some embodiments, the one or more heterologous gene sequence(s) encoding an importer of oxalate is operably linked to the first promoter. In other embodiments, the one or more heterologous gene sequence(s) encoding an importer of oxalate is operably linked to a second promoter. In one embodiment, the one or more gene sequence(s) encoding an importer of oxalate is directly operably linked to the second promoter. In another embodiment, the one or more gene sequence(s) encoding an importer of oxalate is indirectly operably linked to the second promoter.

In some embodiments, expression of one or more gene sequence(s) encoding an importer of oxalate is controlled by a different promoter than the promoter that controls expression of the gene sequence(s) encoding the one or more oxalate catabolism enzyme(s). In some embodiments, expression of the one or more gene sequence(s) encoding an importer of oxalate is controlled by the same promoter that controls expression of the one or more oxalate catabolism enzyme(s). In some embodiments, one or more gene sequence(s) encoding an importer of oxalate and the oxalate catabolism enzyme are divergently transcribed from a promoter region. In some embodiments, expression of each of genes encoding the gene sequence(s) encoding an importer of oxalate and the gene sequence(s) encoding the one or more oxalate catabolism enzyme(s) is controlled by different promoters.

In one embodiment, the promoter is not operably linked with the one or more gene sequence(s) encoding an importer of oxalate in nature. In some embodiments, the one or more gene sequence(s) encoding an importer of oxalate is controlled by its native promoter. In some embodiments, the one or more gene sequence(s) encoding an importer of oxalate is controlled by an inducible promoter. In some embodiments, the one or more gene sequence(s) encoding the importer of oxalate is controlled by a promoter that is stronger than its native promoter. In some embodiments, the one or more gene sequence(s) encoding an importer of oxalate is controlled by a constitutive promoter.

In another embodiment, the promoter is an inducible promoter. Inducible promoters are described in more detail infra.

In one embodiment, the one or more gene sequence(s) encoding an importer of oxalate is located on a plasmid in the bacterial cell. In another embodiment, the one or more gene sequence(s) encoding an importer of oxalate is located in the chromosome of the bacterial cell. In yet another embodiment, a native copy of the one or more gene sequence(s) encoding an importer of oxalate is located in the chromosome of the bacterial cell, and a copy of one or more gene sequence(s) encoding an importer of oxalate from a different species of bacteria is located on a plasmid in the bacterial cell. In yet another embodiment, a native copy of the one or more gene sequence(s) encoding an importer of oxalate is located on a plasmid in the bacterial cell, and a copy of one or more gene sequence(s) encoding an importer of oxalate from a different species of bacteria is located on a plasmid in the bacterial cell. In yet another embodiment, a native copy of the one or more gene sequence(s) encoding an importer of oxalate is located in the chromosome of the bacterial cell, and a copy of the one or more gene sequence(s) encoding an importer of oxalate from a different species of bacteria is located in the chromosome of the bacterial cell.

In some embodiments, the at least one native gene encoding the importer of oxalate in the bacterial cell is not modified, and one or more additional copies of the native importer of oxalate are inserted into the genome. In one embodiment, the one or more additional copies of the native importer that is inserted into the genome are under the control of the same inducible promoter that controls expression of the one or more gene sequence(s) encoding the oxalate catabolism enzyme, e.g., the FNR responsive promoter, or a different inducible promoter than the one that controls expression of the at least one oxalate catabolism enzyme, or a constitutive promoter. In alternate embodiments, the at least one native gene encoding the importer is not modified, and one or more additional copies of the importer from a different bacterial species is inserted into the genome of the bacterial cell. In one embodiment, the one or more additional copies of the importer inserted into the genome of the bacterial cell are under the control of the same inducible promoter that controls expression of the one or more gene sequence(s) encoding the oxalate catabolism enzyme, e.g., the FNR responsive promoter, or a different inducible promoter than the one that controls expression of the gene sequence(s) encoding the one or more oxalate catabolism enzyme(s), or a constitutive promoter.

In one embodiment, when the importer of oxalate is expressed in the recombinant bacterial cells of the invention, the bacterial cells import 10% more oxalate into the bacterial cell when the importer is expressed than unmodified bacteria of the same bacterial subtype under the same conditions. In another embodiment, when the importer of oxalate is expressed in the recombinant bacterial cells of the invention, the bacterial cells import 20%, 30%, 40%, 50%, 60%, 70%, 80%, 90% or 100% more oxalate into the bacterial cell when the importer is expressed than unmodified bacteria of the same bacterial subtype under the same conditions. In yet another embodiment, when the importer of oxalate is expressed in the recombinant bacterial cells of the invention, the bacterial cells import two-fold more oxalate into the cell when the importer is expressed than unmodified bacteria of the same bacterial subtype under the same conditions. In yet another embodiment, when the importer of oxalate is expressed in the recombinant bacterial cells of the invention, the bacterial cells import three-fold, four-fold, five-fold, six-fold, seven-fold, eight-fold, nine-fold, or ten-fold more oxalate into the cell when the importer is expressed than unmodified bacteria of the same bacterial subtype under the same conditions.

In some embodiments, the bacterial cell comprises a genetic mutation in one or more endogenous gene(s) encoding a transporter (importer) of formate, wherein the genetic mutation reduces influx of formate into the bacterial cell. Without wishing to be bound by theory, such mutations may decrease intracellular formate concentrations and increase the flux through oxalate catabolism pathways. FocA of E. coli catalyzes bidirectional formate transport and may function by a channel-type mechanism (Flake et al., Unexpected oligomeric structure of the FocA formate channel of Escherichia coli: a paradigm for the formate-nitrite transporter family of integral membrane proteins”. FEMS microbiology letters. 303 (1): 69-75). FocA may be able to switch its mode of operation from a passive export channel at high external pH to a secondary active formate/H importer at low pH. In a non-limiting example, the genetically engineered bacteria may comprise a mutation and/or deletion in FocA, rendering it non-functional.

Exporters of Formate

Formate is a major metabolite in the anaerobic fermentation of glucose by many intestinal bacteria. Several types of formate import and export proteins are known in the art. For example, formate is translocated across cellular membranes by the pentameric ion channel/transporter FocA in E. coli and other Enterobacteriaceae. FocA acts as a passive exporter for formate anions generated in the cytoplasm. In the periplasm, formate is subsequently reduced by formate dehydrogenase into carbon dioxide. Another form of formate dehydrogenase and/or formate lyase also exists in the cytoplasm in E. coli. A functional switch of transport mode occurs when the pH of the growth medium drops below 6.8. With ample protons available in the periplasm, the cell switches to active import of formate and again uses FocA for the task.

In another example, as mentioned above, the uptake of oxalate into the anaerobic bacterium, Oxalobacter formigenes, has been found to occur via the oxalate transporter OxlT. OxlT allows the exchange of oxalate with the intracellular formate derived from oxalate decarboxylation. The overall effect of these associated activities (exchange and decarboxylation) is generation of a proton-motive force to support membrane functions, including ATP synthesis, accumulation of growth substrates and extrusion of waste products. As such, “exporter of formate” in some embodiments also encompasses a transporter of oxalate, e.g., as in the case of OxlT, the formate:oxalate antiporter.

Formate exporters and/or formate exporters with coupled oxalate import functions may be expressed or modified in the bacteria in order to enhance formate export (and in cases when coupled to oxalate import, thereby enhance oxalate import). Specifically, in some embodiments, when the exporter of formate is expressed in the engineered bacterial cells, the bacterial cells export more formate outside of the cell when the exporter is expressed than unmodified bacteria of the same bacterial subtype under the same conditions. In one embodiment, the bacterial cell comprises one or more gene sequence(s) encoding an exporter of formate. In one embodiment, the bacterial cell comprises a heterologous gene encoding an exporter of formate and at least one heterologous gene or gene cassette encoding at least one oxalate catabolism enzyme.

Thus, in some embodiments, the disclosure provides a bacterial cell that comprises one or more gene sequence(s) encoding one or more oxalate catabolism enzyme(s) operably linked to a first promoter and one or more gene sequence(s) encoding an exporter of formate. In some embodiments, the one or more gene sequence(s) encoding an exporter of formate is operably linked to the first promoter. In another embodiment, the one or more gene sequence(s) encoding one or more oxalate catabolism enzyme(s) is operably is linked to a first promoter, and the one or more gene sequence(s) encoding an exporter of formate is operably linked to a second promoter. In one embodiment, the first promoter and the second promoter are separate copies of the same promoter. In another embodiment, the first promoter and the second promoter are different promoters.

In one embodiment, the bacterial cell comprises one or more gene sequence(s) encoding an exporter of formate from a different organism, e.g., a different species of bacteria. In one embodiment, the bacterial cell comprises at least one native gene sequence(s) encoding an exporter of formate. In some embodiments, the at least one native gene sequence(s) encoding an exporter of formate is not modified. In another embodiment, the bacterial cell comprises more than one copy of at least one gene native sequence(s) encoding an exporter of formate. In yet another embodiment, the bacterial cell comprises a copy one or more gene sequence(s) encoding a native exporter of formate, as well as at least one copy of at least one heterologous gene sequence(s) encoding an exporter of formate from a different bacterial species. In one embodiment, the bacterial cell comprises at least one, two, three, four, five, or six copies of the at least one heterologous gene sequences encoding an exporter of formate. In one embodiment, the bacterial cell comprises multiple copies of one or more heterologous gene sequence(s) encoding an exporter of formate.

In some embodiments, the exporter of formate is encoded by an exporter of formate gene derived from a bacterial genus or species, including but not limited to, Bifidobacterium, Bordetella, Bradyrhizobium, Burkholderia, Clostridium, Enterococcus, Escherichia, Eubacterium, Lactobacillus, Magnetospirillium, Mycobacterium, Neurospora, Oxalobacter, e.g., Oxalobacter formigenes, Ralstonia, Rhodopseudomonas, Shigella, Thermoplasma, and Thauera, e.g., Bifidobacterium animalis, Bifidobacterium bifidum, Bifidobacterium infantis, Bifidobacterium lactis, Bifidobacterium longum, Bordatella bronchiseptica, Bordatella parapertussis, Burkholderia fungorum, Burkholderia xenovorans, Bradyrhizobium japonicum, Clostridium acetobutylicum, Clostridium difficile, Clostridium scindens, Clostridium sporogenes, Clostridium tentani, Enterococcus faecalis, Escherichia coli, Eubacterium lentum, Lactobacillus acidophilus, Lactobacillus bulgaricus, Lactobacillus casei, Lactobacillus gasseri, Lactobacillus plantarum, Lactobacillus rhamnosus, Lactococcus lactis, Magnetospirillium magentotaticum, Mycobacterium avium, Mycobacterium intracellulare, Mycobacterium kansasii, Mycobacterium leprae, Mycobacterium smegmatis, Mycobacterium tuberculosis, Mycobacterium ulcerans, Neurospora crassa, Oxalobacter formigenes, Providencia rettgeri, Eubacterium lentum, Ralstonia eutropha, Ralstonia metallidurans, Rhodopseudomonas palustris, Shigella flexneri, Thermoplasma volcanium, and Thauera aromatica.

The present disclosure further comprises genes encoding functional fragments of an exporter of formate or functional variants of an exporter of formate. As used herein, the term “functional fragment thereof” or “functional variant thereof” of an exporter of formate relates to an element having qualitative biological activity in common with the wild-type exporter of formate from which the fragment or variant was derived. For example, a functional fragment or a functional variant of a mutated exporter of formate protein is one which retains essentially the same ability to import formate into the bacterial cell as does the exporter protein from which the functional fragment or functional variant was derived. In one embodiment, the engineered bacterial cell comprises at least one heterologous gene encoding a functional fragment of an exporter of formate. In another embodiment, the engineered bacterial cell comprises at least one heterologous gene encoding a functional variant of an exporter of formate.

Assays for testing the activity of an exporter of formate, an exporter of formate functional variant, or an exporter of formate functional fragment are well known to one of ordinary skill in the art. For example, formate export can be assessed by expressing the protein, functional variant, or fragment thereof, in an engineered bacterial cell that lacks an endogenous formate exporter and assessing formate levels in the media after expression of the protein. Methods for measuring formate export are well known to one of ordinary skill in the art (see, e.g., Wraight et al., Structure and mechanism of a pentameric formate channel Nat Struct Mol Biol. 2010 January; 17(1): 31-37).

In one embodiment the genes encoding the exporter of formate have been codon-optimized for use in the host organism. In one embodiment, the genes encoding the exporter of formate have been codon-optimized for use in Escherichia coli.

The present disclosure also encompasses genes encoding an exporter of formate comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding an exporter of formate is mutagenized; mutants exhibiting increased formate transport are selected; and the mutagenized at least one gene encoding an exporter of formate is isolated and inserted into the bacterial cell. In a non-limiting example, increasing export of formate may also allow increased oxalate import. In some embodiments, the at least one gene encoding an exporter of formate is mutagenized; mutants exhibiting decreased formate transport are selected; and the mutagenized at least one gene encoding an exporter of formate is isolated and inserted into the bacterial cell. The exporter modifications described herein may be present on a plasmid or chromosome.

In one embodiment, the formate exporter is OxlT. In one embodiment, the OxlT gene has at least about 80% identity to SEQ ID NO: 11. Accordingly, in one embodiment, the OxlT gene has at least about 90% identity to SEQ ID NO: 11. Accordingly, in one embodiment, the OxlT gene has at least about 95% identity to SEQ ID NO: 11. Accordingly, in one embodiment, the OxlT gene has at least about 85%, 86%, 87%, 88%, 89%, 90%, 91%, 92%, 93%, 94%, 95%, 96%, 97%, 98%, or 99% identity to SEQ ID NO: 11. In another embodiment, the OxlT gene comprises the sequence of SEQ ID NO: 11. In yet another embodiment the OxlT gene consists of the sequence of SEQ ID NO: 11.

In one embodiment, the OxlT gene encodes a polypeptide which has at least about 80% identity to SEQ ID NO: 12. Accordingly, in one embodiment, the OxlT gene encodes a polypeptide which has at least about 90% identity to SEQ ID NO: 12. Accordingly, in one embodiment, the OxlT gene encodes a polypeptide which has at least about 95% identity to SEQ ID NO: 12. Accordingly, in one embodiment, the OxlT gene encodes a polypeptide which has at least about 85%, 86%, 87%, 88%, 89%, 90%, 91%, 92%, 93%, 94%, 95%, 96%, 97%, 98%, or 99% identity to SEQ ID NO: 12. In another embodiment, the OxlT gene encodes a polypeptide which comprises the sequence of SEQ ID NO: 12. In yet another embodiment the OxlT gene encodes a polypeptide which consists of the sequence of SEQ ID NO: 12.

In some embodiments, the bacterial cell comprises one or more heterologous gene sequence(s) encoding at least one oxalate catabolism enzyme operably linked to a first promoter and one or more heterologous gene sequence(s) encoding an exporter of formate. In some embodiments, the one or more heterologous gene sequence(s) encoding an exporter of formate are operably linked to the first promoter. In other embodiments, the one or more heterologous gene sequence(s) encoding an exporter of formate are operably linked to a second promoter. In one embodiment, one or more heterologous gene sequence(s) encoding an exporter of formate are directly operably linked to the second promoter. In another embodiment, the one or more heterologous gene sequence(s) encoding an exporter of formate are indirectly operably linked to the second promoter.

In some embodiments, expression one or more gene sequence(s) encoding an exporter of formate is controlled by a different promoter than the promoter that controls expression of the at least one gene encoding the at least one oxalate catabolism enzyme. In some embodiments, expression of the one or more gene sequence(s) encoding an exporter of formate is controlled by the same promoter that controls expression of the at least one oxalate catabolism enzyme. In some embodiments, the one or more gene sequence(s) encoding an exporter of formate and the oxalate catabolism enzyme are divergently transcribed from a promoter region. In some embodiments, expression of each of genes encoding the one or more gene sequence(s) encoding an exporter of formate and the one or more gene sequence(s) encoding the at least one oxalate catabolism enzyme is controlled by different promoters.

In one embodiment, the promoter is not operably linked with the one or more gene sequence(s) encoding an exporter of formate in nature. In some embodiments, the one or more gene sequence(s) encoding the exporter of formate is controlled by its native promoter. In some embodiments, the one or more gene sequence(s) encoding the exporter of formate is controlled by an inducible promoter. In some embodiments, the one or more gene sequence(s) encoding the exporter of formate is controlled by a promoter that is stronger than its native promoter. In some embodiments, the one or more gene sequence(s) encoding the exporter of formate is controlled by a constitutive promoter.

In another embodiment, the promoter is an inducible promoter. Inducible promoters are described in more detail infra.

In one embodiment, the one or more gene sequence(s) encoding an exporter of formate is located on a plasmid in the bacterial cell. In another embodiment, the one or more gene sequence(s) encoding an exporter of formate is located in the chromosome of the bacterial cell. In yet another embodiment, a native copy of the one or more gene sequence(s) encoding an exporter of formate is located in the chromosome of the bacterial cell, and a copy of at least one gene encoding an exporter of formate from a different species of bacteria is located on a plasmid in the bacterial cell. In yet another embodiment, a native copy of the one or more gene sequence(s) encoding an exporter of a formate is located on a plasmid in the bacterial cell, and a copy of at least one gene encoding an exporter of formate from a different species of bacteria is located on a plasmid in the bacterial cell. In yet another embodiment, a native copy of the one or more gene sequence(s) encoding an exporter of formate is located in the chromosome of the bacterial cell, and a copy of the one or more gene sequence(s) encoding an exporter of formate from a different species of bacteria is located in the chromosome of the bacterial cell.

In some embodiments, the at least one native gene encoding the exporter in the bacterial cell is not modified, and one or more additional copies of the native exporter are inserted into the genome. In one embodiment, the one or more additional copies of the native exporter that is inserted into the genome are under the control of the same inducible promoter that controls expression of the at least one gene encoding the oxalate catabolism enzyme, e.g., the FNR responsive promoter, or a different inducible promoter than the one that controls expression of the at least one oxalate catabolism enzyme, or a constitutive promoter. In alternate embodiments, the at least one native gene encoding the exporter is not modified, and one or more additional copies of the exporter from a different bacterial species is inserted into the genome of the bacterial cell. In one embodiment, the one or more additional copies of the exporter inserted into the genome of the bacterial cell are under the control of the same inducible promoter that controls expression of the at least one gene encoding the oxalate catabolism enzyme, e.g., the FNR responsive promoter, or a different inducible promoter than the one that controls expression of the at least one gene encoding the at least one oxalate catabolism enzyme, or a constitutive promoter.

In one embodiment, when the exporter of formate is expressed in the engineered bacterial cells, the bacterial cells export 10% more formate out of the bacterial cell when the exporter is expressed than unmodified bacteria of the same bacterial subtype under the same conditions. In another embodiment, when the exporter of formate is expressed in the engineered bacterial cells, the bacterial cells export 20%, 30%, 40%, 50%, 60%, 70%, 80%, 90% or 100% more formate out of the bacterial cell when the exporter is expressed than unmodified bacteria of the same bacterial subtype under the same conditions. In yet another embodiment, when the exporter of formate is expressed in the engineered bacterial cells, the bacterial cells export two-fold more formate out of the cell when the exporter is expressed than unmodified bacteria of the same bacterial subtype under the same conditions. In yet another embodiment, when the exporter of formate is expressed in the engineered bacterial cells, the bacterial cells export three-fold, four-fold, five-fold, six-fold, seven-fold, eight-fold, nine-fold, or ten-fold more formate out of the cell when the exporter is expressed than unmodified bacteria of the same bacterial subtype under the same conditions.

In one embodiment, the bacterial cell comprises a mutation or deletion in an exporter of oxalate, rendering the exporter less functional or non-functional. Such a mutation may prevent intracellular oxalate from being exported and increase the catabolism of oxalate.

In some embodiments, the genetically engineered bacteria further comprise a mutation or deletion in one or more endogenous formate exporters, e.g., FocA. In a non-limiting example, such genetically engineered bacteria comprising a mutation in FocA comprise one or more gene sequence(s) encoding a formate:oxalate antiporter, e.g., OxlT. In a non-limiting example one or more endogenous formate exporter(s) are mutagenized or deleted, e.g., (e.g., FocA) to reduce or prevent the export of formate without the concurrent import of oxalate through a formate: oxalate antiporter, e.g., OxlT. Such a mutation may increase the uptake and catabolism of oxalate in the bacterial cell.

In some embodiments, formate dehydrogenase and/or formate lyase is mutated or deleted, e.g. to prevent the catabolism of formate in the bacterial cell. Without wishing to be bound by theory, such mutations may increase intracellular formate concentrations, allowing an increase in the flux through a formate oxalate antiporter, and thereby allowing increased oxalate uptake.

Phage Deletion

In some embodiments, the genetically engineered bacteria comprise one or more E. coli Nissle bacteriophage, e.g., Phage 1, Phage 2, and Phage 3. In some embodiments, the genetically engineered bacteria comprise one or modifications or mutations in one or more of Phage 1, 2 or 3. In some embodiments, the genetically engineered bacteria comprise a modification or mutation in Phage 3. Non-limiting examples of such mutations or modifications are described in International Patent Application PCT/US2018/038840, filed Aug. 31, 2016 the contents of which is herein incorporated by reference in its entirety. In some embodiments, the mutations include deletions, insertions, substitutions and inversions and are located in or encompass one or more Phage 3 genes. In some embodiments, the one or more insertions comprise an antibiotic cassette. In some embodiments, the mutation is a deletion. In some embodiments, the genetically engineered bacteria comprise one or more deletions, which are located in or comprise one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the genetically engineered bacteria comprise a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 1064 is deleted from the Phage 3 genome. In one embodiment, a sequence comprising SEQ ID NO: 1064 is deleted from the Phage 3 genome.

Colibactin Island (Also Known as pks Island)

In some embodiments, the engineered microorganism, e.g., engineered bacterium, comprises a modified pks island (colibactin island). Non-limiting examples are described in International Patent Application PCT/US2021/061579, filed Dec. 31, 2021, the contents of which is herein incorporated by reference in its entirety. In some embodiments, the engineered microorganism, e.g., engineered bacterium, comprises a modified clb sequence selected from one or more of the clbA, clbB, clbC, clbD, clbE, clbF, clbG, clbH, clbI, clbJ, clbK, clbL, clbM, clbN, clbO, clbP, clbQ, clbR, and clbS gene sequences, as compared to a suitable control, e.g., the native pks island in an unmodified bacterium of the same strain and/or subtype. In some embodiments, the modified clb sequence is an insertion, a substitution, and/or a deletion as compared to the control. In some embodiments, the modified clb sequence is a deletion of the clb island, e.g., clbA, clbB, clbC, clbD, clbE, clbF, clbG, clbH, clbI, clbJ, clbK, clbL, clbM, clbN, clbO, clbP, clbQ, clbR, and clbS. In one embodiment, the colibactin deletion is the whole island except for the clbS gene, e.g., a deletion of clbA, clbB, clbC, clbD, clbE, clbF, clbG, clbH, clbI, clbJ, clbK, clbL, clbM, clbN, clbO, clbP, clbQ, and clbR.

In some embodiments, the modified endogenous colibactin island comprises one or more modified clb sequences selected from clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), clbR (SEQ ID NO: 1082), or clbS (SEQ ID NO: 1803) gene. In some embodiments, the modified endogenous colibactin island comprises a deletion of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), and clbR (SEQ ID NO: 1082).

Inducible Promoters

In some embodiments, the bacterial cell comprises a stably maintained plasmid or chromosome carrying the gene and/or gene cassette encoding one or more oxalate catabolism enzyme(s), e.g., selected from formyl-CoA:oxalate CoA-transferase, e.g., frc (from O. formigenes), oxalyl-CoA synthetase, e.g., ScAAE3 (from S. cerevisiae), oxalyl-CoA decarboxylase, e.g., selected from oxc (from O. formigenes), and/or acetyl-CoA:oxalate CoA-transferase, e.g., YfdE (from E. coli), genes. such that the oxalate catabolism enzyme(s) can be expressed in the host cell, and the host cell is capable of survival and/or growth in vitro, e.g., in medium, and/or in vivo, e.g., in the gut. In some embodiments, bacterial cell comprises two or more distinct oxalate catabolism enzymes, e.g., formyl-CoA:oxalate CoA-transferase, e.g., frc (from O. formigenes), oxalyl-CoA synthetase, e.g., ScAAE3 (from S. cerevisiae), oxalyl-CoA decarboxylase, e.g., oxc (from O. formigenes), and/or acetyl-CoA:oxalate CoA-transferase, e.g., YfdE (from E. coli) genes In some embodiments, the genetically engineered bacteria comprise multiple copies of the same oxalate catabolism enzyme gene and/or gene cassette. In some embodiments, the genetically engineered bacteria comprise multiple copies of different oxalate catabolism enzyme genes. In some embodiments, the gene and/or gene cassette encoding one or more oxalate catabolism enzyme(s) is present on a plasmid and operably linked to a directly or indirectly inducible promoter. In some embodiments, the gene and/or gene cassette encoding one or more oxalate catabolism enzyme(s) is present on a plasmid and operably linked to a promoter that is induced under low-oxygen or anaerobic conditions. In some embodiments, the gene and/or gene cassette encoding one or more oxalate catabolism enzyme(s) is present on a chromosome and operably linked to a directly or indirectly inducible promoter. In some embodiments, the gene and/or gene cassette encoding one or more oxalate catabolism enzyme(s) is present in the chromosome and operably linked to a promoter that is induced under low-oxygen or anaerobic conditions. In some embodiments, the gene and/or gene cassette encoding one or more oxalate catabolism enzyme(s) is present on a plasmid and operably linked to a promoter that is induced by exposure to tetracycline or arabinose.

In some embodiments, the bacterial cell comprises a stably maintained plasmid or chromosome carrying the gene and/or gene cassette encoding one or more transporter(s) of oxalate, e.g., OxlT from O. formigenes, such that the transporter, e.g., OxlT from O. formigenes, can be expressed in the host cell, and the host cell is capable of survival and/or growth in vitro, e.g., in medium, and/or in vivo, e.g., in the gut. In some embodiments, bacterial cell comprises two or more distinct copies of the gene and/or gene cassette encoding one or more transporter(s) of oxalate, e.g., OxlT from O. formigenes. In some embodiments, the genetically engineered bacteria comprise multiple copies of the same gene and/or gene cassette encoding one or more transporter(s) of oxalate, e.g., OxlT from O. formigenes. In some embodiments, the at least one gene and/or gene cassette encoding one or more transporter(s) of oxalate, e.g., OxlT from O. formigenes, is present on a plasmid and operably linked to a directly or indirectly inducible promoter. In some embodiments, the gene and/or gene cassette encoding one or more transporter(s) of oxalate, e.g., OxlT from O. formigenes, is present on a plasmid and operably linked to a promoter that is induced under low-oxygen or anaerobic conditions. In some embodiments, the gene and/or gene cassette encoding one or more transporter(s) of oxalate, e.g., OxlT from O. formigenes, is present on a chromosome and operably linked to a directly or indirectly inducible promoter. In some embodiments, the gene and/or gene cassette encoding one or more transporter(s) of oxalate, e.g., OxlT from O. formigenes, is present in the chromosome and operably linked to a promoter that is induced under low-oxygen or anaerobic conditions. In some embodiments, the gene and/or gene cassette encoding one or more transporter(s) of oxalate, e.g., OxlT from O. formigenes, is present on a plasmid and operably linked to a promoter that is induced by exposure to tetracycline.

In some embodiments, the promoter that is operably linked to the gene and/or gene cassette encoding one or more oxalate catabolism enzyme(s) and the promoter that is operably linked to the gene and/or gene cassette encoding one or more transporter(s) of oxalate, e.g., OxlT from O. formigenes, is directly induced by exogenous environmental conditions. In some embodiments, the promoter that is operably linked to the gene and/or gene cassette encoding one or more oxalate catabolism enzyme(s) and the promoter that is operably linked to the gene and/or gene cassette encoding one or more transporter(s) of oxalate, e.g., OxlT from O. formigenes, is indirectly induced by exogenous environmental conditions. In some embodiments, the promoter is directly or indirectly induced by exogenous environmental conditions specific to the gut of a mammal. In some embodiments, the promoter is directly or indirectly induced by exogenous environmental conditions specific to the small intestine of a mammal. In some embodiments, the promoter is directly or indirectly induced by low-oxygen or anaerobic conditions such as the environment of the mammalian gut. In some embodiments, the promoter is directly or indirectly induced by molecules or metabolites that are specific to the gut of a mammal, e.g., propionate. In some embodiments, the promoter is directly or indirectly induced by a molecule that is co-administered with the bacterial cell.

Oxygen Dependent Regulation

In certain embodiments, the bacterial cell comprises a gene and/or gene cassette encoding one or more oxalate catabolism enzyme(s), e.g., selected from formyl-CoA:oxalate CoA-transferase, e.g., frc (from O. formigenes), oxalyl-CoA synthetase, e.g., ScAAE3 (from S. cerevisiae), oxalyl-CoA decarboxylase, e.g., oxc (from O. formigenes), and/or acetyl-CoA:oxalate CoA-transferase, e.g., YfdE (from E. coli), is expressed under the control of the fumarate and nitrate reductase regulator (FNR) promoter. In certain embodiments, the bacterial cell comprises gene and/or gene cassette encoding one or more transporter(s) of oxalate, e.g., OxlT from O. formigenes, is expressed under the control of the fumarate and nitrate reductase regulator (FNR) promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive.

FNR responsive promoters include, but are not limited to, the FNR responsive promoters listed in the chart, below. Underlined sequences are predicted ribosome binding sites, and bolded sequences are restriction sites used for cloning.

TABLE 5
FNR responsive promoters

| | |
|---|---|
| FNR Responsive Promoter | SEQ ID NO |

SEQ ID NO: 13
SEQ ID NO: 14
SEQ ID NO: 15
SEQ ID NO: 16
SEQ ID NO: 17

In one embodiment, the FNR responsive promoter comprises SEQ ID NO: 13. In one embodiment, the FNR responsive promoter comprises SEQ ID NO: 14. In another embodiment, the FNR responsive promoter comprises SEQ ID NO: 15. In another embodiment, the FNR responsive promoter comprises SEQ ID NO: 16. In another embodiment, the FNR responsive promoter comprises SEQ ID NO: 17. Additional FNR responsive promoters are shown below in Table 6.

TABLE 6
FNR Promoter Sequences

| FNR-responsive regulatory | |
|---|---|
| region Sequence | SEQ ID NO |
| | SEQ ID NO: 18 |
| | SEQ ID NO: 19 |
| nirB1 | SEQ ID NO: 20 |
| nirB2 | SEQ ID NO: 21 |
| nirB3 | SEQ ID NO: 22 |
| ydfZ | SEQ ID NO: 23 |
| nirB + RBS | SEQ ID NO: 24 |
| ydfZ + RBS | SEQ ID NO: 25 |
| fnrS1 | SEQ ID NO: 26 |
| fnrS2 | SEQ ID NO: 27 |
| nirB + crp | SEQ ID NO: 28 |
| fnrS + crp | SEQ ID NO: 29 |

In one embodiment, the FNR responsive promoter comprises SEQ ID NO: 18. In one embodiment, the FNR responsive promoter comprises SEQ ID NO: 19. In another embodiment, the FNR responsive promoter comprises SEQ ID NO: 20. In another embodiment, the FNR responsive promoter comprises SEQ ID NO: 21. In another embodiment, the FNR responsive promoter comprises SEQ ID NO: 22. In one embodiment, the FNR responsive promoter comprises SEQ ID NO: 23. In one embodiment, the FNR responsive promoter comprises SEQ ID NO: 24. In another embodiment, the FNR responsive promoter comprises SEQ ID NO: 25. In another embodiment, the FNR responsive promoter comprises SEQ ID NO: 26. In another embodiment, the FNR responsive promoter comprises SEQ ID NO: 27. In another embodiment, the FNR responsive promoter comprises SEQ ID NO: 28. In another embodiment, the FNR responsive promoter comprises SEQ ID NO: 29.

In some embodiments, multiple distinct FNR nucleic acid sequences are inserted in the genetically engineered bacteria. In alternate embodiments, the genetically engineered bacteria comprise a gene and/or gene cassette(s) encoding one or more oxalate catabolism enzyme(s), e.g., selected from formyl-CoA:oxalate CoA-transferase, e.g., Frc (from O. formigenes), oxalyl-CoA synthetase, e.g., ScAAE3 (from S. cerevisiae), oxalyl-CoA decarboxylase, e.g., Oxc (from O. formigenes), and/or acetyl-CoA:oxalate CoA-transferase, e.g., YfdE (from E. coli) or other enzyme disclosed herein, is expressed under the control of an alternate oxygen level-dependent promoter, e.g., DNR (Trunk et al., 2010) or ANR (Ray et al., 1997). In alternate embodiments, the genetically engineered bacteria comprise a gene and/or gene cassette encoding one or more transporter(s) of oxalate, e.g., OxlT from O. formigenes, which is expressed under the control of an alternate oxygen level-dependent promoter, e.g., DNR (Trunk et al., 2010) or ANR (Ray et al., 1997). In these embodiments, catabolism of oxalate and/or its metabolites, is particularly activated in a low-oxygen or anaerobic environment, such as in the gut. In some embodiments, gene expression is further optimized by methods known in the art, e.g., by optimizing ribosomal binding sites and/or increasing mRNA stability. In one embodiment, the mammalian gut is a human mammalian gut.

In some embodiments, the bacterial cell comprises an oxygen-level dependent transcriptional regulator, e.g., FNR, ANR, or DNR, and corresponding promoter from a different bacterial species. The heterologous oxygen-level dependent transcriptional regulator and promoter increase the transcription of genes operably linked to said promoter, e.g., the gene and/or gene cassette encoding one or more oxalate catabolism enzyme(s), and/or the gene and/or gene cassette encoding one or more transporter(s) of oxalate, e.g., OxlT from O. formigenes in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the genetically engineered bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene and/or gene cassette encoding one or more oxalate catabolism enzyme(s), and/or the gene and/or gene cassette encoding one or more transporter(s) of oxalate, e.g., OxlT from O. formigenes in a low-oxygen or anaerobic environment, as compared to the wild-type promoter under the same conditions. In some embodiments, the genetically engineered bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene and/or gene cassette encoding one or more oxalate catabolism enzyme(s), and/or the gene and/or gene cassette encoding one or more transporter(s) of oxalate, e.g., OxlT from O. formigenes in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., 2006).

In some embodiments, the bacterial cells disclosed herein comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene and/or gene cassette encoding one or more oxalate catabolism enzyme(s) are present on different plasmids. In some embodiments, the gene encoding the oxygen level-sensing transcriptional regulator and the gene and/or gene cassette encoding one or more oxalate catabolism enzyme(s) and/or the gene encoding a transporter of an oxalate are present on different plasmids. In some embodiments, the gene encoding the oxygen level-sensing transcriptional regulator and the gene and/or gene cassette encoding one or more oxalate catabolism enzyme(s) and/or the gene and/or gene cassette encoding one or more transporter(s) of oxalate are present on the same plasmid.

In some embodiments, the gene encoding the oxygen level-sensing transcriptional regulator is present on a chromosome. In some embodiments, the gene encoding the oxygen level-sensing transcriptional regulator and the gene and/or gene cassette encoding one or more oxalate catabolism enzyme(s) and/or the gene and/or gene cassette encoding one or more a transporter(s) of oxalate are present on different chromosomes. In some embodiments, the gene encoding the oxygen level-sensing transcriptional regulator and the gene and/or gene cassette encoding one or more oxalate catabolism enzyme(s) and/or the gene and/or gene cassette encoding one or more transporter(s) of oxalate are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene and/or gene cassette encoding one or more oxalate catabolism enzyme(s) and/or oxalate transporter. In some embodiments, expression of the transcriptional regulator is controlled by the same promoter that controls expression of the oxalate catabolism enzyme(s) and/or Oxalate transporter(s). In some embodiments, the transcriptional regulator and the oxalate catabolism enzyme(s) are divergently transcribed from a promoter region.

RNS-Dependent Regulation

In some embodiments, the genetically engineered bacteria comprise a gene and/or gene cassette encoding one or more oxalate catabolism enzyme(s) that is expressed under the control of an inducible promoter. In some embodiments, the genetically engineered bacterium that expresses one or more oxalate catabolism enzyme(s) and/or oxalate transporter(s) is under the control of a promoter that is activated by inflammatory conditions. In one embodiment, the gene and/or gene cassette for producing the oxalate catabolism enzyme(s) and/or oxalate transporter is expressed under the control of an inflammatory-dependent promoter that is activated in inflammatory environments, e.g., a reactive nitrogen species or RNS promoter.

As used herein, “reactive nitrogen species” and “RNS” are used interchangeably to refer to highly active molecules, ions, and/or radicals derived from molecular nitrogen. RNS can cause deleterious cellular effects such as nitrosative stress. RNS includes, but is not limited to, nitric oxide (NO·), peroxynitrite or peroxynitrite anion (ONOO—), nitrogen dioxide (·NO2), dinitrogen trioxide (N2O3), peroxynitrous acid (ONOOH), and nitroperoxycarbonate (ONOOCO2-) (unpaired electrons denoted by ·). Bacteria have evolved transcription factors that are capable of sensing RNS levels. Different RNS signaling pathways are triggered by different RNS levels and occur with different kinetics.

Examples of transcription factors that sense RNS and their corresponding RNS-responsive genes, promoters, and/or regulatory regions include, but are not limited to, those shown in Table 7.

TABLE 7
Examples of RNS-sensing transcription
factors and RNS-responsive genes

| RNS-sensing | Primarily | |
|---|---|---|
| transcription | capable of | Examples of responsive genes, promoters, |
| factor: | sensing: | and/or regulatory regions: |
| NsrR | NO | norB, aniA, nsrR, hmpA, ytfE, ygbA, hcp, hcr, |
| | | nrfA, aox |
| NorR | NO | norVW, norR |
| DNR | NO | norCB, nir, nor, nos |

In some embodiments, the genetically engineered bacteria of the invention comprise a tunable regulatory region that is directly or indirectly controlled by a transcription factor that is capable of sensing at least one reactive nitrogen species. The tunable regulatory region is operatively linked to a gene and/or gene cassette capable of directly or indirectly driving the expression of one or more oxalate catabolism enzyme(s), oxalate transporter(s), thus controlling expression of the oxalate catabolism enzyme, oxalate transporter(s), relative to RNS levels. For example, the tunable regulatory region is a RNS-inducible regulatory region, and the payload is one or more oxalate catabolism enzyme(s), oxalate transporter(s), such as any of the oxalate catabolism enzymes, and/or oxalate transporter(s) provided herein; when RNS is present, e.g., in an inflamed tissue, a RNS-sensing transcription factor binds to and/or activates the regulatory region and drives expression of the oxalate catabolism enzyme and/or oxalate transporter gene or gene cassette. Subsequently, when inflammation is ameliorated, RNS levels are reduced, and production of the oxalate catabolism enzyme(s) and oxalate transporter(s) is decreased or eliminated.

ROS-Dependent Regulation

In some embodiments, the genetically engineered bacteria comprise a gene and/or gene cassette for producing one or more oxalate catabolism enzyme(s) and/or oxalate transporter(s) that is expressed under the control of an inducible promoter. In some embodiments, the genetically engineered bacterium that expresses one or more oxalate catabolism enzyme(s) and/or oxalate transporter(s) under the control of a promoter that is activated by conditions of cellular damage. In one embodiment, the gene and/or gene cassette for producing one or more oxalate catabolism enzyme(s) is expressed under the control of a cellular damaged-dependent promoter that is activated in environments in which there is cellular or tissue damage, e.g., a reactive oxygen species or ROS promoter.

As used herein, “reactive oxygen species” and “ROS” are used interchangeably to refer to highly active molecules, ions, and/or radicals derived from molecular oxygen. ROS can be produced as byproducts of aerobic respiration or metal-catalyzed oxidation and may cause deleterious cellular effects such as oxidative damage. ROS includes, but is not limited to, hydrogen peroxide (H2O2), organic peroxide (ROOH), hydroxyl ion (OH—), hydroxyl radical (·OH), superoxide or superoxide anion (·O2-), singlet oxygen (1O2), ozone (O3), carbonate radical, peroxide or peroxyl radical (·O2-2), hypochlorous acid (HOCl), hypochlorite ion (OCl—), sodium hypochlorite (NaOCl), nitric oxide (NO·), and peroxynitrite or peroxynitrite anion (ONOO—) (unpaired electrons denoted by ·). Bacteria have evolved transcription factors that are capable of sensing ROS levels. Different ROS signaling pathways are triggered by different ROS levels and occur with different kinetics (Marinho et al., 2014).

As used herein, “ROS-inducible regulatory region” refers to a nucleic acid sequence to which one or more ROS-sensing transcription factors is capable of binding, wherein the binding and/or activation of the corresponding transcription factor activates downstream gene expression; in the presence of ROS, the transcription factor binds to and/or activates the regulatory region. In some embodiments, the ROS-inducible regulatory region comprises a promoter sequence. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; in the presence of ROS, the transcription factor undergoes a conformational change, thereby activating downstream gene expression. The ROS-inducible regulatory region may be operatively linked to a gene sequence or gene sequence, e.g., a sequence or sequences encoding one or more oxalate catabolism enzyme(s). For example, in the presence of ROS, a transcription factor, e.g., OxyR, senses ROS and activates a corresponding ROS-inducible regulatory region, thereby driving expression of an operatively linked gene sequence or gene sequences. Thus, ROS induces expression of the gene or gene cassette.

Examples of transcription factors that sense ROS and their corresponding ROS-responsive genes, promoters, and/or regulatory regions include, but are not limited to, those shown in Table 8.

TABLE 8
Examples of ROS-sensing transcription factors and ROS-responsive genes

| ROS-sensing | Primarily | |
|---|---|---|
| transcription | capable of | Examples of responsive genes, promoters, and/or |
| factor: | sensing: | regulatory regions: |
| OxyR | H₂O₂ | ahpC; ahpF; dps; dsbG; fhuF; flu; fur; gor; grxA; |
| | | hemH; katG; oxyS; sufA; sufB; sufC; sufD; sufE; |
| | | sufS; trxC; uxuA; yaaA; yaeH; yaiA; ybjM; ydcH; |
| | | ydeN; ygaQ; yljA; ytfK |
| PerR | H₂O₂ | katA; ahpCF; mrgA; zoaA; fur; hemAXCDBL; srfA |
| OhrR | Organic peroxides | ohrA |
| | NaOCl | |
| SoxR | •O₂⁻ | soxS |
| | NO• | |
| | (also capable of | |
| | sensing H₂O₂) | |
| RosR | H₂O₂ | rbtT; tnp16a; rluC1; tnp5a; mscL; tnp2d; phoD; |
| | | tnp15b; pstA; tnp5b; xylC; gabD1; rluC2; cgtS9; |
| | | azlC; narKGHJI; rosR |

In some embodiments, the genetically engineered bacteria comprise a tunable regulatory region that is directly or indirectly controlled by a transcription factor that is capable of sensing at least one reactive oxygen species. The tunable regulatory region is operatively linked to a gene or gene cassette capable of directly or indirectly driving the expression of an oxalate catabolism enzyme, thus controlling expression of the oxalate catabolism enzyme(s) relative to ROS levels. For example, the tunable regulatory region is a ROS-inducible regulatory region, and the molecule is an oxalate catabolism enzyme; when ROS is present, e.g., in an inflamed tissue, a ROS-sensing transcription factor binds to and/or activates the regulatory region and drives expression of the gene sequence and/or gene cassette sequence for one or more the oxalate catabolism enzyme(s) and/or oxalate transporter(s) thereby producing the oxalate catabolism enzyme(s) and/or oxalate transporter(s). Subsequently, when inflammation is ameliorated, ROS levels are reduced, and production of the oxalate catabolism enzyme(s) and/or oxalate transporter(s) is decreased or eliminated.

Nucleic acid sequences of several exemplary OxyR-regulated regulatory regions are shown in Table 5. OxyR binding sites are underlined and bolded. In some embodiments, genetically engineered bacteria comprise a nucleic acid sequence that is at least about 80%, at least about 85%, at least about 90%, at least about 95%, or at least about 99% homologous to the DNA sequence of SEQ ID NO: 46, 47, 48, or 49, or a functional fragment thereof.

TABLE 9
Nucleotide sequences of exemplary
OxyR-regulated regulatory regions

| Regulatory sequence | SEQ ID NO |
|---|---|
| katG | SEQ ID NO: 30 |
| dps | SEQ ID NO: 31 |
| ahpC | SEQ ID NO: 32 |
| oxyS | SEQ ID NO: 33 |

In some embodiments, the genetically engineered bacteria of the invention comprise a gene encoding a ROS-sensing transcription factor, e.g., the oxyR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the ROS-sensing transcription factor under the control of an inducible promoter in order to enhance expression stability. In some embodiments, expression of the ROS-sensing transcription factor is controlled by a different promoter than the promoter that controls expression of the therapeutic molecule. In some embodiments, expression of the ROS-sensing transcription factor is controlled by the same promoter that controls expression of the therapeutic molecule. In some embodiments, the ROS-sensing transcription factor and therapeutic molecule are divergently transcribed from a promoter region.

Temperature Dependent Regulation

In some instances, thermoregulators may be advantageous because of strong transcriptional control without the use of external chemicals or specialized media. Thermoregulated protein expression using the mutant c1857 repressor and the pL and/or pR phage λ promoters have been used to engineer recombinant bacterial strains. For example, a gene of interest cloned downstream of the λ promoters can be efficiently regulated by the mutant thermolabile c1857 repressor of bacteriophage λ. At temperatures below 37° C., c1857 binds to the oL or oR regions of the pR promoter and inhibits transcription by RNA polymerase. At higher temperatures, the functional c1857 dimer is destabilized, binding to the oL or oR DNA sequences is abrogated, and mRNA transcription is initiated. In certain instances, it may be advantageous to reduce, diminish, or shut off production of one or more protein(s) of interest. This can be done in a thermoregulated system by growing a bacterial strain at temperatures at which the temperature regulated system is not optimally active. Temperature regulated expression can then be induced as desired by changing the temperature to a temperature where the system is more active or optimally active.

For example, a thermoregulated promoter may be induced in culture, e.g., grown in a flask, fermenter or other appropriate culture vessel, e.g., used during cell growth, cell expansion, fermentation, recovery, purification, formulation, and/or manufacture. Bacteria comprising gene sequences or gene cassettes either indirectly or directly operably linked to a temperature sensitive system or promoter may, for example, could be induced by temperatures between 37° C. and 42° C. In some instances, the cultures may be grown aerobically. Alternatively, the cultures are grown anaerobically.

In some embodiments, the bacteria described herein comprise one or more gene sequence(s) or gene cassette(s) which are directly or indirectly operably linked to a temperature regulated promoter. In some embodiments, the gene sequence(s) or gene cassette(s) are induced in vitro during growth, preparation, or manufacturing of the strain prior to in vivo administration. In some embodiments, the gene sequence(s) are induced upon or during in vivo administration. In some embodiments, the gene sequence(s) are induced during in vitro growth, preparation, or manufacturing of the strain prior to in vivo administration and upon or during in vivo administration. In some embodiments, the genetically engineered bacteria further comprise gene sequence (s) encoding a transcription factor which is capable of binding to the temperature sensitive promoter. In some embodiments, the transcription factor is a repressor of transcription.

In one embodiment, the thermoregulated promoter is operably linked to a construct having gene sequence(s) or gene cassette(s) encoding one or more protein(s) of interest jointly with a second promoter, e.g., a second constitutive or inducible promoter. In some embodiments, two promoters are positioned proximally to the construct and drive its expression, wherein the thermoregulated promoter is induced under a first set of exogenous conditions, and the second promoter is induced under a second set of exogenous conditions. In a non-limiting example, the first and second conditions may be two sequential culture conditions (i.e., during preparation of the culture in a flask, fermenter or other appropriate culture vessel, e.g., thermoregulation and arabinose or IPTG). In another non-limiting example, the first inducing conditions may be culture conditions, e.g., permissive temperature, and the second inducing conditions may be in vivo conditions. Such in vivo conditions include low-oxygen, microaerobic, or anaerobic conditions, presence of gut metabolites, and/or metabolites administered in combination with the bacterial strain. In some embodiments, one or more thermoregulated promoters drive expression of one or more protein(s) of interest in combination with an oxygen regulated promoter, e.g., FNR, driving the expression of the same gene sequence(s).

In some embodiments, the thermoregulated promoter drives the expression of one or more protein(s) of interest from a low-copy plasmid or a high copy plasmid or a biosafety system plasmid described herein. In some embodiments, the thermoregulated promoter drives the expression of one or more protein(s) of interest from a construct which is integrated into the bacterial chromosome. Exemplary insertion sites are described herein.

In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 209. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 213. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 216. In some embodiments, the thermoregulated construct further comprises a gene encoding mutant cI857 repressor, which is divergently transcribed from the same promoter as the one or more one or more protein(s) of interest. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 210. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 212. In some embodiments, the thermoregulated construct further comprises a gene encoding mutant cI38 repressor, which is divergently transcribed from the same promoter as the one or more one or more protein(s) of interest. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with any of the sequences of SEQ ID NO: 214. In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by any of the sequences of SEQ ID NO: 215.

SEQ ID NOs: 209, 210, and 212-16 are shown in Table 10.

TABLE 10
Inducible promoter construct sequences and related elements

| Description | SEQ ID NO |
|---|---|
| Region comprising Temperature sensitive | SEQ ID NO: 209 |
| promoter | |
| mutant cI857 repressor nucleotide sequence | SEQ ID NO: 210 |
| mutant cI857 repressor polypeptide sequence | SEQ ID NO: 212 |
| Pr/Pl promoter | SEQ ID NO: 213 |
| mutant cI38 repressor nucleotide sequence | SEQ ID NO: 214 |
| mutant cI38 repressor polypeptide sequence | SEQ ID NO: 215 |
| Temperature sensitive promoter | SEQ ID NO: 216 |

Essential Genes and Auxotrophs

As used herein, the term “essential gene” refers to a gene which is necessary to for cell growth and/or survival. Bacterial essential genes are well known to one of ordinary skill in the art, and can be identified by directed deletion of genes and/or random mutagenesis and screening (see, for example, Zhang and Lin, 2009, DEG 5.0, a database of essential genes in both prokaryotes and eukaryotes, Nucl. Acids Res., 37:D455-D458 and Gerdes et al., Essential genes on metabolic maps, Curr. Opin. Biotechnol., 17(5):448-456, the entire contents of each of which are expressly incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

An auxotrophic modification is intended to cause bacteria to die in the absence of an exogenously added nutrient essential for survival or growth because they lack the gene(s) necessary to produce that essential nutrient. In some embodiments, any of the genetically engineered bacteria described herein also comprise a deletion or mutation in one or more gene(s) required for cell survival and/or growth.

In some embodiments, the bacterial cell comprises a genetic mutation in one or more endogenous gene(s) encoding an oxalate biosynthesis gene, wherein the genetic mutation reduces biosynthesis of oxalate in the bacterial cell.

In one embodiment, the essential gene is an oligonucleotide synthesis gene, for example, thyA. In another embodiment, the essential gene is a cell wall synthesis gene, for example, dapA. In yet another embodiment, the essential gene is an amino acid gene, for example, serA or MetA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1, as long as the corresponding wild-type gene product is not produced in the bacteria.

Table 11 lists depicts exemplary bacterial genes which may be disrupted or deleted to produce an auxotrophic strain. These include, but are not limited to, genes required for oligonucleotide synthesis, amino acid synthesis, and cell wall synthesis.

TABLE 11
Non-limiting Examples of Bacterial Genes
Useful for Generation of an Auxotroph

| Amino Acid | Oligonucleotide | Cell Wall |
|---|---|---|
| cysE | thyA | dapA |
| glnA | uraA | dapB |
| ilvD | | dapD |
| leuB | | dapE |
| lysA | | dapF |
| serA | | |
| metA | | |
| glyA | | |
| hisB | | |
| ilvA | | |
| pheA | | |
| proA | | |
| thrC | | |
| trpC | | |
| tyrA | | |

Table 12 shows the survival of various amino acid auxotrophs in the mouse gut, as detected 24 hrs and 48 hrs post-gavage. These auxotrophs were generated using BW25113, a non-Nissle strain of E. coli.

TABLE 12
Survival of amino acid auxotrophs in the mouse gut

| Gene | AA Auxotroph | Pre-Gavage | 24 hours | 48 hours |
|---|---|---|---|---|
| argA | Arginine | Present | Present | Absent |
| cysE | Cysteine | Present | Present | Absent |
| glnA | Glutamine | Present | Present | Absent |
| glyA | Glycine | Present | Present | Absent |
| hisB | Histidine | Present | Present | Present |
| ilvA | Isoleucine | Present | Present | Absent |
| leuB | Leucine | Present | Present | Absent |
| lysA | Lysine | Present | Present | Absent |
| metA | Methionine | Present | Present | Present |
| pheA | Phenylalanine | Present | Present | Present |
| proA | Proline | Present | Present | Absent |
| sera | Serine | Present | Present | Present |
| thrC | Threonine | Present | Present | Present |
| trpC | Tryptophan | Present | Present | Present |
| tyrA | Tyrosine | Present | Present | Present |
| ilvD | Valine/Isoleucine/ | Present | Present | Absent |
| | Leucine | | | |
| thyA | Thiamine | Present | Absent | Absent |
| uraA | Uracil | Present | Absent | Absent |
| flhD | FlhD | Present | Present | Present |

For example, thymine is a nucleic acid that is required for bacterial cell growth; in its absence, bacteria undergo cell death. The thyA gene encodes thymidylate synthetase, an enzyme that catalyzes the first step in thymine synthesis by converting dUMP to dTMP (Sat et al., 2003). In some embodiments, the bacterial cell of the disclosure is a thyA auxotroph in which the thyA gene is deleted and/or replaced with an unrelated gene. A thyA auxotroph can grow only when sufficient amounts of thymine are present, e.g., by adding thymine to growth media in vitro, or in the presence of high thymine levels found naturally in the human gut in vivo. In some embodiments, the bacterial cell of the disclosure is auxotrophic in a gene that is complemented when the bacterium is present in the mammalian gut. Without sufficient amounts of thymine, the thyA auxotroph dies. In some embodiments, the auxotrophic modification is used to ensure that the bacterial cell does not survive in the absence of the auxotrophic gene product (e.g., outside of the gut).

Diaminopimelic acid (DAP) is an amino acid synthetized within the lysine biosynthetic pathway and is required for bacterial cell wall growth (Meadow et al., 1959; Clarkson et al., 1971). In some embodiments, any of the genetically engineered bacteria described herein is a dapD auxotroph in which dapD is deleted and/or replaced with an unrelated gene. A dapD auxotroph can grow only when sufficient amounts of DAP are present, e.g., by adding DAP to growth media in vitro. Without sufficient amounts of DAP, the dapD auxotroph dies. In some embodiments, the auxotrophic modification is used to ensure that the bacterial cell does not survive in the absence of the auxotrophic gene product (e.g., outside of the gut).

In other embodiments, the genetically engineered bacterium of the present disclosure is a uraA auxotroph in which uraA is deleted and/or replaced with an unrelated gene. The uraA gene codes for UraA, a membrane-bound transporter that facilitates the uptake and subsequent metabolism of the pyrimidine uracil (Andersen et al., 1995). A uraA auxotroph can grow only when sufficient amounts of uracil are present, e.g., by adding uracil to growth media in vitro. Without sufficient amounts of uracil, the uraA auxotroph dies. In some embodiments, auxotrophic modifications are used to ensure that the bacteria do not survive in the absence of the auxotrophic gene product (e.g., outside of the gut).

In complex communities, it is possible for bacteria to share DNA. In very rare circumstances, an auxotrophic bacterial strain may receive DNA from a non-auxotrophic strain, which repairs the genomic deletion and permanently rescues the auxotroph. Therefore, engineering a bacterial strain with more than one auxotroph may greatly decrease the probability that DNA transfer will occur enough times to rescue the auxotrophy. In some embodiments, the genetically engineered bacteria comprise a deletion or mutation in two or more genes required for cell survival and/or growth.

Other examples of essential genes include, but are not limited to yhbV, yagG, hemB, secD, secF, ribD, ribE, thiL, dxs, ispA, dnaX, adk, hemH, IpxH, cysS, fold, rplT, infC, thrS, nadE, gapA, yeaZ, aspS, argS, pgsA, yefM, metG, folE, yejM, gyrA, nrdA, nrdB, folC, accD, fabB, gltX, ligA, zipA, dapE, dapA, der, hisS, ispG, suhB, tadA, acpS, era, rnc, ftsB, eno, pyrG, chpR, lgt, fbaA, pgk, yqgD, metK, yqgF, plsC, ygiT, pare, ribB, cca, ygjD, tdcF, yraL, yihA, ftsN, murI, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, dnaC, ribF, ispA, ispH, dapB, folA, imp, yabQ, ftsL, ftsI, murE, murF, mraY, murD, ftsW, murG, murC, ftsQ, ftsA, ftsZ, lpxC, secM, secA, can, folK, hemL, yadR, dapD, map, rpsB, injB, nusA, ftsH, obgE, rpmA, rplU, ispB, murA, yrbB, yrbK, yhbN, rpsI, rplM, degS, mreD, mreC, mreB, accB, accC, yrdC, def, fmt, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsJ, fusA, rpsG, rpsL, trpS, yrfF, asd, rpoH, ftsX, ftsE, ftsY, frr, dxr, ispU, rfaK, kdtA, coaD, rpmB, dfp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR, rplF, rpsH, rpsN, rplE, rplX, rplN, rpsQ, rpmC, rplP, rpsC, rplV, rpsS, rplB, cdsA, yaeL, yaeT, lpxD, fabZ, lpxA, lpxB, dnaE, accA, tilS, proS, yafF, tsf, pyrH, olA, rlpB, leuS, lnt, glnS, fldA, cydA, infA, cydC, ftsK, lolA, serS, rpsA, msbA, lpxK, kdsB, mukF, mukE, mukB, asnS, fabA, mviN, rne, yceQ, fabD, fabG, acpP, tmk, holB, lolC, lolD, lolE, purB, ymfK, minE, mind, pth, rsA, ispE, lolB, hemA, prfA, prmC, kdsA, topA, ribA, fabI, racR, dicA, ydfB, tyrS, ribC, ydiL, pheT, pheS, yhhQ, bcsB, glyQ, yibJ, and gpsA. Other essential genes are known to those of ordinary skill in the art.

In some embodiments, the genetically engineered bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain,” ACS Synthetic Biology (2015) DOI: 10.1021/acssynbio.5b00085, the entire contents of which are expressly incorporated herein by reference).

In some embodiments, the SLiDE bacterial cell comprises a mutation in an essential gene. In some embodiments, the essential gene is selected from the group consisting of pheS, dnaN, tyrS, metG and adk. In some embodiments, the essential gene is dnaN comprising one or more of the following mutations: H191N, R240C, 1317S, F319V, L340T, V3471, and S345C. In some embodiments, the essential gene is dnaN comprising the mutations H191N, R240C, 1317S, F319V, L340T, V3471, and S345C. In some embodiments, the essential gene is pheS comprising one or more of the following mutations: F125G, P183T, P184A, R186A, and I188L. In some embodiments, the essential gene is pheS comprising the mutations F125G, P183T, P184A, R186A, and I188L. In some embodiments, the essential gene is tyrS comprising one or more of the following mutations: L36V, C38A and F40G. In some embodiments, the essential gene is tyrS comprising the mutations L36V, C38A and F40G. In some embodiments, the essential gene is metG comprising one or more of the following mutations: E45Q, N47R, I49G, and A51C. In some embodiments, the essential gene is metG comprising the mutations E45Q, N47R, I49G, and A51C. In some embodiments, the essential gene is adk comprising one or more of the following mutations: I4L, L5I and L6G. In some embodiments, the essential gene is adk comprising the mutations 14L, L51 and L6G.

In some embodiments, the genetically engineered bacterium is complemented by a ligand. In some embodiments, the ligand is selected from the group consisting of benzothiazole, indole, 2-aminobenzothiazole, indole-3-butyric acid, indole-3-acetic acid, and L-histidine methyl ester. For example, bacterial cells comprising mutations in metG (E45Q, N47R, I49G, and A51C) are complemented by benzothiazole, indole, 2-aminobenzothiazole, indole-3-butyric acid, indole-3-acetic acid or L-histidine methyl ester. Bacterial cells comprising mutations in dnaN (H191N, R240C, I317S, F319V, L340T, V347I, and S345C) are complemented by benzothiazole, indole or 2-aminobenzothiazole. Bacterial cells comprising mutations in pheS (F125G, P183T, P184A, R186A, and I188L) are complemented by benzothiazole or 2-aminobenzothiazole. Bacterial cells comprising mutations in tyrS (L36V, C38A, and F40G) are complemented by benzothiazole or 2-aminobenzothiazole. Bacterial cells comprising mutations in adk (14L, L51 and L6G) are complemented by benzothiazole or indole.

In some embodiments, the genetically engineered bacterium comprises more than one mutant essential gene that renders it auxotrophic to a ligand. In some embodiments, the bacterial cell comprises mutations in two essential genes. For example, in some embodiments, the bacterial cell comprises mutations in tyrS (L36V, C38A, and F40G) and metG (E45Q, N47R, 149G, and A51C). In other embodiments, the bacterial cell comprises mutations in three essential genes. For example, in some embodiments, the bacterial cell comprises mutations in tyrS (L36V, C38A, and F40G), metG (E45Q, N47R, 149G, and A51C), and pheS (F125G, P183T, P184A, R186A, and I188L).

In some embodiments, the genetically engineered bacterium is a conditional auxotroph whose essential gene(s) is replaced using the arabinose system described herein.

In some embodiments, the genetically engineered bacterium of the disclosure is an auxotroph and also comprises kill-switch circuitry, such as any of the kill-switch components and systems described herein. For example, the recombinant bacteria may comprise a deletion or mutation in an essential gene required for cell survival and/or growth, for example, in a DNA synthesis gene, for example, thyA, cell wall synthesis gene, for example, dapA and/or an amino acid gene, for example, serA or MetA or ilvC, and may also comprise a toxin gene that is regulated by one or more transcriptional activators that are expressed in response to an environmental condition(s) and/or signal(s) (such as the described arabinose system) or regulated by one or more recombinases that are expressed upon sensing an exogenous environmental condition(s) and/or signal(s) (such as the recombinase systems described herein). Other embodiments are described in Wright et al., “GeneGuard: A Modular Plasmid System Designed for Biosafety,” ACS Synthetic Biology (2015) 4: 307-16, the entire contents of which are expressly incorporated herein by reference). In some embodiments, the genetically engineered bacterium of the disclosure is an auxotroph and also comprises kill-switch circuitry, such as any of the kill-switch components and systems described herein, as well as another biosecurity system, such a conditional origin of replication (see Wright et al., supra).

Isolated Plasmids

In other embodiments, the disclosure provides an isolated plasmid comprising a first nucleic acid encoding a first payload operably linked to a first inducible promoter, and a second nucleic acid encoding a second payload operably linked to a second inducible promoter. In other embodiments, the disclosure provides an isolated plasmid further comprising a third nucleic acid encoding a third payload operably linked to a third inducible promoter. In other embodiments, the disclosure provides a plasmid comprising four, five, six, or more nucleic acids encoding four, five, six, or more payloads operably linked to inducible promoters. In any of the embodiments described here, the first, second, third, fourth, fifth, sixth, etc. “payload(s)” can be an oxalate catabolism enzyme, a transporter of oxalate, or other sequence described herein. In one embodiment, the nucleic acid encoding the first payload and the nucleic acid encoding the second payload are operably linked to the first inducible promoter. In one embodiment, the nucleic acid encoding the first payload is operably linked to a first inducible promoter and the nucleic acid encoding the second payload is operably linked to a second inducible promoter. In one embodiment, the first inducible promoter and the second inducible promoter are separate copies of the same inducible promoter. In another embodiment, the first inducible promoter and the second inducible promoter are different inducible promoters. In other embodiments comprising a third nucleic acid, the nucleic acid encoding the third payload and the nucleic acid encoding the first and second payloads are all operably linked to the same inducible promoter. In other embodiments, the nucleic acid encoding the first payload is operably linked to a first inducible promoter, the nucleic acid encoding the second payload is operably linked to a second inducible promoter, and the nucleic acid encoding the third payload is operably linked to a third inducible promoter. In some embodiments, the first, second, and third inducible promoters are separate copies of the same inducible promoter. In other embodiments, the first inducible promoter, the second inducible promoter, and the third inducible promoter are different inducible promoters. In some embodiments, the first promoter, the second promoter, and the optional third promoter, or the first promoter and the second promoter and the optional third promoter, are each directly or indirectly induced by low-oxygen or anaerobic conditions. In other embodiments, the first promoter, the second promoter, and the optional third promoter, or the first promoter and the second promoter and the optional third promoter, are each a fumarate and nitrate reduction regulator (FNR) responsive promoter. In other embodiments, the first promoter, the second promoter, and the optional third promoter, or the first promoter and the second promoter and the optional third promoter are each a ROS-inducible regulatory region. In other embodiments, the first promoter, the second promoter, and the optional third promoter, or the first promoter and the second promoter and the optional third promoter are each a RNS-inducible regulatory region.

In some embodiments, the heterologous gene and/or gene cassette encoding one or more oxalate catabolism enzyme(s) is operably linked to a constitutive promoter. In one embodiment, the constitutive promoter is a lac promoter. In another embodiment, the constitutive promoter is a Tet promoter. In another embodiment, the constitutive promoter is a constitutive Escherichia coli σ³² promoter. In another embodiment, the constitutive promoter is a constitutive Escherichia coli σ⁷⁰ promoter. In another embodiment, the constitutive promoter is a constitutive Bacillus subtilis σ^{A} promoter. In another embodiment, the constitutive promoter is a constitutive Bacillus subtilis σ^{B} promoter. In another embodiment, the constitutive promoter is a Salmonella promoter. In other embodiments, the constitutive promoter is a bacteriophage T7 promoter. In other embodiments, the constitutive promoter is and a bacteriophage SP6 promoter. In any of the above-described embodiments, the plasmid further comprises a heterologous gene encoding a transporter oxalate, and/or a kill switch construct, which may be operably linked to a constitutive promoter or an inducible promoter.

In some embodiments, the isolated plasmid comprises at least one heterologous oxalate catabolism enzyme gene operably linked to a first inducible promoter; a heterologous gene encoding a TetR protein operably linked to a P_{araBAD} promoter, a heterologous gene encoding AraC operably linked to a P_{araC} promoter, a heterologous gene encoding an antitoxin operably linked to a constitutive promoter, and a heterologous gene encoding a toxin operably linked to a P_{TetR} promoter. In another embodiment, the isolated plasmid comprises at least one heterologous gene and/or gene cassette encoding one or more oxalate catabolism enzyme(s) operably linked to a first inducible promoter; a heterologous gene encoding a TetR protein and an anti-toxin operably linked to a P_{araBAD} promoter, a heterologous gene encoding AraC operably linked to a P_{araC} promoter, and a heterologous gene encoding a toxin operably linked to a P_{TetR} promoter.

In some embodiments, a first nucleic acid encoding one or more oxalate catabolism enzyme(s) comprises a Formyl CoA: oxalate CoA transferase (e.g., frc) gene. In one embodiment, the frc gene is from O. formigenes. In one embodiment, the frc gene has at least about 90% identity to SEQ ID NO: 1. In another embodiment, the frc gene comprises SEQ ID NO: 1. In other embodiments, a first nucleic acid encoding one or more oxalate catabolism enzyme(s) comprises an Oxalate-CoA ligase (e.g., ScAAE3) gene. In one embodiment, the ScAAE3 gene is from S. cerevisiae. In one embodiment, the ScAAE3 gene has at least about 90% identity to SEQ ID NO: 3. In another embodiment, the ScAAE3gene comprises SEQ ID NO: 3. In other embodiments, a first nucleic acid encoding one or more oxalate catabolism enzyme(s) comprises an acetyl-CoA:oxalate CoA-transferase (e.g., YfdE) gene. In one embodiment, the YfdE gene is from E. coli. In one embodiment, the YfdE gene has at least about 90% identity to SEQ ID NO: 4. In another embodiment, the YfdE gene comprises SEQ ID NO: 4.

In some embodiments, a first nucleic acid encoding one or more oxalate catabolism enzyme(s) comprises a Oxalyl-CoA Decarboxylase (e.g., oxc) gene. In some embodiments, the frc and/or ScAAE3 and/or YfdE gene(s) are co-expressed with a Oxalyl-CoA Decarboxylase (e.g., oxc) gene. In one embodiment, the oxc gene is from O. formigenes. In one embodiment, the oxc gene has at least about 90% identity to SEQ ID NO: 2. In another embodiment, the oxc gene comprises SEQ ID NO: 2.

In some embodiments, a second nucleic acid encoding a transporter of oxalate comprises OxlT. In one embodiment, the OxlT transporter is from O. formigenes. In another embodiment, the OxlT transporter has at least about 90% identity to SEQ ID NO: 11. In another embodiment, the OxlT transporter comprises SEQ ID NO: 11.

In one embodiment, the plasmid is a high-copy plasmid. In another embodiment, the plasmid is a low-copy plasmid.

In another aspect, the disclosure provides a recombinant bacterial cell comprising an isolated plasmid described herein. In another embodiment, the disclosure provides a pharmaceutical composition comprising the recombinant bacterial cell.

In one embodiment, the bacterial cell further comprises a genetic mutation in an endogenous gene encoding an exporter of oxalate, wherein the genetic mutation reduces export of oxalate from the bacterial cell.

In one embodiment, the bacterial cell further comprises a genetic mutation in an endogenous gene encoding an oxalate biosynthesis gene, wherein the genetic mutation reduces biosynthesis of oxalate in the bacterial cell.

Integration

In some embodiments, any of the gene(s) or gene cassette(s) of the present disclosure may be integrated into the bacterial chromosome at one or more integration sites. One or more copies of the gene (for example, an oxalate catabolism gene, oxalate transporter gene, and/or oxalate binding protein gene) or gene cassette (for example, a gene cassette comprising an oxalate catabolism gene and/or an oxalate transporter gene may be integrated into the bacterial chromosome. Having multiple copies of the gene or gene cassette integrated into the chromosome allows for greater production of the payload, e.g., one or more oxalate catabolism enzyme(s) and/or oxalate transporter gene(s) and other enzymes of a gene cassette, and also permits fine-tuning of the level of expression. Alternatively, different circuits described herein, such as any of the kill-switch circuits, in addition to the therapeutic gene(s) or gene cassette(s) could be integrated into the bacterial chromosome at one or more different integration sites to perform multiple different functions.

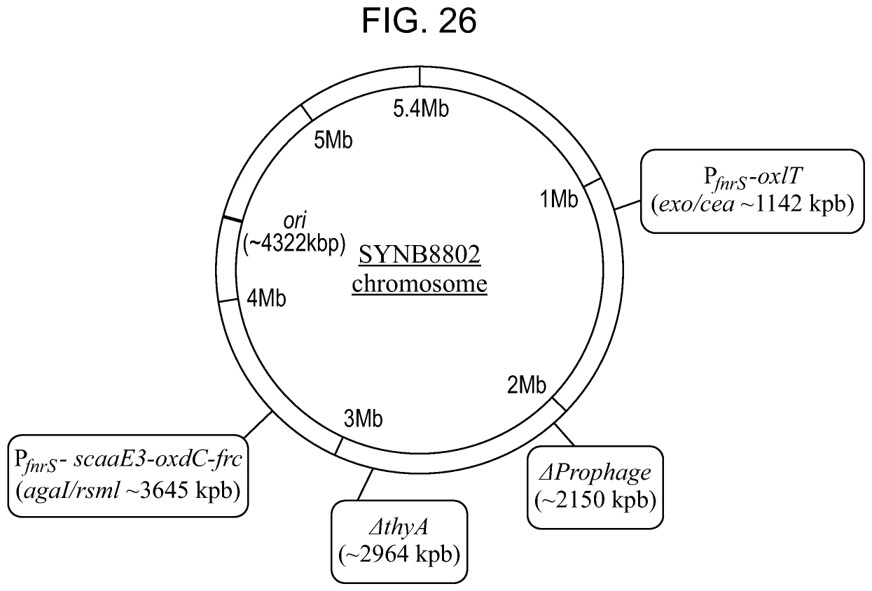

FIG. 26 depicts the genotype of SYNB8802. SYNB8802 is a strain of modified live probiotic bacterium (Escherichia coli Nissle 1917 [EcN]) that has been modified to treat EH by consuming oxalate within the gastrointestinal tract. The locations of the genomic modification sites in SYNB8802 are shown, with kbp designation indicating the chromosomal position relative to the 0/5.4 Mb reference marker. The chromosomal origin of replication is shown as a red line (ori). Italicized gene names in parenthesis refer to the upstream and downstream genes surrounding the inserted genes. SYNB8802 was developed by engineering a pathway for oxalate degradation in a probiotic strain of EcN using the oxalate degradation capabilities of the human commensal microorganism Oxalobacter formigenes. The following modifications to the genome of EcN have been made to enhance oxalate degradation under the low oxygen conditions found in the gut, while augmenting biologic containment through thymidine auxotrophy: (1) Insertion of one gene encoding an oxalate/formate antiporter (OxlT) derived from Oxalobacter formigenes under the regulatory control of an anaerobic-inducible promoter (PfnrS) and the anaerobic-responsive transcriptional activator FNR. (2) Insertion of one operon, encoding three genes under the regulatory control of an anaerobic-inducible promoter (PfnrS) and the anaerobic-responsive transcriptional activator FNR. The first gene is an oxalyl-CoA synthetase (ScaaE3) derived from Saccharomyces cerevisiae. The second gene is an oxalate decarboxylase (OxdC) derived from Oxalobacter formigenes. The third gene (frc) is a formyl-CoA transferase derived from Oxalobacter formigenes. (Deletion of the thymidylate synthase (thyA) gene to create a thymidine auxotroph. (3) Deletion of the thymidylate synthase (thyA) gene to create a thymidine auxotroph. (4) Inactivation of the endogenous Nissle prophage. (5) Additionally, mutation in the pks island.

In some embodiments, the genetically engineered bacteria comprise a stably maintained plasmid or chromosome carrying a gene for producing an oxalate catabolism enzyme and/or oxalate transporter such that the oxalate catabolism enzyme and/or oxalate transporter can be expressed in the host cell, and the host cell is capable of survival and/or growth in vitro, e.g., in medium, and/or in vivo. In some embodiments, a bacterium may comprise multiple copies of the gene and/or gene cassette encoding one or more oxalate catabolism enzyme(s) and/or oxalate transporter(s). In some embodiments, the gene and/or gene cassette encoding one or more oxalate catabolism enzyme(s) and/or oxalate transporter(s) is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, the gene and/or gene cassette encoding one or more oxalate catabolism enzyme(s) and/or oxalate transporter(s) is expressed on a high-copy plasmid. In some embodiments, the high-copy plasmid may be useful for increasing expression of the oxalate catabolism enzyme(s) and/or oxalate transporter(s). In some embodiments, the gene and/or gene cassette encoding one or more oxalate catabolism enzyme(s) and/or oxalate transporter(s) is expressed on a chromosome.

In some embodiments, the bacteria are genetically engineered to include multiple mechanisms of action (MOAs), e.g., circuits producing multiple copies of the same product (e.g., to enhance copy number) or circuits performing multiple different functions. For example, the genetically engineered bacteria may include four copies of the gene and/or gene cassette encoding one or more particular oxalate catabolism enzyme(s) and/or oxalate transporter(s) inserted at four different insertion sites. Alternatively, the genetically engineered bacteria may include three copies of the gene encoding a particular oxalate catabolism enzyme and/or oxalate transporter inserted at three different insertion sites and three copies of the gene encoding a different oxalate catabolism enzyme and/or oxalate transporter inserted at three different insertion sites.

In some embodiments, under conditions where the oxalate catabolism enzyme(s) and/or oxalate transporter is expressed, the genetically engineered bacteria of the disclosure produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of the oxalate catabolism enzyme(s) and/or oxalate transporter(s) and/or transcript of the gene(s) in the operon as compared to unmodified bacteria of the same subtype under the same conditions.

In some embodiments, quantitative PCR (qPCR) is used to amplify, detect, and/or quantify mRNA expression levels of the oxalate catabolism enzyme(s) and/or oxalate transporter(s) gene(s). Primers specific for oxalate catabolism enzyme and/or oxalate transporter gene(s) may be designed and used to detect mRNA in a sample according to methods known in the art. In some embodiments, a fluorophore is added to a sample reaction mixture that may contain oxalate catabolism enzyme mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the oxalate catabolism enzyme and/or oxalate transporter gene(s).

In some embodiments, quantitative PCR (qPCR) is used to amplify, detect, and/or quantify mRNA expression levels of the oxalate catabolism enzyme(s) and/or oxalate transporter gene(s). Primers specific for oxalate catabolism enzyme and/or oxalate transporter gene(s) may be designed and used to detect mRNA in a sample according to methods known in the art. In some embodiments, a fluorophore is added to a sample reaction mixture that may contain oxalate catabolism enzyme and/or oxalate transporter mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the oxalate catabolism enzyme and/or oxalate transporter gene(s).

Pharmaceutical Compositions and Formulations

Pharmaceutical compositions comprising the genetically engineered microorganisms of the invention may be used to treat, manage, ameliorate, and/or prevent diseases or disorders in which oxalate is detrimental in a subject. In another embodiment, the disorder in which oxalate is detrimental is a disorder that results in daily urinary oxalate excretion over 40 mg per 24 hours. Pharmaceutical compositions of the invention comprising one or more genetically engineered bacteria, and/or one or more genetically engineered virus, alone or in combination with prophylactic agents, therapeutic agents, and/or pharmaceutically acceptable carriers are provided.

In certain embodiments, the pharmaceutical composition comprises one species, strain, or subtype of bacteria that are engineered to comprise one or more of the genetic modifications described herein, e.g., selected from expression of at least one oxalate catabolism enzyme, oxalate importer/transporter and/or formate exporter and/or oxalate:formate antiporter, auxotrophy, kill-switch, knock-out, etc. In alternate embodiments, the pharmaceutical composition comprises two or more species, strains, and/or subtypes of bacteria that are each engineered to comprise the genetic modifications described herein, e.g., one oxalate catabolism enzyme, oxalate importer/transporter and/or formate exporter and/or oxalate:formate antiporter, auxotrophy, kill-switch, knock-out, etc.

The pharmaceutical compositions of the disclosure may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, PA). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered microorganisms may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, immediate-release, pulsatile-release, delayed-release, or sustained release). Suitable dosage amounts for the genetically engineered bacteria may range from about 10⁴ to 10¹² bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal.

The genetically engineered bacteria or genetically engineered virus may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20.

In some embodiments, the genetically engineered bacteria of the invention may be formulated in a solution of sodium bicarbonate, e.g., 1 molar solution of sodium bicarbonate or another concentration described herein (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered microorganisms may be administered intravenously, e.g., by infusion or injection.

The genetically engineered microorganisms of the disclosure may be administered intrathecally. In some embodiments, the genetically engineered microorganisms of the invention may be administered orally. The genetically engineered microorganisms disclosed herein may be administered topically and formulated in the form of an ointment, cream, transdermal patch, lotion, gel, shampoo, spray, aerosol, solution, emulsion, or other form well known to one of skill in the art. See, e.g., “Remington's Pharmaceutical Sciences,” Mack Publishing Co., Easton, PA. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the recombinant bacteria of the invention may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The genetically engineered microorganisms disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroxyethylacrylate-methyl methacrylate (HEMA-MMA), multilayered HEMA-MMA-MAA, polyacrylonitrilevinylchloride (PAN-PVC), acrylonitrile/sodium metallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered microorganisms are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

In some embodiments, enteric coating materials may be used, in one or more coating layers (e.g., outer, inner and/o intermediate coating layers). Enteric coated polymers remain unionised at low pH, and therefore remain insoluble. But as the pH increases in the gastrointestinal tract, the acidic functional groups are capable of ionisation, and the polymer swells or becomes soluble in the intestinal fluid.

Materials used for enteric coatings include Cellulose acetate phthalate (CAP), Poly(methacrylic acid-co-methyl methacrylate), Cellulose acetate trimellitate (CAT), Poly(vinyl acetate phthalate) (PVAP) and Hydroxypropyl methylcellulose phthalate (HPMCP), fatty acids, waxes, Shellac (esters of aleurtic acid), plastics and plant fibers. Additionally, Zein, Aqua-Zein (an aqueous zein formulation containing no alcohol), amylose starch and starch derivatives, and dextrins (e.g., maltodextrin) are also used. Other known enteric coatings include ethylcellulose, methylcellulose, hydroxypropyl methylcellulose, amylose acetate phthalate, cellulose acetate phthalate, hydroxyl propyl methyl cellulose phthalate, an ethylacrylate, and a methylmethacrylate.

Coating polymers also may comprise one or more of, phthalate derivatives, CAT, HPMCAS, polyacrylic acid derivatives, copolymers comprising acrylic acid and at least one acrylic acid ester, Eudragit™ S (poly(methacrylic acid, methyl methacrylate)1:2); Eudragit L100™ S (poly(methacrylic acid, methyl methacrylate)1:1); Eudragit L30D™, (poly(methacrylic acid, ethyl acrylate)1:1); and (Eudragit L100-55) (poly(methacrylic acid, ethyl acrylate)1:1) (Eudragit™ L is an anionic polymer synthesized from methacrylic acid and methacrylic acid methyl ester), polymethyl methacrylate blended with acrylic acid and acrylic ester copolymers, alginic acid, ammonia alginate, sodium, potassium, magnesium or calcium alginate, vinyl acetate copolymers, polyvinyl acetate 30D (30% dispersion in water), a neutral methacrylic ester comprising poly(dimethylaminoethylacrylate) (“Eudragit E™), a copolymer of methylmethacrylate and ethylacrylate with trimethylammonioethyl methacrylate chloride, a copolymer of methylmethacrylate and ethylacrylate, Zein, shellac, gums, or polysaccharides, or a combination thereof.

Coating layers may also include polymers which contain Hydroxypropylmethylcellulose (HPMC), Hydroxypropylethylcellulose (HPEC), Hydroxypropylcellulose (HPC), hydroxypropylethylcellulose (HPEC), hydroxymethylpropylcellulose (HMPC), ethylhydroxyethylcellulose (EHEC) (Ethulose), hydroxyethylmethylcellulose (HEMC), hydroxymethylethylcellulose (HMEC), propylhydroxyethylcellulose (PHEC), methylhydroxyethylcellulose (MHEC), hydrophobically modified hydroxyethylcellulose (NEXTON), carboxymethyl hydroxyethylcellulose (CMHEC), Methylcellulose, Ethylcellulose, water soluble vinyl acetate copolymers, gums, polysaccharides such as alginic acid and alginates such as ammonia alginate, sodium alginate, potassium alginate, acid phthalate of carbohydrates, amylose acetate phthalate, cellulose acetate phthalate (CAP), cellulose ester phthalates, cellulose ether phthalates, hydroxypropylcellulose phthalate (HPCP), hydroxypropylethylcellulose phthalate (HPECP), hydroxypropylmethylcellulose phthalate (HPMCP), hydroxypropylmethylcellulose acetate succinate (HPMCAS).

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered microorganisms described herein.

In one embodiment, the genetically engineered microorganisms of the disclosure may be formulated in a composition suitable for administration to pediatric subjects. As is well known in the art, children differ from adults in many aspects, including different rates of gastric emptying, pH, gastrointestinal permeability, etc. (Ivanovska et al., Pediatrics, 134(2):361-372, 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered microorganisms may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria of the invention may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria of the invention are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria of the invention are well known in the art. For example, see U.S. 2015/0359894 and US 2015/0238545, the entire contents of each of which are expressly incorporated herein by reference. In yet another embodiment, the pharmaceutical composition of the invention is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered microorganisms described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered microorganisms may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

In some embodiments, the invention provides pharmaceutically acceptable compositions that are not in the form of or incorporated into a food or edible product.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD50, ED50, EC50, and IC50 may be determined, and the dose ratio between toxic and therapeutic effects (LD50/ED50) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

In one embodiment, the recombinant bacteria are administered at a dose of about 1×10¹¹ live recombinant bacteria, about 2×10¹¹ live recombinant bacteria, about 3×10¹¹ live recombinant bacteria, about 4×10¹¹ live recombinant bacteria, about 4.5×10¹¹ live recombinant bacteria, about 5×10¹¹ live recombinant bacteria, about 6×10¹¹ live recombinant bacteria, about 1×10¹² live recombinant bacteria, or about 2×10¹² live recombinant bacteria. In one embodiment, the recombinant bacteria are administered at a dose of about 6×10¹¹ live recombinant bacteria. In one embodiment, the recombinant bacteria are administered at a dose of about 1×10¹¹ live recombinant bacteria. In one embodiment, the administering is about 4.5×10¹¹ live recombinant bacteria. In one embodiment, the administering is about 5×10¹¹ live recombinant bacteria. In one embodiment, the recombinant bacteria are administered at a dose of about 1×10¹² live recombinant bacteria. In one embodiment, the recombinant bacteria are administered at a dose of about 2×10¹² live recombinant bacteria. In one embodiment, the administering is about 5×10¹¹ live recombinant bacteria with meals three times per day. In one embodiment, the recombinant bacteria are administered at a dose of about 6×10¹¹ live recombinant bacteria with meals three times per day. In one embodiment, the recombinant bacteria are administered at a dose of about 1×10¹¹ live recombinant bacteria with meals three times per day. In one embodiment, the recombinant bacteria are administered at a dose of about 1×10¹² live recombinant bacteria with meals three times per day. In one embodiment, the recombinant bacteria are administered at a dose of about 2×10¹² live recombinant bacteria with meals three times per day. In one embodiment, the recombinant bacteria are administered at a dose of about 4.5×10¹² live recombinant bacteria with meals three times per day.

In some embodiments, a subject may not tolerate twice daily or three times daily dosing, and the dosing frequency may be reduced.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the genetically engineered viruses are prepared for delivery, taking into consideration the need for efficient delivery and for overcoming the host antiviral immune response. Approaches to evade antiviral response include the administration of different viral serotypes as par of the treatment regimen (serotype switching), formulation, such as polymer coating to mask the virus from antibody recognition and the use of cells as delivery vehicles.

In another embodiment, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The genetically engineered bacteria of the invention may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In Vivo Methods

The recombinant bacteria of the invention may be evaluated in vivo, e.g., in an animal model. Any suitable animal model of a disease or condition in which oxalate is detrimental may be used. For example, an alanine glyoxylate aminotransferase-deficient (agxt −/−) mouse model of PHI as described by Salido et al. can be used (see, e.g., Salido et al., Proc. Nat. Acad. Sci. 103: 18249-54 (2006)). A glyoxylate reductase/hydroxypyruvate reductase knock-out (GRHPR −/−) mouse model of PHII can also be used (see, e.g., Knight et al., Am. J. Physiol. Renal. Physiol. 302: F688-93 (2012)). Mice deficient in the oxalate transporter protein SLC26A6 (Slc26a6-null mice) which develop hyperoxaluria can also be used (see, e.g., Jiang et al. Nature Gen. 38: 474-8 (2006)).

Alternatively, a rat model may be used. For example, Canales et al. describe a rat model of Roux-en-Y gastric bypass (RYGB) surgery, in which high fat feeding results in steatorrhea, hyperoxaluria, and low urine pH. RYGB animals on normal fat and no oxalate diets excreted twice as much oxalate as age-matched, sham controls; hyperoxaluria was partially reversible by lowering dietary fat and oxalate content (Canales et al., Steatorrhea And Hyperoxaluria Occur After Gastric Bypass Surgery In Obese Rats Regardless Of Dietary Fat Or Oxalate; J Urol. 2013 September; 190(3): 1102-1109).

The recombinant bacterial cells of the invention may be administered to the animal, e.g., by oral gavage, and treatment efficacy is determined, e.g., by measuring urine levels of oxalic acid before and after treatment. The animal may be sacrificed, and tissue samples may be collected and analyzed.

The following Table 13 includes additional rat models which can be used to assess in vivo activity of the genetically engineered bacteria.

TABLE 13
Rat Models of Calcium Oxalate Nephrolithiasis

| Induction | | Renal changes and | |
|---|---|---|---|
| Technique | Crystal Deposition | urinary changes | Strengths |
| Ethylene glycol in | Intraluminal in renal | Necrotic and apoptotic | Easy to induce |
| drinking water | tubules of both | renal injury, interstitial | consistent |
| | cortex and medulla, | inflammation, increased | hyperoxaluria, |
| | crystals deposit in | synthesis and urinary | crystalluria, and CaOx |
| | association with | excretion of OPN, | nephrolithiasis |
| | cellular degradation | Bikunin, MCP-1, alpha-1- | |
| | products, plaques and | microglobulin, | |
| | stones at papillary | hyperoxaluria, enzymuria, | |
| | tips. | membranuria, and CaOx | |
| | | crystaluria | |
| Hydroxy-L-proline | No crystals in the | Kidneys appear normal, | Simple, more |
| in drinking water | renal fornices and | hyperoxaluria, enzymuria, | physiological than the |
| | pelvis | and CaOx crystalluria, | administration of |
| | | increased synthesis of | ethylene glycol or |
| | | OPN by papillary surface | some other oxalate |
| | | epithelial cells | precursors |
| Hydroxy-L-proline | Intraluminal in renal | Hyperoxaluria, CaOx | Simple, more |
| mixed with food | tubules of both | crystalluria, signs of renal | physiological than the |
| | cortex and medulla, | injury and inflammation in | administration of |
| | plaques and stones at | association with the | ethylene glycol or |
| | papillary tips | crystals | some other oxalate |
| | | | precursors |
| Implantation of | Intraluminal in renal | Hyperoxaluria, CaOx | Reliable and consistent |
| osmotic mini- | tubules of both | crystalluria, upregulation | hyperoxaluria and |
| pumps filled with | cortex and medulla | of TNF receptor kidney | CaOx nephrolithiasis |
| oxalate | | injury marker and OPN | |
| Vitamin B-6- | CaOx crystals | Hyperoxaluria, | |
| deficient diet | intraluminal in | hypercalciuria, enzymuria, | |
| | tubules of medulla, | hypocitraturia, CaOx | |
| | plaques at papillary | crystalluria | |
| | tips, stones in renal | | |
| | fornices, pelvis, | | |
| | ureters, and bladder | | |
| Glycolic acid in | CaOx crystals in | Hyperoxaluria | |
| diet | tubules of renal | | |
| | cortex and medulla, | | |
| | stones in renal pelvis | | |
| Ileal resection and | CaOx crystals mixed | Tubular obstruction and | Models nephrolithiasis |
| feeding of oxalate | with CaP and Ca | interstitial inflammation, | after ileal resection or |
| | carbonate crystals, | hyperoxaluria, | bypass surgery |
| | intraluminal in both | hypocitraturia | |
| | cortex and medulla, | | |
| | interstitial in the | | |
| | papilla, plaques on | | |
| | papillary surface | | |

Methods of Screening

In some embodiments of the invention, the efficacy or activity of any of the importers, exporters, antiporters, and oxalate catabolism enzymes can be improved through mutations in any of these genes. Methods for directed mutation and screening are known in the art.

Methods of Treatment

One aspect of the invention provides methods of treating a disorder in which oxalate is detrimental in a subject, or symptom(s) associated with the disorder in which oxalate is detrimental in a subject. In one embodiment, the disorder in which oxalate is detrimental is a disorder associated with increased levels of oxalate. In one embodiment, a disorder associated with increased levels of oxalate is a disorder in which daily urinary oxalate excretion is 40 mg or higher per 24 hours. Disorders associated with increased levels of oxalate include PHI, PHII, PHIII, secondary hyperoxaluria, enteric hyperoxaluria, dietary hyperoxaluria, idiopathic hyperoxaluria, syndrome of bacterial overgrowth, Crohn's disease, inflammatory bowel disease, hyperoxaluria following renal transplantation, hyperoxaluria after a jejunoileal bypass for obesity, hyperoxaluria after gastric ulcer surgery, chronic mesenteric ischemia, gastric bypass, cystic fibrosis, short bowel syndrome, biliary/pancreatic diseases (e.g., chronic pancreatitis), hyperoxaluria with recurrent kidney stones with relatively preserved renal function, and hyperoxaluria with recurrent kidney stones with severe renal dysfunction (e.g., including patients on hemodialysis). In one embodiment, the disorder in which oxalate is detrimental is PHI. In one embodiment, the disorder in which oxalate is detrimental is PHIL. In another embodiment, the disorder in which oxalate is detrimental is PHIII. In one embodiment, the disorder in which oxalate is detrimental is secondary hyperoxaluria. In another embodiment, the disorder in which oxalate is detrimental is dietary hyperoxaluria. In one embodiment, the disorder in which oxalate is detrimental is idiopathic hyperoxaluria. In another embodiment, the disorder in which oxalate is detrimental is enteric hyperoxaluria. In one embodiment, the disorder in which oxalate is detrimental is the syndrome of bacterial overgrowth. In another embodiment, the disorder in which oxalate is detrimental is Crohn's disease. In one embodiment, the disorder in which oxalate is detrimental is inflammatory bowel disease. In another embodiment, the disorder in which oxalate is detrimental is hyperoxaluria following renal transplantation. In one embodiment, the disorder in which oxalate is detrimental is hyperoxaluria after a jejunoileal bypass for obesity. In another embodiment, the disorder in which oxalate is detrimental is hyperoxaluria after gastric ulcer surgery. In one embodiment, the disorder in which oxalate is detrimental is chronic mesenteric ischemia. In another embodiment, the disorder in which oxalate is detrimental is gastric bypass, e.g., Roux-enY gastric bypass. In another embodiment, the disorder in which oxalate is detrimental is cystic fibrosis. In another embodiment, the disorder in which oxalate is detrimental is short bowel syndrome. In another embodiment, the disorder in which oxalate is detrimental are biliary/pancreatic diseases. In another embodiment, the disorder in which oxalate is detrimental is chronic pancreatitis. In another embodiment, the disorder in which oxalate is detrimental is hyperoxaluria with recurrent kidney stones with relatively preserved renal function. In another embodiment, the disorder in which oxalate is detrimental is hyperoxaluria with recurrent kidney stones with severe renal dysfunction (e.g., including patients on hemodialysis).

The present disclosure surprisingly demonstrates that pharmaceutical compositions comprising the recombinant bacterial cells disclosed herein may be used to treat disorders in which oxalate is detrimental, such as PHI and PHI.

In one embodiment, the subject having PHI has a mutation in a AGXT gene. In another embodiment, the subject having PHII has a mutation in a GRHPR gene. In one embodiment, the subject having PHIII has a mutation in a HOGA1 gene. In another aspect, the invention provides methods for decreasing the plasma level of oxalate and/or oxalic acid in a subject by administering a pharmaceutical composition comprising a bacterial cell of the invention to the subject, thereby decreasing the plasma level of the oxalate and/or oxalic acid in the subject. In one embodiment, the subject has a disease or disorder in which oxalate is detrimental. In one embodiment, the disorder in which oxalate is detrimental is PHI.

In one embodiment, the disorder in which oxalate is detrimental is PHIL. In another embodiment, the disorder in which oxalate is detrimental is PHIII. In one embodiment, the disorder in which oxalate is detrimental is secondary hyperoxaluria. In another embodiment, the disorder in which oxalate is detrimental is dietary hyperoxaluria. In one embodiment, the disorder in which oxalate is detrimental is idiopathic hyperoxaluria. In another embodiment, the disorder in which oxalate is detrimental is enteric hyperoxaluria. In one embodiment, the disorder in which oxalate is detrimental is the syndrome of bacterial overgrowth. In another embodiment, the disorder in which oxalate is detrimental is Crohn's disease. In one embodiment, the disorder in which oxalate is detrimental is inflammatory bowel disease. In another embodiment, the disorder in which oxalate is detrimental is hyperoxaluria following renal transplantation. In one embodiment, the disorder in which oxalate is detrimental is hyperoxaluria after a jejunoileal bypass for obesity. In another embodiment, the disorder in which oxalate is detrimental is hyperoxaluria after gastric ulcer surgery. In one embodiment, the disorder in which oxalate is detrimental is chronic mesenteric ischemia. In another embodiment, the disorder in which oxalate is detrimental is gastric bypass. In another embodiment, the disorder in which oxalate is detrimental is cystic fibrosis. In another embodiment, the disorder in which oxalate is detrimental is short bowel syndrome. In another embodiment, the disorder in which oxalate is detrimental are biliary/pancreatic diseases. In another embodiment, the disorder in which oxalate is detrimental is chronic pancreatitis.

In some embodiments, the disclosure provides methods for reducing, ameliorating, or eliminating one or more symptom(s) associated with these diseases, including but not limited to fever, vomiting, nausea, diarrhea, kidney stones, oxalosis, bone disease, erythropoietin refractory anemia, skin ulcers, digital gangrene, cardiac arrhythmias, and cardiomyopathy. In some embodiments, the disease is secondary to other conditions, e.g., liver disease.

In some embodiments, the human patient to be treated by the methods disclosed herein may meet one or more of the inclusion and exclusion criteria disclosed in the Examples below. For example the human patient may of age ≥18 to ≤74 years. In some embodiments, the human patient has a history of gastric bypass surgery (at least 12 months prior to Day 1) or of short-bowel syndrome.

Alternatively or in addition, the human patient subject to any treatment disclosed herein may be free of or does not have one or more of (1) Acute or chronic medical (including COVID-19 infection), (3) Estimated glomerular filtration rate <45 m/min/1.73 m2 (4) History of kidney stones (5) Inability to discontinue vitamin C supplementation; (6) known primary hyperoxaluria (7) Administration or ingestion of any type of systemic (e.g., oral or intravenous) antibiotic within 5 half-lives of the agent prior to Day 1 (8) Intolerance of, or allergic reaction to, EcN, all PPIs, or any of the ingredients in SYNB8802 or placebo formulations (9) Dependence on alcohol or drugs of abuse (10) Current, immunodeficiency disorder including autoimmune disorders and uncontrolled human immunodeficiency virus (HIV).

In certain embodiments, the bacterial cells disclosed herein are capable of catabolizing oxalate and/or oxalic acid in a subject in order to treat a disorder in which oxalate is detrimental. In these embodiments, a patient suffering from a disorder in which oxalate is detrimental, e.g., PHI or PHII, may be able to resume a substantially normal diet, or a diet that is less restrictive than an oxalate-free or a very low-oxalate diet. In some embodiments, the bacterial cells may be capable of catabolizing oxalate and/or oxalic acid, from additional sources, e.g., the blood, in order to treat a disorder in which oxalate is detrimental.

In some embodiments, dietary uptake of oxalate is suppressed by providing the genetically engineered bacteria described herein. In some embodiments, oxalate generated through metabolic pathways, e.g., in a mammal is reduced.

The method may comprise preparing a pharmaceutical composition with at least one genetically engineered species, strain, or subtype of bacteria described herein, and administering the pharmaceutical composition to a subject in a therapeutically effective amount. In some embodiments, the method of treating a disease or disorder associated with elevated oxalate comprises administering to a subject in need thereof an engineered bacterium comprising gene sequence(s) encoding one or more oxalate catabolism enzyme(s) or a pharmaceutical composition thereof. In some embodiments, the method of treating a disease or disorder associated with elevated oxalate comprises administering to a subject in need thereof an engineered bacterium comprising gene sequence(s) encoding one or more oxalate transporter(s) or a pharmaceutical composition thereof. In some embodiments, the method of treating a disease or disorder associated with elevated oxalate comprises administering to a subject in need thereof an engineered bacterium comprising gene sequence(s) encoding one or more formate importers(s) or a pharmaceutical composition thereof. In some embodiments, the method of treating a disease or disorder associated with elevated oxalate comprises administering to a subject in need thereof an engineered bacterium comprising gene sequence(s) encoding one or more oxalate:formate antiporter(s) or a pharmaceutical composition thereof. In some embodiments, the method of treating a disease or disorder associated with elevated oxalate comprises administering to a subject in need thereof an engineered bacterium comprising gene sequence(s) encoding one or more oxalate catabolism enzyme(s) and gene sequence(s) encoding one or more of the following: (i) one or more oxalate transporter(s); (ii) one or more formate exporter(s); (iii) one or more oxalate:formate antiporter(s); and (iv) combinations thereof or a pharmaceutical composition thereof. In some embodiments, the method of treating a disease or disorder associated with elevated oxalate comprises administering to a subject in need thereof an engineered bacterium SYNB8802. In some embodiments, the bacterial cells disclosed herein are administered orally, e.g., in a liquid suspension. In some embodiments, the bacterial cells disclosed herein are lyophilized in a gel cap and administered orally. In some embodiments, the bacterial cells disclosed herein are administered via a feeding tube or gastric shunt. In some embodiments, the bacterial cells disclosed herein are administered rectally, e.g., by enema. In some embodiments, the genetically engineered bacteria are administered topically, intraintestinally, intrajejunally, intraduodenally, intraileally, and/or intracolically.

In certain embodiments, the administering the pharmaceutical composition described herein is administered to reduce oxalate and/or oxalic acid levels in a subject. In some embodiments, the methods of the present disclosure reduce the oxalate and/or oxalic acid levels in a subject by at least about 10%, 20%, 25%, 30%, 40%, 50%, 60%, 70%, 75%, 80%, 85%, 90%, 95%, or more. In another embodiment, the methods of the present invention reduce the oxalate and/or oxalic acid levels in a subject by at least two-fold, three-fold, four-fold, five-fold, six-fold, seven-fold, eight-fold, nine-fold, or ten-fold. In another embodiment, the methods of the present invention reduce the daily urinary oxalate excretion of a subject to less than 40 mg per 24 hours. In some embodiments, reduction is measured by comparing the oxalate and/or oxalic acid level in a subject before and after administration of the pharmaceutical composition. In one embodiment, the oxalate and/or oxalic acid level is reduced in the gut of the subject. In one embodiment, the oxalate and/or oxalic acid level is reduced in the urine of the subject. In another embodiment, the oxalate and/or oxalic acid level is reduced in the blood of the subject. In another embodiment, the oxalate and/or oxalic acid level is reduced in the plasma of the subject. In another embodiment, the oxalate and/or oxalic acid level is reduced in the fecal matter of the subject. In another embodiment, the oxalate and/or oxalic acid level is reduced in the brain of the subject. Creatinine is measured is used to correct for urine concentration, i.e., in some embodiments, the Uox:Creatinine ratio is measured to assess reduction in urinary oxalate levels.

In one embodiment, the pharmaceutical composition described herein is administered to reduce oxalate and/or oxalic acid levels in a subject to normal levels. In another embodiment, the pharmaceutical composition described herein is administered to reduce oxalate and/or oxalic acid levels in a subject to below a normal level. In another embodiment, the pharmaceutical composition described herein is administered to reduce the daily urinary oxalate excretion of a subject to less than 40 mg per 24 hours.

In certain embodiments, the pharmaceutical composition described herein is administered to reduce oxalate levels in a subject. In some embodiments, the methods of the present disclosure reduce the oxalate levels, in a subject by at least about 10%, 20%, 25%, 30%, 40%, 50%, 60%, 70%, 75%, 80%, 85%, 90%, 95%, or more as compared to levels in an untreated or control subject. In another embodiment, the methods of the present disclosure reduce the oxalate levels, in a subject by at least two-fold, three-fold, four-fold, five-fold, six-fold, seven-fold, eight-fold, nine-fold, or ten-fold. In some embodiments, reduction is measured by comparing the oxalate levels in a subject before and after administration of the pharmaceutical composition. In one embodiment, the oxalate level is reduced in the gut of the subject. In another embodiment, the oxalate level is reduced in the blood of the subject. In another embodiment, the oxalate level is reduced in the plasma of the subject. In another embodiment, the oxalate level is reduced in the liver of the subject. In another embodiment, the oxalate level is reduced in the kidney of the subject.

In one embodiment, the pharmaceutical composition described herein is administered to reduce oxalate in a subject to a normal level.

In some embodiments, the methods provided herein include monitoring of and/or result in changes in one or more endpoints described in Example 10 or other Examples below. In some embodiments, the methods described herein include measurement and recordal of change from baseline in biomarkers associated with increased risk of kidney stones, such as urine supersaturation scores. In some embodiments, the methods provided herein include monitoring for the presence of kidney stones on screening, degree of malabsorption, tolerability profile, and other patient factors. In some embodiments, the methods described herein promote a change in these factors.

In some embodiments, the method of treating the disorder in which oxalate is detrimental, e.g., PHI or PHII, allows one or more symptoms of the condition or disorder to improve by at least about 10%, 20%, 30%, 40%, 50%, 60%, 70%, 80%, 90%, 95%, or more. In some embodiments, the method of treating the disorder in which oxalate is detrimental, e.g., PHI or PHII, allows one or more symptoms of the condition or disorder to improve by at least about two-fold, three-fold, four-fold, five-fold, six-fold, seven-fold, eight-fold, nine-fold, or ten-fold.

Before, during, and after the administration of the pharmaceutical composition, oxalate and/or oxalic acid levels in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, kidney, liver, duodenum, jejunum, ileum, cecum, colon, rectum, and anal canal. In some embodiments, the methods may include administration of the compositions disclosed herein to reduce levels of the oxalate and/or oxalic acid. In some embodiments, the methods may include administration of the compositions of the invention to reduce the oxalate and/or oxalic acid to undetectable levels in a subject. In some embodiments, the methods may include administration of the compositions of the invention to reduce the oxalate and/or oxalic acid concentrations to undetectable levels, or to less than about 1%, 2%, 5%, 10%, 20%, 25%, 30%, 40%, 50%, 60%, 70%, 75%, 80%, 85%, 90%, or 95% of the subject's oxalate and/or oxalic acid levels prior to treatment.

In some embodiments, the recombinant bacterial cells disclosed herein produce an oxalate catabolism enzyme under exogenous environmental conditions, such as the low-oxygen environment of the mammalian gut, to reduce levels of oxalate and/or oxalic acid in the urine, blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

In one embodiment, the bacteria disclosed herein reduce plasma levels of oxalate will be reduced to less than 4 mg/dL. In one embodiment, the bacteria disclosed herein reduce plasma levels of oxalate will be reduced to less than 3.9 mg/dL. In one embodiment, the bacteria disclosed herein reduce plasma levels of oxalate, to less than 3.8 mg/dL, 3.7 mg/dL, 3.6 mg/dL, 3.5 mg/dL, 3.4 mg/dL, 3.3 mg/dL, 3.2 mg/dL, 3.1 mg/dL, 3.0 mg/dL, 2.9 mg/dL, 2.8 mg/dL, 2.7 mg/dL, 2.6 mg/dL, 2.5 mg/dL, 2.0 mg/dL, 1.75 mg/dL, 1.5 mg/dL, 1.0 mg/dL, or 0.5 mg/dL.

In one embodiment, the subject has plasma levels of at least 4 mg/dL oxalate prior to administration of the pharmaceutical composition disclosed herein. In another embodiment, the subject has plasma levels of at least 4.1 mg/dL, 4.2 mg/dL, 4.3 mg/dL, 4.4 mg/dL, 4.5 mg/dL, 4.75 mg/dL, 5.0 mg/dL, 5.5 mg/dL, 6 mg/dL, 7 mg/dL, 8 mg/dL, 9 mg/dL, or 10 mg/dL prior to administration of the pharmaceutical composition disclosed herein.

Certain unmodified bacteria will not have appreciable levels of oxalate or oxalyl-CoA processing. In embodiments using genetically modified forms of these bacteria, processing of oxalate and/or oxalyl-CoA will be appreciable under exogenous environmental conditions.

Oxalate and/or oxalic acid levels may be measured by methods known in the art. For example, plasma oxalate levels can be measured using the spectrophotometric plasma oxalate assay described by Ladwig et al. (Ladwig et. al., Clin. Chem. 51: 2377-80 (2005)). Further, urine oxalate levels can be measured for example, by using a oxalate oxidase colorimetric enzymatic assay (Kasidas and Rose, Ann. Clin. Biochem. 22: 412-9 (1985)). In some embodiments, oxalate catabolism enzyme, e.g., Frc, expression is measured by methods known in the art. In another embodiment, oxalate catabolism enzyme activity is measured by methods known in the art to assess Frc activity (see oxalate catabolism enzyme sections, supra).

In certain embodiments, the recombinant bacteria are E. coli Nissle. The recombinant bacteria may be destroyed, e.g., by defense factors in the gut or blood serum (Sonnenborn et al., 2009) or by activation of a kill switch, several hours or days after administration. Thus, the pharmaceutical composition comprising the recombinant bacteria may be re-administered at a therapeutically effective dose and frequency. In alternate embodiments, the recombinant bacteria are not destroyed within hours or days after administration and may propagate and colonize the gut.

In one embodiment, the bacterial cells disclosed herein are administered to a subject once daily. In another embodiment, the bacterial cells disclosed herein are administered to a subject twice daily. In another embodiment, the bacterial cells disclosed herein are administered to a subject three times daily. In another embodiment, the bacterial cells disclosed herein are administered to a subject in combination with a meal. In another embodiment, the bacterial cells disclosed herein are administered to a subject prior to a meal. In another embodiment, the bacterial cells disclosed herein are administered to a subject after a meal. In another embodiment, the bacterial cells of the invention are not administered in the form of a food or edible product or incorporated into a food or edible product. The dosage of the pharmaceutical composition and the frequency of administration may be selected based on the severity of the symptoms and the progression of the disease. The appropriate therapeutically effective dose and/or frequency of administration can be selected by a treating clinician.

In one embodiment, the recombinant bacteria are administered at a dose of about 1×10¹¹ live recombinant bacteria, about 2×10¹¹ live recombinant bacteria, about 3×10¹¹ live recombinant bacteria, about 4×10¹¹ live recombinant bacteria, about 4.5×10¹¹ live recombinant bacteria, about 5×10¹¹ live recombinant bacteria, about 6×10¹¹ live recombinant bacteria, about 1×10¹² live recombinant bacteria, or about 2×10¹² live recombinant bacteria. In one embodiment, the recombinant bacteria are administered at a dose of about 6×10¹¹ live recombinant bacteria. In one embodiment, the recombinant bacteria are administered at a dose of about 3×10¹¹ live recombinant bacteria. In one embodiment, the recombinant bacteria are administered at a dose of about 1×10¹¹ live recombinant bacteria. In one embodiment, the administering is about 4.5×10¹¹ live recombinant bacteria. In one embodiment, the administering is about 5×10¹¹ live recombinant bacteria. In one embodiment, the recombinant bacteria are administered at a dose of about 1×10¹² live recombinant bacteria. In one embodiment, the recombinant bacteria are administered at a dose of about 2×10¹² live recombinant bacteria. In one embodiment, the administering are about 5×10¹¹ live recombinant bacteria with meals three times per day. In one embodiment, the recombinant bacteria are administered at a dose of about 1×10¹¹ live recombinant bacteria to about 2×10¹² live recombinant bacteria. In one embodiment, the recombinant bacteria are administered at a dose of about 1×10¹² live recombinant bacteria to about 2×10¹² live recombinant bacteria. In one embodiment, the recombinant bacteria are administered at a dose of about 5×10¹¹ live recombinant bacteria to about 2×10¹² live recombinant bacteria. In another embodiment, a proton pump inhibitor (PPI) is administered to the subject. In another embodiment, the PPI is esomeprazole. In another embodiment, esomeprazole is administered at 40 mg once daily. Other suitable PPIs are known in the art and include lansoprazole, pantoprazole, rabeprazole, esomeprazole, and dexlansoprazole. In another embodiment, the administering of the PPI is once a day.

The methods disclosed herein may comprise administration of a composition disclosed herein alone or in combination with one or more additional therapies, e.g., pyridoxine, citrate, orthophosphate, and magnesium, oral calcium supplementation, and bile acid sequestrants, or a low fat and/or low oxalate diet. An important consideration in the selection of the one or more additional therapeutic agents is that the agent(s) should be compatible with the bacteria disclosed herein, e.g., the agent(s) must not interfere with or kill the bacteria. In some embodiments, the genetically engineered bacteria are administered in combination with a low fat and/or low oxalate diet. In some embodiments, administration of the genetically engineered bacteria provides increased tolerance, so that the patient can consume more oxalate and/or fat.

The methods disclosed herein may further comprise isolating a plasma sample from the subject prior to administration of a composition disclosed herein and determining the level of the oxalate and/or oxalic acid in the sample. In some embodiments, the methods disclosed herein may further comprise isolating a plasma sample from the subject after to administration of a composition disclosed herein and determining the level of oxalate and/or oxalic acid in the sample.

The methods of the invention may further comprise isolating a urine sample from the subject prior to administration of a composition of the invention and determining the level of the oxalate and/or oxalic acid in the sample. In some embodiments, the methods of the invention may further comprise isolating a urine sample from the subject after to administration of a composition of the invention and determining the level of oxalate and/or oxalic acid in the sample.

In one embodiment, the methods disclosed herein further comprise comparing the level of the oxalate and/or oxalic acid in the plasma sample from the subject after administration of a composition disclosed herein to the subject to the plasma sample from the subject before administration of a composition disclosed herein to the subject. In one embodiment, a reduced level of the oxalate and/or oxalic acid in the plasma sample from the subject after administration of a composition disclosed herein indicates that the plasma levels of the oxalate and/or oxalic acid are decreased, thereby treating the disorder in which oxalate is detrimental in the subject. In one embodiment, the plasma level of oxalate and/or oxalic acid is decreased at least 10%, 20%, 30%, 40%, 50%, 60%, 70%, 80%, 90%, or 100% in the sample after administration of the pharmaceutical composition as compared to the plasma level in the sample before administration of the pharmaceutical composition. In another embodiment, the plasma level of the oxalate and/or oxalic acid is decreased at least two-fold, three-fold, four-fold, or five-fold in the sample after administration of the pharmaceutical composition as compared to the plasma level in the sample before administration of the pharmaceutical composition.

In one embodiment, the methods of the invention further comprise comparing the level of the oxalate and/or oxalic acid in the urine sample from the subject after administration of a composition of the invention to the subject to the urine sample from the subject before administration of a composition of the invention to the subject. In one embodiment, a reduced level of the oxalate and/or oxalic acid in the urine sample from the subject after administration of a composition of the invention indicates that the urine levels of the oxalate and/or oxalic acid are decreased, thereby treating the disorder in which oxalate is detrimental in the subject. In one embodiment, the urine level of oxalate and/or oxalic acid is decreased at least 10%, 20%, 30%, 40%, 50%, 60%, 70%, 80%, 90%, or 100% in the sample after administration of the pharmaceutical composition as compared to the plasma level in the sample before administration of the pharmaceutical composition. In another embodiment, the urine level of the oxalate and/or oxalic acid is decreased at least two-fold, three-fold, four-fold, or five-fold in the sample after administration of the pharmaceutical composition as compared to the urine level in the sample before administration of the pharmaceutical composition.

In one embodiment, the methods disclosed herein further comprise comparing the level of the oxalate/oxalic acid in the plasma sample from the subject after administration of a composition disclosed herein to a control level of oxalate and/or oxalic acid.

In another embodiment, the methods of the invention further comprise comparing the level of the oxalate/oxalic acid in the urine sample from the subject after administration of a composition of the invention to a control level of oxalate and/or oxalic acid.

### EXAMPLES

The present invention is further illustrated by the following examples which should not be construed as limiting in any way. The contents of all cited references, including literature references, issued patents, and published patent applications, as cited throughout this application are hereby expressly incorporated herein by reference. It should further be understood that the contents of all the figures and tables attached hereto are also expressly incorporated herein by reference.

### Example 1. Genetically Engineered E. coli Nissle Bacterial Strains Decrease Oxalate Concentration Over Time

Both in vitro and in vivo experiments were conducted which demonstrate that E. coli Nissle bacterial strains decrease oxalate concentration over time.

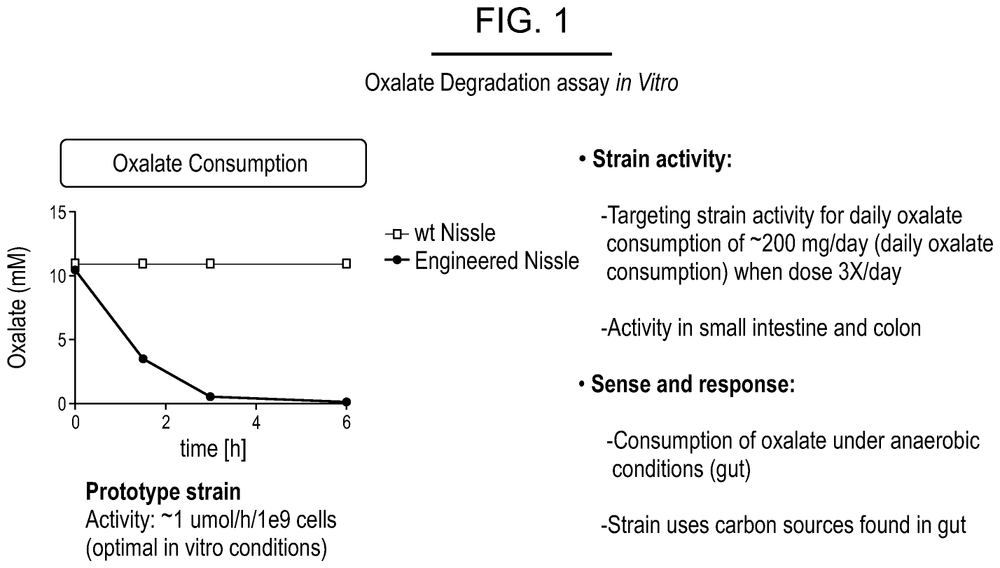

Specifically, a functional in vitro assay was conducted as indicated in FIG. 1. The results from this assay demonstrate that the genetically Engineered E. coli Nissle bacterial strain decreases oxalate concentration over time as compared to the wild type E. coli Nissle bacterial strain (see FIG. 1).

In vivo experiments in animals were also conducted. On day 0, mice were weighed, marked, and randomized into 4 groups. Starting at Day 1, the following protocol was used.
Day 1:
T0:
   PO dose with 100 μL (100 μg) of 13C-Oxalate
   PO dose with 200 μL of Treatment
   T1:
   PO dose with 300 μL of Treatment
   T6: collect urine, feces

The animals were dosed a high dose at 3.12e10 CFU, a mid-dose at 1.04e10 CFU and a low dose at 3.46e9 CFU (total CFUs).

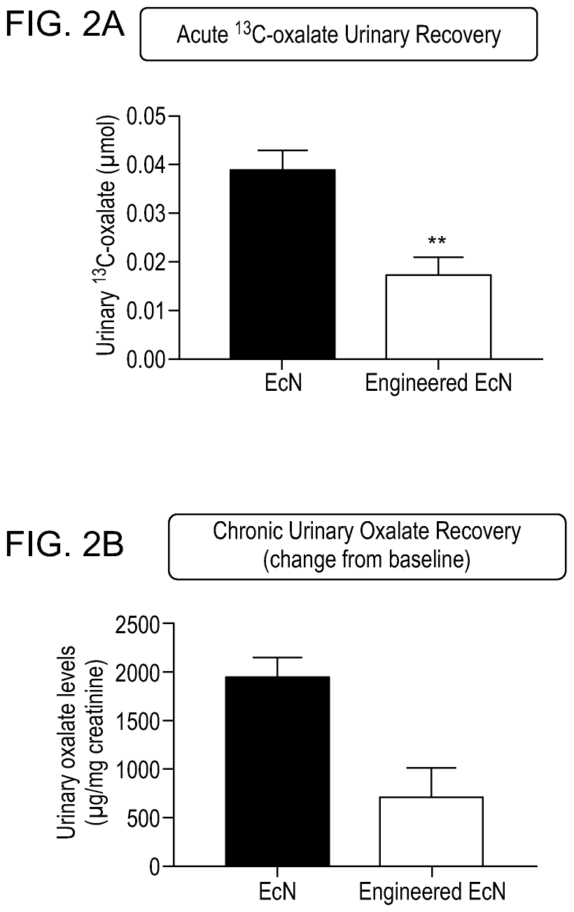

The results depicted in FIG. 2A demonstrate that the genetically Engineered E. coli Nissle bacterial strain reduces acute levels of ¹³C-oxalate, detected in the urine of the treated mice, as compared to the wild type strain.

The results depicted in FIG. 2B demonstrate that the genetically Engineered E. coli Nissle bacterial strain reduces chronic levels of oxalate, detected in the urine of the treated mice, as compared to the wild type strain.

TABLE 14
Construct comprising oxalate catabolism cassette driven by Tet responsive promoter

| Description | SEQ ID NO |
|---|---|
| Construct comprising TetR in reverse orientation, TetR/TetA promoter, and oxalate | SEQ ID NO: 34 |
| catabolism cassette comprising ScAAE3 (oxalate-CoA ligase from S. cerevisiae), | |
| oxy, oxylyl-coA decarboxylase from O. formigenes, and formyl-coA transferase | |
| from O. formigenes, separated by ribosome binding sites | |
| Construct comprising TetR/TetA promoter, and oxalate catabolism cassette | SEQ ID NO: 35 |
| comprising ScAAE3 (oxalate-CoA ligase from S. cerevisiae), oxc, oxylyl-coA | |
| decarboxylase from O. formigenes, and frc, formyl-coA transferase from O. | |
| formigenes, separated by ribosome binding sites | |
| Construct comprising oxalate catabolism cassette comprising ScAAE3 (oxalate- | SEQ ID NO: 36 |
| CoA ligase from S. cerevisiae), oxy, oxylyl-coA decarboxylase from O. formigenes, | |
| and formyl-coA transferase from O. formigenes, separated by ribosome binding | |
| sites | |

Table 15 lists the construct for the chromosomally integrated OxlT at the lacZ locus.

TABLE 15
Construct comprising OxlT (oxalate:formate antiporter) driven by tet-inducible promoter

| Description | SEQ ID NO |
|---|---|
| Construct comprising TetR in reverse orientation, TetR/TetA promoter, driving | SEQ ID NO: 37 |
| OxlT (oxalate:formate antiporter from O. formigenes) | |
| Construct comprising TetR/TetA promoter, driving OxlT (oxalate:formate | SEQ ID NO: 38 |
| antiporter from O. formigenes) | |
| Construct comprising RBS and leader region driving OxlT (oxalate:formate | SEQ ID NO: 39 |
| antiporter from O. formigenes) | |

TABLE 16
Construct comprising oxalate catabolism cassette (pFNRS-
ScAAE3-oxc-frc) under control of a FNR promoter

| Description | SEQ ID NO |
|---|---|
| Construct comprising promoter with FNR binding site driving expression of cassette | SEQ ID NO: 40 |
| comprising ScAAE3 (oxalate-CoA ligase from S. cerevisiae), oxy, oxylyl-coA | |
| decarboxylase from O. formigenes, and formyl-coA transferase from O. formigenes, | |
| separated by ribosome binding sites | |
| FNR promoter with RBS and leader region | SEQ ID NO: 41 |
| FNR promoter without RBS and leader region | SEQ ID NO: 66 |

TABLE 17
Construct comprising OxlT (oxalate:formate
antiporter) under control of a FNR promoter

| Description | SEQ ID NO |
|---|---|
| Construct comprising FNR promoter, driving OxlT | SEQ ID NO: 42 |
| (oxalate:formate antiporter from O. formigenes) | |

TABLE 18
Oxalate catabolism cassette (oxc-frc) driven by FNRS promoter

| Description | SEQ ID NO |
|---|---|
| Construct comprising FNR promoter, driving a | SEQ ID NO: 43 |
| oxalate catabolism cassette comprising oxc-frc. | |
| Construct comprising oxalate catabolism cassette | SEQ ID NO: 44 |
| comprising oxc-frc with RBS and leader region | |

TABLE 19
Oxalate catabolism cassette (yfdE-
oxc-frc) driven by FNRS promoter

| Description | SEQ ID NO |
|---|---|
| Construct comprising FNR promoter, driving a | SEQ ID NO: 45 |
| oxalate catabolism cassette comprising yfdE-oxc-frc. | |
| Construct comprising oxalate catabolism cassette | SEQ ID NO: 46 |
| comprising yfdE-oxc-frc with RBS and leader region | |

Sample Preparation

Oxalic acid stock 10 mg/mL was prepared in water and aliquoted in 1.5 mL microcentrifuge tubes (100 μL), and stored at −20° C. Standards (1000, 500, 250, 100, 20, 4, and 0.8 g/mL) are prepared in water. On ice, 20 μL of sample (and standards) were mixed with 180 μL of H₂O containing 10 g/mL of oxalic acid-d2 in the final solution in a V-bottom 96-well plate. The plate was heat-sealed with a ClearASeal sheet and mix well.

LC-MS/MS method

Oxalate was measured by liquid chromatography coupled to tandem mass spectrometry (LC-MS/MS) using a Thermo TSQ Quantum Max triple quadrupole mass spectrometer. Table 20, Table 21 and Table 22 provide the summary of the LC-MS/MS method.

TABLE 20

| | |
|---|---|
| Column | Synergi Hydro column, 4 μm (75 × 4.6 mm) |
| Mobile Phase A | 5 mM Ammonium acetate |
| Mobile Phase B | Methanol |
| Injection volume | 10 uL |

TABLE 21
HPLC Method:

| | Flow Rate | | |
|---|---|---|---|
| Time (min) | (μL/min) | A % | B % |
| 0.0 | 500 | 100 | 0 |
| 0.5 | 500 | 100 | 0 |
| 1.0 | 500 | 5 | 95 |
| 2.5 | 500 | 5 | 95 |
| 2.51 | 500 | 100 | 0 |
| 2.75 | 500 | 100 | 0 |

TABLE 22
Tandem Mass Spectrometry

| | |
|---|---|
| Ion Source | HESI-II |
| Polarity | Negative |
| SRM transitions | Oxalate: 90.5/61.2 |
| SRM transitions | Oxalate-d2: 92.5/62.2 |

TABLE 23
Primer Sequences

| Name | Description | SEQ ID NO |
|---|---|---|
| SR36 | Round 1: binds on pKD3 | SEQ ID NO: 47 |
| SR38 | Round 1: binds on pKD3 | SEQ ID NO: 48 |
| SR33 | Round 2: binds to round 1 PCR product | SEQ ID NO: 49 |
| SR34 | Round 2: binds to round 1 PCR product | SEQ ID NO: 50 |
| SR43 | Round 3: binds to round 2 PCR product | SEQ ID NO: 51 |
| SR44 | Round 3: binds to round 2 PCR product | SEQ ID NO: 52 |

TABLE 24
Pfnr1-lacZ construct Sequences

| Description | SEQ ID NO |
|---|---|
| Nucleotide sequences of Pfnr1-lacZ construct, | SEQ ID NO: 53 |
| low-copy | |

TABLE 25
Pfnr2-lacZ construct sequences

| Description | SEQ ID NO |
|---|---|
| Nucleotide sequences of Pfnr2-lacZ construct, | SEQ ID NO: 54 |
| low-copy | |

TABLE 27
Pfnr4-lacZ construct Sequences

| Description | SEQ ID NO |
|---|---|
| Nucleotide sequences of Pfnr4-lacZ construct, | SEQ ID NO: 56 |
| low-copy | |

TABLE 26
Pfnr3-lacZ construct Sequences

| Description | SEQ ID NO |
|---|---|
| Nucleotide sequences of Pfnr3-lacZ construct, | SEQ ID NO: 55 |
| low-copy | |

TABLE 28
Pfnrs-lacZ construct Sequences

| Description | SEQ ID NO |
|---|---|
| Nucleotide sequences of Pfnrs-lacZ construct, | SEQ ID NO: 57 |
| low-copy | |

### Example 2. Other Sequences of Interest

TABLE 29
prpR Propionate-Responsive Promoter Sequence

| Description | SEQ ID NO |
|---|---|
| Prp promoter | SEQ ID NO: 58 |

TABLE 30
Wild-type clbA and clbA knock-out

| Description | SEQ ID NO |
|---|---|
| Wild-type clbA | SEQ ID NO: 59 |
| clbA knock-out | SEQ ID NO: 60 |

### Example 3. Reduction of Oxalate Concentrations in Acute Mice Models and Healthy Monkeys

Enteric hyperoxaluria occurs when there is excess absorption of oxalate in the gastrointestinal (GI) tract, which results in an accumulation of oxalate in kidneys, and may lead to recurrent kidney stones and kidney failure. It has been shown that reduction of oxalate in GI track is clinically beneficial for patients. However, there is currently no available therapy, and there are more than 80,0000 severe patients in the United States alone. These patients can have recurrent kidney stones and risk for kidney failure.

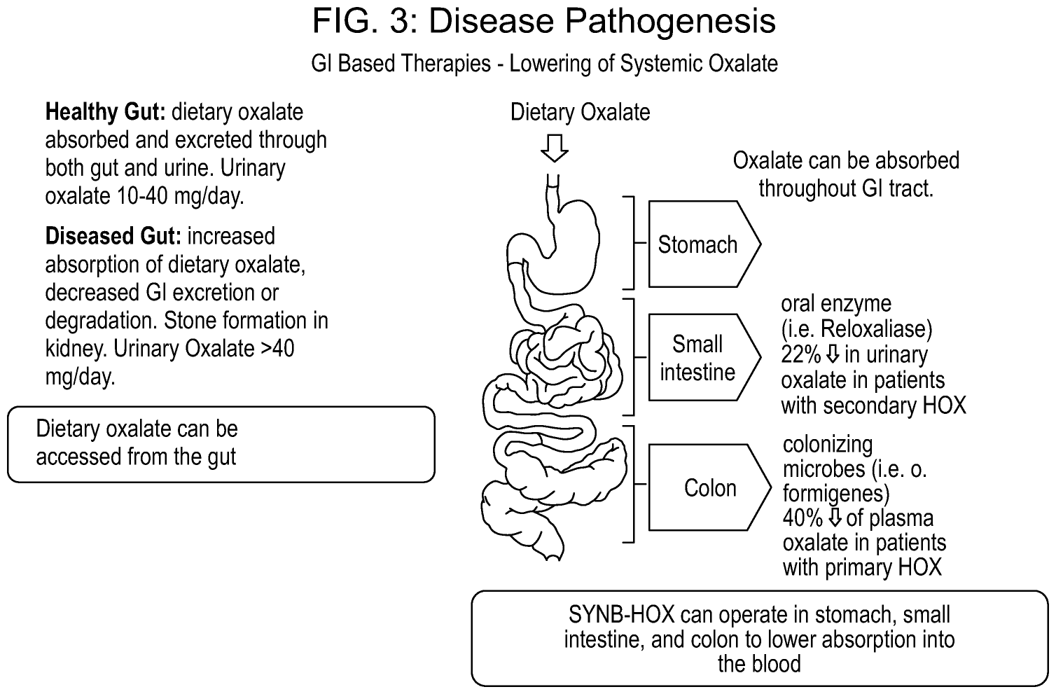

As demonstrated herein, the engineered bacterial strains have a great potential to operate in stomach, small intestine, and colon to lower absorption of oxalate into the blood (FIG. 3).

In vivo experiments were conducted in both acute mouse models and healthy monkeys which demonstrate that E. coli Nissle bacterial strains decrease oxalate concentration over time. Specifically, three engineered E. coli Nissle strains (SYN5752, SYN7169, and SYNB8802) had been constructed. SYN7169 is derived from SYN5752, the only difference is the thyA and phage 3 ko (sequences included in Table 36). Genotypes were shown below.
SYN5752: HA910::FNR_oxlT, HA12::FNR_scaaE3-oxcd-frc
SYN7169: HA910::FNR_oxlT, HA12::FNR_scaaE3-oxcd-frc, ThyA::KanR, phage 3::CamR

SYN7169 and SYNB8802 have identical genetic modifications, but SYN7169 also has a chloramphenicol and kanamycin resistance cassette to aid in isolation on selective mediate.

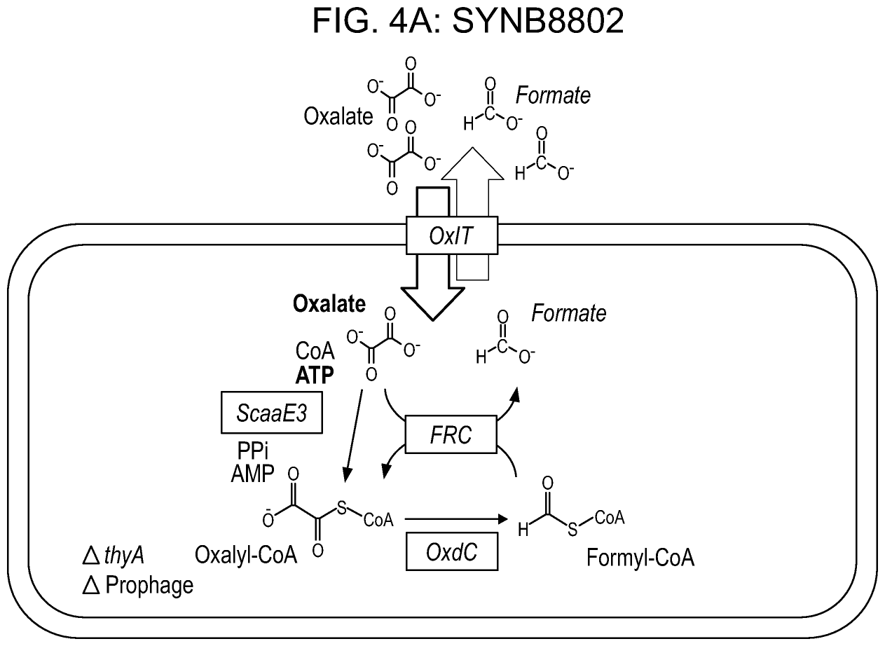

Relevant sequences were included in Tables shown above and Table 36. SYNB8802 (FIG. 4A) includes an insertion of one gene encoding an oxalate antiporter (OxlT) derived from Oxalobacter formigenes under the regulatory control of an anaerobic-inducible promoter (pFnrs) and the anaerobic-responsive transcriptional activator FNR, and insertion of one operon, encoding three genes under the regulatory control of an anaerobic-inducible promoter (pFnrs) and the anaerobic-responsive transcriptional activator FNR (oxalyl-CoA synthetase (scaae3) derived from Saccharomyces cerevisiae, oxalate decarboxylase (oxdc) derived from Oxalobacter formigenes, and formyl-CoA transferase derived from Oxalobacter formigenes, a deletion of the thyA gene that encodes thymidylate synthase to create a thymidine auxotroph, and the endogenous Nissle prophage has been inactivated. Relevant sequences are included in the Tables herein.

In vitro experiments were conducted as described above. The results from this assay demonstrate that the genetically Engineered E. coli Nissle bacterial strain (SYNB8802) decreases oxalate concentration over time as compared to the wild type E. coli Nissle bacterial strain (see FIG. 4B).

Additionally, the genetically engineered E. coli Nissle bacterial strain (SYNB8802) increases formate concentration over time as compared to wild type E. coli Nissle bacterial strain (FIG. 4C). E. coli Nissle (control) and SYNB8802 were grown in shake flasks and subsequently activated in an anaerobic chamber, followed by concentration and freezing at ≤−65° C. in glycerol-based formulation buffer. In assay media containing 10 mM ¹³C-oxalate, optical density=5 activated cells were incubated statically at 37° C. Supernatant samples were removed at 30 and 60 minutes to determine the concentrations of ¹³C-oxalate and ¹³C-formate. The concentrations of ¹³C-oxalate and ¹³C-formate were determined by liquid chromatography-tandem mass spectrometry (LC-MS/MS).

In vitro Gastrointestinal Simulation (IVS)

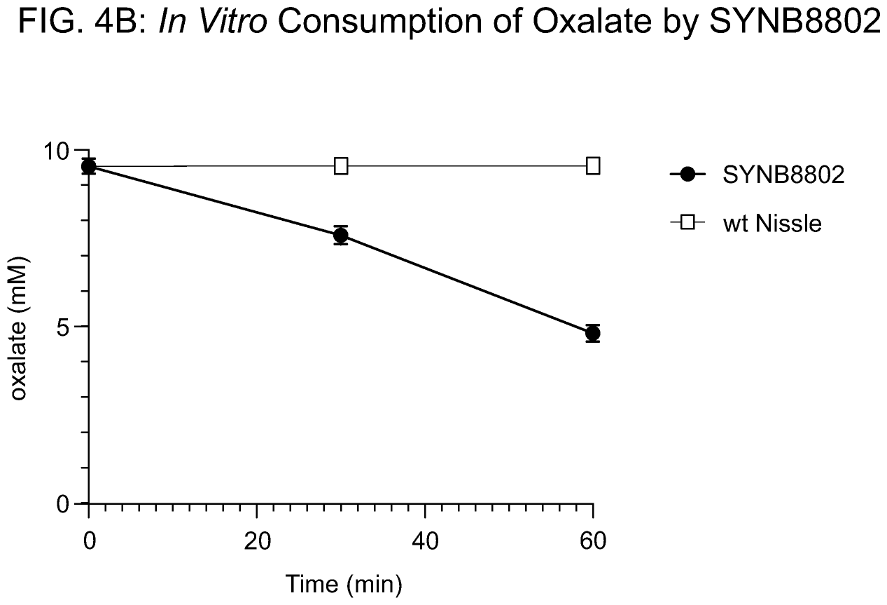

Both stomach and colon simulated gut fluids were capable of activating SYNHOX in simulated in vitro (FIG. 5A). Simulated stomach fluid activated SYNHOX (SYN5752) oxalate consummation activity more than twice as much when compared to oxalate consumption in simulated colon fluid (FIG. 5A).

To characterize the viability and metabolic activity of engineered bacterial strains, and to predict their function in vivo, an in vitro gastrointestinal simulation (IVS) model was designed to simulate key aspects of the human gastrointestinal tract, including oxygen concentration, gastric and pancreatic enzymes, and bile. The IVS model is comprised of a series of incubations in 96-well microplate format designed to simulate stomach, small intestine, and colonic conditions. The stomach, small intestinal, colon portions of the IVS model were adapted from Minekus et al., 2014.

Briefly, frozen aliquots of bacterial cells were first thawed at room temperature and resuspended in 0.077 M sodium bicarbonate buffer at 5.0×10⁹ live cells/mL. This solution was then mixed with equal parts of simulated gastric fluid (SGF; Minekus et al., 2014) containing 10 mM oxalate and incubated for 2 hours at 37° C. with shaking in a Coy microaerobic chamber. The atmosphere within the microaerobic chamber was initially calibrated to 7% oxygen and gradually decreased to 2% oxygen over 2 hours. The cell density in SGF is 2.5×10⁹ live cells/mL. After 2 hours cells were then mixed in volumes of 1:1 with simulated intestinal fluid (SIF; Minekus et al., 2014) and incubated for an additional 2 hours at 37° C. with shaking in a Coy microaerobic chamber. Cell density in SIF is 1.25×10⁹ live cells/mL. After 2 hours, cells were moved into the anaerobic chamber and mixed 1:6 with colon simulated media based off of SGF; Minekus et al., 2014 (CSM). CSM had an additional 10 mM Oxalate and were incubated for 3 hours at 37° C. Cell density in CSM is 2.08×10⁶ live cells/mL.

To determine strain activity over time, aliquots were collected periodically and centrifuged at 4000 rpm for 5 mins using a tabletop centrifuge. Cell free supernatants were collected and stored at −80° C. prior to mass spectrometry analysis of oxalate concentration.

### In Vivo Mouse Model Studies

For in vivo mice studies, on day 0, mice were weighed, marked, and randomized into 4 groups. Starting at Day 1, the following protocol was used.
Day 1:
T0:
   PO dose with 100 μL (100 μg) of ¹³C-Oxalate
   PO dose with 200 μL of Treatment
   T1:
   PO dose with 300 μL of Treatment
   T6: collect urine, feces

The animals were dosed a high dose at 3e10 CFU, a mid-dose at 1e10 CFU and a low dose at 3e9 CFU (total CFUs).

As shown in FIG. 5B, SYN-5752 strain in acute, isotope model demonstrated urinary oxalate consumption in gut. ¹³C-Oxalate consumption had been measured in multiple acute mouse studies and the efficacy of the strains ranged between 50-75% (FIG. 5B). SYN7169 behaved similarly to SYN5752 in this model.

In a different experiment, C57BL/6J male mice were group housed and orally administered a dose of ¹³C-oxalate (100 μg) followed by a dose of vehicle (13.8% w/v Trehalose, 68 mM Tris, 55 mM HCl, 1×PBS) or SYNB8802. Mice were immediately placed into metabolic cages (n=3/cage) and received another dose of vehicle or SYNB8802 1 hour post first dose, for a daily total of 4.7×10⁸, 4.7×10⁹ or 4.7×10¹⁰ live cells. Urine was collected 6 hours following dose 1 and ¹³C-oxalate and creatinine levels were quantitated by liquid chromatography/tandem mass spectrometry (LC-MS/MS). Two studies were conducted, and the results were combined (FIG. 5C).

### In Vivo Monkey Model Studies

For in vivo monkey studies, animals were randomly distributed in 2 groups, vehicle (formulation buffer) and SYN7169 (5e11 cells). N=6 in each group. After overnight fasting, each animal received same amount of spinach smoothie, ¹³C-2 labeled oxalate, sodium bicarbonate (1M) and either vehicle or strain (see Table 32). The spinach smoothie was prepared by blending baby spinach leaves in tap water until smooth at a spinach:water ratio of 60 gm:40 mL.

Specifically, treatments were administered to the appropriate animals by oral gavage on Day 1. Capped bacteria tube was inverted 3 times before each dose administration. Dose formulations were administered by oral gavage using a disposable catheter attached to a plastic syringe. Following dosing, the gavage tube were rinsed with 5 mL of the animal drinking water, into the animal's stomach. Each animal was dosed with a clean gavage tube. The first day of dosing was designated as Day 1.

TABLE 31
Experimental Design

| | No. | | Dose | |
|---|---|---|---|---|
| | Ani- | | Volume | Dose |
| Group | mals | Treatment | (mL) | Regimen |
| 1 | 6 | Spinach smoothie | 25.7 | PO |
| | | Sodium bicarbonate | 1.8 | |
| | | ¹³C2 oxalate (20 mg/ml) | 2.5 | |
| | | Formulation buffer | 5.0 | |
| 2 | 6 | Spinach smoothie | 25.7 | |
| | | Sodium bicarbonate | 1.8 | |
| | | ¹³C2 oxalate (20 mg/ml) | 2.5 | |
| | | Bacteria (SYN7169021920B-001) | 5.0 | |

Urine was collected after 6 hrs of PO dosing. Animals were separated and a clean collection pan was inserted prior to dose to assist in urine collection at room temperature. At conclusion of 6 hours post dose, the total amount of urine were measured and recorded. One aliquot of 1 mL samples was collected in uniquely labeled clear polypropylene tube and immediately frozen on dry ice. A second aliquot of approximately 100 uL was collected in a 96-deep well plate and immediately frozen on dry ice.

Oxalate, ¹³C2-oxalate, and creatinine were measured in monkey or non-human primates (NHP) urine by liquid chromatography (LC) tandem mass spectrometry (MS/MS) with selected reaction monitoring (SRM) of analyte specific fragmentation products using a Thermo Vanquish-TSQ Altis LC-MS/MS system. Urine was diluted tenfold with 10 mM ammonium acetate containing creatinine-d5, 2 uL injected and separated using a Waters Acquity HSS T3 column (2.1×100 mm) at 0.4 uL/min and 50° C. from 0 to 95% B over two minutes (A: 10 mM ammonium acetate; B: acetonitrile). SRM ion transitions were as follows in electrospray negative mode: oxalate 89>61, ¹³C2-oxalate 91>62; electrospray positive mode: creatinine 114>44, creatinine-d5 199>49. Sample concentrations of oxalate and ¹³C2-oxalate were calculated using absolute peak areas and a matrix based standard curve constructed in NHP urine. Creatinine concentrations were calculated using creatinine/creatinine-d5 peak area ratios and a water based standard curve.

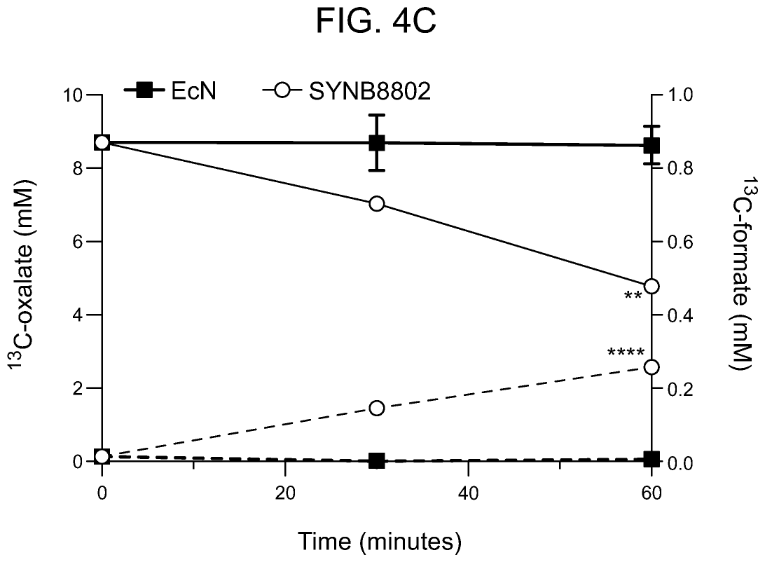

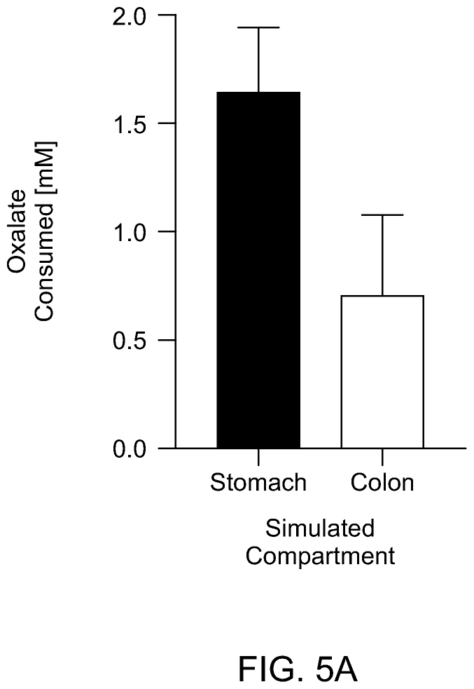

As shown in FIG. 6, while spinach smoothie increased urinary oxalate levels in the treated monkeys, engineering EcNs (SYN7169) attenuated these increase in urinary oxalate significantly. SYN7169 dose-dependently lowered the recovery of urinary oxalate by 45%, 37%, 45% and 75% at 5×10¹⁰, 1×10¹¹, 5×10¹¹ or 1×10¹² CFU as compared to vehicle, respectively (see FIG. 7A). The effects of SYN7169 on ¹³C-oxalate followed the same trends (see FIG. 7B). In conclusion, these studies indicate that SYN7169 was capable of consuming oxalate in monkeys with acute hyperoxaluria.

In a second study with 12 male cynomolgus monkeys receiving both vehicle and SYNB8802. Animals were fasted the night prior to the study for approximately 16-18 hours. On the morning of the experiment, each monkey was removed from its cage and administered vehicle (water) or spinach suspension (39 g), sodium bicarbonate (1.8 mmol), ¹³C-oxalate (50 mg), and vehicle (13.8% w/v Trehalose, 68 mM Tris, 55 mM HCl, 1×PBS) or SYNB8802 (1×10¹² live cells). Animals were then returned to their cages and a clean urine collection pan was placed at the bottom of each cage. Urine was collected at 6 hours post dosing, and the levels of oxalate, ¹³C-oxalate and creatinine were quantitated by liquid chromatography/tandem mass spectrometry (LC-MS/MS) (FIG. 7C).

Viable SYNHOX from Fecal Samples

Viable SYNHOX was recovered in feces 6 and 24 hours after oral doses were administered to both mice and non-human primates (NHP). A significant number of viable SYNHOX (SYN7169) and SYNB8802 were recovered at both time points.

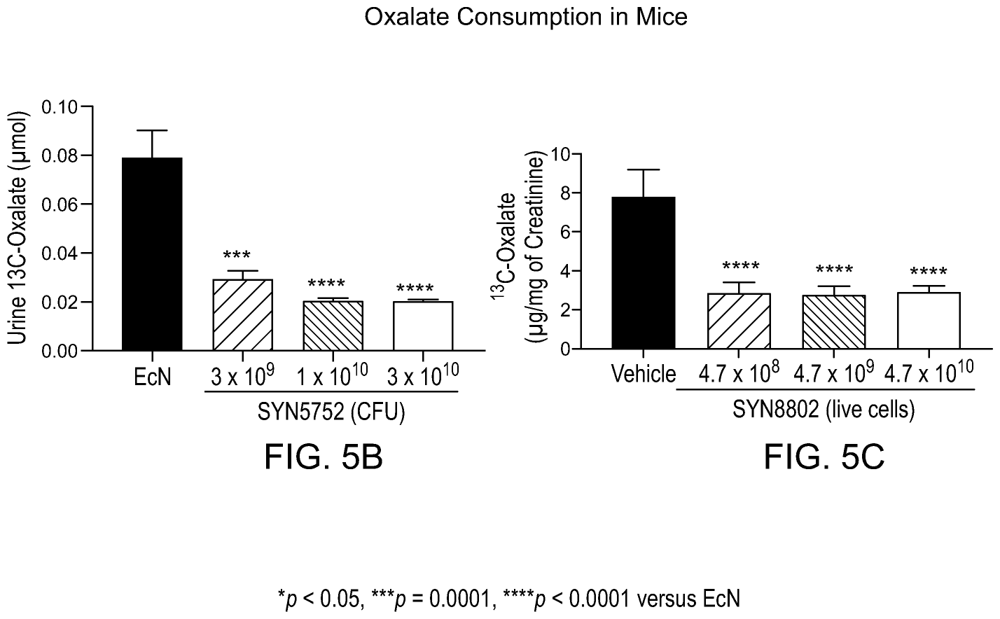

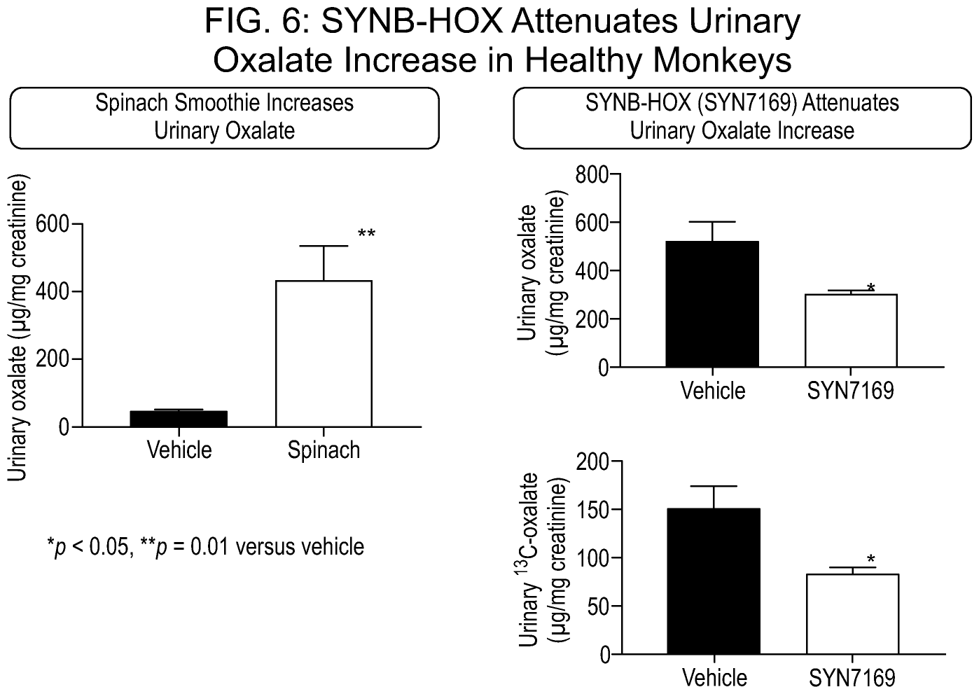

Viable SYNB8802 and wild type E. coli were recovered from mouse feces and were viable at multiple time points over 72 hours after ingestion (FIG. 8A). SYNB8802 cleared from feces after 24 hours while the wild type strain cleared at 72 hours after ingestion. Briefly, C57BL/6J mice were group housed and assigned to groups (n=16) based on average cage body weight. Mice received a single oral dose of the treatment (1.3×10¹⁰ CFU) and feces were collected fresh by free catch and placed into pre-weighed BeadBug tubes containing 500 mL of PBS, weighed, and then processed for serial dilution plating to determine viable colony-forming units (CFUs) immediately after collection. Data presented as mean bacterial strain fecal recovery±standard error of the mean. CFU=colony forming unit, SYNB8802*=antibiotic-resistant SYNB8802.

In a separate experiment, twelve male monkeys were fasted the night prior to the study. On the morning of the study, each monkey was removed from its cage and administered a spinach suspension, sodium bicarbonate, ¹³C-oxalate, and formulation buffer or bacteria. Feces were collected 6 and 24 hours post dosing, and the total weight was recorded. Fecal samples were homogenized with phosphate-buffered saline (PBS; 10 times sample weight), and the final volume of buffer added was recorded. Fecal sample suspensions were serially diluted in PBS and plated on selective LB agar media to enumerate SYN7169 or SYNB8802 colony-forming units (CFU) (FIG. 8B).

### Example 4: Formulations and Human Treatment

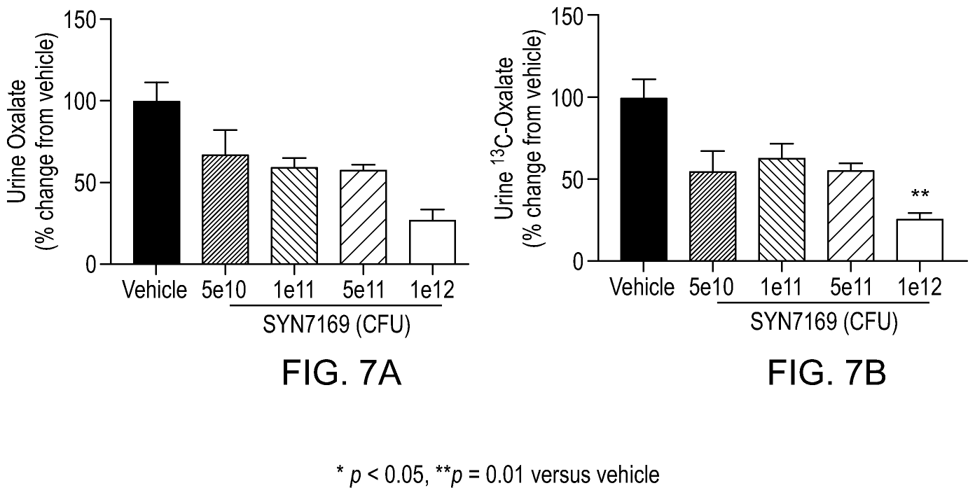

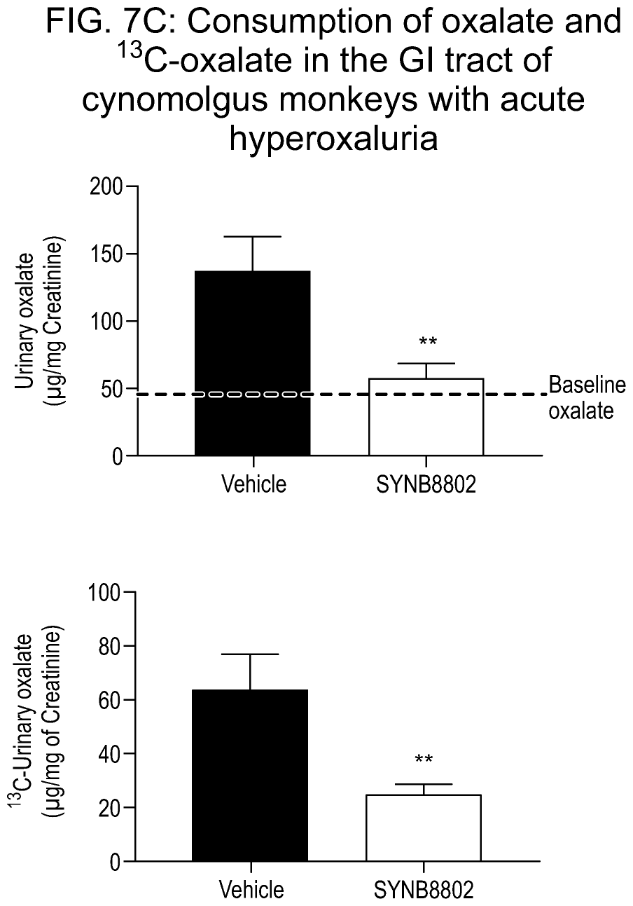

As shown in FIGS. 9-10, oxalate consumption with SYNB8802 and SYN7169, respectively, lyophilized formulations versus frozen liquid formulations was tested. Male cynomolgus monkeys (approximately 2-5 years of age and average weight of 3.3 kg) were fasted overnight. The cynomolgus monkeys (n=12) received a spinach preparation containing approximately 400 mg of oxalate (including labeled ¹³C-oxalate). One group received 5×10¹¹ live cells of SYNB8802 frozen liquid (n=6) and the other group received 5×10¹¹ live cells of SYNB8802 lyophilized material (n=6). Urine was collected for 6 hours and cumulative urinary oxalate and creatinine levels were measured via liquid chromatography/mass spectrometry. Strains and amounts tested in the NHP “spinach smoothie” model disclosed above are disclosed in Table 32, below. Live cell determination was calculated as described at least in PCT International Application No. PCT/US2020/030468, entitled “Enumeration of Genetically Engineered Microorganisms by Live Cell Counting Techniques,” the entire contents of which are expressly incorporated herein by reference.

TABLE 32

| Study # | Description | Strain Info and Amount Used |
|---|---|---|
| NHP HOX#7 | Dose response of 7169 FL, | SYN7169021920B-001 |
| (20247591) | 1e12 cells (CFU) | 1.01e11 cfu/mL |
| | | Total volume: 60 ml |
| NHP HOX#8 | Dose response of 7169 FL, | SYN7169021920B-001 |
| (20248321) | 5e10 cells (CFU) | 1.01e11 cfu/mL |
| | | Total volume: 30 ml |
| NHP HOX#9 | Efficacy of 8802 FL and | SYN7182032620C-003 (1.33 g vial) |
| (20249257) | Lyo, 5e11 live cells | 1.2e+11 live cells/ml |
| | | Total amount: 6 vials |
| | | SYN7182032420H-001 |
| | | 1.41e+11 live cells/ml |
| | | Total volume: 30 ml |
| NHP HOX#10 | Dose response of 8802 FL, | SYN7182032420C-001 |
| (20250016) | 1e12 cells (CFU) | 1.3e+11 cfu/ml |
| | | Total volume: 60 ml |
| NHP HOX#11 | Dose response of 8802 FL, | SYN7182032420C-001 |
| (20250590) | 1e11 cells (CFU) | 1.3e+11 cfu/ml |
| | | Total volume: 10 ml |
| NHP HOX#12 | Dose response of 8802 Lyo, | SYN7182032620C-003 (1.33 g vial) |
| | 1e12 live cells | 1.2e+11 live cells/ml |
| | | Total amount: 10 vials |

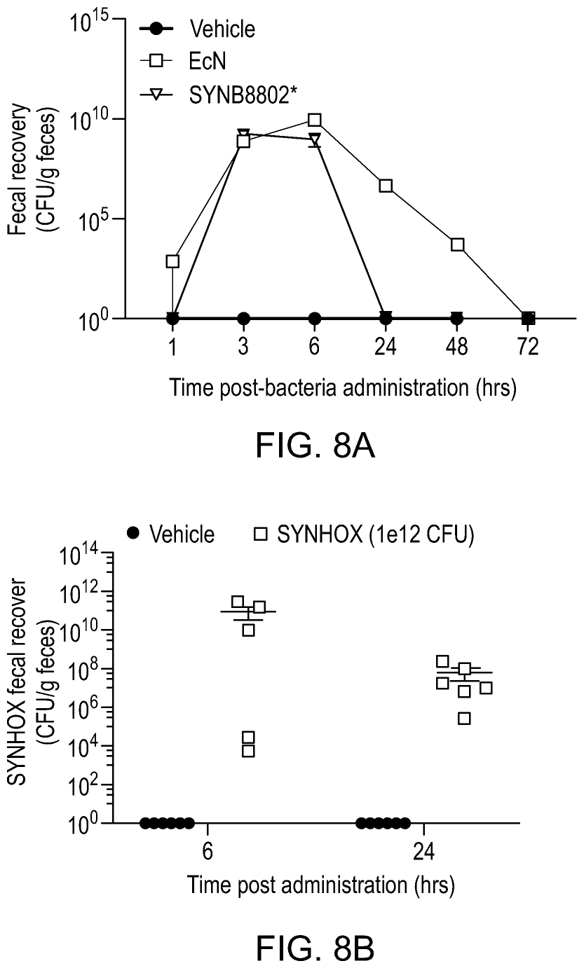

As shown in FIG. 11A, modeling predicts SYNB8802 has potential to achieve 20%-50% urinary oxalate lowering at target dose ranges. Modeling incorporates strain activity assessments in simulated conditions within different gut compartments, known levels of dietary oxalate consumption, oxalate absorption levels with the GI tract, and urinary oxalate excretion. Accordingly, in one embodiment, the dosage of SYNB8802 is 5×10¹¹ cells. In one embodiment, the dosage of SYNB8802 is 2×10¹¹ cells. In one embodiment, the dosage of SYNB8802 is 1×10¹¹ cells.

In silico stimulation (ISS) connects in vitro strain activity knowledge to host and disease biology. The strain-side model simulates the consumption of oxalate by SYNB8802 within the gastrointestinal physiology (FIG. 11B). The host-side model (overall schematic) simulates the impact of consumption by SYNB8802 on the distribution of oxalate throughout the body (FIG. 11B). The model assumes SYNB8802 is dosed with a meal and predicts consumption of gut oxalate and reduction of its absorption into the blood. ISS predicts that SYNB8802 has the potential to achieve greater than 20% urinary oxalate lowering in patients at doses greater than 1×10¹¹ cells.

ISS predicts a dose-dependent lowering of urinary oxalate (FIG. 11C). Data presented as baseline assumption of increased dietary oxalate absorption in HOX patients (4× healthy absorption); bounded region represents the range of the assumption (3×-5× healthy absorption).

### Example 5: Clinical Trials

Clinical trial, Part I, is designed to test SYNB8802 in an inpatient, double-blind, randomized, placebo-controlled, multiple ascending dose (MAD) study in healthy volunteers (HV). SYNB8802 will be administered orally at multiple ascending doses, for example 1×10¹¹ cells, 3×10¹¹ cells, 4.5×10¹¹ cells, and 1×10¹² cells, preferably at doses of 6×10¹¹ cells and 2×10¹² cells, or placebo. Dose of SYNB8802 will preferably not exceed 2×10¹² cells. Doses will be administered three times daily (TID) for 5 days. During this time a high-oxalate, low calcium diet is followed. Multiple ascending doses will be administered to reach the final dose concentration. Optional cohorts in Part I will include healthy volunteers receiving SYNB8802 at a dose to be determined based on the data from the first cohorts tested administered three times a day for 5 days.

Clinical trial, part II, is designed to test SYNB8802 is an outpatient, double-blind, randomized, placebo-controlled, crossover study in patients with enteric hyperoxaluria. Optionally the enteric hyperoxaluria is a result of gastric bypass surgery, i.e., enteric hyperoxaluria secondary to Roux-en-Y bariatric surgery. If SYNB8802 appears to be well tolerated and safe in this study, subsequent studies will be performed to evaluate the safety and efficacy of SYNB8802 in patients with EH secondary to additional GI disorders.

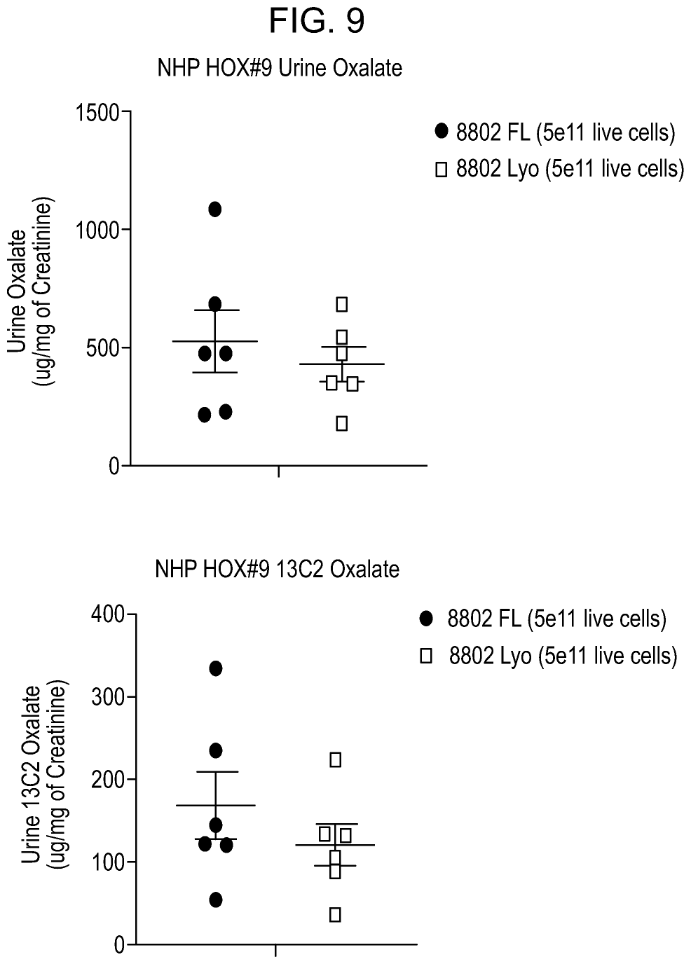

In order to determine baseline UOx levels for Period 1, 24-hour urine samples will be collected for 3 days, within 7 days of the first dose of investigational medicinal product (IMP) (FIG. 12). IMP at doses 1×10¹¹, 3×10¹¹, or 1×10¹² and not to exceed 2×10¹² will be administered TID. Subjects will take a proton pump inhibitor (PPI; esomeprazole) once a day, 60-90 minutes before the meal of their choosing, starting four days prior to the first IMP dose of each period through the last IMP dose of each period. Subjects will be randomized on Day 1 to receive SYNB8802 at or below the maximum tolerated dose (MTD) determined in Part 1 or placebo and will then be dosed three times daily (TID) with meals on Days 1-6. Four days prior, multiple ascending doses will be administered to reach the final dose concentration. Urine samples for determination of 24-hour oxalate levels will be collected on Days 4-6. After a washout period of at least 2 weeks and no more than 4 weeks, subjects will crossover and begin the second period. In order to determine baseline UOx levels for Period 2, 24 hour urine samples will be collected for 3 days, within 7 days of the first dose of Period 2 IMP. Subjects will then crossover to dosing with SYNB8802 or placebo for 6 days. Urine samples for 24-hour oxalate levels will again be collected on the fourth, fifth, and sixth days of Period 2. A Safety Follow-up Visit (or telemedicine) will occur 7 days after last dose of IMP. Subjects will collect weekly fecal samples for 4 weeks after last dose of IMP.

TABLE 33

| Arm | Intervention/Treatment |
|---|---|
| Experimental: MAD HV: SYNB8802 (1 × | Drug: SYNB8802 |
| 10\hat{ }11 live cells) | SYNB8802 is formulated as a non-sterile |
| HV subjects receive SYNB8802 (1 × 10\hat{ }11 live | solution intended for oral administration |
| cells) TID for 5 days in the MAD study (Part 1). | |
| Experimental: MAD HV: SYNB8802 (3 × | Drug: SYNB8802 |
| 10\hat{ }11 live cells) | SYNB8802 is formulated as a nonsterile |
| HV subjects receive SYNB8802 (3 × 10\hat{ }11 live | solution intended for oral administration |
| cells) TID for 5 days in the MAD study (Part 1). | |
| Experimental: MAD HV: SYNB8802 (1 × | Drug: SYNB8802 |
| 10\hat{ }12 live cells) | SYNB8802 is formulated as a nonsterile |
| HV subjects receive SYNB8802 (1 × 10\hat{ }12 live | solution intended for oral administration |
| cells) TID for 5 days in the MAD study (Part 1). | |
| Experimental: MAD HV: SYNB8802 (optional | Drug: SYNB8802 |
| cohort 1) | SYNB8802 is formulated as a nonsterile |
| HV subjects receive SYNB8802 (at a dose to be | solution intended for oral administration |
| determined based on the data from the first 3 | |
| cohorts) | |
| TID for 5 days in the MAD study (Part 1). | |
| Experimental: MAD HV: SYNB8802 (optional | Drug: SYNB8802 |
| cohort 2) | SYNB8802 is formulated as a nonsterile |
| HV subjects receive SYNB8802 (at a dose to be | solution intended for oral administration |
| determined based on the data from the first 3 | |
| cohorts) | |
| TID for 5 days in the MAD study (Part 1). | |
| Placebo Comparator: MAD HV: Placebo | Drug: Placebo |
| HV subjects receive placebo TID for 5 days in | In order to maintain study blinding, matching |
| the MAD study (Part 1). | placebo in identical packaging will be |
| | manufactured using an inactive powder |
| Crossover Arm 1: SYNB8802 crossover to | Drug: SYNB8802 |
| Placebo | SYNB8802 is formulated as a nonsterile |
| | solution intended for oral administration |
| | Drug: Placebo |
| In Part 2 subjects will be randomized (1:1) to | In order to maintain study blinding, matching |
| receive SYNB8802 TID for 6 days and then, | placebo in identical packaging will be |
| following a | manufactured using an inactive powder |
| washout period, receive Placebo TID for 6 days. | |

Part 2: Pharmacodynamic Effects of SYNB8802 in Subjects with Enteric Hyperoxaluria

Part 2 is a double-blind (sponsor-open), outpatient, placebo-controlled crossover study of SYNB8802 in subjects with enteric hyperoxaluria. All subject evaluations and assessments throughout this study may be conducted either at the clinical site or by a home healthcare professional at an alternative location (e.g., subject's home, hotel). Subjects will maintain their normal diet throughout the study, which they will record on those days requiring 24-hour urine collection during the baseline UOx and treatment periods using a daily diary. To determine baseline UOx levels for dosing Period 1, 24-hour urine samples will be collected for 3 days, within 7 days of starting dosing Period 1. Subjects will take a PPI (esomeprazole) QD, 60-90 minutes before the meal of their choosing, starting 4 days prior to the first IMP dose of each period through the last IMP dose of each period. Subjects will be randomized between Day −7 to −4 to receive SYNB8802 at or below the MTD defined in Part 1a or placebo. Subjects will be dosed with IMP up to 3 times per day with meal(s) for up to 10 days during dosing Period 1. Subjects who in the opinion of the investigator cannot progress beyond QD or BID dosing, may remain at QD or BID dosing. Subjects who dose at TID but cannot tolerate it, can de-escalate to QD or BID dosing. Urine samples for determination of 24-hour oxalate levels will be collected on Days 4-6 of treatment Period 1. This will be followed by a washout period of at least 2 weeks and no more than 4 weeks. After the washout period, subjects will crossover and begin Period 2. To determine baseline UOx levels for dosing Period 2, 24-hour urine samples will be collected for 3 days, within 7 days of starting dosing Period 2. Subjects will then crossover to dosing with SYNB8802 or placebo for up to TID for up to 10 days during dosing Period 2. Urine samples for 24-hour oxalate levels will be collected on Days 4-6 of treatment Period 2. A safety follow-up visit by a home healthcare professional or by telemedicine will occur 7 days after the last dose of IMP. Subjects will collect a fecal sample at baseline and weekly fecal samples for up to 4 weeks after the last dose of IMP.

Primary outcome measure will be number of subjects with treatment-emergent adverse events. Toxicity will be graded in accordance with National Cancer Institute Common Terminology for Adverse Events (CTCAE0, version 5.0. Adverse events (AEs) are reported based on clinical laboratory tests, vital signs, physical examination, electrocardiograms, and other medically indicated assessments from the time informed consent is signed throughout the end of the safety follow-up period. AEs are considered to be treatment emergent (TEAE) if they occur or worsen in severity after the first dose of study treatment. TEAEs are considered treatment-related if relationship to study drug is possibly related, probably related, or definitely related.

### Dose Cohorts and Dose Escalation:

The starting dose of SYNB8802 in Part 1a of the study will be 1×10\hat{ }11 live cells, based on clinical and nonclinical safety and tolerability of previously tested similar E. coli Nissle-based products. Dose escalation will be approximately 3-fold and up to 5-fold per cohort and an optional dose ramp may be instituted. The maximum dose will not exceed 2×10\hat{ }12 live cells. Doses may be adjusted up or down and a dose ramp instituted based on ongoing assessments. Dose adjustment decisions will be made based on tolerability (observed adverse events [AEs]), clinical observations, safety laboratory assessments, and optionally, on pharmacodynamics (PD)). The MTD for Part 1a is defined as the dose immediately preceding the dose level at which ≥4 subjects experience an IMP-related Common Terminology Criteria for Adverse Events (CTCAE) Grade 2 or ≥2 subjects experience a treatment-related Grade 3 or higher toxicity.

Before proceeding to the next dose, there must be agreement that the safety and tolerability data support dose escalation. A dose level expansion maybe recommended at the current dose, escalation to the next higher dose, decrease to a lower dose, or declaration that the MTD has been achieved. Additionally, a dose-ramp period of up to 8 days may be added to improve tolerability. Following the dose ramp (if applicable), subjects will continue dosing at the target dose level for 5 days (i.e., total dosing period may extend up to 13 days). The dose in Part 1b and Part 2 will be at or below the MTD defined in Part 1a.

In Part 1a, approximately 90 subjects are planned to be enrolled (6 treated with SYNB8802, 3 treated with placebo in each cohort). In Part 1b, up to 60 subjects (Group 1: 16 subjects; Group 2: 32 subjects; and Group 3: 12 subjects) are planned to be enrolled in Part 1b. In Part 2 up to 20 subjects are planned to be enrolled (each subject will receive SYNB8802 and placebo).

### Eligibility Criteria

### Part I: Inclusion Criteria

1. Age ≥18 to ≤64 years.

2. Body mass index (BMI) 18.5 to 28 kg/m².

3. Able and willing to voluntarily complete the informed consent process.

4. Available for and agree to all study procedures, including feces, urine, and blood collection and adherence to diet control, inpatient monitoring, follow-up visits, and compliance with all study procedures.

5. Male subjects who are sexually abstinent or surgically sterilized (vasectomy), or those who are sexually active with a female partner(s) and agree to use an acceptable method of contraception (such as a condom with spermicide) combined with an acceptable method of contraception for their non-pregnant female partner(s) (as defined in Inclusion Criterion #6) after informed consent, throughout the study, and for a minimum of 3 months after the last dose of IMP, and who do not intend to donate sperm in the period from Screening until 3 months following administration of the investigational medical product.

6. Female subjects who meet 1 of the following:

a. Woman of childbearing potential (WOCBP) must have a negative pregnancy test (human chorionic gonadotropin) at Screening and at baseline prior to the start of IMP and must agree to use

acceptable method(s) of contraception, combined with an acceptable method of contraception for their male partner(s) (as defined in Inclusion Criterion #5) after informed consent, throughout the study and for a minimum of 3 months after the last dose of IMP. Acceptable methods of contraception include hormonal contraception, hormonal or non-hormonal intrauterine device, bilateral tubal occlusion, complete abstinence, vasectomized partner with documented azoospermia 3 months after procedure, diaphragm with spermicide, cervical cap with spermicide, vaginal sponge with spermicide, or male or female condom with or without spermicide.

b. Premenopausal woman with at least 1 of the following:
i. Documented hysterectomy ii. Documented bilateral salpingectomy iii. Documented bilateral oophorectomy iv. Documented tubal ligation/occlusion v. Sexual abstinence is preferred or usual lifestyle of the subject

c. Postmenopausal women (12 months or more amenorrhea verified by follicle-stimulating hormone [FSH] assessment and over 45 years of age in the absence of other biological or physiological causes).

### Part I: Exclusion Criteria

1. Acute or chronic medical (including COVID-19 infection), surgical, psychiatric, or social condition or laboratory abnormality that may increase subject risk associated with study participation, compromise adherence to study procedures and requirements, or may confound interpretation of study safety or PD results and, in the judgment of the investigator, would make the subject inappropriate for enrollment.

2. Body mass index (BMI)<18.5 or >28 kg/m2.

3. Oxalobacter formigenes carrier.

4. Pregnant (self or partner), or lactating.

5. Unable or unwilling to discontinue vitamin C supplementation for the study duration.

6. History of or current immunodeficiency disorder including autoimmune disorders and human immunodeficiency virus (HIV) antibody positivity.

7. Hepatitis B surface antigen positivity (subjects with hepatitis B surface antibody positivity and hepatitis B core antibody positivity are not excluded, provided that the hepatitis B surface antigen is negative).

8. Hepatitis C antibody positivity, unless a hepatitis C virus ribonucleic acid test is performed, and the result is negative.

9. History of febrile illness, confirmed bacteremia, or other active infection deemed clinically significant by the investigator within 30 days prior to the anticipated first dose of IMP.

10. History of (within the past month) passage of 3 or more loose stools per day; where ‘loose stool’ is defined as a Type 6 or Type 7 on the Bristol Stool Chart (see Appendix 1: Bristol Stool Chart).

11. History of kidney stones, renal or pancreatic disease.

12. GI disorder (including inflammatory or irritable bowel disorder of any grade and surgical removal of bowel sections) that could be associated with increased UOx levels.

13. Active or past history of GI bleeding within 60 days prior to the Screening Visit as confirmed by hospitalization-related event(s) or medical history of hematemesis or hematochezia.

14. Intolerance of or allergic reaction to EcN, esomeprazole or PPIs in general, or any of the ingredients in SYNB8802 or placebo formulations.

15. Any condition (e.g., celiac disease, gastrectomy, bypass surgery, ileostomy), prescription medication, or over-the-counter product that may possibly affect absorption of medications or nutrients.

16. Currently taking or plans to take any type of systemic (e.g., oral or intravenous) antibiotic within 30 days prior to Day 1 through the final day of inpatient monitoring. Exception: topical antibiotics are allowed.

17. Major surgery (an operation upon an organ within the cranium, chest, abdomen, or pelvic cavity) or inpatient hospital stay within the past 3 months prior to Screening.

18. Planned surgery, hospitalizations, dental work, or interventional studies between Screening and last anticipated visit that might require antibiotics.

19. Taking or planning to take probiotic supplements (enriched foods excluded) within 30 days prior to Day −1 and for the duration of participation and follow-up.

20. Dependence on alcohol or drugs of abuse.

21. Administration or ingestion of an investigational drug within 30 days or 5 half-lives, whichever is longer, prior to Screening Visit; or current enrollment in an investigational study.

22. Screening laboratory parameters (e.g., chemistry panel, hematology, coagulation) and ECG outside of the normal limits based on standard ranges, or as defined in Table 34 below, or as judged to be clinically significant by the investigator. A single repeat evaluation is acceptable.

TABLE 34

| Lab Parameter | Acceptable Range |
|---|---|
| White blood cells | 3.0-14.0 × 10⁹/L |
| Platelets | >100 × 10⁹/L |
| Hemoglobin | >10 g/dL |
| Estimated glomerular filtration rate (eGFR) by | >60 mL/min/1.73 m² |
| the Chronic Kidney Disease Epidemiology | |
| Collaboration equation | |
| Aspartate aminotransferase (AST) | ≤2× upper limit of normal (ULN) |
| Alanine aminotransferase (ALT) | <2× ULN |
| Bilirubin | ≤ULN, unless diagnosed with Gilbert's syndrome |

Part II: Inclusion Criteria

1. Age ≥18 to ≤74 years.

2. Able and willing to voluntarily complete the informed consent process.

3. Available for and agree to all study procedures, including feces, urine, and blood collection and adherence to diet control, follow-up visits, and compliance with all study procedures.

4. Enteric hyperoxaluria secondary to Roux-en-Y bariatric surgery (at least 12 months post-surgery).

5. Urinary oxalate ≥70 mg/24 hours (mean of at least 2 urine collections during Screening).

6. Male subjects who are sexually abstinent or surgically sterilized (vasectomy), or those who are sexually active with a female partner(s) and agree to use an acceptable method of contraception (such as condom with spermicide) combined with an acceptable method of contraception for their non-pregnant female partner(s) (as defined in Inclusion Criterion #7) after informed consent, throughout the study, and for a minimum of 3 months after the last dose of IMP, and who do not intend to donate sperm in the period from Screening until 3 months following administration of the investigational medical product.

7. Female subjects who meet 1 of the following:

a. Woman of childbearing potential (WOCBP) must have a negative pregnancy test (human chorionic gonadotropin) at Screening and at baseline prior to the start of IMP and must agree to use acceptable method(s) of contraception, combined with an acceptable method of contraception for their male partner(s) (as defined in Inclusion Criterion #6) after informed consent, throughout the study and for a minimum of 3 months after the last dose of IMP. Acceptable methods of contraception include hormonal contraception, hormonal or non-hormonal intrauterine device, bilateral tubal occlusion, complete abstinence, vasectomized partner with documented azoospermia 3 months after procedure, diaphragm with spermicide, cervical cap with spermicide, vaginal sponge with spermicide, or male or female condom with or without spermicide.

b. Premenopausal woman with at least 1 of the following:
i. Documented hysterectomy ii. Documented bilateral salpingectomy iii. Documented bilateral oophorectomy iv. Documented tubal ligation/occlusion v. Sexual abstinence is preferred or usual lifestyle of the subject

c. Postmenopausal women (12 months or more amenorrhea verified by FSH assessment and over 45 years of age in the absence of other biological or physiological causes).

8. Screening laboratory evaluations (e.g., chemistry panel, complete blood count with differential, prothrombin time [PT]/activated partial thromboplastin time [aPTT], urinalysis) and electrocardiogram (ECG) must be within normal limits or judged to be not clinically significant by the investigator.

### Part II: Exclusion Criteria

1. Acute or chronic medical (including COVID-19 infection), surgical, psychiatric, or social condition or laboratory abnormality that may increase subject risk associated with study participation, compromise adherence to study procedures and requirements, or may confound interpretation of study safety or PD results and, in the judgment of the investigator, would make the subject inappropriate for enrollment.

2. Acute renal failure or eGFR<45 mL/min/1.73 m². A single repeat evaluation is acceptable.

3. Unable or unwilling to discontinue vitamin C supplementation for the study duration.

4. Diagnosis of primary hyperoxaluria or any other cause of hyperoxaluria.

5. Oxalobacter formigenes carrier.

6. Pregnant (self or partner), or lactating.

7. Currently taking or plans to take any type of systemic (e.g., oral or intravenous) antibiotic within 30 days prior to Day 1 through the final safety assessment. Exception: topical antibiotics are allowed.

8. Major surgery (an operation upon an organ within the cranium, chest, abdomen, or pelvic cavity) or inpatient hospital stay within the past 3 months prior to Screening.

9. Planned surgery, hospitalizations, dental work, or interventional studies between Screening and last anticipated visit.

10. Taking or planning to take probiotic supplements (enriched foods excluded) within 30 days prior to Day −1 and for the duration of participation.

11. Intolerance of or allergic reaction to EcN, esomeprazole or PPIs in general, or any of the ingredients in SYNB8802 or placebo formulations.

12. Dependence on alcohol or drugs of abuse.

13. History of or current immunodeficiency disorder including autoimmune disorders and HIV antibody positivity.

14. Hepatitis B surface antigen positivity (subjects with hepatitis B surface antibody positivity and hepatitis B core antibody positivity are not excluded, provided that the hepatitis B surface antigen is negative).

15. Hepatitis C antibody positivity, unless a hepatitis C virus ribonucleic acid test is performed, and the result is negative.

16. Administration or ingestion of an investigational drug within 30 days or 5 half-lives, whichever is longer, prior to Screening Visit; or current enrollment in an investigational study.

17. History of bacteremia within 30 days prior to the anticipated first dose of IMP.

18. History of inflammatory bowel disease.

### Summary of Clinical Protocol

Part 1 (Healthy Volunteers):

Primary objective is to evaluate the safety and tolerability of SYNB8802. Secondary objective is to evaluate the microbial kinetics of SYNB8802 in feces. To assess the effect of SYNB8802 on urinary oxalate (UOx) excretion after an average-oxalate low-calcium (AOLC) diet.

Exploratory objectives include (i) assess the effect of SYNB8802 on urinary oxalate (UOx) amount excreted and, in Part 1b only, to compare this effect with and without concomitant administration of proton pump inhibitor (PPI) and with and without galactose, (ii) assess the effect of SYNB8802 on UOx:creatinine ratios, (iii) assess the effect of SYNB8802 on urinary biomarkers (potassium, calcium, phosphorus, uric acid, citrate, magnesium, sodium, chloride, sulfate, ammonium, urea nitrogen, and pH), (iv) assess the effect of SYNB8802 on plasma oxalate (POx) levels, and (v) assess the effect of SYNB8802 on fecal oxalate levels (Part 1a only).

Additional exploratory objectives include (i) To assess the effect of SYNB8802 on biomarkers associated with increased risk of kidney stones. (ii) To assess the effect of SYNB8802 on fecal oxalate levels. (iii) To assess the effect of SYNB8802 on plasma oxalate (POx) levels. (iv) To assess potential factors that predict oxalate responses. (v) To explore potential biomarkers of tolerability

Part 2 (Patients with Enteric Hyperoxaluria):

Primary objective is to assess the effect of SYNB8802 on UOx amount excreted. Secondary objective is to assess the effect of SYNB8802 on the UOx:creatinine ratio, to evaluate the microbial kinetics of SYNB8802 in feces, and to evaluate the safety and tolerability of SYNB8802.

Exploratory objectives are to assess (i) the effect of SYNB8802 on POx levels, (ii) the effect of SYNB8802 on serum phosphorus levels, and (iii) the effect of SYNB8802 on urinary biomarkers (potassium, calcium, phosphorus, uric acid, citrate, magnesium, sodium, chloride, sulfate, ammonium, and pH).

TABLE 35
Overview of Study Cohorts

| | | | SYNB8802 | | | Treatment |
|---|---|---|---|---|---|---|
| | | | Dose (# live | Diet | Dose Ramp | Period |
| Study Parts | | Cohort Details | cells) | Run-In | (Optional) | (TP)^{a} |
| Part | Part 1a | Cohort 1 (HV) | 1 × 10¹¹ | 4 Days | N/A | 5 Days |
| 1 | (MAD) | Cohort 2 (HV) | 3 × 10¹¹ | 4 Days | N/A | 5 Days |
| | | Cohort 3 (HV) | 1 × 10¹² | 4 Days | Up to 4 | 5 Days |
| | | | | | Days | |
| | | Cohort 4 (HV) | TBD ≤ 2 × 10¹² | 4 Days | Up to 4 | 5 Days |
| | | | | | Days | |
| | | Cohort 5 (HV) | TBD ≤ 2 × 10¹² | 4 Days | Up to 4 | 5 Days |
| | | | | | Days | |
| | | Cohort 6-10 | TBD ≤ 2 × 10¹² | 5 Days | Up to 8 | 5 Days |
| | | (optional) (HV) | | | Days | |
| | Part 1b | Proton pump | MTD or | 5 Days | Up to 4 | 4 Days (×3 |
| | | inhibitor and | lower | (×3) | Days | TPs) |
| | | galactose crossover | dose | | | |
| | | cohort (HV) | | | | |
| | | SYNB8802 | | | | |
| | | containing | | | | |
| | | galactose with PPI. | | | | |
| | | SYNB8802 | | | | |
| | | containing | | | | |
| | | galactose without | | | | |
| | | PPI. | | | | |
| | | SYNB8802 without | | | | |
| | | galactose and with | | | | |
| | | PPI | | | | |
| Part | | PD effects of | MTD or | N/A | Up to 4 | 6 Days (×2 |
| 2^{b} | | SYNB8802 in | lower | | Days | TPs) |
| | | subjects with enteric | dose | | | |
| | | hyperoxaluria | | | | |

BID = twice a day; HV = healthy volunteer; IMP = investigational medicinal product; MTD = maximum; tolerated dose; QD = once daily; TBD = to be determined; TID = three times per day; TP = treatment period; N/A = not applicable.
^{a}For Part 1a, the treatment period includes 5 days of IMP dosing and 1 day for assessments prior to discharge.
^{b}Subjects who in the opinion of the investigator cannot progress beyond QD or BID dosing may remain at QD or BID dosing.
Subjects who dose at TID but cannot tolerate it can de-escalate to QD or BID dosing.

Investigational Medicinal Product(s):

SYNB8802 (with or without galactose) at 1×10¹¹, 3×10¹¹, or 1×10¹² live cells (may be adjusted up or down based on ongoing assessments but will not exceed 2×10¹²), orally, up to 3 times per day (TID) or per dose-ramp schedule, with meals. Placebo to match SYNB8802, orally, up to TID or per dose-ramp schedule, with meals.

Duration of Treatment

The maximum time of study participation for a subject in Part 1a is planned to be up to 132 days: including (i) Screening period: up to 90 days (including a 4-day or 5-day diet run-in); (ii) Treatment period: up to 14 days (up to 10 dosing days including optional dose-ramp period with discharge from the CRU on the following day); and (iii) Safety follow-up period (including fecal assessments): 28 days.

The maximum time of study participation for a subject in Part 1b is planned to be 156 days: including, (i) Screening period: Up to 90 days (including a 5-day diet run-in), (ii) Dosing and washout periods: Up to 52 days (e.g., Dosing period 1: Up to 8 days, including optional dose-ramp period; Washout period: 14 days (including a 5-day diet run-in); Dosing period 2: Up to 8 days, including optional dose-ramp period; Washout period: 14 days (including a 5-day diet run-in)); and Dosing period 3: Up to 8 days, including optional dose-ramp period); (iii) Safety follow-up period (including fecal assessments): 14 days.

The maximum time of study participation for a subject in Part 2 is planned to be 135 days: including (i) Screening period: Up to 52 days (including baseline UOx for dosing Period 1); (ii) Dosing periods 1 and 2, washout, and baseline UOx for dosing period 2: Up to 55 days (e.g., Dosing period 1: Up to 10 days, including optional dose-ramp period; Washout period: ≥14 days and no more than 28 days; Baseline UOx for dosing period 2: Up to 7 days before start of dosing period 2; and Dosing period 2: Up to 10 days, including optional dose-ramp period); and (iii) Safety follow-up period (including fecal assessments): 28 days.

### Study Endpoints

Part 1: Primary Endpoint
Safety and tolerability of SYNB8802, as assessed by adverse events, clinical laboratory tests, and vital sign measurements

Secondary Endpoint
Microbial kinetics of SYNB8802, measured from feces with quantitative polymerase chain reaction (qPCR) following dosing

Exploratory Endpoints
Change from baseline in 24-hour UOx amount excreted in SYNB8802-treated subjects: (i) versus placebo (Part 1a, only); (ii) with or without PPI (Part 1b, only); and (iii) with or without galactose (Part 1b, only)
Change from baseline in UOx:creatinine ratio in SYNB8802-treated subjects: (i) versus placebo (Part 1a, only); (ii) with or without PPI (Part 1b, only); and (iii) with or without galactose (Part 1b, only)
Change from baseline in urinary biomarkers (potassium, calcium, phosphorus, uric acid, citrate, magnesium, sodium, chloride, sulfate, ammonium, urea nitrogen, and pH) in SYNB8802-treated subjects: (i) versus placebo (Part 1a, only); (ii) with or without PPI (Part 1b, only); (iii) with or without galactose (Part 1b, only).
Change from baseline in POx levels in SYNB8802-treated subjects: (i) versus placebo (Part 1a, only); (ii) with or without PPI (Part 1b, only); (iii) with or without galactose (Part 1b, only).
Change from baseline in fecal oxalate levels in SYNB8802-treated subjects versus placebo (Part 1a only).

Part 2: Primary Endpoint
Change from baseline in 24-hour UOx amount excreted with SYNB8802 treatment versus placebo treatment.

Secondary Endpoints
Change from baseline in UOx:creatinine ratio with SYNB8802 treatment versus placebo treatment;
Microbial kinetics of SYNB8802, measured from feces with qPCR;
Safety and tolerability of SYNB8802, as assessed by adverse events, clinical laboratory tests, and vital signs measurements.

Exploratory Endpoints
Change from baseline in POx levels with SYNB8802 treatment versus placebo treatment;
Change from baseline in serum phosphorus with SYNB8802 treatment versus placebo treatment;
Change from baseline in urinary biomarkers (potassium, calcium, phosphorus, uric acid, citrate, magnesium, sodium, chloride, sulfate, ammonium, and pH) with SYNB8802 treatment versus placebo treatment.

Study Suspension: Enrollment into any part of the study will be suspended for the following reasons: (i) One or more subjects experience an SAE that is possibly, probably, or definitely related to the IMP as assessed by the investigator; (ii) One or more subjects experience an AE≥Grade 3 in severity that is possibly, probably, or definitely related to the IMP using the National Cancer Institute (NCI) CTCAE≥Grade 3 as assessed by the investigator. (Note that Grade 3 AEs related to nausea, vomiting, and diarrhea may suspend dosing at the current dose, but will not suspend the study overall.); (iii) a determination is made that an event or current data warrant further evaluation.

Study Stop: The occurrence of the following events will require that further enrollment in the study be stopped: (i) Two or more subjects in a cohort experience SAEs that are possibly, probably, or definitely related to the IMP as assessed by the investigator; (ii) Death occurs at any time during the study and is considered by the investigator to be related to the IMP; (iii) Clinical infection with SYNB8802 in a sterile space confirmed by clinical culture and/or qPCR; and (iv) a determination is made that an event or current data warrant stopping the study.

Part 1a: MAD Cohorts

Part 1a is an inpatient, placebo-controlled, MAD study in HVs. Subjects will report to the clinical research unit (CRU) on Day −4 or Day −5. Subjects in cohorts 1-5 will complete a 4-day diet run-in (Days −4 to −1), during which they will consume a highoxalate, low-calcium diet (details will be provided in the Diet Manual). Subjects in Cohorts 6-10 will complete a 5-day diet run-in (Days −5 to −1), during which they will consume a highoxalate, low-calcium diet (details will be provided in the Diet Manual). Dietary oxalate and calcium will be distributed across 3 meals per day. On the morning of the first day of the diet run-in, a forced-void urine sample will be collected. Daily 24-hour urine collection will then be started to determine UOx levels. On Day 1, subjects will be randomly assigned to treatment with SYNB8802 or placebo (collectively referred to as “investigational medicinal product” [IMP]). Subjects will then begin oral dosing with IMP up to 3 times per day, with meals, for up to a total of 13 days during the optional Dose-Ramp and treatment periods. Subjects will maintain the high-oxalate, low-calcium diet during the dosing period and fecal samples will be collected on days of IMP dosing. Subjects will take a PPI (esomeprazole) once a day, 60-90 minutes before breakfast, starting on the first day of the diet run-in until the last day of IMP dosing. (see Section 5.3.2.1 for details). Subjects will be released from the CRU on the day after completion of the dosing period following completion of safety assessments. A safety follow-up visit will occur 7 days after last dose of IMP. Subjects will collect weekly fecal samples for 4 weeks after the last dose of IMP.

Part 1b: Proton Pump Inhibitor and Galactose Crossover Cohort

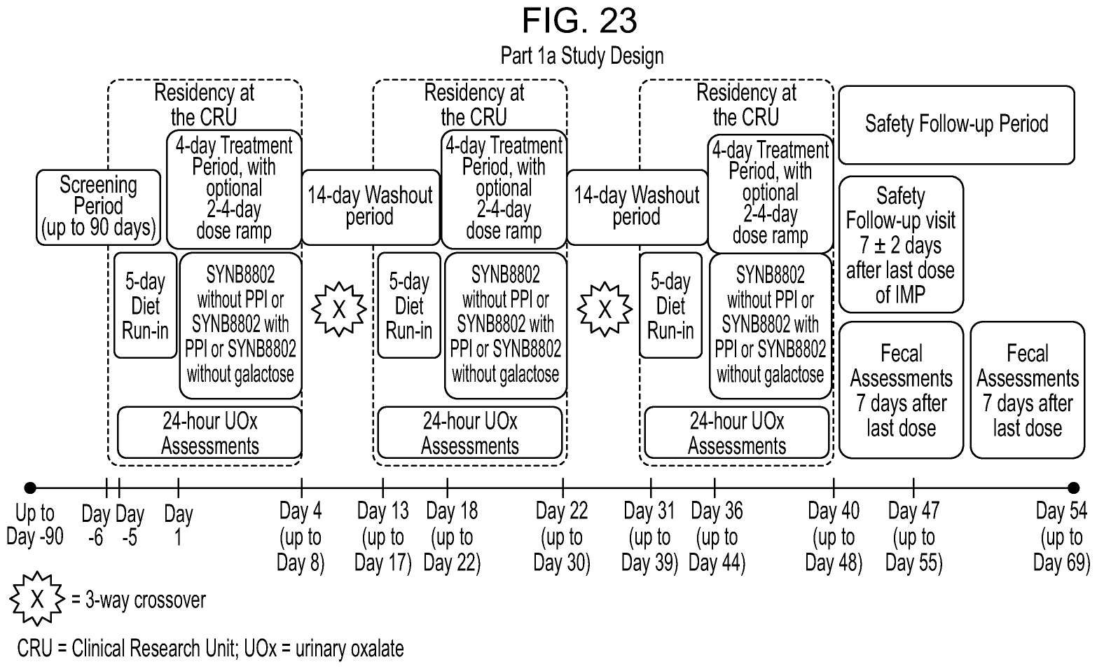

The PPI is administered to protect SYNB8802, a live biotherapeutic, from the acidic environment in the stomach. D-galactose has been included in the formulation for SYNB8802, including the formulation used in Part 1a cohorts and Part 2, to enhance its cellular activity. In Part 1b (FIG. 23), the effects of concomitant PPI administration and galactose as part of the formulation on the PD of SYNB8802 will be evaluated using a crossover design. On Day −5 prior to the first dosing period, subjects will be randomly assigned to receive a sequence of three different treatments at the Part 1a MTD or lower tolerated dose of SYNB8802 defined in Part 1a during the three dosing periods in a crossover manner. The 3 treatments in Part 1b are:
(i) SYNB8802 containing galactose with concomitant PPI
(ii) SYNB8802 containing galactose without PPI
(iii) SYNB8802 without galactose and with concomitant PPI.

During dosing periods that require concomitant PPI administration, subjects will start taking esomeprazole once a day, 60-90 minutes before breakfast on Day −5 and continue until the last day of IMP dosing in each dosing period. Subjects will complete a 5-day diet run-in prior to each dosing period, during which they will consume a high-oxalate low-calcium diet (refer to Diet Manual for details). Dietary oxalate and calcium will be distributed across 3 meals per day. Subjects will maintain this diet throughout each dosing period. On the morning of Day −5 prior to each dosing period, a forced-void urine sample will be collected. Daily 24-hour urine collections will then be started and continued for the duration of each inpatient stay. During each dosing period, subjects will be treated with SYNB8802 (with or without galactose) up to 3 times per day with meals for up to 8 days, including the optional dose-ramp and treatment periods. Subjects will be released from the CRU on the day following the last dose of IMP, after completion of safety assessments. There will be a 14-day washout between treatment periods. The use of subjects as their own controls will enable a comparative evaluation of the safety, tolerability, and PD of SYNB8802 with and without concomitant PPI as well as with and without galactose. Subjects will collect weekly fecal samples for 2 weeks after the final dose of IMP.

Part 2: Pharmacodynamic Effects of SYNB8802 in Subjects with Enteric Hyperoxaluria

Part 2 (FIG. 12) is a double-blind (sponsor-open), outpatient, placebo-controlled crossover study of SYNB8802 in subjects with EH. All subject evaluations and assessments throughout this study may be conducted either at the clinical site or by a home healthcare professional at an alternative location (e.g., subject's home, hotel). Subjects will maintain their normal diet throughout the study, which they will record using a daily diary on those days requiring 24-hour urine collection during the baseline and treatment periods. To determine baseline UOx levels for dosing period 1, 24-hour urine samples will be collected for 3 days, within 7 days of starting dosing Period 1. Subjects will take a PPI (esomeprazole) QD, 60-90 minutes before the meal of their choosing, starting 4 days prior to the first IMP dose of each dosing period through the last IMP dose of each dosing period. Subjects will be randomized between Day −7 to −4 to receive SYNB8802 at or below the MTD defined in Part 1a or placebo. Subjects will be dosed with IMP up to 3 times per day with meal(s) for up to 10 days during dosing Period 1. Subjects who in the opinion of the investigator cannot progress beyond QD or twice a day (BID) dosing, may remain at QD or BID dosing. Subjects who dose at TID but cannot tolerate it, can de-escalate to QD or BID dosing. Urine samples for 24-hour oxalate levels will be collected on Days 4-6 of treatment Period 1. This will be followed by a washout period of at least 2 weeks and no more than 4 weeks. After the washout period, subjects will crossover and begin dosing Period 2. To determine baseline UOx levels for dosing Period 2, 24-hour urine samples will be collected for 3 days, within 7 days of starting dosing Period 2. Subjects will then crossover to dosing with SYNB8802 or placebo for up to 3 times per day with meal(s) for up to 10 days during dosing Period 2. Urine samples for 24-hour oxalate levels will again be collected on Day 4-6 of treatment Period 2. A safety follow-up visit by a home healthcare professional or by telemedicine will occur 7 days after the last dose of IMP. Subjects will collect a fecal sample at baseline and weekly fecal samples for up to 4 weeks after the last dose of IMP.

Dose and Dose Escalation in Part 1a

The starting dose of SYNB8802 in Part 1a of the study will be 1×10¹¹ live cells, orally, TID, based on clinical and nonclinical safety and tolerability of previously tested EcN-based genetically modified organisms. Dose escalation will be approximately 3-fold and up to 5-fold per cohort and a dose-ramp may be instituted. Decisions will be made based on tolerability (observed AEs), clinical observations, safety laboratory assessments, and, optionally, on PD assessments. Doses may be adjusted up or down and a dose-ramp instituted based on emergent data. Doses will not be escalated more than 5-fold between cohorts, and the maximum dose will not exceed 2×10¹² live cells. Dose escalation decisions will be made in Part 1a of the study once the last subject in a cohort has been dosed and has had at least 24 hours of post dose observation. Decisions will be made based on tolerability (observed AEs), clinical observations, safety laboratory assessments, and optionally PD assessments. Before proceeding to the next dose there must be agreement that the safety and tolerability data support dose escalation. A dose level expansion maybe be recommended at the current dose level, escalation to the next higher dose level, decrease to a lower dose level, institution of a dose-ramp, or declaration that the MTD has been achieved. The MTD for Part 1a is defined as the dose immediately preceding the dose level at which ≥4 subjects experience an IMP-related Common Terminology Criteria for Adverse Events (CTCAE) Grade 2 or ≥2 subjects experience a treatment-related Grade 3 or higher toxicity.

Washout Periods

In Part 1b of the study, between each dosing period, subjects will undergo washout for 14 days during which they will not receive IMP before crossing over to the subsequent dosing period. The diet run-in period may overlap with the last 5 days of the washout period. A fecal sample should be collected within 2 days of the last day of each washout period.

TABLE 36
Other Sequences Related to SYN7169 and SYNB8802

| Description | SEQ ID NO |
|---|---|
| >gnl\|ECOLI\|THYMIDYLATESYN-MONOMER thymidylate synthase | SEQ ID NO: 61 |
| (complement(2964361 . . . 2965155)) Escherichia coli K-12 substr. MG1655 | |
| >gnl\|ECOLI\|EG11002 thyA THYMIDYLATESYN-MONOMER | SEQ ID NO: 62 |
| (complement(2964361 . . . 2965155)) Escherichia coli K-12 substr. MG1655 | |
| phage 3 ko sequence | SEQ ID NO: 63 |

### Example 6: SYNB8802 Proof of Mechanism in Dietary Hyperoxaluria

Healthy volunteers on a high oxalate and low calcium diet were treated with multiple ascending doses of SYNB8802. In the study's efficacy analysis, the percent change from baseline urinary oxalate levels were −28.6% (90% CI: −42.4 to −11.6), compared to placebo, at the 3e11 live cell dose. This dose was well tolerated and will be used in Part B of the study.

Part B of the study will assess urinary oxalate lowering potential of SYNB8802 in patients with Enteric Hyperoxaluria following Roux-en-Y gastric bypass surgery.

### SYNB8802 Phase 1A Study: Design and Results

The primary outcome of Part A of the Phase 1 study was safety and tolerability, with results used to select a dose for further study in patients with Enteric Hyperoxaluria in Part B of the trial. Dosing of five cohorts in part A, 45 total subjects has been completed. Findings include:

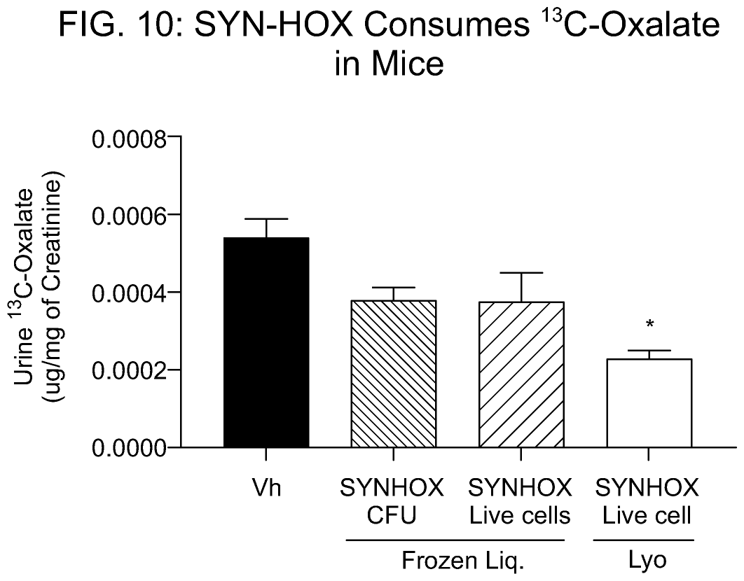

SYNB8802 was generally well tolerated in healthy volunteers. There were no serious or systemic adverse events. The most frequent adverse events were mild or moderate, transient, and GI-related. Dietary Hyperoxaluria was successfully induced in Healthy Volunteers. Subjects placed on 600 mg of daily dietary oxalate, e.g., high oxalate, low calcium, had urinary oxalate levels of 44.8 mg/24 h at baseline (FIG. 13). Urinary oxalate levels elevated to >1.5× typically observed in healthy volunteers. Dietary intake was carefully measured on an in-patient basis, including weighing of meals consumed by volunteers.

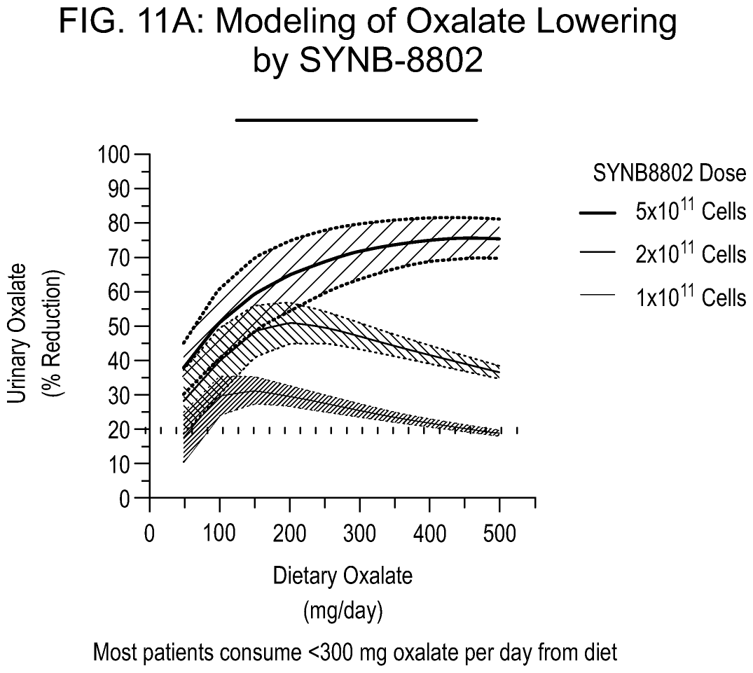

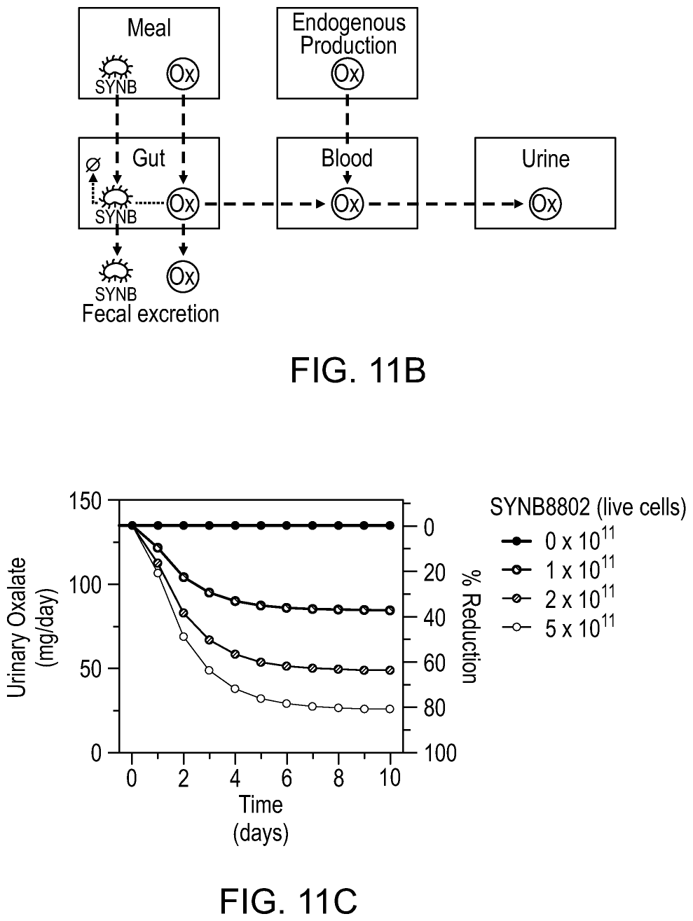

Dose responsive changes in urinary oxalate levels were observed with a significant reduction in urinary oxalate relative to placebo across three dose levels (FIGS. 14A and 14B). A dose of 3e11 live cells administered three times daily with meals was selected as the dose for part B of the study.

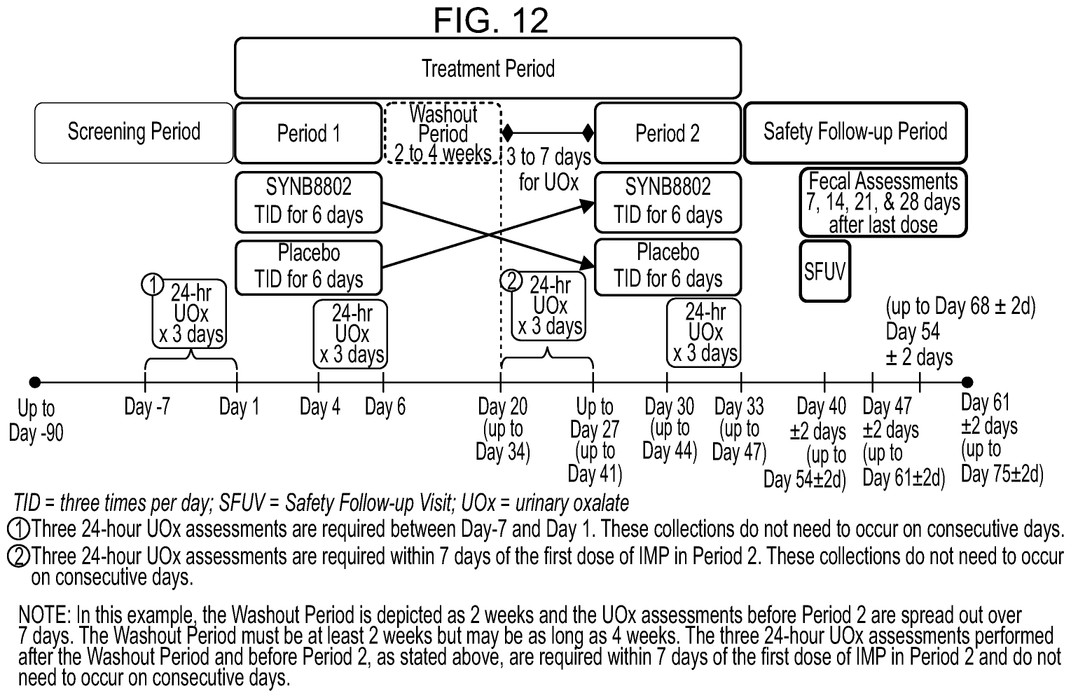

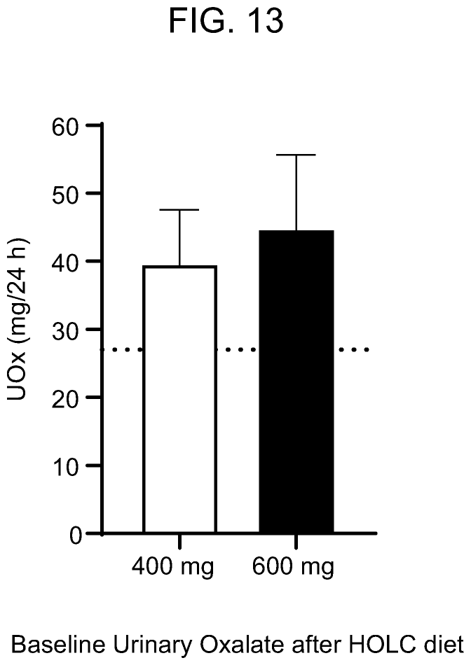

This dose was well-tolerated and resulted in a change from baseline urinary oxalate reduction of 28.6% (90% CI: −42.4 to −11.6), compared to placebo, and 32% as compared to placebo (FIGS. 15A and 16, respectively).

At the end of dosing, the mean 24-hour urinary oxalate level was 40.1 mg for subjects treated with SYNB8802 3e11 live cells, compared to 58.1 mg for placebo subjects (FIG. 15B). Upper limit of normal urinary oxalate levels are 45 mg per 24 hours. 3e11 live cells dose is advancing to patient studies.

Further Interim results from the Study indicate that doses ≥1×10¹¹ live cells SYNB8802 TID lower urinary oxalate approximately 20% to 40% in healthy subjects on a high-oxalate, low-calcium diet targeting 400 or 600 mg daily dietary oxalate (Table 37).

Additional interim results from the same study indicate that SYNB8802 dose-responsively reduces fecal oxalate concentration with a >50% reduction at doses ≥3×10¹¹ live cells SYNB8802 TID.

TABLE 37
Placebo-adjusted Change in Urine Oxalate

| Target Daily | Number | | % Urine Oxalate | |
|---|---|---|---|---|
| Dietary | of | | LoweringLSM | |
| Oxalate (mg) | Subjects | Treatment Description | (90% CI) | |
| 400 | 6 | 1 × 10¹¹ live cells SYNB8802 TID | −32.5 | [−46.9, −14.2] |
| 400 | 6 | 3 × 10¹¹ SYNB8802 TID | −23.3 | [−39.7, −2.39] |
| 600 | 6 | 5 × 10¹¹ SYNB8802 titrated to TID | 12.5 | [−13.9, 47] |
| 600 | 6 | 3 × 10¹¹ SYNB8802 titrated to TID | −26.3 | [−43.6, −3.7] |
| 600 | 6 | 4.5 × 10¹¹ SYNB8802 titrated to TID | −21.3 | [−39.7, 2.89] |
| 600 | 6 | 6 × 10¹¹ SYNB8802 titrated to TID | −41.4 | [−56, −21.9] |

TID: 3 times daily
Note:
subjects were healthy volunteers on a high-oxalate, low-calcium diet.

### Example 7: SYNB8802 Activity Under Conditions Representing the GI Lumen

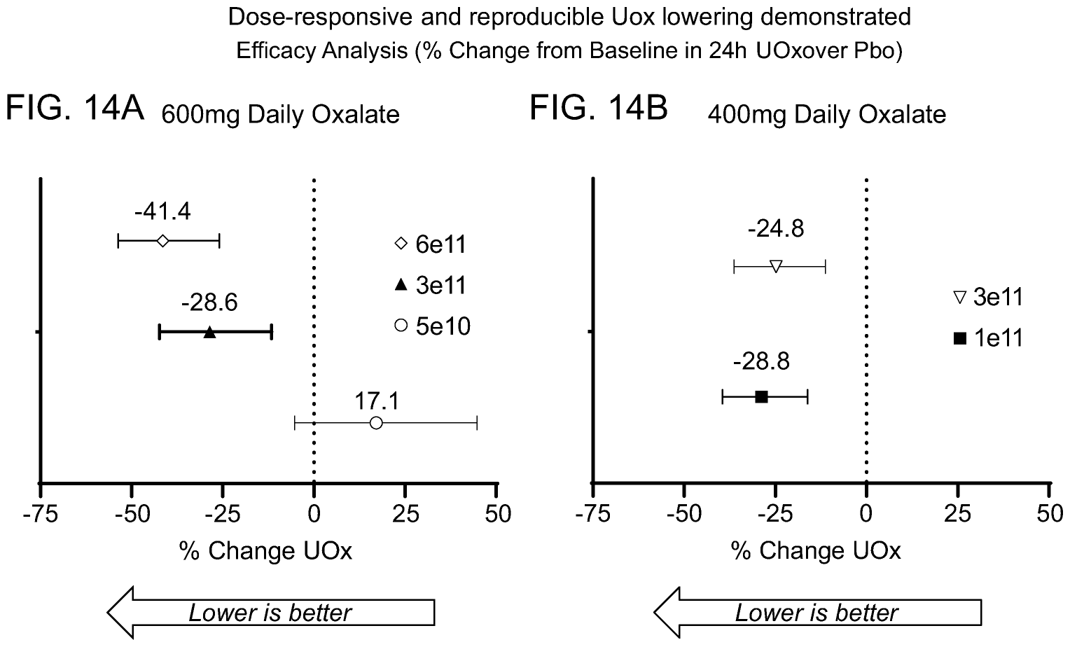

To estimate SYNB8802 activity under conditions representing the GI lumen, an in vitro simulation (IVS) system was developed, comprising a series of incubations in media representing human stomach, small intestine, and colon compartments by simulating luminal pH and oxygen, gastric and pancreatic enzymes, and GI transit times. The rate of oxalate degradation was estimated in each simulated compartment (FIG. 17). Oxalate consumption was highest in simulated gastric fluid (SGF) (1.35±0.04 and 1.52±0.08 μmol oxalate/hr*10⁹ cells at one and two hours post inoculation, respectively) and remained at similar levels after 1 h incubation in simulated small intestinal fluid (SIF). Oxalate consumption decreased to 0.88±0.04 μmol oxalate/hr*10⁹ cells after 2 h incubation in SIF. SYNB8802 activity further decreased to 0.2±0.14 μmol oxalate/hr*10⁹ cells in the completely anaerobic conditions of simulated colonic fluid (SCF), where it remained relatively stable over the 48 h incubation period. These data suggest that SYNB8802 has the potential to metabolize oxalate throughout the human GI tract.

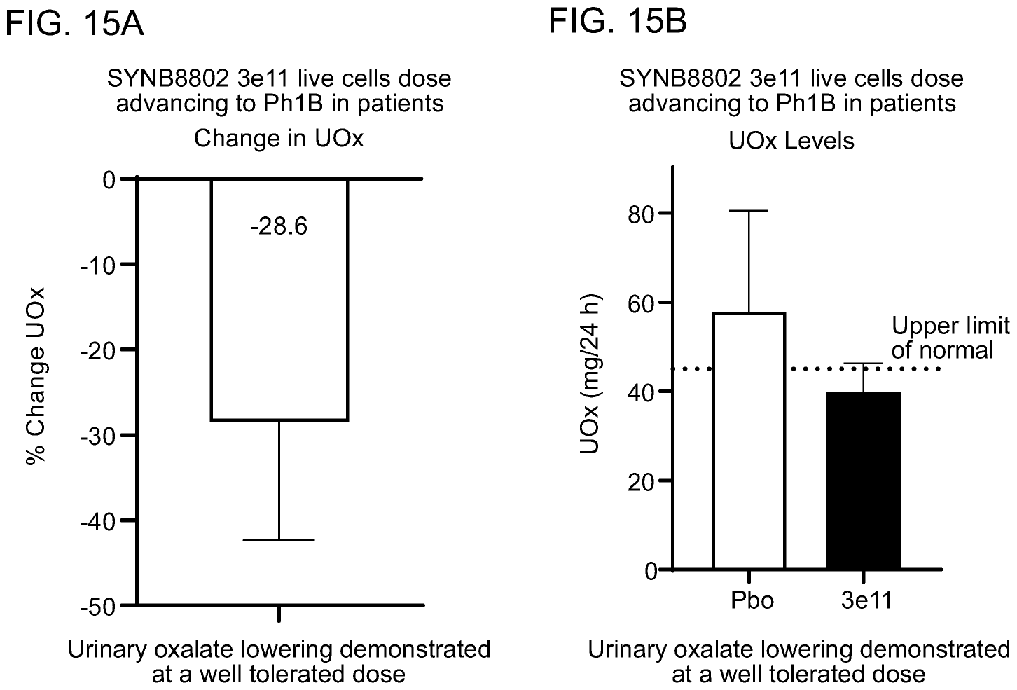

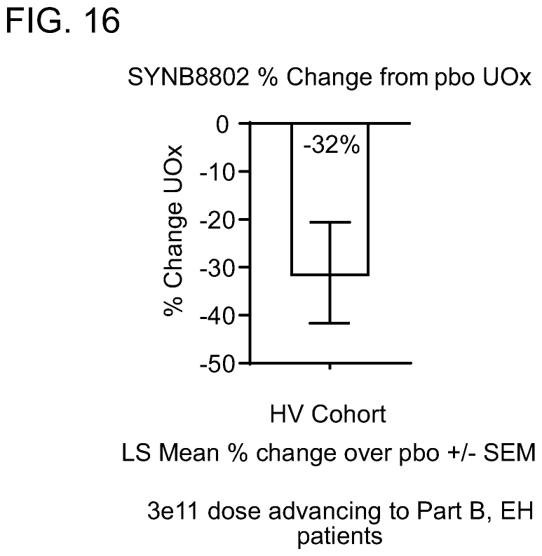

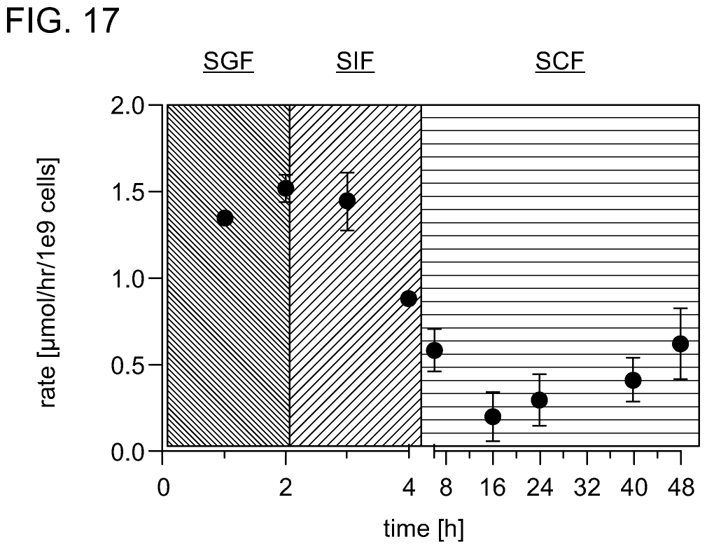

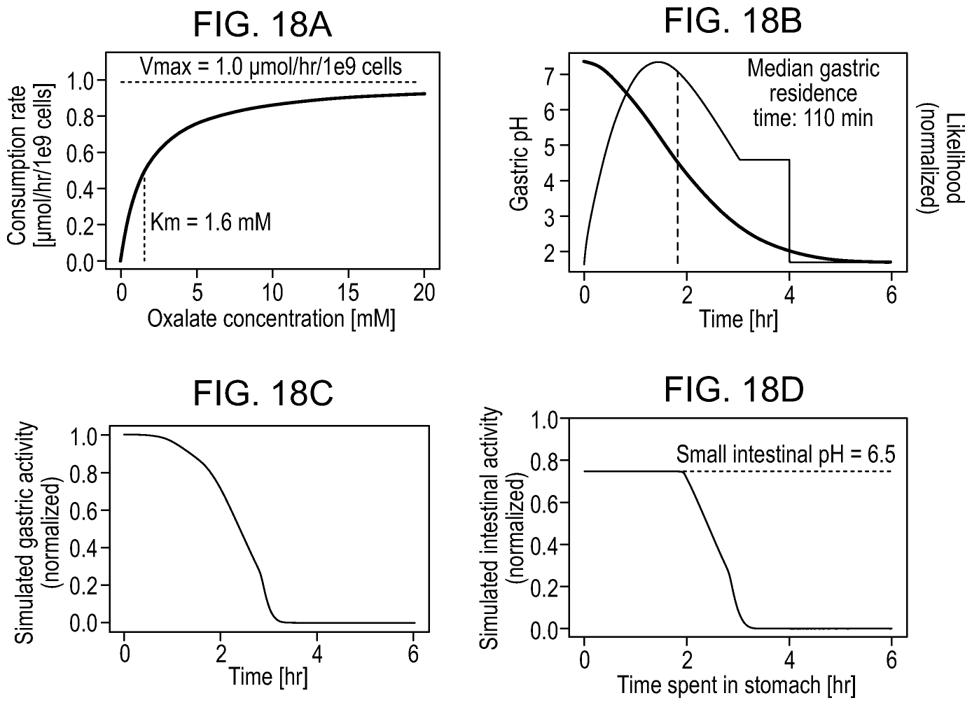

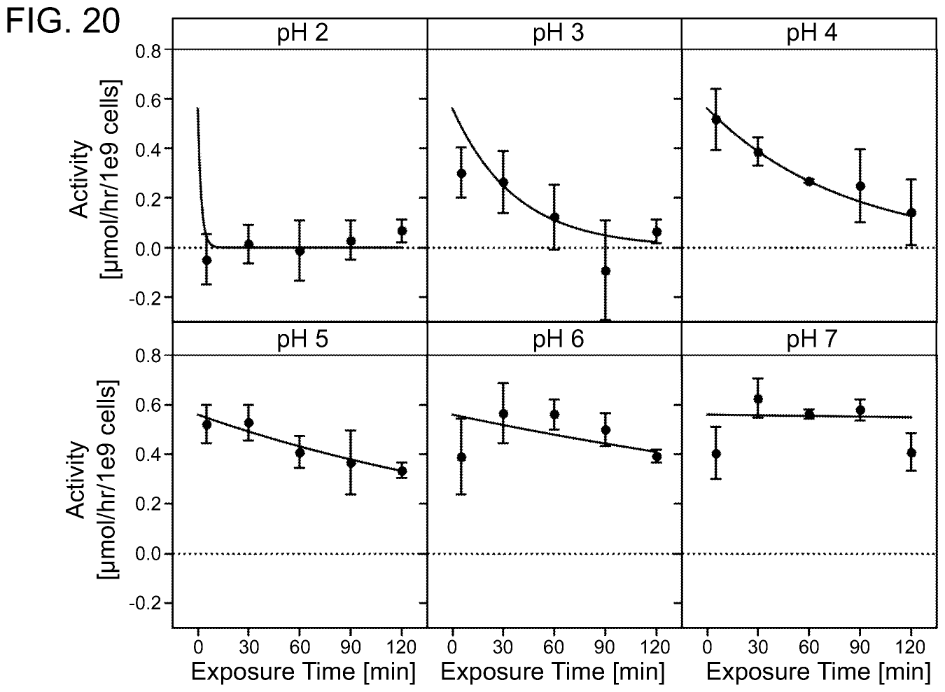

Oxalate consumption by SYNB8802 was modeled according to Michaelis-Menten kinetics by fitting to data from IVS (FIG. 18A) while accounting for conditions within the GI tract that may affect strain function. Specifically, oral administration of SYNB8802 involves transient exposure to low pH within the stomach. Human gastric pH is dynamic, increasing after a meal, then decreasing to ˜2 in subsequent hours (FIG. 18B). In addition, the ISS model of GI transit indicates that a population of SYNB8802 cells in a single dose follows a distribution of gastric residence times (FIG. 18B), suggesting that some cells spend more time in the acidic environment of the stomach than others. To understand the effects of environmental pH on oxalate consumption by SYNB8802, an in vitro simulation was performed in which SYNB8802 oxalate consumption was determined at a variety of pH levels over time (FIG. 20). Consumption decreased as a function of lower pH and longer exposure times. To account for this observation in the ISS model, an exponential decay function was fit to each pH level (FIG. 20), and these pH effect models were mapped to the dynamic pH of the human stomach in silico, such that reduction in strain activity was estimated as a function of time spent in the stomach (FIG. 4C). Inhibition of activity due to gastric residence time was then retained for SYNB8802 cells as they transited through the remainder of the GI tract (FIG. 4D). Thus, intestinal and colonic activity of SYNB8802 was informed by how long each individual cell spent in the stomach. Collectively, the ISS model provides a mathematical framework incorporating SYNB8802 activity and information regarding strain and substrate transit through the GI tract to enable physiological estimation of strain performance in vivo.

### In Silico Simulation (ISS)

The modeling approach integrates SYNB8802 activity informed by in vitro studies with the gastrointestinal and circulation physiology to predict urinary oxalate lowering by oral administration of SYNB8802. A multi-compartment approach was taken wherein volume dynamics were modeled alongside SYNB8802 and oxalate dynamics. In contrast to a typical approach assuming static compartment volumes, the volume of chyme, or partially digested food, within each gut organ was considered as the compartment, rather than the organ itself. Plasma oxalate dynamics were modeled as an initial serum level and an eventual steady state resulting from any change in the amount of oxalate absorbed from the gut. This framework allowed for simulation of either increased gut absorption (e.g., introduction of a high-oxalate diet) or decreased gut absorption (e.g., introduction of SYNB8802). SYNB8802 and oxalate were simulated to enter the stomach with a meal three times per day and progress through the stomach, small intestine, and colon concurrently with chyme. The processes governing oxalate abundance in the gut were described using material balances implemented as ordinary differential equations (ODEs) (Equations 1-9). Each ODE describes the rate of change of a state variable from its initial value to the end of the simulation time (48 hours). The initial values of all state variables can be found in Table 38 and the parameter values can be found in Table 39. The initial value of the gastric chyme volume state variable was equal to the total gastric emptying volume, taken as the volume of food eaten and fluid drunk per day for a typical human (Sherwood. Human Physiology: From Cells to Systems. s.l.: Wadsworth publishing company 3rd edition, 1997. pp. 590; Sandle, G. Salt and water absorption in the human colon: a modern appraisal. 1998, Gut.; Thomas A., Gut motility, sphincters and reflex control. 2006, Anaesthesia Intens Care Med.; Southwell, B R., Colonic transit studies: normal values for adults and children with comparison of radiological and scintigraphic methods. 2009, Pediatr Surg Int.) divided by the number of meals per day. The total secretions volume was taken as the volume of plasma secretions into the small intestine per day for a typical human divided by the number of meals per day. The total intestinal and colonic emptying volumes were based on the values reported by Sherwood et. al. and those reported by Sandle. The intestinal transit time was taken from a study on chyme transit in the gut. The colonic transit time was taken from a study on methods for measuring colonic transit.

TABLE 38
Initial Values of ISS State Variables

| State | | Initial | | |
|---|---|---|---|---|
| Variable | Description | Value | Units | Source |
| V_{gastric} | Volume of chyme in the stomach | 833 | mL | Sherwood |
| | | | | 1997 |
| V_{SI} | Volume of chyme in the small | 0 | mL | — |
| | intestine | | | |
| V_{colon} | Volume of chyme in the colon | 0 | mL | — |
| Ox_{gastric} | Abundance of oxalate in the | Dietary | μmol | — |
| | stomach | intake-dependent | | |
| Ox_{SI} | Abundance of oxalate in the small | 0 | μmol | — |
| | intestine | | | |
| Ox_{colon} | Abundance of oxalate in the colon | 0 | μmol | — |
| CFU_{gastric} | SYNB8802 population in the | Dose-dependent | Cells | — |
| | stomach | | | |
| CFU_{SI} | SYNB8802 population in the small | 0 | Cells | — |
| | intestine | | | |
| CFU_{colon} | SYNB8802 population in the colon | 0 | Cells | — |

TABLE 39
ISS Parameter Values

| Parameter | Description | Value | Units | Source |
|---|---|---|---|---|
| β | Gastric emptying curve shape | 1.81 | unitless | Elashoff |
| | parameter | | | 1982 |
| τ_{1/2} | Half gastric emptying time | 110 | min | Elashoff |
| | | | | 1982 |
| τ_{gastric} | Duration of gastric emptying | 240 | min | Calibrated |
| τₗᵢₙₑₐᵣ | Beginning of linear gastric | 182 | min | Calibrated |
| | emptying | | | |
| V_{total gastric emptying} | Total gastric emptying volume | 833 | mL | Sherwood |
| | | | | 1997 |
| V_{total secretions} | Total secretions volume | 2333 | mL | Sherwood |
| | | | | 1997 |
| V_{total SI emptying} | Total intestinal emptying | 312 | mL | Sherwood |
| | volume | | | 1997, |
| | | | | Sandle |
| | | | | 1998 |
| τ_{SI} | Intestinal transit time | 240 | min | Thomas |
| | | | | 2006 |
| V_{total colon emptying} | Total colonic emptying volume | 62 | mL | Sherwood |
| | | | | 1997, |
| | | | | Sandle |
| | | | | 1998 |
| τ_{colon} | Colonic transit time | 1897 | min | Southwell |
| | | | | 2009 |
| k_{SI fluid abs} | First-order rate constant for | 9.4 × 10⁻³ | min⁻¹ | Calibrated |
| | intestinal fluid absorption | | | |
| k_{colon fluid abs} | First-order rate constant for | 8.5 × 10⁻⁴ | min⁻¹ | Calibrated |
| | colonic fluid absorption | | | |
| k_{SI oxalate abs} | First-order rate constant for | Dietary intake- | min⁻¹ | Calibrated |
| | intestinal oxalate absorption | dependent | | |
| k_{colon oxalate abs} | First-order rate constant for | Dietary intake- | min⁻¹ | Calibrated |
| | colonic oxalate absorption | dependent | | |
| Vₘₐₓ | Maximal enzyme velocity | 1.6 | μmol/min/ | In vitro |
| | | | 1 × 10⁹ | simulation |
| | | | cells | |
| K_{M} | Michaelis constant | 0.017 | mM | In vitro |
| | | | | simulation |
| K_{gastric pH inhibition} | Gastric pH inhibition function | Time-dependent | unitless | In vitro |
| | | | | simulation |
| K_{gastric O}_{₂} _{inhibition} | Gastric oxygen inhibition | 0.82 | unitless | In vitro |
| | function | | | simulation |
| K_{SI pH inhibition} | Intestinal pH inhibition function | 0.54 | unitless | In vitro |
| | | | | simulation |
| K_{SI O}_{₂} inhibition | Intestinal pH inhibition function | 0.82 | unitless | In vitro |
| | | | | simulation |
| K_{colon pH inhibition} | Colonic pH inhibition function | 0.54 | unitless | In vitro |
| | | | | simulation |
| K_{colon O}_{₂} _{inhibition} | Colonic oxygen inhibition | 0.77 | unitless | In vitro |
| | function | | | simulation |
| K_{extended colonic activity} | Extended colonic activity term | Time-dependent | unitless | In vitro |
| | | | | simulation |
| f_{abs} | Fraction of dietary oxalate | Dietary intake- | unitless | Holmes |
| | absorbed | dependent | | 2001 |
| f_{SI abs healthy} | Fraction of oxalate absorption | 0.37 | unitless | Morris |
| | occurring in the small intestine | | | 1993 |
| N_{daily meals} | Number of meals per day | 3 | day⁻¹ | — |
| r_{endogenous} | Endogenous production rate | 14.5 | mg/day | Chadwick |
| | | | | 1973 |
| k_{urinary} | Urinary excretion rate constant | 0.63 | day⁻¹ | Holmes |
| | | | | 2001 |

The gastric chyme volume balance is given by Equation 1, where

$$\frac{{dV}_{gastric}}{dt}$$

defines the rate of change of the gastric chyme volume and r_{gastric emptying} defines the rate of chyme emptying from the stomach into the small intestine. The intestinal chyme volume balance is given by Equation 2, where

$$\frac{{dV}_{SI}}{dt}$$

defines the rate of change of the intestinal chyme volume, r_{secretions} defines the rate of fluid secretion from the plasma into the small intestine, r_{sI fluid abs} defines the rate of fluid absorption from the small intestine into plasma, and r_{SI emptying} defines the rate of chyme emptying from the small intestine into the colon. The colonic chyme volume balance is given by Equation 3, where

$$\frac{{dV}_{colon}}{dt}$$

defines the rate of change of the colonic chyme volume, r_{colon fluid abs} defines the rate of fluid absorption from the colon into plasma, and r_{colon emptying} defines the rate of chyme emptying from the colon into feces. All terms in all chyme balances are defined in units of mL/min.

$$1. \frac{{dV}_{gastric}}{dt} = {r}_{gastric emptying}$$

$$2. \frac{{dV}_{SI}}{dt} = {r}_{gastric emptying} + {r}_{seceretions} - {r}_{SI fluid abs} - {r}_{SI emptying}$$

$$3. \frac{{dV}_{colon}}{dt} = {r}_{SI emptying} - {r}_{colon fluid abs} - {r}_{colon emptying}$$

The gastric oxalate balance is given by Equation 4, where

$$\frac{{dOx}_{gastric}}{dt}$$

defines the rate of change of the gastric oxalate, r_{gastric oxalate emptying} defines the rate of oxalate emptying from the stomach into the small intestine, and r_{gastric oxalate cons} defines the rate of oxalate consumption by SYNB8802 in the stomach. The intestinal oxalate balance is given by Equation 5, where

$$\frac{{dOx}_{SI}}{dt}$$

defines the rate of change of the intestinal oxalate, r_{SI oxalate cons} defines the rate of oxalate consumption by SYNB8802 in the small intestine, r_{SI oxalate abs} defines the rate of oxalate absorption from the small intestine into plasma, and r_{SI oxalate emptying} defines the rate of oxalate emptying from the small intestine into the colon. The colonic oxalate balance is given by Equation 6, where

$$\frac{{dOx}_{colon}}{dt}$$

defines the rate of change of the colonic oxalate, r_{colon oxalate cons} defines the rate of oxalate consumption by SYNB8802 in the colon, r_{colon oxalate abs} defines the rate of oxalate absorption from the colon into plasma, and r_{colon oxalate emptying} defines the rate of oxalate emptying from the colon into feces. All terms in all oxalate balances are defined in units of mmol/min.

$$4. \frac{{dOx}_{gastric}}{dt} = - {r}_{gastric oxalate emptying} - {r}_{gastric oxalate cons}$$

$$5. \frac{{dOx}_{SI}}{dt} = {r}_{gastric oxalate emptying} - {r}_{SI oxalate cons} - {r}_{SI oxalate abs} - {r}_{SI oxalate emptying}$$

$$6. \frac{{dOx}_{colon}}{dt} = {r}_{SI oxalate emptying} - {r}_{colon oxalate cons} - {r}_{colon oxalate abs} - {r}_{colon oxalate emptying}$$

The gastric SYNB8802 balance is given by Equation 7, where

$$\frac{{dCFU}_{gastric}}{dt}$$

defines the rate of change of the gastric SYNB8802 population and r_{gastric CFU emptying} defines the rate of SYNB8802 emptying from the stomach into the small intestine. The intestinal SYNB8802 balance is given by Equation 8, where

$$\frac{{dCFU}_{SI}}{dt}$$

defines the rate of change of the intestinal SYNB8802 population and r_{SI CFU emptying} defines the rate of SYNB8802 emptying from the small intestine into the colon. The colonic oxalate balance is given by Equation 9, where

$$\frac{{dCFU}_{colon}}{dt}$$

defines the rate of change of the colonic SYNB8802 population and r_{colon CFU emptying} defines the rate of SYNB8802 emptying from the colon into feces. All terms in all SYNB8802 balances are defined in units of cells/min.

$$7. \frac{{dCFU}_{gastric}}{dt} = - {r}_{gastric CFU emptying}$$

$$8. \frac{{dCFU}_{SI}}{dt} = {r}_{gastric CFU emptying} - {r}_{SI CFU emptying}$$

$$9. \frac{{dCFCU}_{colon}}{dt} = {r}_{SI CFU emptying} - {r}_{colon CFU emptying}$$

Chyme transit from the stomach to small intestine was modeled according to a power exponential decay function for stomach volume based on the work of Elashoff et al. (Table 1; Analysis of Gastric Emptying Data. Elashoff, Janet D. 1982, Gastroenterology). The rate of gastric emptying was given by

$$- {V}_{total gastric emptying} * \frac{β * ln 2 * {t}^{β - 1}}{{τ}_{1 / 2}^{β}} * {2}^{- {( \frac{t}{{τ}_{1 / 2}} )}^{β}} ,$$

equal to the product of the total gastric emptying volume V_{total gastric emptying} and the time derivative of the power exponential defined by the half gastric emptying time τ_{1/2} and the shape parameter β. The gastric emptying function was then modified to terminate in a finite amount of time given by τ_{gastric}; to achieve this, gastric emptying was switched from power exponential to linear at time τₗᵢₙₑₐᵣ, then defined as zero from τ_{gastric} onward (Equation 10). The rate of fluid secretion from the plasma into the small intestine was modeled as proportional to the gastric chyme emptying rate and the ratio of the total secretions volume V_{total secretions} to the total gastric emptying volume (Equation 11). The first portion of chyme to exit the stomach was assumed to also be the first to reach the ileocecal valve and empty from the small intestine into the colon; therefore, intestinal emptying begins at the intestinal transit time τ_{SI}. Likewise, the last portion of chyme to exit the stomach was last to empty from the small intestine and marked the end of the intestinal emptying window at time τ_{SI}+τ_{gastric}. The timeframe of colonic emptying was similarly defined as τ_{SI}+τ_{colon}≤t<τ_{SI}+τ_{colon}+τ_{gastric}, where τ_{colon} defines the colonic transit time. Intestinal and colonic chyme emptying were assumed to be constant during the relevant timeframes and zero otherwise, with the magnitude of the emptying rate equal to the total intestinal or colonic emptying volume V_{total SI emptying}, V_{total colon emptying} divided by the length of the timeframe (Equations 12-13).

$$10. {r}_{gastric emptying} = \{ \begin{matrix}- {V}_{total gastric emptying} * \frac{β * ln 2 * {t}^{β - 1}}{{τ}_{1 / 2}^{β}} * {2}^{- {( \frac{t}{{τ}_{1 / 2}} )}^{β}} & if t < {τ}_{linear} \\ \frac{{V}_{gastric} ( t = {τ}_{linear} )}{{τ}_{gastric} - {τ}_{linear}} & if {τ}_{linear} \leq t < {τ}_{gastric} \\ 0 & if t \geq {τ}_{gastric}\end{matrix}$$

$$11. {r}_{secretions} = {r}_{gastric emptying} * \frac{{V}_{total secretions}}{{V}_{total gastric emptying}}$$

$$12. {r}_{SI emptying} = \{ \begin{matrix}\frac{{V}_{total SI emptying}}{{τ}_{gastric}} & if {τ}_{SI} \leq t < {τ}_{SI} + {τ}_{gastric} \\ 0 & otherwise\end{matrix}$$

$$13. {r}_{colon emptying} = \{ \begin{matrix}\frac{{V}_{total SI emptying}}{{τ}_{gastric}} & if {τ}_{SI} + {τ}_{colon} \leq t < {τ}_{SI} + {τ}_{colon} + {τ}_{gastric} \\ 0 & otherwise\end{matrix}$$

Oxalate emptying from the stomach, small intestine, and colon were defined as the product of the chyme emptying rate in each compartment and the oxalate concentration, equal to the oxalate abundance in mmol divided by the chyme volume in mL (Equations 14-16). SYNB8802 emptying was also defined as proportional to chyme transit and SYNB8802 concentration, equal to the SYNB8802 abundance in cells/min divided by the chyme volume in mL (Equations 17-19). The rates of fluid absorption from the small intestine and colon into plasma were modeled as first-order, as the product of the chyme volume and a first-order kinetic rate constant for fluid absorption k_{SI/colon fluid abs} (Equations 20-21). The rates of oxalate absorption from the small intestine and colon into plasma were also modeled as first-order, as the product of the oxalate abundance and a first-order kinetic rate constant for oxalate absorption k_{SI/colon oxalate abs} (Equations 22-23).

$$14. {r}_{gastric oxalate emptying} = {r}_{gastric emptying} * \frac{{Ox}_{gastric}}{{V}_{gastric}}$$

$$15. {r}_{SI oxalate emptying} = {r}_{SI emptying} * \frac{{Ox}_{SI}}{{V}_{SI}}$$

$$16. {r}_{colon oxalate emptying} = {r}_{colon emptying} * \frac{{OX}_{colon}}{{V}_{colon}}$$

$$17. {r}_{gastric CFU emptying} = {r}_{gastric emptying} * \frac{{CFU}_{gastric}}{{V}_{gastric}}$$

$$18. {r}_{SI CFU emptying} = {r}_{SI emptying} * \frac{{CFU}_{SI}}{{V}_{SI}}$$

$$19. {r}_{colon CFU emptying} = {r}_{colon emptying} * \frac{{CFU}_{colon}}{{V}_{colon}}$$

$$20. {r}_{SI fluid abs} = {k}_{SI fluid abs} * {V}_{SI}$$

$$21. {r}_{colon fluid abs} = {k}_{colon fluid abs} * {V}_{colon}$$

$$22. {r}_{SL oxalate abs} = {k}_{SI oxalate abs} * {Ox}_{SI}$$

$$23. {r}_{colon oxalate abs} = {k}_{colon oxalate abs} * {Ox}_{colon}$$

Oxalate consumption by SYNB8802 in each gut compartment was simulated according to the Michaelis-Menten model of enzyme kinetics (FIG. 18A). This model defines the rate of consumption as a maximal enzyme velocity Vₘₐₓ times the substrate concentration divided by the sum of a Michaelis constant K_{M} and the substrate concentration; this quantity was then multiplied by the SYNB8802 abundance in each compartment. SYNB8802 activity in the stomach was further modified by a gastric pH inhibition function K_{gastric pH inhibition} and a gastric oxygen inhibition function K_{gastric O}_{₂} inhibition (Equation 24). SYNB8802 activity in the small intestine was modified by an intestinal pH inhibition function K_{SI pH inhibition} and an intestinal oxygen inhibition function K_{SI O}_{₂} _{inhibition} (Equation 25). SYNB8802 activity in the colon was modified by a colonic pH inhibition function K_{colon pH inhibition}, a colonic oxygen inhibition function K_{colon O}_{₂} _{inhibition}, and an extended colonic activity term K_{extended colonic activity} (Equation 26). A physiological function of gastric pH decline following a meal was modeled as a power exponential decay function (FIG. 18B, dark blue). A half-time parameter described the time for half of the total pH decline to occur, and a shape parameter described the degree of variance from a simple exponential model. SYNB8802 cells were modeled to follow a gastric residence time distribution truncated to a maximum of 4 hours, with a median gastric residence time of 110 minutes (FIG. 18B, light blue). The pH inhibition functions were informed by the SGF experiment (FIG. 18C). An exponential decay function was fit to each pH trial and decay constants were interpolated to simulate non-integer pH values. The relationship between pH and activity decay was then applied to the function of gastric pH dynamics to yield the gastric pH inhibition function (FIG. 18D). The model of gastric emptying dynamics was then used to construct a distribution of gastric residence times which, in combination with the gastric pH inhibition function, was used to simulate lowering of intestinal and colonic SYNB8802 activity due to lasting acid damage, yielding the intestinal and colonic pH inhibition functions (FIG. 4D). Cells that spent longer in the stomach were considered less active while in the small intestine and colon. A cap of 75% of maximal activity was imposed on the simulated activity of all cells while in the small intestine and colon, regardless of the time spent in the stomach. This was due to the intestinal/colonic pH of 6.5 and was informed by a function describing instantaneous rather than lasting pH effects, fit to in-house in vitro simulations. Normalized SYNB8802 activities of 4±0.2%, 4±0.3%, 24±0.7%, 28±0.6%, 30±0.2%, 47±2%, 52±2%, 71±11%, and 100±3% were observed at a pH of 3.0, 3.5, 4.0, 4.5, 5.0, 5.5, 6.0, 6.5, and 7.0, respectively. The oxygen inhibition functions in all gut compartments were modeled according to linear decline from the maximal strain activity observed at 21% oxygen, fit to in-house in vitro simulations. Normalized SYNB8802 activities of 74±5%, 79±29%, and 100±1% were observed at 0%, 7%, and 21% oxygen, respectively. The extended colonic activity term was informed by the SCF in vitro simulation via direct interpolation of consumption rate over time (FIG. 17).

$$24. {r}_{gastric oxalate cons} = \frac{{V}_{max} * [ {Ox}_{gastric} ]}{{K}_{M} + [ {Ox}_{gastric} ]} * {CFU}_{gastric} * {K}_{gastric pH inhibition} * {K}_{gastric {O}_{2} inhibition}$$

$$25. {r}_{SI oxalate cons} = \frac{{V}_{max} * [ {Ox}_{SI} ]}{{K}_{M} + [ {Ox}_{SI} ]} * {CFU}_{SI} * {K}_{SI pH inhibition} * {K}_{SI {O}_{2} inhibition}$$

$$26. {r}_{colon oxalate cons} = \frac{{V}_{max} * [ {Ox}_{colon} ]}{{K}_{M} + [ {Ox}_{colon} ]} * {CFU}_{colon} * {K}_{colon pH inhibition} * {K}_{colon {O}_{2} inhibition} * {K}_{extended colonic activity}$$

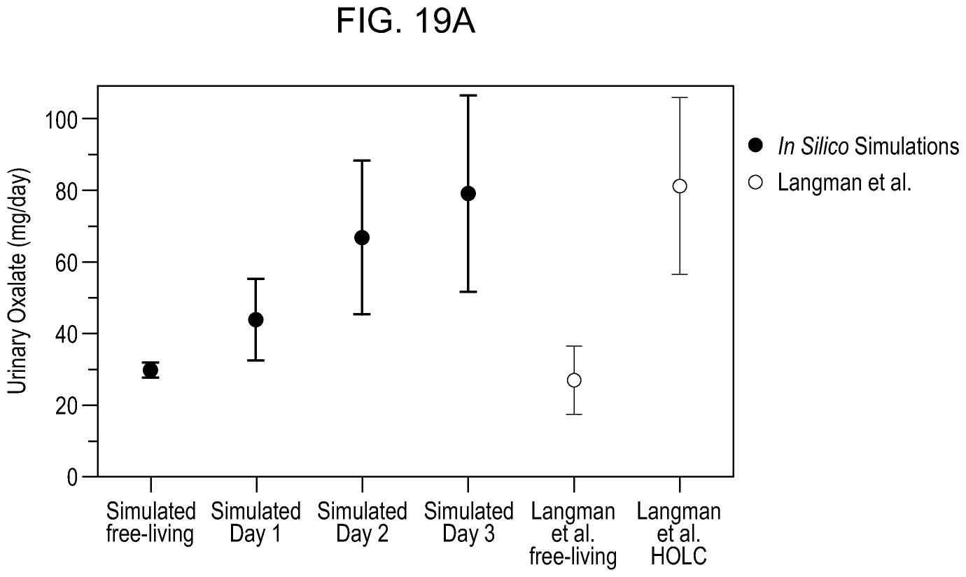

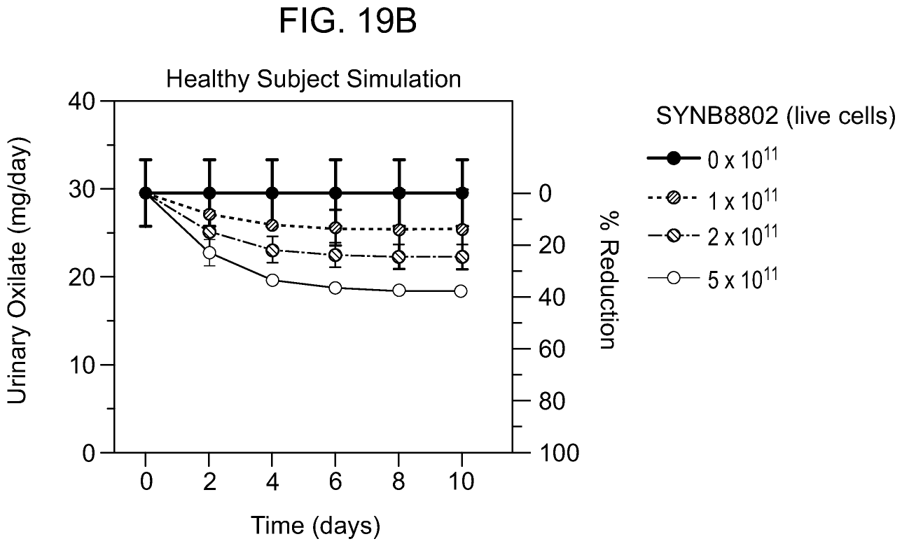

The total gut absorption into plasma was equal to the sum of intestinal and colonic absorption; gastric absorption was not modeled (Equation 27). The first-order oxalate absorption rate constants in Equations 22 and 23 were calibrated such that the total absorption in absence of SYNB8802 M_{oxalate abs}(SYNB8802 absent) was equal to the dietary intake M_{dietary oxalate} times a dietary absorption fraction f_{abs} (Equation 28), and that the intestinal portion thereof M_{SI oxalate abs}(SYNB8802 absent) was equal to the total times an intestinal fraction f_{SI abs} describing the site of absorption (Equation 29). The dietary absorption fraction for healthy subjects was based on the work of Holmes et al., who observed the relationship between dietary oxalate intake and urinary excretion (FIGS. 19A and 19B; Contribution of dietary oxalate to urinary oxalate excretion. Holmes, R. P., Goodman, H. O., & Assimos, D. G. 2001, Kidney international, pp. 270-276.). Twelve healthy individuals were placed on an oxalate-free diet for five days to establish the urinary excretion under endogenous production alone, then switched to a higher-oxalate diet ranging from 10 to 250 mg/day. The fraction of dietary oxalate absorbed was calculated as the difference between urinary excretion on the oxalate-containing and oxalate-free diets divided by the oxalate content of the diet. The ISS approach presented here fit an exponential function to the observed data to determine dietary absorption fraction for healthy subjects as a function of dietary intake. The dietary absorption fraction for EH patients was assumed to be from 3 to 5 times greater than that in healthy subjects (Equation 30) (Contribution of dietary oxalate to urinary oxalate excretion. Holmes, R. P., Goodman, H. O., & Assimos, D. G. 2001, Kidney international, pp. 270-276; Mechanism for Hyperoxaluria in Patients with Ileal Dysfunction. Chadwick, et al. 1973, N Engl J Med.; Hyperoxaluria in Patients with Ileal Resection: An Abnormality in Dietary Oxalate Absorption. Earnest, et al. 1974, Gastroenterology; Evidence for excessive absorption of oxalate by the colon in enteric hyperoxaluria. R. Modigliani, D. Labayle, C. Aymes, R. Denvil. 1978, Scand J Gastroent, pp. 187-192). The site of absorption for healthy subjects was informed by a study of oxalate transport across sections of the mouse gut, which yielded permeability constants for the duodenum, jejunum, ileum, proximal colon, and distal colon. (Physiological parameters in laboratory animals and humans. B Davies, T Morris. 1993, Pharmaceutical Research.) The ratio of the intestinal to colonic permeability constants was assumed to be equal between mice and humans. The site of absorption for EH patients was assumed such that all additional oxalate absorption occurred in the colon; that is, such that intestinal absorption was equal to that in healthy subjects (Equation 31).

M_{oxalate abs}=M_{SI oxalate abs}+M_{colon oxalate abs} 27.

M_{oxalate abs}(SYNB8802 absent)=M_{dietary oxalate}*f_{abs} 28.

M_{SI oxalate abs}(SYNB8802 absent)=M_{oxalate abs}(SYNB8802 absent)*f_{SI abs} 29.

3*f_{abs healthy}≤f_{abs EH}≤5*f_{abs healthy} 30.

M_{SI oxalate abs EH}(SYNB8802 absent)=M_{SI oxalate abs healthy}(SYNB8802 absent) 31.

The dynamics of oxalate abundance in the plasma were described using a material balance implemented as an ODE, where

$$\frac{{dOx}_{plasma}}{dt}$$

defines the rate of change of the plasma oxalate abundance, r_{plasma influx} defines the rate of oxalate influx into plasma and r_{urinary} defines the rate of urinary excretion of oxalate (Equation 32). Oxalate influx into plasma was defined as the total gut absorption per meal, as calculated in Equation 27, times the number of meals per day N_{daily meals} plus the rate of endogenous production of oxalate r_{endogenous} (Equation 33). The rate of endogenous production of oxalate was calculated based on the work of Chadwick et al., who observed the urinary excretion of EH patients while on several days of an oxalate-free diet (FIGS. 18A-18D; Mechanism for Hyperoxaluria in Patients with Ileal Dysfunction. Chadwick, et al. 1973, N Engl J Med.). Urinary excretion was modeled using first-order kinetics, as the product of the plasma oxalate abundance and a first-order kinetic rate constant for urinary excretion (Equation 34). The urinary excretion rate constant was based on the work of Holmes et al., who observed the urinary oxalate excretion dynamics for healthy subjects transitioning from self-selected diets to an oxalate-free diet (FIG. 17; 3. Contribution of dietary oxalate to urinary oxalate excretion. Holmes, R. P., Goodman, H. O., & Assimos, D. G. 2001, Kidney international, pp. 270-276.). The plasma oxalate ODE simplified into an exponential decay form describing how the plasma level Oxₚₗₐₛₘₐ(t) changed from an initial steady state Ox_{plasma init} to a new steady state Ox_{plasma SS} (Equation 35). The new steady state was defined as the plasma influx divided by the urinary excretion rate constant (Equation 36). The initial steady state was similarly defined as a previous plasma influx r_{plasma influx init} (e.g., before dosing with SYNB8802, under a different dietary intake, or both) divided by the urinary excretion rate constant (Equation 37). By combining Equations 34 and 35, it can be shown that urinary excretion followed the same dynamics as plasma level, changing from an initial steady state r_{plasma influx init} to a new steady state r_{plasma influx} (Equation 38).

$$32. \frac{{dOx}_{plasma}}{dt} = {r}_{plasma influx} - {r}_{urinary}$$

$$33. {r}_{plasma influx} = {M}_{oxalate abs} * {N}_{daily meals} + {r}_{endogenous}$$

$$34. {r}_{urinary} = {k}_{urinary} * {Ox}_{plasma}$$

$$35. {Ox}_{plasma} ( t ) = {Ox}_{plasma SS} + ( {Ox}_{plasma init} - {Ox}_{plasma SS} ) * {e}^{- {k}_{urinary} * t}$$

$$36. {Ox}_{plasma SS} = \frac{{r}_{plasma influx}}{{k}_{urinary}}$$

$$37. {Ox}_{plasma init} = \frac{{r}_{plasma influx init}}{{k}_{urinary}}$$

$$38. {r}_{urinary} ( t ) = {r}_{plasma influx} + ( {r}_{plasma influx init} - {r}_{plasma influx} ) * {e}^{- {k}_{urinary} * t}$$

### Software

In silico simulations were all implemented in Python 3.7.6, using Jupyter version 6.0.3 (jupyter.org). Ordinary differential equations were solved using SciPy version 1.4.1 (scipy.org). Statistical analysis was performed using Prism 9.1.0 (GraphPad, San Diego, CA).

### IVS and In Vivo Studies

Cells were thawed from a frozen (<−65° C.) cell bank and grown overnight in fermentation media, which was prepared as followed: Yeast extract (40 g/L), K2HPO4 (5 g/L), KH2PO4 (3.5 g/L), (NH4)2HPO4 (3.5 g/L), MgSO4*7H2O (0.5 g/L), FeCl3 (1.6 mg/L), CoCl2*6H2O (0.2 mg/mL), CuCl2 (0.1 mg/L), ZnCl2 (0.2 mg/L), NaMoO4 (0.2 mg/L), H3BO3 (0.05 mg/L), Antifoam 204 (125 μL/L), Galactose (30 g/L), Thymidine (20 mM). Cells were grown at 37° C. with shaking at 350 rpm. The next day cultures were back diluted to a starting OD of 0.18 and grown in modified fermentation media (Yeast extract (40 g/L), K2HPO4 (5 g/L), KH2PO4 (3.5 g/L), (NH4)2HPO4 (3.5 g/L), MgSO4*7H2O (0.5 g/L), FeCl3 (1.6 mg/L), CoCl2*6H2O (0.2 mg/mL), CuCl2 (0.1 mg/L), ZnCl2 (0.2 mg/L), NaMoO4 (0.2 mg/L), H3BO3 (0.05 mg/L), Antifoam 204 (125 μL/L), Galactose (30 g/L), Thymidine (20 mM), Sodium Formate (0.35 g/L), Sodium Furmarate (6 g/L)) and induced for activity in a fully controlled fermenter system to high cell density followed by washing, concentration, reformulation, and lyophilization.

### Example 8. In Vitro Activity Profile of ΔPks Strains Comprising a Gene Expression System for the Degradation of Oxalate

A Δpks EcN strain (deleted clbA-clbR gene and promoter sequences, intact clbS gene sequence with the operably linked promoter deleted) is engineered to further comprise an expression systems for the degradation of oxalate, as described in PCT/US2016/049781 filed Aug. 31, 2016, the contents of which is herein incorporated by reference in its entirety, to assess the effect of Δpks on the ability of the strains to consume oxalate.

Strains are grown in shake flasks and subsequently activated in an anaerobic chamber followed by concentration and freezing at ≤−65° C. in glycerol-based formulation buffer (PBS+25% Glycerol). In assay media containing 10 mM oxalate, activated cells are resuspended to OD600=5 and incubated statically at 37° C. Supernatant samples are removed at 30 and 60 min to determine the concentrations of oxalate. Concentrations are determined by liquid chromatography-tandem mass spectrometry (LC-MS/MS).

Oxalate is quantitated in bacterial supernatant by LC-MS/MS using a Thermo Vanquish UHPLC-Altis TSQ MS system. Standards are prepared at 0.8 to 1000 μg/mL in water. Samples and standards are diluted ten-fold with 10 mM ammonium acetate that includes 1 μg/mL 13C2-oxalate as an internal standard. Ten microliters are injected onto a Waters Acquity HSS T3 1.8 um 100 A 2.1×100 mm column using 10 mM ammonium acetate (A) and methanol (B) at 0.4 mL/min and 50° C. Analytes are separated after an initial 100% A hold for 0.5 minutes using a gradient from 0 to 95% B over 1.5 minutes followed by wash and equilibration steps. Compounds are detected by tandem mass spectroscopy with selected reaction monitoring in electrospray negative ion mode using the following ion pairs: Oxalate 89/61, ¹³C2-oxalate 91/62. Chromatograms are integrated and oxalate/¹³C2-oxalate (analyte/internal standard) peak area ratios are used to calculate unknown concentrations.

### Example 9. Pharmacodynamics of a Δpks Strain Capable of Catabolizing Oxalate Following Administration to Non-Human Primates

The in vivo activity of a Δpks EcN strain (SYNB8802v1), engineered to further comprise an expression system for the degradation of oxalate, was compared to a strain comprising the expression system for the degradation of oxalate but not the Δpks deletion (SYNB8802).

In vivo activity single dose cross-over studies were performed to assess the ability of the strains to metabolize gastrointestinal and diet-derived oxalate and 13 C2-oxalate in a nonhuman primate model of acute hyperoxaluria. Urinary recovery of oxalate and 13 C2-oxalate was significantly decreased regardless of the presence of the pks deletion, as compared to vehicle control, indicating that both strains are capable of consuming oxalate in nonhuman primates with acute hyperoxaluria. Results are shown in Table 40 and Table 41.

TABLE 40
Percent change in Urine Oxalate as compared to vehicle control

| | | % change as |
|---|---|---|
| Strain | Timepoint | compared to vehicle |
| SYNB8802 | 6 h | 73% |
| SYNB8802v1 (SYNB8802, | 8 h | 65% |
| further having Δpks) | | |

TABLE 41
Percent change in Urine 13 C2-oxalate
as compared to vehicle control

| | | % change as |
|---|---|---|
| Strain | Timepoint | compared to vehicle |
| SYNB8802 | 6 h | 75% |
| SYNB8802v1 (SYNB8802 | 8 h | 82% |
| further having Δpks) | | |

### Example 10. A Double-blind, Randomized, Placebo-controlled Study to Assess the Safety, Tolerability, and Pharmacodynamics of SYNB8802 in Subjects with History of Gastric Bypass Surgery or Short-Bowel Syndrome

This is a first-in-human study of SYNB8802,002 is designed to assess safety, tolerability, and oxalate lowering, and in subjects with a history of gastric bypass surgery or short-bowel syndrome. In addition, this study will explore other pharmacodynamic (PD) effects relative to baseline in healthy subjects as well as predictors of efficacy and in subjects with EH tolerability.

The duration of the study was selected based on data from the current ongoing study demonstrating steady-state oxalate lowering in healthy subjects, and the time course of oxalate lowering is anticipated to be similar in subjects with a history of gastric bypass surgery or short-bowel syndrome.

### Study Objectives

Primary objectives include to evaluate the safety and tolerability of SYNB8802. Secondary objectives include to assess the effect of SYNB8802 on urinary oxalate (UOx) excretion after an average-oxalate low-calcium (AOLC) diet. Exploratory objectives include (i) To assess the effect of SYNB8802 on biomarkers associated with increased risk of kidney stones. (ii) To assess the effect of SYNB8802 on fecal oxalate levels. (iii) To assess the effect of SYNB8802 on plasma oxalate (POx) levels. (iv) To assess potential factors that predict oxalate responses. (v) To explore potential biomarkers of tolerability.

### Methodology

This is a double-blind, randomized (3:2), placebo-controlled, inpatient study evaluating the safety and tolerability of SYNB8802 in patients with a history of gastric bypass surgery or short-bowel syndrome. The study includes the following periods: (1) Screening (27 days); (2) Diet run in (3 days); (3) Dosing Period (12 days); (4) Safety follow up (28 days). The maximum duration of the inpatient stay will be 17 days (Day −4 to Day 13).

Subjects will report to the clinical research unit (CRU) on Day −4 and will complete a 3-day diet run-in (Days −3 to −1) during which they will consume an AOLC diet. Dietary oxalate and calcium will be distributed across 3 meals per day and subjects will maintain this diet until the end of the dosing period. A proton pump inhibitor (PPI, esomeprazole) will be administered once daily (QD), 60-90 minutes before breakfast, from the start of the diet run-in until the end of the dosing period. if a subject is already regularly taking a different PPI, that agent may be continued, or a different PPI may be given if needed due to allergy or drug interaction.

On Day 1, subjects will be randomly assigned to treatment with SYNB8802 or placebo (collectively referred to as investigational medicinal product [IMP]). The dosing period consists of 12 days following a dose escalation plan from 1×10\hat{ }11 cells QD to 3×10\hat{ }11 cells 3 times daily (TID); the dosing period for each dose level includes a 2-day dose ramp and a 3-day steady-state period. IMP will be administered orally with meals according to the dosing schedule. If a subject does not tolerate BID or TID dosing, the dosing frequency may be reduced.

On the morning of the first day of the diet run-in (Day −3), a forced void urine sample will be collected to completely empty the bladder. A 24-hour urine collection will then be started and will continue throughout the in-patient period. In addition, daily 24-hour fecal samples will be collected.

Subjects will be released from the CRU upon the completion of safety assessments on Day 13 (the day after the last dose of IMP). Safety follow-up visits (calls) will occur every 7 (±2) days until 28 days after the last dose of IMP.

Subjects will consume an AOLC diet (300 mg oxalate and 400 mg calcium daily, refer to Diet Manual for details) throughout the inpatient stay with fixed calories and fluid volume adjusted for stable body weight. They will consume all meals provided to them, and all dietary intake will be recorded from the start of each diet run-in until the end of IMP dosing. Approved snacks to meet caloric balance requirements will be allowed and also required.

Systemic (oral or intravenous) antibiotics are not allowed for the duration of the study (topical antibiotics are allowed).

### Study Inclusion and Exclusion Criteria

### Inclusion Criteria

(1) Age ≥18 to ≤74 years.

(2) Able and willing to voluntarily complete the informed consent process.

(3) Available for, and agree to, all study procedures, including fixed diet, feces, urine, and blood collection, follow-up visits, and compliance with all study procedures.

(4) History of gastric bypass surgery (at least 12 months prior to Day 1) or short-bowel syndrome.

(5) If taking probiotic supplements (enriched foods excluded), has been on a stable, well-tolerated dose for at least 2 weeks prior to Day 1.

(6) Women of childbearing potential must have a negative pregnancy test (human chorionic gonadotropin) at screening and at baseline prior to the start of IMP.

(7) Screening laboratory evaluations (e.g., chemistry panel, complete blood count with differential, prothrombin time, urinalysis) and electrocardiogram (ECG) must be within normal limits or judged not to be clinically significant by the investigator. In subjects with known diabetes, an abnormal glucose value is acceptable. A single repeat evaluation is acceptable.

(8) Agree to abstain from tobacco/nicotine use for the duration of the inpatient stay.

### Exclusion Criteria

(1) Acute or chronic medical (including COVID-19 infection), surgical, psychiatric, or social condition or laboratory abnormality (except those that can be explained by malabsorption) that may increase subject risk associated with study participation, compromise adherence to study procedures and requirements, or may confound interpretation of results and, in the judgment of the investigator, would make the subject inappropriate for enrollment.

(2) Estimated glomerular filtration rate <45 mL/min/1.73 m2.

(3) History of kidney stones.

(4) Unable or unwilling to discontinue vitamin C supplementation for the study duration.

(5) Known primary hyperoxaluria.

(6) Pregnant or lactating.

(7) Administration or ingestion of any type of systemic (e.g., oral or intravenous) antibiotic within 5 half-lives of the agent-prior to Day 1. Exception: topical antibiotics are allowed.

(8) Any co-morbid condition that may necessitate antibiotic use or disrupt the controlled diet during the dosing period.

(9) Intolerance of, or allergic reaction to, Escherichia coli Nissle 1917 (EcN), all PPIs, or any of the ingredients in SYNB8802 or placebo formulations.

(10) Dependence on alcohol or drugs of abuse.

(11) Current immunodeficiency disorder including autoimmune disorders and uncontrolled human immunodeficiency virus (HIV). Subjects who are HIV positive on therapy with normal CD4 counts can be included.

(12) Administration or ingestion of an investigational drug within 30 days or 5 half-lives of the agent, whichever is longer, prior to screening visit, or current enrollment in an investigational study.

(13) History of inflammatory bowel disease.

### Investigational Medicinal Product(s):

SYNB8802 at 1×10\hat{ }11 or 3×10\hat{ }11 live cells administered orally with meals according to the dosing schedule.

Placebo to match SYNB8802 administered orally with meals according to the dosing schedule.

### Duration of Treatment

The maximum time of study participation for a subject is planned to be up to 70 days:

Screening period: up to 27 days.

Diet run-in period: 3 days.

Dosing period: 12 days. Discharge from the CRU will occur on the day following last IMP administration.

Safety follow-up period: 28 days.

### Study Endpoints

Primary endpoint: Safety and tolerability of SYNB8802, as assessed by adverse events (AEs), clinical laboratory tests, and vital sign measurements.

Secondary endpoint: Change from baseline in 24-hour UOx amount excreted among SYNB8802-treated subjects versus those treated with placebo.

Exploratory Endpoints:

(1) Change from baseline in biomarkers associated with increased risk of kidney stones, such as urine supersaturation scores, among SYNB8802-treated subjects versus those treated with placebo.

(2) Change from baseline in fecal oxalate levels among SYNB8802-treated subjects versus those treated with placebo.

(3) Change from baseline in POx levels among SYNB8802-treated subjects versus those treated with placebo.

(4) Correlation of change from baseline in UOx with other baseline and study factors, such as presence of kidney stones on screening, degree of malabsorption, tolerability profile, and other patient factors.

(5) Correlation of tolerability data with exploratory biomarkers, such as ghrelin, CRP, and IL-6.

(6) Correlation of efficacy with exploratory biomarkers, such as the fecal microbiome.

### Removal From IMP Treatment and Withdrawal of Consent

Reasons for discontinuation of IMP may include the following:

(1) The subject withdraws consent.

(2) The investigator or sponsor notes a significant noncompliance with protocol procedures.

(3) The subject develops an intolerable toxicity including but not limited to Grade ≥3 AE or serious adverse event (SAE) assessed as related to the IMP by the investigator.

(4) The subject requires treatment with systemic antibiotics.

(5) The investigator determines that the subject must discontinue further study dosing for medical reasons.

(6) Subjects may withdraw their consent at any time for any reason without prejudice to their future medical care by their physician(s) or at their medical institution(s). Subject data collected up to the date of consent withdrawal will be included in the analyses.

### Pharmacodynamics Analysis

Urine, blood, and fecal samples will be collected at screening, and then daily throughout the diet run in and dosing period. The following assessments will be performed to evaluate the preliminary PD of SYNB8802:

24-Hour UOx.

Urinary supersaturation indices including volume and creatinine.

Plasma and 24-hour fecal oxalate.

Exploratory biomarkers of efficacy, tolerability, and pharmacodynamics.

TABLE 42
Schedule of Events

| | Pre-screening Period | | | | Dosing Period | | | | | |
|---|---|---|---|---|---|---|---|---|---|---|
| | Screening | Diet Run-in | | | Dose Level 1 | | Dose Level 2 | | Safety Follow-upPeriod | |
| | Study Days | | | | | | | | | |
| | | | | | Dose | Steady | Dose | Steady | | EOS (Every7 ± 2 days |
| | | | | | Ramp | State | Ramp | State | | until 28 daysfollowing |
| Study Procedure | −30 to −4 | −3 | −2 | −1 | 1-2 | 3-6 | 7-8 | 9-12 | 13 | last IMP dose)^{a} |
| Informed consent | ● | | | | | | | | | |
| Medical history | ● | | | | | | | | | |
| Fecal sample for O. formigenes qPCR | ● | | | | | | | | | |
| Abdominal radiograph to exclude | ● | | | | | | | | | |
| kidney stones^{b} | | | | | | | | | | |
| Height and weight | ● | ●^{c} | | | | | | | ● | |
| Screening urine drug screen | ● | | | | | | | | | |
| Screening for acute respiratory | ● | | | | | | | | | |
| infections (such as COVID-19) ^{d} | | | | | | | | | | |
| Vital signs ^{e} | ● | ● | | | ● | ● | ● | ● | ● | |
| (SBP/DBP, pulse, body temperature) | | | | | | | | | | |
| Physical examination | ● | ● | | | | | | | | |
| ECG | ● | | | | | | | | | |
| Serology/screening infectious disease | ● | | | | | | | | | |
| (HIV/hepatitis B, C) | | | | | | | | | | |
| Pregnancy test (WOCBP only) ^{f} | ● | | | | | | | | | |
| Record concomitant medications | ● | ● | ● | ● | ● | ● | ● | ● | ● | ● |
| Adverse event reporting ^{g} | | ● | ● | ● | ● | ● | ● | ● | ● | ● |
| Check in to CRU^{h} | ● | | | | | | | | | |
| Check out of CRU | | | | | | | | | ● | |
| Controlled diet and daily diet recording | | ● | ● | ● | ● | ● | ● | ● | | |
| Randomization | | | | | ●ⁱ | | | | | |
| Administer IMP (immediately after meals) | | | | | ● | ● | ● | ● | | |
| Administer PPI (once per day before | | ● | ● | ● | ● | ● | ● | ● | | |
| breakfast) | | | | | | | | | | |
| Laboratory tests (hematology/CBC | ●^{j} | ● | | | ● | | ●^{k} | | ● | |
| with differential, serum chemistry, | | | | | | | | | | |
| coagulation, FSH, urinalysis) | | | | | | | | | | |
| Fasting blood sample for plasma | | ● | | | ● | | ●^{k} | ●^{l} | | |
| oxalate | | | | | | | | | | |
| Forced-void urine sample for spot | | ● | | | | | | | | |
| oxalate:creatinine | | | | | | | | | | |
| 24-Hr urine collection for oxalate and | ●ⁿ | ●ⁿ | ● | ● | ● | ● | ● | ● | | |
| supersaturation panel | | | | | | | | | | |
| 24-Hr fecal collection for fecal | ●ⁿ | ●ⁿ | ● | ● | ● | ● | ● | ● | | |
| oxalate ^{m} | | | | | | | | | | |
| Fecal sample for exploratory | ●ⁿ | ●ⁿ | ● | ● | ● | ● | ● | ● | | |
| microbiome analysis ⁿ | | | | | | | | | | |
| Blood sample for exploratory | | | | ● | ● | ● | ● | ● | | |
| biomarkers of tolerability ^{o} | | | | | | | | | | |

Abbreviations: CBC = complete blood count; CRP = C-reactive protein; CRU = clinical research unit; DBP = diastolic blood pressure; ECG = electrocardiogram; FSH = follicle-stimulating hormone; FU = follow-up; HIV = human immunodeficiency virus; ICF = informed consent form; IMP = investigational medicinal product; POx = plasma oxalate; PPI = proton pump inhibitor; qPCR = quantitative polymerase chain reaction; SBP = systolic blood pressure; WOCBP = women of childbearing potential.
NOTE:
Subjects with documentation of negative stool samples for O. formigenes by qPCR within 90 days of signing the ICF do not need to repeatfecal samples to test for O. formigenes.
^{a}All subjects, whether they complete treatment or discontinue early, will be contacted to record concomitant medications and AEs. The safety follow-up visits may be conducted via telemedicine.
^{b}Abdominal radiograph performed within 6 months prior to screening to exclude presence of kidney stones is acceptable.
^{c}Weight only.
^{d} Conducted according to current local guidelines. May be repeated during the inpatient stay, as necessary.
^{e} Subjects are required to remain sitting for at least 5 minutes prior to obtaining vital signs.
^{f} For WOCBP only: Serum pregnancy test at screening; urine pregnancy test at check-in on Day −4
^{a}Adverse events will be assessed continuously by direct observation and subject event recording and interviews. “Continuous” is defined as solicitation of AEs after each dose administration of IMP and as may be reported by subjects at any point in time. All AEs occurring fromthe start of the PPI, through 28 days after last dose of IMP should be recorded.
^{b}Subject will check into the CRU on Day −4.
^{c}Randomization will occur on Day 1.
^{d} Coagulation tests and FSH (for post-menopausal women) measured only at screening.
^{e} Day 7 predose only.
^{f} Day 12 predose only.
^{g} Feces will be collected daily during the inpatient stay with the objective of being able to assess fecal pharmacodynamics and biomarkers. Asubset of the fecal collections may be analyzed rather than every sample.
^{h}Baseline samples to be collected following admission to the CRU.
ⁱA baseline blood sample for exploratory biomarkers should be collected 3 hours after breakfast on Day −1. During the dosing period, should asubject experience gastrointestinal symptoms (e.g., nausea, vomiting, diarrhea, abdominal cramping etc.) after IMP dosing on any day, a single blood sample should be collected while the subject is experiencing symptoms. Repeat samples on following days are not required unless the symptoms are different.

Vital Signs and Physical Examination: Resting vital signs (systolic blood pressure, diastolic blood pressure, pulse, and body temperature) will be collected as specified in Table 42. Subjects are required to remain in the sitting position for at least 5 minutes prior to obtaining vital signs.

A symptom-directed physical examination will be performed by trained medical personnel as specified in Table 42. Symptom-directed PEs may be performed at the investigator's discretion at nonscheduled times if warranted. Abnormal findings observed after the start of the PPI should be recorded as AEs.

### Clinical Laboratory Measurements

The clinical laboratory tests listed in Table 43 will be performed at the time points specified in Table 42. Screening results will be assessed by the investigator for inclusion of subjects in the study. Additionally, unscheduled clinical laboratory tests may be obtained at any time during the study at the investigator's discretion. The diagnosis corresponding to any clinically significant abnormality must be recorded as an AE.

TABLE 43
Clinical Laboratory Tests

| | | | |
|---|---|---|---|
| Hematology | Basophils % | Coagulation ^{a} | Prothrombin time |
| (CBC with | Basophils | Serum | Glucose |
| differential) | Eosinophils % | Chemistry | Urea nitrogen (BUN) |
| | Eosinophils | | Creatinine with eGFR |
| | Hematocrit | | Sodium |
| | Hemoglobin | | Potassium |
| | Lymphocytes % | | Chloride |
| | Lymphocytes | | Carbon dioxide |
| | Mean corpuscular hemoglobin | | Calcium |
| | Mean corpuscular volume | | Total protein |
| | Monocytes % | | Albumin |
| | Monocytes | | Fractionated bilirubin (total direct and |
| | Neutrophils % | | indirect) |
| | Neutrophils | | Alkaline phosphatase |
| | Platelet count | | Aspartate aminotransferase |
| | Red blood cells | | Alanine aminotransferase |
| | White blood cells | Pregnancy | Serum Follicle-stimulating hormone |
| Urinalysis | Specific gravity | Related | (for post-menopausal women only) ^{a} |
| | pH | Laboratory | Serum Pregnancy (for WOCBP only) ^{b} |
| | Glucose | Tests | Urine pregnancy (for WOCBP only) ^{b} |
| | Bilirubin | | |
| | Ketones | | |
| | Occult blood | | |
| | Protein | | |
| | Nitrite | | |
| | Leukocyte esterase | | |
| | Urine oxalate ^{c} | | |
| | Urine creatinine ^{c} | | |
| | Potassium ^{c} | | |
| | Calcium ^{c} | | |
| | Phosphorous ^{c} | | |
| | Uric acid ^{c} | | |
| | Citrate ^{c} | | |
| | Magnesium ^{c} | | |
| | Sodium ^{c} | | |
| | Chloride ^{c} | | |
| | Sulfate ^{c} | | |
| | Ammonium ^{c} | | |
| | Urea nitrogen ^{c} | | |

Abbreviations: BUN = blood urea nitrogen; CBC = complete blood count; CRP = C-reactive protein; eGFR = estimated glomerular filtration rate; HIV = human immunodeficiency virus; WOCBP = women ofchildbearing potential.
^{a} Performed at screening only.
^{b} Serum pregnancy at screening; urine pregnancy at all other timepoints.
^{c} Not considered clinical safety laboratory test.

Electrocardiograms: Supine single 12-lead ECGs will be performed as part of screening. ECG parameters to be evaluated include the RR, QT, QRS, and PR intervals. In addition, Fridericia's formula should be used to calculate the QT interval corrected for heart rate (QTcF). Subjects are required to remain in the supine position for at least 5 minutes prior to obtaining ECGs.

Exploratory Microbiome Samples: An aliquot of each 24-hour fecal sample will be sent for exploratory microbiome analysis will be obtained as detailed in Table 42. The analyses of these samples may be conditional based on the results of this or other clinical studies, and samples may be selected based on PD response. The results of these analyses may be reported separately from the main clinical study report.

Exploratory Tolerability Samples: A baseline blood sample for exploratory biomarkers should be collected 3 hours after breakfast on Day −1. During the dosing period, should a subject experience gastrointestinal symptoms (e.g., nausea, vomiting, diarrhea, abdominal cramping, etc.) after IMP dosing on any day, a single blood sample should be collected while the subject is experiencing symptoms. Repeat samples on following days are not required unless the symptoms change. These samples may be analyzed at a later date and results added to study report as an Appendix. If not analyzed within a year after initial study report, the samples will be discarded.

Safety Follow-up Assessments: All subjects, whether they complete treatment or discontinue early, will complete a safety follow-up visit every 7 (±2) days for 28 (±2) days after last dose of IMP, as detailed in Table 42. The safety follow-up visit may be conducted via telemedicine.

TABLE 44
Colibactin Nucleotide Sequences

| SEQ ID NO: | Description |
|---|---|
| SEQ ID NO: 1065 | clbA |
| SEQ ID NO: 1066 | clbB |
| SEQ ID NO: 1067 | clbC |
| SEQ ID NO: 1068 | clbD |
| SEQ ID NO: 1069 | clbE |
| SEQ ID NO: 1070 | clbF |
| SEQ ID NO: 1071 | clbG |
| SEQ ID NO: 1072 | clbH |
| SEQ ID NO: 1073 | clbI |
| SEQ ID NO: 1074 | clbJ |
| SEQ ID NO: 1075 | clbK |
| SEQ ID NO: 1076 | clbL |
| SEQ ID NO: 1077 | clbM |
| SEQ ID NO: 1078 | clbN |
| SEQ ID NO: 1079 | clbO |
| SEQ ID NO: 1080 | clbP |
| SEQ ID NO: 1081 | clbQ |
| SEQ ID NO: 1082 | clbR |
| SEQ ID NO: 1083 | clbS |

TABLE 45
Colibactin Amino Acid Sequences

| SEQ ID NO: | Description |
|---|---|
| SEQ ID NO: 1084 | clbA |
| SEQ ID NO: 1085 | clbB |
| SEQ ID NO: 1086 | clbC |
| SEQ ID NO: 1087 | clbD |
| SEQ ID NO: 1088 | clbE |
| SEQ ID NO: 1089 | clbF |
| SEQ ID NO: 1090 | clbG |
| SEQ ID NO: 1091 | clbH |
| SEQ ID NO: 1092 | clbI |
| SEQ ID NO: 1093 | clbJ |
| SEQ ID NO: 1094 | clbK |
| SEQ ID NO: 1095 | clbL |
| SEQ ID NO: 1096 | clbM |
| SEQ ID NO: 1097 | clbN |
| SEQ ID NO: 1098 | clbO |
| SEQ ID NO: 1099 | clbP |
| SEQ ID NO: 1100 | clbQ |
| SEQ ID NO: 1101 | clbR |
| SEQ ID NO: 1102 | clbS |

TABLE 46
Exemplary Sequences

| Description | |
|---|---|
| SEQ ID NO | Sequences |
| LacI in | TCACTGCCCGCTTTCCAGTCGGGAAACCTGTCGTGCCAGCTGCATTA |
| reverse | ATGAATCGGCCAACGCGCGGGGAGAGGCGGTTTGCGTATTGGGCGC |
| orientation | CAGGGTGGTTTTTCTTTTCACCAGTGAGACTGGCAACAGCTGATTGC |
| SEQ ID NO: | CCTTCACCGCCTGGCCCTGAGAGAGTTGCAGCAAGCGGTCCACGCTG |
| 1105 | GTTTGCCCCAGCAGGCGAAAATCCTGTTTGATGGTGGTTAACGGCGG |
| | GATATAACATGAGCTATCTTCGGTATCGTCGTATCCCACTACCGAGA |
| | TATCCGCACCAACGCGCAGCCCGGACTCGGTAATGGCGCGCATTGC |
| | GCCCAGCGCCATCTGATCGTTGGCAACCAGCATCGCAGTGGGAACG |
| | ATGCCCTCATTCAGCATTTGCATGGTTTGTTGAAAACCGGACATGGC |
| | ACTCCAGTCGCCTTCCCGTTCCGCTATCGGCTGAATTTGATTGCGAG |
| | TGAGATATTTATGCCAGCCAGCCAGACGCAGACGCGCCGAGACAGA |
| | ACTTAATGGGCCCGCTAACAGCGCGATTTGCTGGTGACCCAATGCGA |
| | CCAGATGCTCCACGCCCAGTCGCGTACCGTCCTCATGGGAGAAAAT |
| | AATACTGTTGATGGGTGTCTGGTCAGAGACATCAAGAAATAACGCC |
| | GGAACATTAGTGCAGGCAGCTTCCACAGCAATGGCATCCTGGTCATC |
| | CAGCGGATAGTTAATGATCAGCCCACTGACGCGTTGCGCGAGAAGA |
| | TTGTGCACCGCCGCTTTACAGGCTTCGACGCCGCTTCGTTCTACCATC |
| | GACACCACCACGCTGGCACCCAGTTGATCGGCGCGAGATTTAATCG |
| | CCGCGACAATTTGCGACGGCGCGTGCAGGGCCAGACTGGAGGTGGC |
| | AACGCCAATCAGCAACGACTGTTTGCCCGCCAGTTGTTGTGCCACGC |
| | GGTTGGGAATGTAATTCAGCTCCGCCATCGCCGCTTCCACTTTTTCCC |
| | GCGTTTTCGCAGAAACGTGGCTGGCCTGGTTCACCACGCGGGAAAC |
| | GGTCTGATAAGAGACACCGGCATACTCTGCGACATCGTATAACGTTA |
| | CTGGTTTCAT |
| LacI | MKPVTLYDVAEYAGVSYQTVSRVVNQASHVSAKTREKVEAAMAELN |
| SEQ ID NO: | YIPNRVAQQLAGKQSLLIGVATSSLALHAPSQIVAAIKSRADQLGASVV |
| 1106 | VSMVERSGVEACKAAVHNLLAQRVSGLIINYPLDDQDAIAVEAACTNV |
| | PALFLDVSDQTPINSIIFSHEDGTRLGVEHLVALGHQQIALLAGPLSSVSA |
| | RLRLAGWHKYLTRNQIQPIAEREGDWSAMSGFQQTMQMLNEGIVPTA |
| | MLVANDQMALGAMRAITESGLRVGADISVVGYDDTEDSSCYIPPLTTIK |
| | QDFRLLGQTSVDRLLQLSQGQAVKGNQLLPVSLVKRKTTLAPNTQTAS |
| | PRALADSLMQLARQVSRLESGQ |
| Lac operator | aattgtgagcgctcacaatt |
| SEQ ID NO: | |
| 1107 | |
| Promoter | ATTCACCACCCTGAATTGACTCTCTTCCGGGCGCTATCATGCCATAC |
| comprising Lac | CGCGAAAGGTTTTGCGCCATTCGATGGCGCGCCGCTTCGTCAGGCCA |
| operon | CATAGCTTTCTTGTTCTGATCGGAACGATCGTTGGCTGtgttgacaattaatcat |
| SEQ ID NO: | cggctcgtataatgtgtggaattgtgagcgctcacaattagctgtcaccggatgtgctttccggtctgatgagtccgt |
| 1108 | gaggacgaaacagcctctacaaataattttgtttaa |
| RBS | gaccagaggtaaggaggtaacaaccatgcgagtgttgaagaaacatcttaatcatgctggggagggtttcta |
| SEQ ID NO: | |
| 1109 | |
| Construct | TCACTGCCCGCTTTCCAGTCGGGAAACCTGTCGTGCCAGCTGCATTA |
| comprising LacI | ATGAATCGGCCAACGCGCGGGGAGAGGCGGTTTGCGTATTGGGCGC |
| in reverse | CAGGGTGGTTTTTCTTTTCACCAGTGAGACTGGCAACAGCTGATTGC |
| orientation, | CCTTCACCGCCTGGCCCTGAGAGAGTTGCAGCAAGCGGTCCACGCTG |
| IPTG inducible | GTTTGCCCCAGCAGGCGAAAATCCTGTTTGATGGTGGTTAACGGCGG |
| promoter, RBS, | GATATAACATGAGCTATCTTCGGTATCGTCGTATCCCACTACCGAGA |
| and gene | TATCCGCACCAACGCGCAGCCCGGACTCGGTAATGGCGCGCATTGC |
| sequences | GCCCAGCGCCATCTGATCGTTGGCAACCAGCATCGCAGTGGGAACG |
| encoding | ATGCCCTCATTCAGCATTTGCATGGTTTGTTGAAAACCGGACATGGC |
| oxalate | ACTCCAGTCGCCTTCCCGTTCCGCTATCGGCTGAATTTGATTGCGAG |
| catabolism | TGAGATATTTATGCCAGCCAGCCAGACGCAGACGCGCCGAGACAGA |
| enzymes, RBS | ACTTAATGGGCCCGCTAACAGCGCGATTTGCTGGTGACCCAATGCGA |
| sites are | CCAGATGCTCCACGCCCAGTCGCGTACCGTCCTCATGGGAGAAAAT |
| present between | AATACTGTTGATGGGTGTCTGGTCAGAGACATCAAGAAATAACGCC |
| ORF1 and ORF2 | GGAACATTAGTGCAGGCAGCTTCCACAGCAATGGCATCCTGGTCATC |
| and between | CAGCGGATAGTTAATGATCAGCCCACTGACGCGTTGCGCGAGAAGA |
| ORF2 and ORF3 | TTGTGCACCGCCGCTTTACAGGCTTCGACGCCGCTTCGTTCTACCATC |
| The three RBS | GACACCACCACGCTGGCACCCAGTTGATCGGCGCGAGATTTAATCG |
| sequences | CCGCGACAATTTGCGACGGCGCGTGCAGGGCCAGACTGGAGGTGGC |
| present in the | AACGCCAATCAGCAACGACTGTTTGCCCGCCAGTTGTTGTGCCACGC |
| constructs, | GGTTGGGAATGTAATTCAGCTCCGCCATCGCCGCTTCCACTTTTTCCC |
| located 5′ of | GCGTTTTCGCAGAAACGTGGCTGGCCTGGTTCACCACGCGGGAAAC |
| ORF1, ORF2, | GGTCTGATAAGAGACACCGGCATACTCTGCGACATCGTATAACGTTA |
| and ORF3, | CTGGTTTCATATTCACCACCCTGAATTGACTCTCTTCCGGGCGCTATC |
| respectively, | ATGCCATACCGCGAAAGGTTTTGCGCCATTCGATGGCGCGCCGCTTC |
| are underlined | GTCAGGCCACATAGCTTTCTTGTTCTGATCGGAACGATCGTTGGCTGt |
| SEQ ID NO: | gttgacaattaatcatcggctcgtataatgtgtggaattgtgagcgctcacaattagctgtcaccggatgtgctttccg |
| 1110 | gtctgatgagtccgtgaggacgaaacagcctctacaaataattttgtttaagaccagaggtaaggaggtaacaacc |
| | atgcgagtgttgaagaaacatcttaatcatgctggggagggtttctaatgaccagtgcagctacggtgaccgcgag |
| | ctttaatgacactttttctgtgagcgataatgtcgcggtaatcgtaccggaaaccgatacgcaggtcacctaccgtga |
| | tctttcccacatggtaggacactttcaaacaatgttcacgaacccgaatagtcctctgtacggggcggtctttcgtca |
| | agacacggtagcgattagcatgcgtaacggccttgaatttattgtggctttccttggagccacgatggatgcgaaaa |
| | ttggtgcgccactgaatcccaattataaagagaaggagtttaatttttacctgaatgacttaaagtccaaagccatctg |
| | cgtgccgaaaggcaccaccaaactgcaaagttcagaaattcttaagagtgcgtccacgttcgggtgctttattgtgg |
| | aactggcgtttgacgccacccgttttcgtgttgaatatgacatttactccccggaggacaattataaacgtgtgatcta |
| | ccgcagccttaacaatgctaagtttgtcaacacaaaccctgtcaagttcccgggtttcgcccgcagctcggatgttg |
| | cacttattttgcatacctcaggcaccactagtaccccaaagaccgtacccctcttgcatctgaatattgtccgttcaac |
| | cctgaatatcgccaacacttacaaacttaccccgctggatcgctcctatgttgtaatgccgctgtttcatgtacatgga |
| | ttaatcggcgtcttactgagtacgttccgcacccagggcagtgtagtcgtcccggacggctttcatccgaagctctt |
| | ctgggatcagtttgttaaatataactgcaattggtttagttgcgtcccaacgatctctatgattatgttgaatatgcccaa |
| | accgaatccgtttccgcacattcgctttatccgctcatgtagcagcgcgctggcgccagcaacgtttcacaagctg |
| | gaaaaagaatttaatgccccagttctggaagcgtacgcgatgacagaagcatctcatcagatgaccagtaacaatc |
| | tgcctcccggtaaacgtaaaccggggaccgtgggccaacctcaaggtgtaaccgtagtaatcctggatgacaac |
| | gataacgttctgccgcccggcaaagttggcgaggtgtcgatccgtggggagaacgtcaccctgggctacgctaa |
| | taacccgaaagctaacaaagaaaacttcactaaacgtgaaaactatttccgtaccggggatcagggctacttcgac |
| | ccggagggctttctcgtgctgaccggccgcattaaagaattgatcaatcgcggtggtgaaaaaattagtcctattga |
| | actggacggaatcatgctctcgcatcctaaaatcgacgaggcggtggcgttcggcgttccagatgatatgtatggc |
| | caagtcgttcaggcggcaatcgtgttgaaaaagggggaaaagatgacctatgaagaattagtgaatttcctgaaaa |
| | agcatttagcaagctttaaaatcccaaccaaagtctactttgtggataagctgcctaaaacggccaccgggaagatt |
| | caacgtcgcgtaatcgccgaaaccttcgcgaaatctagtcgcaacaaaagcaaactttaagtagcttataaaggag |
| | gataaccgcgcatggtgcttggcaaaaaacatcttaatcatgcgaaggacagtttctaatgagtaacgacgacaat |
| | gtagagttgactgatggctttcatgttttgatcgatgccctgaaaatgaatgacatcgataccatgtatggtgttgtcg |
| | gcattcctatcacgaacctggctcgtatgtggcaagatgacggtcagcgtttttacagcttccgtcacgaacaacac |
| | gcaggttatgcagcttctatcgccggttacatcgaaggaaaacctggcgtttgcttgaccgtttccgcccctggcttc |
| | ctgaacggcgtgacttccctggctcatgcaaccaccaactgcttcccaatgatcctgttgagcggttccagtgaac |
| | gtgaaatcgtcgatttgcaacagggcgattacgaagaaatggatcagatgaatgttgcacgtccacactgcaaag |
| | cttctttccgtatcaacagcatcaaagacattccaatcggtatcgctcgtgcagttcgcaccgctgtatccggacgtc |
| | caggtggtgtttacgttgacttgccagcaaaactgttcggtcagaccatttctgtagaagaagctaacaaactgctct |
| | tcaaaccaatcgatccagctccggcacagattcctgctgaagacgctatcgctcgcgctgctgacctgatcaagaa |
| | cgccaaacgtccagttatcatgctgggtaaaggcgctgcatacgcacaatgcgacgacgaaatccgcgcactgg |
| | ttgaagaaaccggcatcccattcctgccaatgggtatggctaaaggcctgctgcctgacaaccatccacaatccgc |
| | tgctgcaacccgtgctttcgcactggcacagtgtgacgtttgcgtactgatcggcgctcgtctgaactggctgatgc |
| | agcacggtaaaggcaaaacctggggcgacgaactgaagaaatacgttcagatcgacatccaggctaacgaaat |
| | ggacagcaaccagcctatcgctgcaccagttgttggtgacatcaagtccgccgtttccctgctccgcaaagcactg |
| | aaaggcgctccaaaagctgacgctgaatggaccggcgctctgaaagccaaagttgacggcaacaaagccaaac |
| | tggctggcaagatgactgccgaaaccccatccggaatgatgaactactccaattccctgggcgttgttcgtgacttc |
| | atgctggcaaatccggatatttccctggttaacgaaggcgctaatgcactcgacaacactcgtatgattgttgacatg |
| | ctgaaaccacgcaaacgtcttgactccggtacctggggtgttatgggtattggtatgggctactgcgttgctgcagc |
| | tgctgttaccggcaaaccggttatcgctgttgaaggcgatagcgcattcggtttctccggtatggaactggaaacca |
| | tctgccgttacaacctgccagttaccgttatcatcatgaacaatggtggtatctataaaggtaacgaagcagatccac |
| | aaccaggcgttatctcctgtacccgtctgacccgtggtcgttacgacatgatgatggaagcatttggcggtaaaggt |
| | tatgttgccaatactccagcagaactgaaagctgctctggaagaagctgttgcttccggcaaaccatgcctgatcaa |
| | cgcgatgatcgatccagacgctggtgtcgaatctggccgtatcaagagcctgaacgttgtaagtaaagttggcaa |
| | gaaataaaataattttgtttaactttaagaaggaggtatatccatggctagcatgactaaacatcttaatcatgcggag |
| | gagggtttctaatgaataatccacaaacaggacaatcaacaggcctcttgggcaatcgttggttctacttggtattag |
| | cagttttgctgatgtgtatgatctcgggtgtccaatattcctggacactgtacgctaacccggttaaagacaaccttgg |
| | cgtttctttggctgcggttcagacggctttcacactctctcaggtcattcaagctggttctcagcctggtggtggttact |
| | tcgttgataaattcggtccaagaattccattgatgttcggtggtgcgatggttctcgctggctggaccttcatgggtat |
| | ggttgacagtgttcctgctctgtatgctctttatactctggccggtgcaggagttggtatcgtttacggtatcgcgatg |
| | aacacggctaacagatggttcccggacaaacgcggtctggcttccggtttcaccgctgccggttacggtctgggt |
| | gttctgccgttcctgccactgatcagctccgttctgaaagttgaaggtgttggcgcagcattcatgtacaccggtttg |
| | atcatgggtatcctgattatcctgatcgctttcgttatccgtttccctggccagcaaggcgccaaaaaacaaatcgttg |
| | ttaccgacaaggatttcaattctggcgaaatgctgagaacaccacaattctgggttctgtggaccgcattcttttccgt |
| | taactttggtggtttgctgctggttgccaacagcgtcccttacggtcgcagcctcggtcttgccgcaggagtgctga |
| | cgatcggtgtttcgatccagaacctgttcaatggtggttgccgtcctttctggggtttcgtttccgataaaatcggccg |
| | ttacaaaaccatgtccgtcgttttcggtatcaatgctgttgttctcgcacttttcccgacgattgctgccttgggcgatgt |
| | agcctttatcgccatgttggcaatcgcattcttcacatggggggtagctacgctctgttcccatcgaccaacagcga |
| | tattttcggtacggcatactctgccagaaactatggtttcttctgggctgcaaaagcaactgcctcgatcttcggtggt |
| | ggtctgggtgctgcaattgcaaccaacttcggatggaataccgctttcctgattactgcgattacttctttcatcgcatt |
| | tgctctggctaccttcgttattccaagaatgggccgtccagtcaagaaaatggtcaaattgtctccagaagaaaaag |
| | ctgtacattaa |
| Operator 1 | taacaccgtgcgtgttg |
| SEQ ID NO: | |
| 1111 | |
| Operator 2 | tacctctggcggtgata |
| SEQ ID NO: | |
| 1112 | |
| Construct | tcagccaaacgtctcttcaggccactgactagcgataactttccccacaacggaacaactctcattgcatgggatca |
| comprising | ttgggtactgtgggtttagtggttgtaaaaacacctgaccgctatccctgatcagtttcttgaaggtaaactcaccacc |
| CI38 in reverse | cccgagtctggctatgcagaaatcacctggctcaacagcctgctcagggtcaacgagaattaacattccgtcagg |
| orientation, | aaagcttggcttggagcctgttggtgctgtcatggaattaccttcaacctcaagccagaatgcagaatcactggcttt |
| temperature | tttggttgtgcttacccacctttccgcaccacctttggtaaaggttctaagctcaggtgagagcatccctgcctgaac |
| sensitive | atgagaaaaaacagggtactcatactcacttctaagtgacggctgcatactaaccgcttcatacatctcgtagatttct |
| inducible | ctggcgattgaagggctaaattcttcaacgctaactttgagaattcttgtaagcgatgcggcgttataagcatttaatg |
| promoter, RBS, | cattgatgccattaaataaggcaccaacgcctgactgccccatccccatcttgtctgcgacagattcctgggataagcc |
| and gene | aagttcatttttctttttttcataaattgctttaaggcgacgtgcgtcctcaagctgctcttgtgttaatggtttcttttt |
| sequences | tgtgctcatacgttaaatctatcaccgcaagggataaatatctaacaccgtgcgtgttgactattttacctctggcggtga |
| encoding | taatggttgcatagctgtcaccggatgtgctttccggtctgatgagtccgtgaggacgaaacagcctctacaaataa |
| oxalate | ttttgtttaagaccagaggtaaggaggtaacaaccatgcgagtgttgaagaaacatcttaatcatgctggggagggt |
| catabolism | ttctaatgaccagtgcagctacggtgaccgcgagctttaatgacactttttctgtgagcgataatgtcgcggtaatcg |
| enzymes, RBS | taccggaaaccgatacgcaggtcacctaccgtgatctttcccacatggtaggacactttcaaacaatgttcacgaac |
| sites are | ccgaatagtcctctgtacggggcggtctttcgtcaagacacggtagcgattagcatgcgtaacggccttgaatttatt |
| present | gtggctttccttggagccacgatggatgcgaaaattggtgcgccactgaatcccaattataaagagaaggagttta |
| between ORF1 | atttttacctgaatgacttaaagtccaaagccatctgcgtgccgaaaggcaccaccaaactgcaaagttcagaaatt |
| and ORF2 and | cttaagagtgcgtccacgttcgggtgctttattgtggaactggcgtttgacgccacccgttttcgtgttgaatatgaca |
| between ORF2 | tttactccccggaggacaattataaacgtgtgatctaccgcagccttaacaatgctaagtttgtcaacacaaaccctg |
| and ORF3 | tcaagttcccgggtttcgcccgcagctcggatgttgcacttattttgcatacctcaggcaccactagtaccccaaag |
| The three RBS | accgtacccctcttgcatctgaatattgtccgttcaaccctgaatatcgccaacacttacaaacttaccccgctggatc |
| sequences | gctcctatgttgtaatgccgctgtttcatgtacatggattaatcggcgtcttactgagtacgttccgcacccagggca |
| present in the | gtgtagtcgtcccggacggctttcatccgaagctcttctgggatcagtttgttaaatataactgcaattggtttagttgc |
| constructs, | gtcccaacgatctctatgattatgttgaatatgcccaaaccgaatccgtttccgcacattcgctttatccgctcatgtag |
| located 5′ of | cagcgcgctggcgccagcaacgtttcacaagctggaaaaagaatttaatgccccagttctggaagcgtacgcgat |
| ORF1, ORF2, | gacagaagcatctcatcagatgaccagtaacaatctgcctcccggtaaacgtaaaccggggaccgtgggccaac |
| and ORF3, | ctcaaggtgtaaccgtagtaatcctggatgacaacgataacgttctgccgcccggcaaagttggcgaggtgtcgat |
| respectively, | ccgtggggagaacgtcaccctgggctacgctaataacccgaaagctaacaaagaaaacttcactaaacgtgaaa |
| are underlined | actatttccgtaccggggatcagggctacttcgacccggagggctttctcgtgctgaccggccgcattaaagaatt |
| SEQ ID NO: | gatcaatcgcggtggtgaaaaaattagtcctattgaactggacggaatcatgctctcgcatcctaaaatcgacgag |
| 1113 | gcggtggcgttcggcgttccagatgatatgtatggccaagtcgttcaggcggcaatcgtgttgaaaaagggggaa |
| | aagatgacctatgaagaattagtgaatttcctgaaaaagcatttagcaagctttaaaatcccaaccaaagtctactttg |
| | tggataagctgcctaaaacggccaccgggaagattcaacgtcgcgtaatcgccgaaaccttcgcgaaatctagtc |
| | gcaacaaaagcaaactttaagtagcttataaaggaggataaccgcgcatggtgcttggcaaaaaacatcttaatca |
| | tgcgaaggacagtttctaatgagtaacgacgacaatgtagagttgactgatggctttcatgttttgatcgatgccctg |
| | aaaatgaatgacatcgataccatgtatggtgttgtcggcattcctatcacgaacctggctcgtatgtggcaagatga |
| | cggtcagcgtttttacagcttccgtcacgaacaacacgcaggttatgcagcttctatcgccggttacatcgaaggaa |
| | aacctggcgtttgcttgaccgtttccgcccctggcttcctgaacggcgtgacttccctggctcatgcaaccaccaac |
| | tgcttcccaatgatcctgttgagcggttccagtgaacgtgaaatcgtcgatttgcaacagggcgattacgaagaaat |
| | ggatcagatgaatgttgcacgtccacactgcaaagcttctttccgtatcaacagcatcaaagacattccaatcggtat |
| | cgctcgtgcagttcgcaccgctgtatccggacgtccaggtggtgtttacgttgacttgccagcaaaactgttcggtc |
| | agaccatttctgtagaagaagctaacaaactgctcttcaaaccaatcgatccagctccggcacagattcctgctgaa |
| | gacgctatcgctcgcgctgctgacctgatcaagaacgccaaacgtccagttatcatgctgggtaaaggcgctgca |
| | tacgcacaatgcgacgacgaaatccgcgcactggttgaagaaaccggcatcccattcctgccaatgggtatggct |
| | aaaggcctgctgcctgacaaccatccacaatccgctgctgcaacccgtgctttcgcactggcacagtgtgacgttt |
| | gcgtactgatcggcgctcgtctgaactggctgatgcagcacggtaaaggcaaaacctggggcgacgaactgaa |
| | gaaatacgttcagatcgacatccaggctaacgaaatggacagcaaccagcctatcgctgcaccagttgttggtga |
| | catcaagtccgccgtttccctgctccgcaaagcactgaaaggcgctccaaaagctgacgctgaatggaccggcg |
| | ctctgaaagccaaagttgacggcaacaaagccaaactggctggcaagatgactgccgaaaccccatccggaat |
| | gatgaactactccaattccctgggcgttgttcgtgacttcatgctggcaaatccggatatttccctggttaacgaagg |
| | cgctaatgcactcgacaacactcgtatgattgttgacatgctgaaaccacgcaaacgtcttgactccggtacctggg |
| | gtgttatgggtattggtatgggctactgcgttgctgcagctgctgttaccggcaaaccggttatcgctgttgaaggcg |
| | atagcgcattcggtttctccggtatggaactggaaaccatctgccgttacaacctgccagttaccgttatcatcatga |
| | acaatggtggtatctataaaggtaacgaagcagatccacaaccaggcgttatctcctgtacccgtctgacccgtgg |
| | tcgttacgacatgatgatggaagcatttggcggtaaaggttatgttgccaatactccagcagaactgaaagctgctc |
| | tggaagaagctgttgcttccggcaaaccatgcctgatcaacgcgatgatcgatccagacgctggtgtcgaatctgg |
| | ccgtatcaagagcctgaacgttgtaagtaaagttggcaagaaataaaataattttgtttaactttaagaaggaggtat |
| | atccatggctagcatgactaaacatcttaatcatgcggaggagggtttctaatgaataatccacaaacaggacaatc |
| | aacaggcctcttgggcaatcgttggttctacttggtattagcagttttgctgatgtgtatgatctcgggtgtccaatattc |
| | ctggacactgtacgctaacccggttaaagacaaccttggcgtttctttggctgcggttcagacggctttcacactctc |
| | tcaggtcattcaagctggttctcagcctggtggtggttacttcgttgataaattcggtccaagaattccattgatgttcg |
| | gtggtgcgatggttctcgctggctggaccttcatgggtatggttgacagtgttcctgctctgtatgctctttatactctg |
| | gccggtgcaggagttggtatcgtttacggtatcgcgatgaacacggctaacagatggttcccggacaaacgcggt |
| | ctggcttccggtttcaccgctgccggttacggtctgggtgttctgccgttcctgccactgatcagctccgttctgaaa |
| | gttgaaggtgttggcgcagcattcatgtacaccggtttgatcatgggtatcctgattatcctgatcgctttcgttatccg |
| | tttccctggccagcaaggcgccaaaaaacaaatcgttgttaccgacaaggatttcaattctggcgaaatgctgaga |
| | acaccacaattctgggttctgtggaccgcattcttttccgttaactttggtggtttgctgctggttgccaacagcgtccc |
| | ttacggtcgcagcctcggtcttgccgcaggagtgctgacgatcggtgtttcgatccagaacctgttcaatggtggtt |
| | gccgtcctttctggggtttcgtttccgataaaatcggccgttacaaaaccatgtccgtcgttttcggtatcaatgctgtt |
| | gttctcgcacttttcccgacgattgctgccttgggcgatgtagcctttatcgccatgttggcaatcgcattcttcacatg |
| | gggtggtagctacgctctgttcccatcgaccaacagcgatattttcggtacggcatactctgccagaaactatggttt |
| | cttctgggctgcaaaagcaactgcctcgatcttcggtggtggtctgggtgctgcaattgcaaccaacttcggatgga |
| | ataccgctttcctgattactgcgattacttctttcatcgcatttgctctggctaccttcgttattccaagaatgggccgtc |
| | cagtcaagaaaatggtcaaattgtctccagaagaaaaagctgtacattaa |

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. A method for reducing the levels of oxalate in a subject, the method comprising administering to the subject a pharmaceutical composition comprising a recombinant bacterium comprising:
one or more gene sequences encoding one or more oxalate catabolism enzymes operably linked directly or indirectly to a first promoter that is not associated with the oxalate catabolism enzyme gene in nature, wherein the one or more gene sequences comprise a scaaE3 gene, an frc gene, and an oxdC gene, wherein the scaaE3 gene comprises a sequence having at least 90%, 91%, 92%, 93%, 94%, 95%, 96%, 97%, 98%, or 99% identity to, comprises, or consists of SEQ ID NO: 3, wherein the frc gene comprises a sequence having at least 90%, 91%, 92%, 93%, 94%, 95%, 96%, 97%, 98%, or 99% identity to, comprises, or consists of SEQ ID NO: 1, and wherein the oxdC gene comprises a sequence having at least 90%, 91%, 92%, 93%, 94%, 95%, 96%, 97%, 98%, or 99% identity to, comprises, or consists of SEQ ID NO: 2,
a gene encoding an oxalate importer, wherein the gene encoding the oxalate importer is an oxlT gene, and wherein the oxlT gene comprises a sequence having at least 90%, 91%, 92%, 93%, 94%, 95%, 96%, 97%, 98%, or 99% identity to, comprises, or consists of SEQ ID NO: 11,
a Δ thyA auxotrophy,
a deletion in an endogenous phage,
a modified endogenous colibactin island,
thereby reducing the levels of oxalate in the subject.

2. The method of claim 1, wherein the endogenous phage comprises a sequence having at least 90%, 91%, 92%, 93%, 94%, 95%, 96%, 97%, 98%, or 99% identity to, comprises, or consists of SEQ ID NO: 63.

3. The method of claim 1 or claim 2, wherein the modified endogenous colibactin island comprises one or more modified clb sequences selected from the group consisting of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), clbR (SEQ ID NO: 1082), and clbS (SEQ ID NO: 1803).

4. The method of any one of claims 1-3, wherein the modified endogenous colibactin island comprises a deletion of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), and clbR (SEQ ID NO: 1082).

5. The method of any one of the previous claims, wherein the recombinant bacterium does not comprise a gene encoding for antibiotic resistance.

6. The method of any one of claims 1-5, wherein the recombinant bacterium has an oxalate consumption activity of at least about 1 μmol/1×10⁹ cell.

7. The method of any one of claims 1-6, wherein the recombinant bacterium has an oxalate consumption activity of about 50 to about 600 mg/day under anaerobic conditions.

8. The method of claim 7, wherein the recombinant bacterium has an oxalate consumption activity of about 211 mg/day under anaerobic conditions.

9. The method of any one of the previous claims, wherein the recombinant bacterium has an oxalate consumption activity of about 211 mg/day under anaerobic conditions when administered to the subject three times per day.

10. The method of any one of claims 7-9, wherein the anaerobic conditions are conditions in the intestine and/or colon of the subject.

11. The method of any one of the previous claims, wherein the method reduces acute levels of oxalate in the subject by about two fold.

12. The method of any one of claims 1-11, wherein the method reduces acute levels of oxalate in the subject by about three fold.

13. The method of any one of claims 1-11, wherein the method reduces chronic levels of oxalate in the subject by about two fold.

14. The method of any one of claims 1-11 or 13, wherein the method reduces chronic levels of oxalate in the subject by about three fold.

15. The method of any one of claims 1-10, wherein the method reduces acute levels of oxalate in the subject to about 25 mg/day, about 30 mg/day, about 40 mg/day, about 50 mg/day, about 60 mg/day, about 70 mg/day, about 80 mg/day, about 90 mg/day, or about 100 mg/day.

16. The method of any one of claims 1-10, wherein the method reduces chronic levels of oxalate in the subject to about 25 mg/day, about 30 mg/day, about 40 mg/day, about 50 mg/day, about 60 mg/day, about 70 mg/day, about 80 mg/day, about 90 mg/day, or about 100 mg/day.

17. The method of any one of the previous claims, wherein the recombinant bacterium is of the genus Escherichia.

18. The method of claim 17, wherein the recombinant bacterium is of the species Escherichia coli strain Nissle.

19. The method of any one of the previous claims, wherein the pharmaceutical composition is administered orally.

20. The method of any one of the previous claims, wherein the subject is fed a meal within one hour of administering the pharmaceutical composition.

21. The method of any one of claims 1-19, wherein the subject is fed a meal concurrently with administering the pharmaceutical composition.

22. The method of any one of the previous claims, wherein the subject is a human subject.

23. The method of any one of the previous claims, wherein the first promoter is an inducible promoter, optionally when the inducible promoter is a FNR promoter, optionally wherein the FNR promoter comprises a sequence having at least 90%, 91%, 92%, 93%, 94%, 95%, 96%, 97%, 98%, or 99% identity to, comprises, or consists of any one of SEQ ID NOs: 13-29.

24. The method of any one of claims 1-23, wherein the recombinant bacterium is SYNB8802v1.

25. The method of any one of the previous claims, wherein the subject has hyperoxaluria.

26. The method of claim 25, wherein the hyperoxaluria is primary hyperoxaluria, dietary hyperoxaluria, or enteric hyperoxaluria.

27. The method of claim 25 or claim 26, wherein the subject has short bowel syndrome, chronic pancreatitis, inflammatory bowel disease (IBD), cystic fibrosis, kidney disease, and/or Roux-en-Y gastric bypass.

28. The method of claim 27, wherein the subject has short bowel syndrome and/or Roux-en-Y gastric bypass.

29. The method of any one of the previous claims, wherein the subject has urinary oxalate (Uox) levels of at least 70 mg/day prior to the administering.

30. The method of any one of the previous claims, wherein the subject exhibits a decrease in Uox levels of at least 10%, 15%, 20%, 25%, 30%, 35%, 40%, 45%, 50%, 55%, 60%, 65%, 70%, 75%, or 80% after the administering.

31. The method of any one of the previous claims, wherein the subject has eGFR<30 mL/min/1.73 m², requires hemodialysis, or has systemic oxalosis prior to the administering.

32. The method of any one of the previous claims, wherein the recombinant bacteria is administered at a dose of about 1×10¹¹ live recombinant bacteria, about 3×10¹¹ live recombinant bacteria, about 4.5×10¹¹ live recombinant bacteria, about 5×10¹¹ live recombinant bacteria, about 6×10¹¹ live recombinant bacteria, about 1×10¹² live recombinant bacteria, or about 2×10¹² live recombinant bacteria.

33. The method of any one of the previous claims, wherein the recombinant bacteria is administered once daily, twice daily, or three times daily.

34. The method of any one of the previous claims, wherein the administering is about 5×10¹¹ live recombinant bacteria with meals three times per day.

35. The method of any one of the previous claims, further comprising administering a proton pump inhibitor (PPI) to the subject.

36. The method of claim 35, wherein the PPI is esomeprazole.

37. The method of claim 35 or claim 36, wherein the administering of the PPI is once a day.

38. The method of any one of the previous claims, wherein the pharmaceutical composition further comprises galactose.

39. The method of claim 38, wherein galactose is D-galactose.

40. A recombinant bacterium comprising:
one or more gene sequences encoding one or more oxalate catabolism enzymes operably linked directly or indirectly to a first promoter that is not associated with the oxalate catabolism enzyme gene in nature, wherein the one or more gene sequences comprise a scaaE3 gene, an frc gene, and an oxdC gene, wherein the scaaE3 gene comprises a sequence having at least 90%, 91%, 92%, 93%, 94%, 95%, 96%, 97%, 98%, or 99% identity to, comprises, or consists of SEQ ID NO: 3, wherein the frc gene comprises a sequence having at least 90%, 91%, 92%, 93%, 94%, 95%, 96%, 97%, 98%, or 99% identity to, comprises, or consists of SEQ ID NO: 1, and wherein the oxdC gene comprises a sequence having at least 90%, 91%, 92%, 93%, 94%, 95%, 96%, 97%, 98%, or 99% identity to, comprises, or consists of SEQ ID NO: 2,
a gene encoding an oxalate importer, wherein the gene encoding the oxalate importer is an oxlT gene, and wherein the oxlT gene comprises a sequence having at least 90%, 91%, 92%, 93%, 94%, 95%, 96%, 97%, 98%, or 99% identity to, comprises, or consists of SEQ ID NO: 11,
a Δ thyA auxotrophy,
a deletion in an endogenous phage,
a modified endogenous colibactin island.

41. The recombinant bacterium of claim 40, wherein the endogenous phage comprises a sequence having at least 90%, 91%, 92%, 93%, 94%, 95%, 96%, 97%, 98%, or 99% identity to, comprises, or consists of SEQ ID NO: 63.

42. The recombinant bacterium of claim 40 or claim 41, wherein the modified endogenous colibactin island comprises one or more modified clb sequences selected from the group consisting of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), clbR (SEQ ID NO: 1082), and clbS (SEQ ID NO: 1803).

43. The recombinant bacterium of any one of claims 40-42, wherein the modified endogenous colibactin island comprises a deletion of clbA (SEQ ID NO: 1065), clbB (SEQ ID NO: 1066), clbC (SEQ ID NO: 1067), clbD (SEQ ID NO: 1068), clbE (SEQ ID NO: 1069), clbF (SEQ ID NO: 1070), clbG (SEQ ID NO: 1071), clbH (SEQ ID NO: 1072), clbI (SEQ ID NO: 1073), clbJ (SEQ ID NO: 1074), clbK (SEQ ID NO: 1075), clbL (SEQ ID NO: 1076), clbM (SEQ ID NO: 1077), clbN (SEQ ID NO: 1078), clbO (SEQ ID NO: 1079), clbP (SEQ ID NO: 1080), clbQ (SEQ ID NO: 1081), and clbR (SEQ ID NO: 1082).

44. The recombinant bacterium of any one of claims 40-43, wherein the recombinant bacterium does not comprise a gene encoding for antibiotic resistance.

45. The recombinant bacterium of any one of claims 40-44, wherein the first promoter is an inducible promoter, optionally when the inducible promoter is a FNR promoter, optionally wherein the FNR promoter comprises a sequence having at least 90%, 91%, 92%, 93%, 94%, 95%, 96%, 97%, 98%, or 99% identity to, comprises, or consists of any one of SEQ ID NOs: 13-29.

46. The recombinant bacterium of any one of claims 40-45, wherein the recombinant bacterium has an oxalate consumption activity of at least about 1 μmol/1×10⁹ cell.

47. The recombinant bacterium of any one of claims 40-46, wherein the recombinant bacterium has an oxalate consumption activity of about 50-600 mg/day under anaerobic conditions.

48. The recombinant bacterium of any one of claims 40-47, wherein the recombinant bacterium is SYNB8802v1.

49. The recombinant bacterium of claim 48, wherein the recombinant bacterium is SYNB8802.

50. The recombinant bacterium of any one of claims 40-49, wherein the recombinant bacterium has an oxalate consumption activity of about 0.2 μmole/hr to about 1.6 μmole/hr, about 0.5 μmole/hr to about 1.5 μmole/hr, or about 1.0 μmole/hr to about 1.5 μmole/hr under anaerobic conditions.

51. The recombinant bacterium of claim 50, wherein the recombinant bacterium has an oxalate consumption activity of about 0.5 μmole/hr to about 1.5 μmole/hr under anaerobic conditions.

52. The method of any one of claims 1-39, wherein the recombinant bacterium has an oxalate consumption activity of about 0.2 μmole/hr to about 1.6 μmole/hr, about 0.5 μmole/hr to about 1.5 μmole/hr, or about 1.0 μmole/hr to about 1.5 μmole/hr under anaerobic conditions.

53. The method of claim 52, wherein the recombinant bacterium has an oxalate consumption activity of about 0.5 μmole/hr to about 1.5 μmole/hr under anaerobic conditions.

54. The method of any one of claims 1-39, wherein the recombinant bacterium is capable of decreasing urinary oxalate in the subject after administration by at least 20%, at least 25%, at least 30%, at least 35%, at least 40%, or at least 45%.

55. The method of any one of claims 1-39, wherein the recombinant bacterium is capable of decreasing fecal oxalate in the subject after administration by at least 20%, at least 30%, at least 40%, at least 50%, at least 60%, at least 70%, at least 80%, or at least 85%.


SEQUENCE LISTING (1104 sequences)

SEQ ID NO: 1 - DNA, 1287 bp, Oxalobacter formigenes
atgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 2 - DNA, 1707 bp, Oxalobacter formigenes
atgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataa

SEQ ID NO: 3 - DNA, 1632 bp, Saccharomyces cerevisiae
atgaccagtgcagctacggtgaccgcgagctttaatgacactttttctgtgagcgataatgtcgcggtaatcgtaccggaaaccgatacgcaggtcacctaccgtgatctttcccacatggtaggacactttcaaacaatgttcacgaacccgaatagtcctctgtacggggcggtctttcgtcaagacacggtagcgattagcatgcgtaacggccttgaatttattgtggctttccttggagccacgatggatgcgaaaattggtgcgccactgaatcccaattataaagagaaggagtttaatttttacctgaatgacttaaagtccaaagccatctgcgtgccgaaaggcaccaccaaactgcaaagttcagaaattcttaagagtgcgtccacgttcgggtgctttattgtggaactggcgtttgacgccacccgttttcgtgttgaatatgacatttactccccggaggacaattataaacgtgtgatctaccgcagccttaacaatgctaagtttgtcaacacaaaccctgtcaagttcccgggtttcgcccgcagctcggatgttgcacttattttgcatacctcaggcaccactagtaccccaaagaccgtacccctcttgcatctgaatattgtccgttcaaccctgaatatcgccaacacttacaaacttaccccgctggatcgctcctatgttgtaatgccgctgtttcatgtacatggattaatcggcgtcttactgagtacgttccgcacccagggcagtgtagtcgtcccggacggctttcatccgaagctcttctgggatcagtttgttaaatataactgcaattggtttagttgcgtcccaacgatctctatgattatgttgaatatgcccaaaccgaatccgtttccgcacattcgctttatccgctcatgtagcagcgcgctggcgccagcaacgtttcacaagctggaaaaagaatttaatgccccagttctggaagcgtacgcgatgacagaagcatctcatcagatgaccagtaacaatctgcctcccggtaaacgtaaaccggggaccgtgggccaacctcaaggtgtaaccgtagtaatcctggatgacaacgataacgttctgccgcccggcaaagttggcgaggtgtcgatccgtggggagaacgtcaccctgggctacgctaataacccgaaagctaacaaagaaaacttcactaaacgtgaaaactatttccgtaccggggatcagggctacttcgacccggagggctttctcgtgctgaccggccgcattaaagaattgatcaatcgcggtggtgaaaaaattagtcctattgaactggacggaatcatgctctcgcatcctaaaatcgacgaggcggtggcgttcggcgttccagatgatatgtatggccaagtcgttcaggcggcaatcgtgttgaaaaagggggaaaagatgacctatgaagaattagtgaatttcctgaaaaagcatttagcaagctttaaaatcccaaccaaagtctactttgtggataagctgcctaaaacggccaccgggaagattcaacgtcgcgtaatcgccgaaaccttcgcgaaatctagtcgcaacaaaagcaaactttaa

SEQ ID NO: 4 - DNA, 1146 bp, Escherichia coli
atgacaaataatgaaagcaaagggccgtttgaaggcttattagttatcgatatgacacatgtccttaatggacctttcggaactcaacttctttgtaatatgggcgcaagggtaattaaagttgagccgccgggtcatggtgatgatacccgcacatttggtccctatgtggatggacagtcactctattacagttttattaatcatggcaaagagagtgtggttcttgatttaaagaatgatcacgataaaagtatatttataaatatgctcaaacaagctgatgtattagctgagaattttcgcccaggtacaatggaaaaactggggttttcatgggaaacgcttcaagaaatcaacccgcgcctcatatatgcttcatcgtcaggtttcggacataccggtccgctaaaagatgctcctgcctacgataccatcattcaggcaatgagcgggataatgatggaaacaggatatcctgatgctccgccagtgcgcgttggtacatctcttgcggatctatgcggcggtgtctatttattcagcggaatagtgagtgcactttatggccgcgaaaagagccagagaggggcgcatgtcgatatagcgatgtttgatgccacgctgagttttctggagcatggtctgatggcatatatcgcaactgggaagtcaccacaacgtctgggaaatcgccatccctacatggcaccttttgatgttttcaatactcaggataagccgattacgatttgttgtggtaatgacaagcttttttctgcgttatgccaggcactggagcttacggaactggttaatgatccccgatttagcagcaatattttacgcgtacaaaaccaggctattcttaaacaatatattgagcggacgttaaaaacgcaggcagctgaagtttggttagccagaatacatgaagttggtgtacccgtcgcgccgttattaagtgtggctgaggccattaaattgccacaaactcaggcgagaaatatgttgattgaagccgggggaataatgatgccgggtaatccgataaaaatcagcggctgcgcggacccgcatgttatgccgggagcggcaacgctcgaccagcatggggaacaaattcgccaggagttctcatcataa

SEQ ID NO: 5 - protein, 428 aa, Oxalobacter formigenes
MTKPLDGINVLDFTHVQAGPACTQMMGFLGANVIKIERRGSGDMTRGWLQDKPNVDSLYFTMFNCNKRSIELDMKTPEGKELLEQMIKKADVMVENFGPGALDRMGFTWEYIQELNPRVILASVKGYAEGHANEHLKVYENVAQCSGGAAATTGFWDGPPTVSGAALGDSNSGMHLMIGILAALEMRHKTGRGQKVAVAMQDAVLNLVRIKLRDQQRLERTGILAEYPQAQPNFAFDRDGNPLSFDNITSVPRGGNAGGGGQPGWMLKCKGWETDADSYVYFTIAANMWPQICDMIDKPEWKDDPAYNTFEGRVDKLMDIFSFIETKFADKDKFEVTEWAAQYGIPCGPVMSMKELAHDPSLQKVGTVVEVVDEIRGNHLTVGAPFKFSGFQPEITRAPLLGEHTDEVLKELGLDDAKIKELHAKQVV

SEQ ID NO: 6 - protein, 568 aa, Oxalobacter formigenes
MSNDDNVELTDGFHVLIDALKMNDIDTMYGVVGIPITNLARMWQDDGQRFYSFRHEQHAGYAASIAGYIEGKPGVCLTVSAPGFLNGVTSLAHATTNCFPMILLSGSSEREIVDLQQGDYEEMDQMNVARPHCKASFRINSIKDIPIGIARAVRTAVSGRPGGVYVDLPAKLFGQTISVEEANKLLFKPIDPAPAQIPAEDAIARAADLIKNAKRPVIMLGKGAAYAQCDDEIRALVEETGIPFLPMGMAKGLLPDNHPQSAAATRAFALAQCDVCVLIGARLNWLMQHGKGKTWGDELKKYVQIDIQANEMDSNQPIAAPVVGDIKSAVSLLRKALKGAPKADAEWTGALKAKVDGNKAKLAGKMTAETPSGMMNYSNSLGVVRDFMLANPDISLVNEGANALDNTRMIVDMLKPRKRLDSGTWGVMGIGMGYCVAAAAVTGKPVIAVEGDSAFGFSGMELETICRYNLPVTVIIMNNGGIYKGNEADPQPGVISCTRLTRGRYDMMMEAFGGKGYVANTPAELKAALEEAVASGKPCLINAMIDPDAGVESGRIKSLNVVSKVGKK

SEQ ID NO: 7 - protein, 543 aa, Saccharomyces cerevisiae
MTSAATVTASFNDTFSVSDNVAVIVPETDTQVTYRDLSHMVGHFQTMFTNPNSPLYGAVFRQDTVAISMRNGLEFIVAFLGATMDAKIGAPLNPNYKEKEFNFYLNDLKSKAICVPKGTTKLQSSEILKSASTFGCFIVELAFDATRFRVEYDIYSPEDNYKRVIYRSLNNAKFVNTNPVKFPGFARSSDVALILHTSGTTSTPKTVPLLHLNIVRSTLNIANTYKLTPLDRSYVVMPLFHVHGLIGVLLSTFRTQGSVVVPDGFHPKLFWDQFVKYNCNWFSCVPTISMIMLNMPKPNPFPHIRFIRSCSSALAPATFHKLEKEFNAPVLEAYAMTEASHQMTSNNLPPGKRKPGTVGQPQGVTVVILDDNDNVLPPGKVGEVSIRGENVTLGYANNPKANKENFTKRENYFRTGDQGYFDPEGFLVLTGRIKELINRGGEKISPIELDGIMLSHPKIDEAVAFGVPDDMYGQVVQAAIVLKKGEKMTYEELVNFLKKHLASFKIPTKVYFVDKLPKTATGKIQRRVIAETFAKSSRNKSKL

SEQ ID NO: 8 - protein, 381 aa, Escherichia coli
MTNNESKGPFEGLLVIDMTHVLNGPFGTQLLCNMGARVIKVEPPGHGDDTRTFGPYVDGQSLYYSFINHGKESVVLDLKNDHDKSIFINMLKQADVLAENFRPGTMEKLGFSWETLQEINPRLIYASSSGFGHTGPLKDAPAYDTIIQAMSGIMMETGYPDAPPVRVGTSLADLCGGVYLFSGIVSALYGREKSQRGAHVDIAMFDATLSFLEHGLMAYIATGKSPQRLGNRHPYMAPFDVFNTQDKPITICCGNDKLFSALCQALELTELVNDPRFSSNILRVQNQAILKQYIERTLKTQAAEVWLARIHEVGVPVAPLLSVAEAIKLPQTQARNMLIEAGGIMMPGNPIKISGCADPHVMPGAATLDQHGEQIRQEFSS

SEQ ID NO: 9 - protein, 437 aa, Escherichia coli
SYYHHHHHHLESTSLYKKAGLMSTPLQGIKVLDFTGVQSGPSCTQMLAWFGADVIKIERPGVGDVTRHQLRDIPDIDALYFTMLNSNKRSIELNTKTAEGKEVMEKLIREADILVENFHPGAIDHMGFTWEHIQEINPRLIFGSIKGFDECSPYVNVKAYENVAQAAGGAASTTGFWDGPPLVSAAALGDSNTGMHLLIGLLAALLHREKTGRGQRVTMSMQDAVLNLCRVKLRDQQRLDKLGYLEEYPQYPNGTFGDAVPRGGNAGGGGQPGWILKCKGWETDPNAYIYFTIQEQNWENTCKAIGKPEWITDPAYSTAHARQPHIFDIFAEIEKYTVTIDKHEAVAYLTQFDIPCAPVLSMKEISLDPSLRQSGSVVEVEQPLRGKYLTVGCPMKFSAFTPDIKAAPLLGEHTAAVLQELGYSDDEIAAMKQNHAI

SEQ ID NO: 10 - protein, 564 aa, Escherichia coli
MSDQLQMTDGMHIIVEALKQNNIDTIYGVVGIPVTDMARHAQAEGIRYIGFRHEQSAGYAAAASGFLTQKPGICLTVSAPGFLNGLTALANATVNGFPMIMISGSSDRAIVDLQQGDYEELDQMNAAKPYAKAAFRVNQPQDLGIALARAIRVSVSGRPGGVYLDLPANVLAATMEKDEALTTIVKVENPSPALLPCPKSVTSAISLLAKAERPLIILGKGAAYSQADEQLREFIESAQIPFLPMSMAKGILEDTHPLSAAAARSFALANADVVMLVGARLNWLLAHGKKGWAADTQFIQLDIEPQEIDSNRPIAVPVVGDIASSMQGMLAELKQNTFTTPLVWRDILNIHKQQNAQKMHEKLSTDTQPLNYFNALSAVRDVLRENQDIYLVNEGANTLDNARNIIDMYKPRRRLDCGTWGVMGIGMGYAIGASVTSGSPVVAIEGDSAFGFSGMEIETICRYNLPVTIVIFNNGGIYRGDGVDLSGAGAPSPTDLLHHARYDKLMDAFRGVGYNVTTTDELRHALTTGIQSRKPTIINVVIDPAAGTESGHITKLNPKQVAGN

SEQ ID NO: 11 - DNA, 1257 bp, Oxalobacter formigenes
atgaataatccacaaacaggacaatcaacaggcctcttgggcaatcgttggttctacttggtattagcagttttgctgatgtgtatgatctcgggtgtccaatattcctggacactgtacgctaacccggttaaagacaaccttggcgtttctttggctgcggttcagacggctttcacactctctcaggtcattcaagctggttctcagcctggtggtggttacttcgttgataaattcggtccaagaattccattgatgttcggtggtgcgatggttctcgctggctggaccttcatgggtatggttgacagtgttcctgctctgtatgctctttatactctggccggtgcaggtgttggtatcgtttacggtatcgcgatgaacacggctaacagatggttcccggacaaacgcggtctggcttccggtttcaccgctgccggttacggtctgggtgttctgccgttcctgccactgatcagctccgttctgaaagttgaaggtgttggcgcagcattcatgtacaccggtttgatcatgggtatcctgattatcctgatcgctttcgttatccgtttccctggccagcaaggcgccaaaaaacaaatcgttgttaccgacaaggatttcaattctggcgaaatgctgagaacaccacaattctgggttctgtggaccgcattcttttccgttaactttggtggtttgctgctggttgccaacagcgtcccttacggtcgcagcctcggtcttgccgcaggtgtgctgacgatcggtgtttcgatccagaacctgttcaatggtggttgccgtcctttctggggtttcgtttccgataaaatcggccgttacaaaaccatgtccgtcgttttcggtatcaatgctgttgttctcgcacttttcccgacgattgctgccttgggcgatgtagcctttatcgccatgttggcaatcgcattcttcacatggggtggtagctacgctctgttcccatcgaccaacagcgatattttcggtacggcatactctgccagaaactatggtttcttctgggctgcaaaagcaactgcctcgatcttcggtggtggtctgggtgctgcaattgcaaccaacttcggatggaataccgctttcctgattactgcgattacttctttcatcgcatttgctctggctaccttcgttattccaagaatgggccgtccagtcaagaaaatggtcaaattgtctccagaagaaaaagctgtacattaa

SEQ ID NO: 12 - protein, 418 aa, Oxalobacter formigenes
MNNPQTGQSTGLLGNRWFYLVLAVLLMCMISGVQYSWTLYANPVKDNLGVSLAAVQTAFTLSQVIQAGSQPGGGYFVDKFGPRIPLMFGGAMVLAGWTFMGMVDSVPALYALYTLAGAGVGIVYGIAMNTANRWFPDKRGLASGFTAAGYGLGVLPFLPLISSVLKVEGVGAAFMYTGLIMGILIILIAFVIRFPGQQGAKKQIVVTDKDFNSGEMLRTPQFWVLWTAFFSVNFGGLLLVANSVPYGRSLGLAAGVLTIGVSIQNLFNGGCRPFWGFVSDKIGRYKTMSVVFGINAVVLALFPTIAALGDVAFIAMLAIAFFTWGGSYALFPSTNSDIFGTAYSARNYGFFWAAKATASIFGGGLGAAIATNFGWNTAFLITAITSFIAFALATFVIPRMGRPVKKMVKLSPEEKAVH

SEQ ID NO: 13 - DNA, 290 bp, Unknown
  Description of Unknown: FNR responsive promoter sequence
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaa

SEQ ID NO: 14 - DNA, 173 bp, Unknown
  Description of Unknown: FNR responsive promoter sequence
atttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacct

SEQ ID NO: 15 - DNA, 305 bp, Unknown
  Description of Unknown: FNR responsive promoter sequence
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacat

SEQ ID NO: 16 - DNA, 180 bp, Unknown
  Description of Unknown: FNR responsive promoter sequence
catttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacat

SEQ ID NO: 17 - DNA, 199 bp, Unknown
  Description of Unknown: FNR responsive promoter sequence
agttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacat

SEQ ID NO: 18 - DNA, 117 bp, Unknown
  Description of Unknown: FNR promoter sequence
atccccatcactcttgatggagatcaattccccaagctgctagagcgttaccttgcccttaaacattagcaatgtcgatttatcagagggccgacaggctcccacaggagaaaaccg

SEQ ID NO: 19 - DNA, 108 bp, Unknown
  Description of Unknown: FNR promoter sequence
ctcttgatcgttatcaattcccacgctgtttcagagcgttaccttgcccttaaacattagcaatgtcgatttatcagagggccgacaggctcccacaggagaaaaccg

SEQ ID NO: 20 - DNA, 290 bp, Unknown; identical to SEQ ID NO: 13
  Description of Unknown: FNR promoter sequence

SEQ ID NO: 21 - DNA, 433 bp, Unknown
  Description of Unknown: FNR promoter sequence
cggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaaatgtttgtttaactttaagaaggagatatacat

SEQ ID NO: 22 - DNA, 290 bp, Unknown
  Description of Unknown: FNR promoter sequence
gtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaa

SEQ ID NO: 23 - DNA, 173 bp, Unknown; identical to SEQ ID NO: 14
  Description of Unknown: FNR promoter sequence

SEQ ID NO: 24 - DNA, 305 bp, Artificial Sequence; identical to SEQ ID NO: 15
  Description of Artificial Sequence: Synthetic polynucleotide

SEQ ID NO: 25 - DNA, 180 bp, Artificial Sequence; identical to SEQ ID NO: 16
  Description of Artificial Sequence: Synthetic polynucleotide

SEQ ID NO: 26 - DNA, 199 bp, Unknown; identical to SEQ ID NO: 17
  Description of Unknown: FNR promoter sequence

SEQ ID NO: 27 - DNA, 207 bp, Unknown
  Description of Unknown: FNR promoter sequence
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacat

SEQ ID NO: 28 - DNA, 390 bp, Artificial Sequence
  Description of Artificial Sequence: Synthetic polynucleotide
tcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtagaaatgtgatctagttcacatttgcggtaatagaaaagaaatcgaggcaaaaatgtttgtttaactttaagaaggagatatacat

SEQ ID NO: 29 - DNA, 200 bp, Artificial Sequence
  Description of Artificial Sequence: Synthetic polynucleotide
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcaaatgtgatctagttcacattttttgtttaactttaagaaggagatatacat

SEQ ID NO: 30 - DNA, 355 bp, Unknown
  Description of Unknown: OxyR-regulated regulatory region sequence
tgtggcttttatgaaaatcacacagtgatcacaaattttaaacagagcacaaaatgctgcctcgaaatgagggcgggaaaataaggttatcagccttgttttctccctcattacttgaaggatatgaagctaaaacccttttttataaagcatttgtccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagaggggagcacattgatgcgaattcattaaagaggagaaaggtacc

SEQ ID NO: 31 - DNA, 228 bp, Unknown
  Description of Unknown: OxyR-regulated regulatory region sequence
ttccgaaaattcctggcgagcagataaataagaattgttcttatcaatatatctaactcattgaatctttattagttttgtttttcacgcttgttaccactattagtgtgataggaacagccagaatagcggaacacatagccggtgctatacttaatctcgttaattactgggacataacatcaagaggatatgaaattcgaattcattaaagaggagaaaggtacc

SEQ ID NO: 32 - DNA, 334 bp, Unknown
  Description of Unknown: OxyR-regulated regulatory region sequence
gcttagatcaggtgattgccctttgtttatgagggtgttgtaatccatgtcgttgttgcatttgtaagggcaacacctcagcctgcaggcaggcactgaagataccaaagggtagttcagattacacggtcacctggaaagggggccattttactttttatcgccgctggcggtgcaaagttcacaaagttgtcttacgaaggttgtaaggtaaaacttatcgatttgataatggaaacgcattagccgaatcggcaaaaattggttaccttacatctcatcgaaaacacggaggaagtatagatgcgaattcattaaagaggagaaaggtacc

SEQ ID NO: 33 - DNA, 134 bp, Unknown
  Description of Unknown: OxyR-regulated regulatory region sequence
ctcgagttcattatccatcctccatcgccacgatagttcatggcgataggtagaatagcaatgaacgattatccctatcaagcattctgactgataattgctcacacgaattcattaaagaggagaaaggtacc

SEQ ID NO: 34 - DNA, 5409 bp, Artificial Sequence
  Description of Artificial Sequence: Synthetic polynucleotide
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgaccagtgcagctacggtgaccgcgagctttaatgacactttttctgtgagcgataatgtcgcggtaatcgtaccggaaaccgatacgcaggtcacctaccgtgatctttcccacatggtaggacactttcaaacaatgttcacgaacccgaatagtcctctgtacggggcggtctttcgtcaagacacggtagcgattagcatgcgtaacggccttgaatttattgtggctttccttggagccacgatggatgcgaaaattggtgcgccactgaatcccaattataaagagaaggagtttaatttttacctgaatgacttaaagtccaaagccatctgcgtgccgaaaggcaccaccaaactgcaaagttcagaaattcttaagagtgcgtccacgttcgggtgctttattgtggaactggcgtttgacgccacccgttttcgtgttgaatatgacatttactccccggaggacaattataaacgtgtgatctaccgcagccttaacaatgctaagtttgtcaacacaaaccctgtcaagttcccgggtttcgcccgcagctcggatgttgcacttattttgcatacctcaggcaccactagtaccccaaagaccgtacccctcttgcatctgaatattgtccgttcaaccctgaatatcgccaacacttacaaacttaccccgctggatcgctcctatgttgtaatgccgctgtttcatgtacatggattaatcggcgtcttactgagtacgttccgcacccagggcagtgtagtcgtcccggacggctttcatccgaagctcttctgggatcagtttgttaaatataactgcaattggtttagttgcgtcccaacgatctctatgattatgttgaatatgcccaaaccgaatccgtttccgcacattcgctttatccgctcatgtagcagcgcgctggcgccagcaacgtttcacaagctggaaaaagaatttaatgccccagttctggaagcgtacgcgatgacagaagcatctcatcagatgaccagtaacaatctgcctcccggtaaacgtaaaccggggaccgtgggccaacctcaaggtgtaaccgtagtaatcctggatgacaacgataacgttctgccgcccggcaaagttggcgaggtgtcgatccgtggggagaacgtcaccctgggctacgctaataacccgaaagctaacaaagaaaacttcactaaacgtgaaaactatttccgtaccggggatcagggctacttcgacccggagggctttctcgtgctgaccggccgcattaaagaattgatcaatcgcggtggtgaaaaaattagtcctattgaactggacggaatcatgctctcgcatcctaaaatcgacgaggcggtggcgttcggcgttccagatgatatgtatggccaagtcgttcaggcggcaatcgtgttgaaaaagggggaaaagatgacctatgaagaattagtgaatttcctgaaaaagcatttagcaagctttaaaatcccaaccaaagtctactttgtggataagctgcctaaaacggccaccgggaagattcaacgtcgcgtaatcgccgaaaccttcgcgaaatctagtcgcaacaaaagcaaactttaaaagaaggagatatacatatgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataataagaaggagatatacatatgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 35 - DNA, 4785 bp, Artificial Sequence
  Description of Artificial Sequence: Synthetic polynucleotide
cattaattcctaatttttgttgacactctatcattgatagagttattttaccactccctatcagtgatagagaaaagtgaactctagaaataattttgtttaactttaagaaggagatatacatatgaccagtgcagctacggtgaccgcgagctttaatgacactttttctgtgagcgataatgtcgcggtaatcgtaccggaaaccgatacgcaggtcacctaccgtgatctttcccacatggtaggacactttcaaacaatgttcacgaacccgaatagtcctctgtacggggcggtctttcgtcaagacacggtagcgattagcatgcgtaacggccttgaatttattgtggctttccttggagccacgatggatgcgaaaattggtgcgccactgaatcccaattataaagagaaggagtttaatttttacctgaatgacttaaagtccaaagccatctgcgtgccgaaaggcaccaccaaactgcaaagttcagaaattcttaagagtgcgtccacgttcgggtgctttattgtggaactggcgtttgacgccacccgttttcgtgttgaatatgacatttactccccggaggacaattataaacgtgtgatctaccgcagccttaacaatgctaagtttgtcaacacaaaccctgtcaagttcccgggtttcgcccgcagctcggatgttgcacttattttgcatacctcaggcaccactagtaccccaaagaccgtacccctcttgcatctgaatattgtccgttcaaccctgaatatcgccaacacttacaaacttaccccgctggatcgctcctatgttgtaatgccgctgtttcatgtacatggattaatcggcgtcttactgagtacgttccgcacccagggcagtgtagtcgtcccggacggctttcatccgaagctcttctgggatcagtttgttaaatataactgcaattggtttagttgcgtcccaacgatctctatgattatgttgaatatgcccaaaccgaatccgtttccgcacattcgctttatccgctcatgtagcagcgcgctggcgccagcaacgtttcacaagctggaaaaagaatttaatgccccagttctggaagcgtacgcgatgacagaagcatctcatcagatgaccagtaacaatctgcctcccggtaaacgtaaaccggggaccgtgggccaacctcaaggtgtaaccgtagtaatcctggatgacaacgataacgttctgccgcccggcaaagttggcgaggtgtcgatccgtggggagaacgtcaccctgggctacgctaataacccgaaagctaacaaagaaaacttcactaaacgtgaaaactatttccgtaccggggatcagggctacttcgacccggagggctttctcgtgctgaccggccgcattaaagaattgatcaatcgcggtggtgaaaaaattagtcctattgaactggacggaatcatgctctcgcatcctaaaatcgacgaggcggtggcgttcggcgttccagatgatatgtatggccaagtcgttcaggcggcaatcgtgttgaaaaagggggaaaagatgacctatgaagaattagtgaatttcctgaaaaagcatttagcaagctttaaaatcccaaccaaagtctactttgtggataagctgcctaaaacggccaccgggaagattcaacgtcgcgtaatcgccgaaaccttcgcgaaatctagtcgcaacaaaagcaaactttaaaagaaggagatatacatatgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataataagaaggagatatacatatgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 36 - DNA, 4661 bp, Artificial Sequence
  Description of Artificial Sequence: Synthetic polynucleotide
atgaccagtgcagctacggtgaccgcgagctttaatgacactttttctgtgagcgataatgtcgcggtaatcgtaccggaaaccgatacgcaggtcacctaccgtgatctttcccacatggtaggacactttcaaacaatgttcacgaacccgaatagtcctctgtacggggcggtctttcgtcaagacacggtagcgattagcatgcgtaacggccttgaatttattgtggctttccttggagccacgatggatgcgaaaattggtgcgccactgaatcccaattataaagagaaggagtttaatttttacctgaatgacttaaagtccaaagccatctgcgtgccgaaaggcaccaccaaactgcaaagttcagaaattcttaagagtgcgtccacgttcgggtgctttattgtggaactggcgtttgacgccacccgttttcgtgttgaatatgacatttactccccggaggacaattataaacgtgtgatctaccgcagccttaacaatgctaagtttgtcaacacaaaccctgtcaagttcccgggtttcgcccgcagctcggatgttgcacttattttgcatacctcaggcaccactagtaccccaaagaccgtacccctcttgcatctgaatattgtccgttcaaccctgaatatcgccaacacttacaaacttaccccgctggatcgctcctatgttgtaatgccgctgtttcatgtacatggattaatcggcgtcttactgagtacgttccgcacccagggcagtgtagtcgtcccggacggctttcatccgaagctcttctgggatcagtttgttaaatataactgcaattggtttagttgcgtcccaacgatctctatgattatgttgaatatgcccaaaccgaatccgtttccgcacattcgctttatccgctcatgtagcagcgcgctggcgccagcaacgtttcacaagctggaaaaagaatttaatgccccagttctggaagcgtacgcgatgacagaagcatctcatcagatgaccagtaacaatctgcctcccggtaaacgtaaaccggggaccgtgggccaacctcaaggtgtaaccgtagtaatcctggatgacaacgataacgttctgccgcccggcaaagttggcgaggtgtcgatccgtggggagaacgtcaccctgggctacgctaataacccgaaagctaacaaagaaaacttcactaaacgtgaaaactatttccgtaccggggatcagggctacttcgacccggagggctttctcgtgctgaccggccgcattaaagaattgatcaatcgcggtggtgaaaaaattagtcctattgaactggacggaatcatgctctcgcatcctaaaatcgacgaggcggtggcgttcggcgttccagatgatatgtatggccaagtcgttcaggcggcaatcgtgttgaaaaagggggaaaagatgacctatgaagaattagtgaatttcctgaaaaagcatttagcaagctttaaaatcccaaccaaagtctactttgtggataagctgcctaaaacggccaccgggaagattcaacgtcgcgtaatcgccgaaaccttcgcgaaatctagtcgcaacaaaagcaaactttaaaagaaggagatatacatatgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataataagaaggagatatacatatgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 37 - DNA, 2005 bp, Artificial Sequence
  Description of Artificial Sequence: Synthetic polynucleotide
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgaataatccacaaacaggacaatcaacaggcctcttgggcaatcgttggttctacttggtattagcagttttgctgatgtgtatgatctcgggtgtccaatattcctggacactgtacgctaacccggttaaagacaaccttggcgtttctttggctgcggttcagacggctttcacactctctcaggtcattcaagctggttctcagcctggtggtggttacttcgttgataaattcggtccaagaattccattgatgttcggtggtgcgatggttctcgctggctggaccttcatgggtatggttgacagtgttcctgctctgtatgctctttatactctggccggtgcaggtgttggtatcgtttacggtatcgcgatgaacacggctaacagatggttcccggacaaacgcggtctggcttccggtttcaccgctgccggttacggtctgggtgttctgccgttcctgccactgatcagctccgttctgaaagttgaaggtgttggcgcagcattcatgtacaccggtttgatcatgggtatcctgattatcctgatcgctttcgttatccgtttccctggccagcaaggcgccaaaaaacaaatcgttgttaccgacaaggatttcaattctggcgaaatgctgagaacaccacaattctgggttctgtggaccgcattcttttccgttaactttggtggtttgctgctggttgccaacagcgtcccttacggtcgcagcctcggtcttgccgcaggtgtgctgacgatcggtgtttcgatccagaacctgttcaatggtggttgccgtcctttctggggtttcgtttccgataaaatcggccgttacaaaaccatgtccgtcgttttcggtatcaatgctgttgttctcgcacttttcccgacgattgctgccttgggcgatgtagcctttatcgccatgttggcaatcgcattcttcacatggggtggtagctacgctctgttcccatcgaccaacagcgatattttcggtacggcatactctgccagaaactatggtttcttctgggctgcaaaagcaactgcctcgatcttcggtggtggtctgggtgctgcaattgcaaccaacttcggatggaataccgctttcctgattactgcgattacttctttcatcgcatttgctctggctaccttcgttattccaagaatgggccgtccagtcaagaaaatggtcaaattgtctccagaagaaaaagctgtacattaa

SEQ ID NO: 38 - DNA, 1381 bp, Artificial Sequence
  Description of Artificial Sequence: Synthetic polynucleotide
cattaattcctaatttttgttgacactctatcattgatagagttattttaccactccctatcagtgatagagaaaagtgaactctagaaataattttgtttaactttaagaaggagatatacatatgaataatccacaaacaggacaatcaacaggcctcttgggcaatcgttggttctacttggtattagcagttttgctgatgtgtatgatctcgggtgtccaatattcctggacactgtacgctaacccggttaaagacaaccttggcgtttctttggctgcggttcagacggctttcacactctctcaggtcattcaagctggttctcagcctggtggtggttacttcgttgataaattcggtccaagaattccattgatgttcggtggtgcgatggttctcgctggctggaccttcatgggtatggttgacagtgttcctgctctgtatgctctttatactctggccggtgcaggtgttggtatcgtttacggtatcgcgatgaacacggctaacagatggttcccggacaaacgcggtctggcttccggtttcaccgctgccggttacggtctgggtgttctgccgttcctgccactgatcagctccgttctgaaagttgaaggtgttggcgcagcattcatgtacaccggtttgatcatgggtatcctgattatcctgatcgctttcgttatccgtttccctggccagcaaggcgccaaaaaacaaatcgttgttaccgacaaggatttcaattctggcgaaatgctgagaacaccacaattctgggttctgtggaccgcattcttttccgttaactttggtggtttgctgctggttgccaacagcgtcccttacggtcgcagcctcggtcttgccgcaggtgtgctgacgatcggtgtttcgatccagaacctgttcaatggtggttgccgtcctttctggggtttcgtttccgataaaatcggccgttacaaaaccatgtccgtcgttttcggtatcaatgctgttgttctcgcacttttcccgacgattgctgccttgggcgatgtagcctttatcgccatgttggcaatcgcattcttcacatggggtggtagctacgctctgttcccatcgaccaacagcgatattttcggtacggcatactctgccagaaactatggtttcttctgggctgcaaaagcaactgcctcgatcttcggtggtggtctgggtgctgcaattgcaaccaacttcggatggaataccgctttcctgattactgcgattacttctttcatcgcatttgctctggctaccttcgttattccaagaatgggccgtccagtcaagaaaatggtcaaattgtctccagaagaaaaagctgtacattaa

SEQ ID NO: 39 - DNA, 1300 bp, Artificial Sequence
  Description of Artificial Sequence: Synthetic polynucleotide
ctctagaaataattttgtttaactttaagaaggagatatacatatgaataatccacaaacaggacaatcaacaggcctcttgggcaatcgttggttctacttggtattagcagttttgctgatgtgtatgatctcgggtgtccaatattcctggacactgtacgctaacccggttaaagacaaccttggcgtttctttggctgcggttcagacggctttcacactctctcaggtcattcaagctggttctcagcctggtggtggttacttcgttgataaattcggtccaagaattccattgatgttcggtggtgcgatggttctcgctggctggaccttcatgggtatggttgacagtgttcctgctctgtatgctctttatactctggccggtgcaggtgttggtatcgtttacggtatcgcgatgaacacggctaacagatggttcccggacaaacgcggtctggcttccggtttcaccgctgccggttacggtctgggtgttctgccgttcctgccactgatcagctccgttctgaaagttgaaggtgttggcgcagcattcatgtacaccggtttgatcatgggtatcctgattatcctgatcgctttcgttatccgtttccctggccagcaaggcgccaaaaaacaaatcgttgttaccgacaaggatttcaattctggcgaaatgctgagaacaccacaattctgggttctgtggaccgcattcttttccgttaactttggtggtttgctgctggttgccaacagcgtcccttacggtcgcagcctcggtcttgccgcaggtgtgctgacgatcggtgtttcgatccagaacctgttcaatggtggttgccgtcctttctggggtttcgtttccgataaaatcggccgttacaaaaccatgtccgtcgttttcggtatcaatgctgttgttctcgcacttttcccgacgattgctgccttgggcgatgtagcctttatcgccatgttggcaatcgcattcttcacatggggtggtagctacgctctgttcccatcgaccaacagcgatattttcggtacggcatactctgccagaaactatggtttcttctgggctgcaaaagcaactgcctcgatcttcggtggtggtctgggtgctgcaattgcaaccaacttcggatggaataccgctttcctgattactgcgattacttctttcatcgcatttgctctggctaccttcgttattccaagaatgggccgtccagtcaagaaaatggtcaaattgtctccagaagaaaaagctgtacattaa

SEQ ID NO: 40 - DNA, 4874 bp, Artificial Sequence
  Description of Artificial Sequence: Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatatgaccagtgcagctacggtgaccgcgagctttaatgacactttttctgtgagcgataatgtcgcggtaatcgtaccggaaaccgatacgcaggtcacctaccgtgatctttcccacatggtaggacactttcaaacaatgttcacgaacccgaatagtcctctgtacggggcggtctttcgtcaagacacggtagcgattagcatgcgtaacggccttgaatttattgtggctttccttggagccacgatggatgcgaaaattggtgcgccactgaatcccaattataaagagaaggagtttaatttttacctgaatgacttaaagtccaaagccatctgcgtgccgaaaggcaccaccaaactgcaaagttcagaaattcttaagagtgcgtccacgttcgggtgctttattgtggaactggcgtttgacgccacccgttttcgtgttgaatatgacatttactccccggaggacaattataaacgtgtgatctaccgcagccttaacaatgctaagtttgtcaacacaaaccctgtcaagttcccgggtttcgcccgcagctcggatgttgcacttattttgcatacctcaggcaccactagtaccccaaagaccgtacccctcttgcatctgaatattgtccgttcaaccctgaatatcgccaacacttacaaacttaccccgctggatcgctcctatgttgtaatgccgctgtttcatgtacatggattaatcggcgtcttactgagtacgttccgcacccagggcagtgtagtcgtcccggacggctttcatccgaagctcttctgggatcagtttgttaaatataactgcaattggtttagttgcgtcccaacgatctctatgattatgttgaatatgcccaaaccgaatccgtttccgcacattcgctttatccgctcatgtagcagcgcgctggcgccagcaacgtttcacaagctggaaaaagaatttaatgccccagttctggaagcgtacgcgatgacagaagcatctcatcagatgaccagtaacaatctgcctcccggtaaacgtaaaccggggaccgtgggccaacctcaaggtgtaaccgtagtaatcctggatgacaacgataacgttctgccgcccggcaaagttggcgaggtgtcgatccgtggggagaacgtcaccctgggctacgctaataacccgaaagctaacaaagaaaacttcactaaacgtgaaaactatttccgtaccggggatcagggctacttcgacccggagggctttctcgtgctgaccggccgcattaaagaattgatcaatcgcggtggtgaaaaaattagtcctattgaactggacggaatcatgctctcgcatcctaaaatcgacgaggcggtggcgttcggcgttccagatgatatgtatggccaagtcgttcaggcggcaatcgtgttgaaaaagggggaaaagatgacctatgaagaattagtgaatttcctgaaaaagcatttagcaagctttaaaatcccaaccaaagtctactttgtggataagctgcctaaaacggccaccgggaagattcaacgtcgcgtaatcgccgaaaccttcgcgaaatctagtcgcaacaaaagcaaactttaaaagaaggagatatacatatgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataataagaaggagatatacatatgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 41 - DNA, 213 bp, Artificial Sequence
  Description of Artificial Sequence: Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacat

SEQ ID NO: 42 - DNA, 1470 bp, Artificial Sequence
  Description of Artificial Sequence: Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatatgaataatccacaaacaggacaatcaacaggcctcttgggcaatcgttggttctacttggtattagcagttttgctgatgtgtatgatctcgggtgtccaatattcctggacactgtacgctaacccggttaaagacaaccttggcgtttctttggctgcggttcagacggctttcacactctctcaggtcattcaagctggttctcagcctggtggtggttacttcgttgataaattcggtccaagaattccattgatgttcggtggtgcgatggttctcgctggctggaccttcatgggtatggttgacagtgttcctgctctgtatgctctttatactctggccggtgcaggtgttggtatcgtttacggtatcgcgatgaacacggctaacagatggttcccggacaaacgcggtctggcttccggtttcaccgctgccggttacggtctgggtgttctgccgttcctgccactgatcagctccgttctgaaagttgaaggtgttggcgcagcattcatgtacaccggtttgatcatgggtatcctgattatcctgatcgctttcgttatccgtttccctggccagcaaggcgccaaaaaacaaatcgttgttaccgacaaggatttcaattctggcgaaatgctgagaacaccacaattctgggttctgtggaccgcattcttttccgttaactttggtggtttgctgctggttgccaacagcgtcccttacggtcgcagcctcggtcttgccgcaggtgtgctgacgatcggtgtttcgatccagaacctgttcaatggtggttgccgtcctttctggggtttcgtttccgataaaatcggccgttacaaaaccatgtccgtcgttttcggtatcaatgctgttgttctcgcacttttcccgacgattgctgccttgggcgatgtagcctttatcgccatgttggcaatcgcattcttcacatggggtggtagctacgctctgttcccatcgaccaacagcgatattttcggtacggcatactctgccagaaactatggtttcttctgggctgcaaaagcaactgcctcgatcttcggtggtggtctgggtgctgcaattgcaaccaacttcggatggaataccgctttcctgattactgcgattacttctttcatcgcatttgctctggctaccttcgttattccaagaatgggccgtccagtcaagaaaatggtcaaattgtctccagaagaaaaagctgtacattaa

SEQ ID NO: 43 - DNA, 3225 bp, Artificial Sequence
  Description of Artificial Sequence: Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatatgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataataagaaggagatatacatatgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 44 - DNA, 3055 bp, Artificial Sequence
  Description of Artificial Sequence: Synthetic polynucleotide
ctctagaaataattttgtttaactttaagaaggagatatacatatgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataataagaaggagatatacatatgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 45 - DNA, 4388 bp, Artificial Sequence
  Description of Artificial Sequence: Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatatgacaaataatgaaagcaaagggccgtttgaaggcttattagttatcgatatgacacatgtccttaatggacctttcggaactcaacttctttgtaatatgggcgcaagggtaattaaagttgagccgccgggtcatggtgatgatacccgcacatttggtccctatgtggatggacagtcactctattacagttttattaatcatggcaaagagagtgtggttcttgatttaaagaatgatcacgataaaagtatatttataaatatgctcaaacaagctgatgtattagctgagaattttcgcccaggtacaatggaaaaactggggttttcatgggaaacgcttcaagaaatcaacccgcgcctcatatatgcttcatcgtcaggtttcggacataccggtccgctaaaagatgctcctgcctacgataccatcattcaggcaatgagcgggataatgatggaaacaggatatcctgatgctccgccagtgcgcgttggtacatctcttgcggatctatgcggcggtgtctatttattcagcggaatagtgagtgcactttatggccgcgaaaagagccagagaggggcgcatgtcgatatagcgatgtttgatgccacgctgagttttctggagcatggtctgatggcatatatcgcaactgggaagtcaccacaacgtctgggaaatcgccatccctacatggcaccttttgatgttttcaatactcaggataagccgattacgatttgttgtggtaatgacaagcttttttctgcgttatgccaggcactggagcttacggaactggttaatgatccccgatttagcagcaatattttacgcgtacaaaaccaggctattcttaaacaatatattgagcggacgttaaaaacgcaggcagctgaagtttggttagccagaatacatgaagttggtgtacccgtcgcgccgttattaagtgtggctgaggccattaaattgccacaaactcaggcgagaaatatgttgattgaagccgggggaataatgatgccgggtaatccgataaaaatcagcggctgcgcggacccgcatgttatgccgggagcggcaacgctcgaccagcatggggaacaaattcgccaggagttctcatcataaaagaaggagatatacatatgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataataagaaggagatatacatatgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 46 - DNA, 4388 bp, Artificial Sequence; identical to SEQ ID NO: 45
  Description of Artificial Sequence: Synthetic polynucleotide

SEQ ID NO: 47 - DNA, 60 bp, Artificial Sequence
  Description of Artificial Sequence: Synthetic primer
tagaactgatgcaaaaagtgctcgacgaaggcacacagatgtgtaggctggagctgcttc

SEQ ID NO: 48 - DNA, 60 bp, Artificial Sequence
  Description of Artificial Sequence: Synthetic primer
gtttcgtaattagatagccaccggcgctttaatgcccggacatatgaatatcctccttag

SEQ ID NO: 49 - DNA, 52 bp, Artificial Sequence
  Description of Artificial Sequence: Synthetic primer
caacacgtttcctgaggaaccatgaaacagtatttagaactgatgcaaaaag

SEQ ID NO: 50 - DNA, 46 bp, Artificial Sequence
  Description of Artificial Sequence: Synthetic primer
cgcacactggcgtcggctctggcaggatgtttcgtaattagatagc

SEQ ID NO: 51 - DNA, 36 bp, Artificial Sequence
  Description of Artificial Sequence: Synthetic primer
atatcgtcgcagcccacagcaacacgtttcctgagg

SEQ ID NO: 52 - DNA, 47 bp, Artificial Sequence
  Description of Artificial Sequence: Synthetic primer
aagaatttaacggagggcaaaaaaaaccgacgcacactggcgtcggc

SEQ ID NO: 53 - DNA, 3383 bp, Artificial Sequence
  Description of Artificial Sequence: Synthetic polynucleotide
ggtaccgtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaaatgagcaaagtcagactcgcaattatggatcctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 54 - DNA, 3258 bp, Artificial Sequence
  Description of Artificial Sequence: Synthetic polynucleotide
ggtacccatttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacctatgacgacctacgatcgggatcctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 55 - DNA, 3386 bp, Artificial Sequence
  Description of Artificial Sequence: Synthetic polynucleotide
ggtaccgtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacatatgactatgattacggattctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 56 - DNA, 3261 bp, Artificial Sequence
  Description of Artificial Sequence: Synthetic polynucleotide
ggtacccatttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacatatgactatgattacggattctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 57 - DNA, 3279 bp, Artificial Sequence
  Description of Artificial Sequence: Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacatatgctatgattacggattctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 58 - DNA, 1921 bp, Artificial Sequence
  Description of Artificial Sequence: Synthetic polynucleotide
ttacccgtctggattttcagtacgcgcttttaaacgacgccacagcgtggtacggctgatccccaaataacgtgcggcggcgcgcttatcgccattaaagcgtgcgagcacctcctgcaatggaagcgcttctgctgacgagggcgtgatttctgctgtggtccccaccagttcaggtaataattgccgcataaattgtctgtccagtgttggtgcgggatcgacgcttaaaaaaagcgccaggcgttccatcatattccgcagttcgcgaatattaccgggccaatgatagttcagtagaagcggctgacactgcgtcagcccatgacgcaccgattcggtaaaagggatctccatcgcggccagcgattgttttaaaaagttttccgccagaggcagaatatcaggctgtcgctcgcgcaaggggggaagcggcagacgcagaatgctcaaacggtaaaacagatcggtacgaaaacgtccttgcgttatctcccgatccagatcgcaatgcgtggcgctgatcacccggacatctaccgggatcggctgatgcccgccaacgcgggtgacggctttttcctccagtacgcgtagaaggcgggtttgtaacggcagcggcatttcgccaatttcgtcaagaaacagcgtgccgccgtgggcgacctcaaacagccccgcacgtccacctcgtcttgagccggtaaacgctccctcctcatagccaaacagttcagcctccagcaacgactcggtaatcgcgccgcaattaacggcgacaaagggcggagaaggcttgttctgacggtggggctgacggttaaacaacgcctgatgaatcgcttgcgccgccagctctttcccggtccctgtttccccctgaatcagcactgccgcgcgggaacgggcatagagtgtaatcgtatggcgaacctgctccatttgtggtgaatcgccgaggatatcgctcagcgcataacgggtctgtaatcccttgctggaggtatgctggctatactgacgccgtgtcaggcgggtcatatccagcgcatcatggaaagcctgacgtacggtggccgctgaataaataaagatggcggtcattcctgcctcttccgccaggtcggtaattagtcctgccccaattacagcctcaatgccgttagctttgagctcgttaatttgcccgcgagcatcctcttcagtgatatagcttcgctgttcaagacggaggtgaaacgttttctgaaaggcgaccagagccggaatggtctcctgataggtcacgattcccattgaggaagtcagctttcccgcttttgccagagcctgtaatacatcgaatccgctgggtttgatgaggatgacaggtaccgacagtcggctttttaaataagcgccgttggaacctgccgcgataatcgcgtcgcagcgttcggttgccagttttttgcgaatgtaggctactgccttttcaaaaccgagctgaataggcgtgatcgtcgccagatgatcaaactccaggctgatatcccgaaatagttcgaacaggcgcgttaccgagaccgtccagatcaccggtttatcgctattatcgcgcgaagcgctatgcacagtaaccatcgtcgtagattcatgtttaaggaacgaattcttgttttatagatgtttcgttaatgttgcaatgaaacacaggcctccgtttcatgaaacgttagctgactcgtttttcttgtgactcgtctgtcagtattaaaaaagatttttcatttaactgattgtttttaaattgaattttatttaatggtttctcggtttttgggtctggcatatcccttgctttaatgagtgcatcttaattaacaattcaataacaagagggctgaatagtaatttcaacaaaataacgagcattcgaatg

SEQ ID NO: 59 - DNA, 967 bp, Unknown
  Description of Unknown: clbA sequence
caaatatcacataatcttaacatatcaataaacacagtaaagtttcatgtgaaaaacatcaaacataaaatacaagctcggaatacgaatcacgctatacacattgctaacaggaatgagattatctaaatgaggattgatatattaattggacatactagtttttttcatcaaaccagtagagataacttccttcactatctcaatgaggaagaaataaaacgctatgatcagtttcattttgtgagtgataaagaactctatattttaagccgtatcctgctcaaaacagcactaaaaagatatcaacctgatgtctcattacaatcatggcaatttagtacgtgcaaatatggcaaaccatttatagtttttcctcagttggcaaaaaagattttttttaacctttcccatactatagatacagtagccgttgctattagttctcactgcgagcttggtgtcgatattgaacaaataagagatttagacaactcttatctgaatatcagtcagcatttttttactccacaggaagctactaacatagtttcacttcctcgttatgaaggtcaattacttttttggaaaatgtggacgctcaaagaagcttacatcaaatatcgaggtaaaggcctatctttaggactggattgtattgaatttcatttaacaaataaaaaactaacttcaaaatatagaggttcacctgtttatttctctcaatggaaaatatgtaactcatttctcgcattagcctctccactcatcacccctaaaataactattgagctatttcctatgcagtcccaactttatcaccacgactatcagctaattcattcgtcaaatgggcagaattgaatcgccacggataatctagacacttctgagccgtcgataatattgattttcatattccgtcggtggtgtaagtatcccgcataatcgtgccattcacatttag

SEQ ID NO: 60 - DNA, 424 bp, Unknown
  Description of Unknown: clbA knock-out sequence
ggatggggggaaacatggataagttcaaagaaaaaaacccgttatctctgcgtgaaagacaagtattgcgcatgctggcacaaggtgatgagtactctcaaatatcacataatcttaacatatcaataaacacagtaaagtttcatgtgaaaaacatcaaacataaaatacaagctcggaatacgaatcacgctatacacattgctaacaggaatgagattatctaaatgaggattgatgtgtaggctggagctgcttcgaagttcctatactttctagagaataggaacttcggaataggaacttcggaataggaactaaggaggatattcatatgtcgtcaaatgggcagaattgaatcgccacggataatctagacacttctgagccgtcgataatattgattttcatattccgtcggtgg

SEQ ID NO: 61 - protein, 264 aa, Escherichia coli
MKQYLELMQKVLDEGTQKNDRTGTGTLSIFGHQMRFNLQDGFPLVTTKRCHLRSIIHELLWFLQGDTNIAYLHENNVTIWDEWADENGDLGPVYGKQWRAWPTPDGRHIDQITTVLNQLKNDPDSRRIIVSAWNVGELDKMALAPCHAFFQFYVADGKLSCQLYQRSCDVFLGLPFNIASYALLVHMMAQQCDLEVGDFVWTGGDTHLYSNHMDQTHLQLSREPRPLPKLIIKRKPESIFDYRFEDFEIEGYDPHPGIKAPVAI

SEQ ID NO: 62 - DNA, 795 bp, Escherichia coli
atgaaacagtatttagaactgatgcaaaaagtgctcgacgaaggcacacagaaaaacgaccgtaccggaaccggaacgctttccatttttggtcatcagatgcgttttaacctgcaagatggattcccgctggtgacaactaaacgttgccacctgcgttccatcatccatgaactgctgtggtttctgcagggcgacactaacattgcttatctacacgaaaacaatgtcaccatctgggacgaatgggccgatgaaaacggcgacctcgggccagtgtatggtaaacagtggcgcgcctggccaacgccagatggtcgtcatattgaccagatcactacggtactgaaccagctgaaaaacgacccggattcgcgccgcattattgtttcagcgtggaacgtaggcgaactggataaaatggcgctggcaccgtgccatgcattcttccagttctatgtggcagacggcaaactctcttgccagctttatcagcgctcctgtgacgtcttcctcggcctgccgttcaacattgccagctacgcgttattggtgcatatgatggcgcagcagtgcgatctggaagtgggtgattttgtctggaccggtggcgacacgcatctgtacagcaaccatatggatcaaactcatctgcaattaagccgcgaaccgcgtccgctgccgaagttgattatcaaacgtaaacccgaatccatcttcgactaccgtttcgaagactttgagattgaaggctacgatccgcatccgggcattaaagcgccggtggctatctaa

SEQ ID NO: 63 - DNA, 10859 bp, Artificial Sequence
  Description of Artificial Sequence: Synthetic polynucleotide
ttacgctgcagttacggtaactttgcagaccgcaacgaaattaccgtcgctggtcataacaataacgtcagcggtgcctgccgccacgccggtgacggtgatcgcattaccgctaacggtgaccgttgcttttgccccgtctgaggttgccacacggaacgaggtatctgaggcactggctgggttaaccgtcacattgagcgttgtggttgcgccgacggccacgcttgccgtggctttatcgagcgtaacgcctgtcacggggatattcggggtcccgctttcttctgccagttccggcttgccggtattggtaattttcgctgtacgggttatgacctcttttgccggaatggctttacccaggctgctgcaccagccgcggaaaacgtcgacggtaccgttcgggtatttgattttgtaatagcgtactgagccatcaataaaccatgcgacaaggtctttttgcccttcttcgcccggcttccaggcgagggtgaacgaggtatcgccagcagattttgccccctgggccgtcgcgttccagtcggcatcctcgtcgtcgaggtaagtgtcgtcatacgattcggcggtcatttcgcccggcgtcagctctttaattttcgccaggcggttccagtcgatatccgagagtgggttagcgaaagcgttgcccgttccggtgtaaagccagagggtggtaccggcacctttcacaggggccagcgggtttggagtaggcataagtacctcttaaattgaataggtgattaagtacgtgaaatcgactgaaccccaggtggccatttcatcatcccgctgatagtcataaccctgcggggtgaacgtctcgaccagttcggtcagacctgggatgaaggccattgccggatacactttctcttccatccaggaatcaagcgcgctgtcggggctggaggctttaagaaatacctcgatgtgaacaaccgcctgccacgaatcttcgtcaagcgaatcgccggtgtactccgcgtcagaaaggtatacagccacggcagggagatcctgctcttcaagaaaaacagggcgcccgtcaaaccaggtgaccgtgtcggtgatctcggctttcagtttggccagaatggctgcacgaattgcgctgtgtctgttcatcgcttcaggtggatcctcagttggtttttcagggctgcggaaagttctttgggcatatcgctttcaataaggcgctttgaaatagcggtgaaggccacggtgagcggtgtctcaagaggaactttgaccacatcaatcggataacgggcctgacctacgcgccgcatgacctgccagcgcccgttcgcaagctgttggataaaagcgttacgaaaggtatagggcccgattttaaggacgctgcccgctccgtttctggccccttttttacgcgagagcctgacgcgcgccgtgccgagctttatcgcaggaagattaccgcggttgatttttatcgacgcgaccgggcgatcgtgacgggccttgcgcagacgggaacgctggcggaccagacgaaccggaagcccctttttccggttatcatcaactgttgcttctttcgctacagctttgctcccctggcttatcgttcttctggccaccctgttaagtgcttttgcggttgcctcaggaacgattaaccggctgaggctgttcaggttctgaatagccctttccagtcctttcacagacatagcgcctcctcattcgagatggatgcggggttttccgttgaacatgtcatagcgggtaacgatcaggttcttaccgtcgtagtcgacgctgtcgtttcggcgtggctggtaaagctcagagaaaaccaccagcgaagtacctgttcccgacaatggccccatttcctcgagttgctcggcgggaacaacgtcatagctgctgccattgatgatcgctgtctttcccatcttttttatagtggccgcgtccatgcgcgccgccatccggtcaaaggagttaggcattgatcttaacttcaacaacggtggtgtttgcccctgcatcttcccaggcgatgcccgcggcaacggcgtccgtttcttcgatcgtgattttgccgtccttcagatacacctgcgccccggcagtaaccgcatctgcggatacttttggcaggaggaaaacaccctcagtaaaaccgtccccggtatcgccagccgggatatcggtaattgccaccgcgataagttttccaacaacaaccgggtcgccgctgtgaacatcggttgcaccactgtttaccagagggatcgttttcccgtcctgcgcatagttcttagccataacttctccattcagcccctttcgaggctggtttcaggtataaaaaaagcccttacgggcgtctgtttgtcaggactgttttttactgaccagaggatttggtcatgccgcgatagtccagcggcgccacgccagcatcaatacgcactttcgtggcgataccatcagtggtgaagccttcctgctgatcgatgtatggcgtgtcgacgccgttgagataagcgacctcaatggtgtcggtgcccttcgcggcagccagataccaggctttcgcatcagcttcatccagacgtggttcggcaatgacttctgcaaagttctggatagggttaacgatcccggcattgatgtctgcacctttaacactggccgacttgatggtctgatttgccagagtttccagggcgacgggcaccagcatgtaggccggacggatattcagggttcgctccccctccttctgcagacgcatcagcttgcgcgattcgtccaggctggccacagaaattgcacccgagctcaggttcttgtgatcggcatggaacagcgcctttccgtctgagagtttcgggtttttggtcagaatggcgtaaaccagatcgccaatcgttgctttcgccgcgcgccccatcttcatcggtacgtcggtaagctggttcagatcgtcgttgatgatcgcctggcgagttactgagaagatttcaccatacgtggcaagcgcgatggtttcgcctttgtcactggtagtgatgtacttgtactcagccccttcgcgaacctgtcgcagagaagggaacccacccataccgacacgatgcgccgttttgaagtccgacagctggccttttttggtccactgctcgaaggtttcctgcgcctcgtcccagccctgaatcagcgctttgttcgcaacatcaagcagaatgttgccaaagtcagaggtgctgtgggtcagcgccaggccaaccatctgcatcgggttgtagctggccacgccgatacctttttctgtcagggccatacgcgcatactcgcgcagcgtcataccgttataaacgttatcccgctcctgaccttcgaacccggcacgcgccatcagtgcctggcgaataccatccgcgacgaagttaccgttgcccgcatgaatatgcggctgagtggttttattggacggcgtggccgttttaccgagttctgccagcagcaaatctttcgccttatcgacggagcaatcagggtcggccacacactgattctgcagttccatgtgcttattaccgaacatggcaaagagatcgccgatagcgttaacacgggttttctgctcagccaacacctgcgcgcggatcgcattttcatccggtgccgggtctgtttttgcctgcggtgcctgaggctgggtaataaccgggtcacgctgggtagtgttgcgcggcggggtgatcatgttgcgaatgctttttggcattttttcaaattcctcaatacgttttgaatgaatacaggccatagcctgaagggatggtgtcacctggtcggcaaaacccagttcaaggcactcgctgccgttcatccaggtttcgtcctccagcattaccgcaatttcttcggtggattttccggttttctgtgcataagccgggataagaacggattcaaccttgtcgagaagatccgcatagtcgcgcatatcgctcgcgtcaccaccagcaaacccccagggcttatggatcatcatcatcgtgttttcaggcatgatgaccggattgcctaccatcgcaatcaccgaggccatggaggccgccagaccgtcgatatgtacggtaatcgccgcgccgtggtgcttcagcgcgttataaatagcaattccgtcgaagacatcaccaccgggcgagttgatataaaggttgatgtgggtgacgtccccaagtgcccggagatcattgacgaactgtttcgccgttacgccccagtacccgatttcgtcataaataaaaatgtcggcctcactgttattgctggcctgcatgcggaaccacgaattactttttgcgctggctttcggacggtggcgcgcccggttctttggcttcggcactggtgcctcctttatcattggcggggtcggtgtcaaacaccaggccctgttcacggttctcgtcaacctcagctttacggcgtgacttaacatcatccgggttgcgaccgctggcacgtatccagtcggattcagtagcagcaccgccgcggatctgcgttttccaggcattcgcttctttaacgggatcaatccacggcataacgggccccgaataaaccgcgttataaagcgagtccatatcaatgcctctcggcagcttgatttctccggcagcaatagccatcttgagccaggctcggtacatgggccgggtcactgaaccgatgaaccagtcctgaagaatcagatagccgtcggttgactcgacaagctcctgccgctgggcactgtacgttccgttgtagtttctggatgtgctggaaaagctgaggcgactgccggcggacacggcacgcagctgtccgttacgaaaagattcgaggttagggttcgggcgatcggatttaatcatcccgatttcttccccggcctgcagttcgtcatagagcataccgggctgaatcatcagctcgcggtcatcgctgcttgaatcagaatcgaagctctgtccgtcgccttttttgatatacatgccgagtgccgcagcaattctggcagcagtaagctccgagtcctcgtattctttcagcgcgctcagacgcatcagaacaccagacaaaagagacgttccgcgggtctggtgcaggcgtcgggtgaatttgagatgcagcatgttctctgcatctatctctttggtatcaaactgacgcccggatactggcaggcttttatagacctgatattttttcgggcgtccccagttatcgacaaaaacgccctgattgagctgggtggcagcatcgctgttcatcggcacaaagtccggctccagcgcttccagccagaacggcacgccagcaaccggctgaagaccatttccggtaccgcgaaccagctgagcaaatacctcaccgtcccggagccacgttcgcagcatcagccgctccagcattgggcgggtaaactgggttgtgacatctggccttacggaccattcgccccactttcggcggatatcagtggccagctttttagcgatcttcccgttactcagcatcggatgcggttcaactatgatgcccttcgcacccaccaccctttcttccagcttgtcgaaaacgccgatcaccagatcgtggttgttatccagccagcgcgcctgctgcctcagcgaaaccgcccccatctggctgagctgatcggctgaacgattttccttctgggctttgtgggtacgcgtttgctttaccgcctcatacgctttaataactgcgcgggcacgcaggcgtgaggctttccagcctggtgaaaacaggccaatcgcatcatctaaaaaactcatccaaacctcgccagcctgtagccgggtcgcccacggcgtttgttattgagcgttgccagtcgtcgctcccattcctgacggccttttctgatttccgacaggttttcgagcgtcatctgctgcccgttgaaagtgattgatttcccctccagaacagacagctcggctgcagcatagcggtcgatcatgttttgaatatctgctggattcacacccaacctcctgacgaagaccacggattagcctgctcggttacgggcttctcacgttttggttttggtttagatttcggcgcaggcggcggggatggcatttcgccagcttccgtctgcgtgtcctcgatccacgtttcccgccgtgcccactcaggagctgacggccatttgattttttcgtaaccactaaggatggcgagcgcgtcggcataaacgagcaggtcaaatgcttcgtttgcgccccggccgggcttactccatttcccttcattcgagcgttcctcatacgtcagttcgtcatagaaccagctgcccagccaggcggggaaatgcacatagccagggccgggtgaatcacgccacagcgcattattcacccggtctttaagggcatcggtctggagaagataaagaggcacatcaccagtcgcctgtgcgcggcgcgttgatctgcccgtgttgtcgggaaacgttcgctggataagtttgctgcgcctgacgctgtcccctttgaagagatagatacgcttacccagcccctcacggcgacatctgcgccagaacttgtaggcattatccgtcacgccatcttcgccccctgagtccacggccatcgacatcagccgcatgccctttgatgggtcagctgcgagcggccacgttttatcaaagacgtcagtgagtaaaagatcccagtcctccggatagctcgccggatccacctgaatgctttcaccgttgccgtcgcagcgcagcgaatgccggatgttgtaacggtcaactatccagcgctcacccatacttccataacccgtaatctgcacaacaaagcgccggttgcgcccggcctgcacgtccacggtcgcagtgagaaactgcacgccgttcggtaccgaacgttttgggacgtcttcggcacgctgctcgagcaattcacttttacgctgctccatgctggctcgcggcaaatagggcctgccgaaatcggtgttgatcaccgtcttcagggtttcttcgctgcgcgtggattcatattcctgctcggcggtcagaaacttataaataagctgcgcccaggtctggtaagcagctgccggaccttccatccagaaggaggcaatacgggaacgacggccatcaccgctaaccaggcctttcctgtcgatggtttgcccgtcccggagccagacacatttcatgttaagcgcacgcttcatgtccggtgtgatcctgcctttacaggcagggcactgaagaaacgccgcttcgctggcaagcacaggatcgctgctgtcgcggtatccggtcatattgtccatttccggctggaaatattcgccgcaatgcgggcatggccagtaaagacgacggcggtcaccacggttatagagcgataaaattccggtggtcggaggggcttcatggggcgtggagcgccgccattttgtgtctctgatatccctcccgggcgagctctcaaccagcgtcatcccggaggacatgaatgtcgtggttcgtttcgatgccagtgaaaaagcatccccctccccgtcgatatcttccggaaagcggtcataatccgtcagcgccacacttttatagtccgaggacgacatgatattgacggatggccagcccagcttcagatagttaccggcgcggaatgtacggtcgtagacgttgttatcgttacgtcttgggcttagccgggttttaacttcagggctacagcgaaaagtacggtccaggcgttttttggaatgctcgcgcgctttttcctcagatacctgaattacaagcatatctgccggatcgcagacaatgttataaacgatccagccgtcaatcagcccgatggttttacccgttcgcgctgggcccacaaacacaaccgcatcgtattcacgcgatgccagacagttcatcggctcaatcacatagggtgccagatccggatcccacggaactgagtttcccgcccccattggcacgcgcatataagtactgaccgcatcggccaccggcatacgacgcggggctcgtaaaataccggaaacatcgcggcggatgtccctggcggatgcccgctttgccatcagtcctcctcaggctgctcctcctcttttccagcgtcctgcaccttctccgccatctggtcgcgcagatcatcgataacgctttgcacacgaactaccgcagcaggcgttaaagcacagtcgcgctcgagcacatccgggagggtttcaagtaccatgacgacggctttcgccatcaatgagaattctcgcgccacttcatctgcgggtattaactgccccgtatcctgttcgaacttcagcctctcgttctctgctttccagtgggacagcctgtcagaagggggcatatcgtcgatgttggccgaaacggtagggatcatcagttcggtcagaatgtcggtcaccagatagagctttaacttgctattgctgcctggagcaggttcaacatttttcagtctcgcggcaaccgtctgacggtgtacgccggttatccctgccagctggttgatattgagttttaaagtggcaatttcctggtccatgatggtgaacactttttgaacgattcgacatgttgcgaaaatggcctctaattaaatcaaagacctgcgcacatgatgatgatgaccctggatccgaaaaactagccgtttcccgcgagcacgccgccccgtggcagggtccccctccgggagtaccttttgataataattatcaattgcacactatcgacggcactgctgccagataacaccaccggggaaacattccatcatgatggccgtgcggacataggaagccagttcatccatcgctttcttgtctgctgccatttgctttgtgacatccagcgccgcacattcagcagcgtttttcagcgcgttttcgatcaacgtttcaatgttggtatcaacaccaggtttaactttgaacttatcggcactgacggttaccttgttctgcgctggctcatcacgctggataccaaggctgatgttgtagatattggtcaccggctgaggtgtttcgattgccgctgcgtggatagcaccatttgcgatagcggcgtccttgatgaatgacactccattgcgaataagttcgaaggagacggtgtcacgaatgcgctggtccagctcgtcgattgccttttgtgcagcagaggtatcaatctcaacgccaagcgtcatcgaagcgcaatattgctgctcaccaaaacgcgtattgaccaggtgttcaacggcaaatttctgcccttctgatgtcagaaaggtaaagtgattttctttctggtattcagttgctgtgtgtctggtttcagcaaaaccaagctcgcgcaattcggctgtgccagatttagaaggcagatcaccagacagcaacgcgccacggaaaaacagcgcataaagcacttcattagcagcgccagatagcgtaatgattttgttactcatggaatatttccttttaggcgtgagcctgtcgcacggcaatgccgcccgagaggtaaacgcaacctaacggcatcacccaggctcactactgaaagactctctttgatgtgcgcgtgcgatgcgcgtagaagactgatttatcaacctgtctttatatcaggattcattacctgactatttgtgggtaaagttcgtagtgcgctgatcgtgcaaaatgattttagttgggaacagttcgcaactctgtcccataaaaatcagcatattcccatctatcccatatccagcgcattgaccatcgggatactgaagggagattccatcatctcttagaaagatcaccatctcttttgtttcaatttgcatatagctacctggaggatttatgaatgcaaggattttcatggactattaccatgagattgattttccatctttattcgcgagagcagtggaaagcgatgacgatgtgggtactacattgcgcattcacctactttgtgagcgcatggtcgaagcatggatatgcgcatgctgtgactgccaagatctctttggaagagataaaaacaaacttttaatcgaatgtaatactaaaatatccatggcgggaaacctgggaatccccccggaacttatgaaatcacttaaaaccatcaactcaatgcgtaatgaccttgcacacaatccatcaatacaaagcattgctgattcaaggatccagagcctgaaggatactctgactgaatactttaaacagcatccaacggaacccagcatggaagaatcaaaactgggtatttttaacgccgagaatcaattaaccgaagaagtttccttagatagtgacagttcaaaaaacagacttaagttaatcttgctgttcagcaagttaatgcaggcgttaatgcaattagttgcagctaatcataatgggcgctgggataaccaatttagccaattcgtttaccatgtgaccatgaacgcaacaaagagataaatccaagcccgttttgtacgggctgttgcattatcacaggcactcagtgaatgcctgctgtaatgccgctagtcgtcgagttgcaacacaccgtgatccagtgattctgaataggcgataagtccggtataaccggggataatctcaccattatcagcttcaaattcaggaattgtgccggtggtgatggtgtattgaggctggccatcttccttcgcgaaggctgccaggtcttcaatctgcttagctgtaagaactactgtcatgctcat

SEQ ID NO: 64 - DNA, 18 bp, Artificial Sequence
  Description of Artificial Sequence: Synthetic oligonucleotide
ttgttgayryrtcaacwa

SEQ ID NO: 65 - DNA, 15 bp, Artificial Sequence
  Description of Artificial Sequence: Synthetic oligonucleotide
  modified_base (8)..(8): a, c, t, g, unknown or other
ttataatnattataa

SEQ ID NO: 66 - DNA, 170 bp, Artificial Sequence
  Description of Artificial Sequence: Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaa

SEQ ID NO: 67-208 - skipped (142 entries)

SEQ ID NO: 209 - DNA, 222 bp, Artificial Sequence
  Description of Artificial Sequence: Synthetic polynucleotide
acgttaaatctatcaccgcaagggataaatatctaacaccgtgcgtgttgactattttacctctggcggtgataatggttgcatagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 210 - DNA, 714 bp, Artificial Sequence
  Description of Artificial Sequence: Synthetic polynucleotide
tcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcat

SEQ ID NO: 211 - skipped

SEQ ID NO: 212 - protein, 237 aa, Artificial Sequence
  Description of Artificial Sequence: Synthetic polypeptide
MSTKKKPLTQEQLEDARRLKAIYEKKKNELGLSQESVADKMGMGQSGVGALFNGINALNAYNAALLTKILKVSVEEFSPSIAREIYEMYEAVSMQPSLRSEYEYPVFSHVQAGMFSPKLRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 213 - DNA, 84 bp, Artificial Sequence
  Description of Artificial Sequence: Synthetic oligonucleotide
acgttaaatctatcaccgcaagggataaatatctaacaccgtgcgtgttgactattttacctctggcggtgataatggttgcat

SEQ ID NO: 214 - DNA, 714 bp, Artificial Sequence
  Description of Artificial Sequence: Synthetic polynucleotide
atgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgccttatttaatggcatcaatgcattaaatgcttataacgccgcatcgcttacaagaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgctctcacctgagcttagaacctttaccaaaggtggtgcggaaaggtgggtaagcacaaccaaaaaagccagtgattctgcattctggcttgaggttgaaggtaattccatgacagcaccaacaggctccaagccaagctttcctgacggaatgttaattctcgttgaccctgagcaggctgttgagccaggtgatttctgcatagccagactcgggggtggtgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctga

SEQ ID NO: 215 - protein, 237 aa, Artificial Sequence
  Description of Artificial Sequence: Synthetic polypeptide
MSTKKKPLTQEQLEDARRLKAIYEKKKNELGLSQESVADKMGMGQSGVGALFNGINALNAYNAASLTRILKVSVEEFSPSIAREIYEMYEAVSMQPSLRSEYEYPVFSHVQAGMLSPELRTFTKGGAERWVSTTKKASDSAFWLEVEGNSMTAPTGSKPSFPDGMLILVDPEQAVEPGDFCIARLGGGEFTFKKLIRDSGQVFLQPLNPQYPMIPCNESCSVVGKVIASQWPEETFG

SEQ ID NO: 216 - DNA, 183 bp, Artificial Sequence
  Description of Artificial Sequence: Synthetic polynucleotide
aaatctatcaccgcaagggataaatatctaacaccgtgcgtgttgactattttacctctggcggtgataatggttgcatagctgtcaccggatgtgctttccggtctgatgagtccgtgaggacgaaacagcctctacaaataattttgtttaaaacaacacccactaagataaggtagaaac

SEQ ID NO: 217-1064 - skipped (848 entries)

SEQ ID NO: 1065 - DNA, 735 bp, Escherichia coli
atgaggattgatatattaattggacatactagtttttttcatcaaaccagtagagataacttccttcactatctcaatgaggaagaaataaaacgctatgatcagtttcattttgtgagtgataaagaactctatattttaagccgtatcctgctcaaaacagcactaaaaagatatcaacctgatgtctcattacaatcatggcaatttagtacgtgcaaatatggcaaaccatttatagtttttcctcagttggcaaaaaagattttttttaacctttcccatactatagatacagtagccgttgctattagttctcactgcgagcttggtgtcgatattgaacaaataagagatttagacaactcttatctgaatatcagtcagcatttttttactccacaggaagctactaacatagtttcacttcctcgttatgaaggtcaattacttttttggaaaatgtggacgctcaaagaagcttacatcaaatatcgaggtaaaggcctatctttaggactggattgtattgaatttcatttaacaaataaaaaactaacttcaaaatatagaggttcacctgtttatttctctcaatggaaaatatgtaactcatttctcgcattagcctctccactcatcacccctaaaataactattgagctatttcctatgcagtcccaactttatcaccacgactatcagctaattcattcgtcaaatgggcagaattga

SEQ ID NO: 1066 - DNA, 9621 bp, Escherichia coli
atggataatacctctggagattttccatgtaataagatggacacgcgtaagcagttaccgctaacaccaagtcaacaggggtttttattccattccttaaaggataagaaaaggagtaactaccatgagcattttacatgcattttttctcagcatgtagatagcgcccacttcaagtgggcgctggaaacgttatttcgaaagcatgagtgttttcgcactgattataactgggagattgatgagcgcccttgtcaggtggtgaagaccgatgtgttgccggatatatatgtgttagactgtgagcaagaggaaatacgttttctactagctaatgatgacattatcattcctgtcccgcaggatgacggtattgatgctataattcctcaactgctacaggctgatttaaaatacccattttccttgaaaacgatcccagtccgggcctaccttattcagtcaacgaaagaaagtgcttttatactatcataccatcatattgtgatggatggctggagcttatcccttttcattaaacagttgctccaactctatggagcggctgtggtcagtggtgtgagggatgatagcgccattatcccctcatctctgaaaccccttgtagacacactgtcggcccgacgtcacacctttcagcacgactattgggctgcatatcttcgggagggaacaccaacttgtatcgtgccgctgtcacaatatcacacagatactgaagccgagaacaattcttacgttaatcaaacaaatcatgtggagatcaatttgtctccggatgtgtgtcagaaaatacagacgctatgcagcgattatcgtatcacccccgcagtaatcttctatgtggcctggggcatcctgctacaacgttggtgctatgctgacgatgtgttattcggcgcgacaatatcagggcgaaatataccaattgatggtatagaagaaacactagggctatttattaacacgttgccgctgcgtctgcgtgatgatggggcgacactgttgcaacacttacaacgtatgcaccaaacactaatagctcactacagcaatgaacatgatgccttagccagcatacaacggttggtacataaagaaggtcatgctggggatctttttaataccttagtggtgttggagaattatcccgttgatatgacattattgtcatgcgcgtcgcccgtggcaattcgccatctcagtgtacatgaacaaacgcactatccgctgaccttgaccatcactcaacagaagggattccgtttcagtattgcatatgcccttaactacctgaccaacaacatggcgcaagcgttgctgatgcacctgagttatctgcttgaacaactggtggacaatccgcagcgccccattgctgcgttggtaaacttgtccccatgccagcaggcgcaggtacttcaaccctatctggaacgcatggcatgccgggattgggatagtcaatccaacgtcatcgaacaatttcatcaagttgctgcgacttcgccagcacaggtcgcagtggttgatgaattgtgcgcgttgacctattcggagttggcagcacaagctgagcagctcgcggcttatttggtacagcagggtgttatggttggcgatactgtaggcataattagcgaacgccgggtaaacaccgtggttgccatcatcgccatcatgttgatcggggctgcttatgtgcccatcagccccgactacccagtgggtaggatgcaggaaattattgatgacagcggcttggcgctgttgctggtacatggcaaaccgctagatgcattaaacgttgcgcagagtgacctctgtgcatttcccgtcgcgccctcggtggtatttccggttatcacaccagattctcgcgcttatgtgatttattcatcggggtcgacgggtaagccaaaaggtatcgcggtggcgcaccgcggtttattgcgcctgatacaaggcgacagcccgctgaaggtggagagcggtgagacaacgctgctgacctgcccatttgagtttgatgtttcggtgtttgagatgtggtctaccttgctcaaccacggcaaactagtattactcagcaaacaggcattacttgatatcaatcacattcgccgcacgatcgctgatgaacaggtggcgcgcgcctggtttacctcatccttgtttaactcctatgtggcagaaggtgccgatttcttcggtatgttacaacacattacggtgggcggcgaagcagtatctgcgtggcatgtcaacgacgtgatgcaaaaatacccgcatctggtggtgacaaacggttatgggccgacggaaaacactatttttaccaccgcatatcgtttcaacgggttgcaacccgcccgagtcccgataggatacgcggtaccgggcacctcgctctacattaccgatctccatgggcatttgttgcctatcggtgccaccggtgaactagtggcgggtggagtgggggtcgccatcggctatcagaacaacccggcgctaagtgcgacggtttttgtccctgatccttttattcccggcggtatgatgtacaaaactggcgattatgcccggttgctggatgatggctgtgttgactgttttgggcgtaaagacggtcaaattaagatcaatggacaacgaattgaaaccggagagatcgagcagcgcctgctggagtgctccggcattatcgaggcggtagtggttccttaccgcgtacgtgaaacgctgcatattgcggcagtggtctgcgtcaatgatagctatgatgaggtggaagttcgagggcaattggctgacagattgccgccattcgctattccggaatccttggtggtggtgacggagattgcaaaaagccatagtggcaaggccgacttggcgcagttgcggtatctcctgcccgcaactcagtgcaacgccgtgtccaccacgatatcagaggtgcatagtgacatggaacatgcgctgcatgctatctggcaacgcgtacttgatagacaagacattgatagcaatgcctcctttttcgccctcggtggcacctcattggataccatcagggttaaaggggatattaagcggcaacttggcttggagattgatattaccgatctctttaagtacccaacgctcacggcgttagcacattttctcgatactgccgtatcgccggaggatgcaattccaacgcgtgctgttgtctacagcgacatgccggtggcgattgtcggtatggcgggacgtttccccggtgcggcgaatattgcagcgttgtggacgctggttgtaggaggggaatcgggattaacactgttcagtgatgaagagttgcgcgcgcatggtgtgacacctgacacgcttaaacaagcgaattatataaaaaccaaagggattgttgatgatcacgaatggtttgatgcggatttctttggttatactcccaacgaggcggaatgtatggatccgcaaattcgcttattgcatcagtgctgctggcaaactctggaacacgctgggtgcgatcctgccacctttactggtgcgattggcatttatgccggactgctgacatccccccactggcttaatgcggtaatgcaagacactaccgactctaccgccctgtacaaggccagtatcctgaatatccattccgtcacagcattgattgcccatgcgcttaacctcaccggccctgccgtgacgcttgacaccgcctgctccacttcggcagtggctatccaccaggcctgcatcgcactacgtaaccgggattgcgatgcggcattggcgggcggcgtttctatcgagatgcctgcgtaccggggctatgaatatcatgaaggcatgatcaatgcgcgagatggtgtctgtcgtcctttcgatagccaagcctctggcacagtcaccggtgatggcttaggaatgctgctgctaaaacgtctggacgacgcgctggcggatcgggactgcatctatggtgtgatcaaaggttcggcggtcaataatgatggcaataacaaaatcggctataccgcgccgagtgtgatcgggcaatcaacggtaatccgcaccagtttgcgccgtgccggttttgatagcgacagtatcggattggtggaagcgcacggcaccggtacggtgctgggtgatcctattgagttacgtgcgcttaatgaggtgtttggcccaacacctgttccgttttgtgtggtttcagcgctgaaaagtaatattggccacctcaactccgctgcgggagtggcgggcgtcatcaaaacaacgctcgccctgcatcatcaagtgttgccaccaacagcgcactttcgccaactcaatcctgctatagatctatcgcgttctgcattgtacgttaaccagcaagtccaaccgtggccgtcgacgcgtcctcgtcgtgcactggtgagctccttcggcatcggcggcaccaatgccagcatcgcactagaagcacatcaacatgaggacgacccttcagcgacgggggtacgcgacagctatctgttgttgttctccgctaaaacacccgctgcgttagagttgcgcgtggcctccacactggaatatgtcaagcatggagtaggggtgcgcctgccggatgtggcttatacattgcaaactggacgcacagcctttgaccatcggcgggcttatttggtgagtcgtgggtcgaaaatcgatctctcctgtgccacgatattgcaagcggaaatcttcaatggtcagcgcacgacagcggagatctgcttcatgtttcctggtcaaggtagccaatatcacggcatggccagcgcgctctatgctcatcaacccatgtttcgccagcacatggatcgctgctttgctgcattccaacgctattcgacggtcgatctcaaggcgttgttgtttgacgatgaggatacgcgggatattgatcaaacgcagttcacacaaccggcgttgttctgtgtcgaatacagcctagcgcgcaccttgattgatctggggattacgccggacagtatgatcgggcacagtctgggcgagtatgttgcggcctgtattgctggcgtatttactcttgaggatgcgctgcacgtcattgaggcgcgcggacgtttgatgcagtccatgcgtcccggtagcatgatggcggtctaccttagtcgcgaacagttgaccccatggctagctgcagaacggggtattgaactggcagctaataatagcgcgcatttttgcgtagtcgcgggcgagcaggcggccatttcccgtttgagcacacgcttagtcgagggcgggatacagcacaggcgcctgaaaacctctcacgcattccactcggccatgatgacgccgatgctgcacgattttgcacagttgctggggcaaatcccgatgcacgcgccgcacaagcgctttatatctaatgtaagcggtacatggattactgaggagcaagctacctcgccggattactgggtgcagcaagtgcgcaacgcggtgctgtttagcgaaggtgcggcgcaactgttggtacaacccacgctgtttatcgaatgtgggccgggtaatacgctctctacctttattcaaggacataaccaatacagcgatcagccgaccctgttgacgctacgcaaagccaacgcggcgatcgatgatgagcacatgttgcatcgtacgctggcggcgctgtgggtcaggggggagaatattgattggagacgctttaaccagacggcactcggcaagcacattccattgccggattaccccttcgaacagacttattactatcgctatggtgctgcactttccggttatcgccagtatccaaatcctctgcgccgtccgcaagatgagtggctccagcgtgtgctgtggcggatgcacgacacatccttgcgggaggcgttctatgcgccgggcgaattgatcatcattattagtgctgacggcgacaagttacagcagacgctgatgagtagtggtgtcgacagcatcacaatgccgctgccgatatcgtcagaggacgacgtgtgggataatgaccgtattctgacgcatttccacgacatctgcgcacttttagcacacaaaacctaccgacagttacattgcttgtatgctcccggtgcggaggcaggatcatcgttgacacagtcgctctcgggactttatcgtgttgctcgctggtgtatgcacagcaccacgccgctggcgtccttaacagtattgacccatggtgcgtttcgcgtacaggaagaggataatcccgaaccgacgctggcagcgttgtcgggcgcagtaaacgtgtttgcccaagagctgcacccgaccgaagtacgattgatcgatatcgatgcgcagagcagtgatgagaatctgaacttgctaacccagcgtctggccccgaaacaagaaacggtaatggcgctcaggcaggggatgctttacctgcgacgctttatcccgacacggctgttggcgcacttaccccctcaaacagggtgtataccgggcaacgtactgtggattatcggcggtgaaaaggggattggccgcatgatcggcgaagcgcttgctcagcgtgagggagtccgtgtggtactgagcagccgcacaggttatcaccatgaagcggtgcagcaggacgcattagacgttatccactgcgacgtgacgcaggcggaggcggttagagcttgtctggcaactctgctcgaacgctatggacggttggatggcgtgatttttgctgccgacgctaccaccacattgacactgcatcaattgagcgaatctgcgctacgcgacacgctaacggtgaaagaacggggtactgctaatgtgctgcatgcgttagcgcaacggaatctgctggatgagcgtctgctactgctgttctgtaactcgttggctgccgtgaatgcggagattggccagacaggctatgctaccgccagcgcctatttggatgcactggcacagcaactgcgtacccgctacaaggtgaatgcgctcagtatcgggttggatgcattgcgtgagcagggcatgttgttggatgctataaacggcagtgaatacgatgtcttgcgtggactgcgcccattgatgacgggaacgttgctacaggcttataaacaacaaggggctgacaccagctactacgcgcgattatcccccgagtccgattggctgctggatgagcaccggatatccggtatcgccacgctgccgggaaccggctatctggcgctggcgtatgaagctctgcgccattactttgtgcaagaccaaatctgcattgatgaattggtctttttggcaccgttgactgtgatggacaactgcagtgttgacgtttttgttgacatttcacctaacgggcaaggagttagtgtcgaggtgaaatcaatgacggagcgctttagtggcacgttaacaacccatgccagaggcagggcgacgcgtctgatggtagacgataatgttgtgtgcgatctcacggggctgatgcgcgagatgcacactatcactcctccaacaaaggaattatcgagcacgcacttccactatgggccgcgctggcacagcgtacaacaactgtatggcaataccgcccagactcaggttttcgcaacgctggccctgcccaccgttgccgctaatgatacgatcgcactgcaccctgcgctgttggacatcgccagcagtgttgtcgaacaactgcctggttttcatactgattcggtacctttcctttatcaggatttacgcctgtaccgcccgttgccgaacaccttacatgtggcgctgactgtcaatcggcacgatgaggagggtgacagctacgctttcacgctctacgacatggcaggcgagatggttgcccgctgtgcggcaatggtgaagcgcaaggtacagctccacatacaagatgtcgatgacgacacgcgactgcgcgtgcccagtgccgataactaccaactgcggctggccgctgagggggagggggcaggaaagctagcgttgtgccctacgccgcgcttagcgctgggggattcacaagtagagattgaggtactggccaccggactgaactttaaggatgtgctgttcaccacgggattgctccggcagcagccgggtgaggctccgctgcaattgggactggagtgcgccggacgcattactcgcgtgggtaaaaatgtcactgaatttgccccgggagaggatgttatggcggtgctaaacggtggttttgtccagtacgcacgggtagaaagcgattgcgtagtgagaaaaccagcccattgtcgcatcgaacaggcggctgcgctgcctatcgcatacctcaccgcctattacgcactggtggtgcgcgctaatttgcaacccggagaacgagtattgatccacagtgcggcggggggcgttggcttggctgcgctacatattgccaaacgctgcggagcacagattttcgccacagcaggtagcgagcagaaacgcgattacttgctttcgctaggcgtacatgccgtagctgattcacacgacgaacagttcgctgccactctgctgaccgcatcggacggacaggggatggatgtgatccttaactccctcacaggccgtctgcttgacgctagcctcgcgctgctggcaccgctgggccgttttcttgagctgggcagcaaggacattgtggaagacaaagcgctaccgatgcgtttcttcgcccaaggcggcacctttattccgattaattttcacgcggcgcatggtgcgtttagtcgctacctgcaacagattgtcgcttggatagatgataacacgctaccgctccttccatgcaaatccgtaccattgcccgaggttgcacgcgccttcgccaccctgaccacgccgcagcatattggcaaggtggtggtaacgcatcgcactgcggcaggcatggaccggctgaacgccatgatagcagaaaggcgcctcggcggctatgcgctcagcatgagcaatgccgaggtgatgcgccaattgtggccgatactgaacactcgcagtccgtgggcgcaactgctgctctcacctcgggcgatcgatagattagcgcggggcaaccgggtggatcgcggtgtaccgtctgccgctaacgatacgattactcagcagacagtgaaaaagcggccccgtccggaaattggcgtgccttacagccccgcgacacgtgaagtggaacgcgtgctctgccaaatcctggaagagtatctgggtctggatagagtaggcattgacgacaactatgccgaattaggggcaacctcactcgatatggtgcaactgagtgggcaaatggcgcgtcactatccgcaagtgagcgtggtgtcgctgtacaaccacgccaccgtgcgccagctggcgacgttttgccagcccccggagggcgagtcaaatgcgccatcaccacagcctgcagtacagacgaatacgcgcgcgaatcagatagcaaaacgtgctttgcaaattgcgaaaaatactgcgcgcagtcacacgtctttgcattaa

SEQ ID NO: 1067 - DNA, 2601 bp, Escherichia coli
atggaatacgcaagcgaaatgaacggcatggaaatcgccattattggtatggcggtccgtttcccgcagtcccggacgttacacgaattctggcataacatcgttcagggtaaagagtgcgtcaccttctttagcgaggaggagttgctggccgaaggcgtcgaacagagtactctggacaacccggcctatgtacgggccaagccctatatcgagggcatatgcgactttgatgctgcatttttcggttacagtcacaaagaggcgcagactctggatccgaaatcccgcgtattacatgaagttgcctaccatgcgctggaagatgcaggctatgcccaacgtaccagcgatctgatcaccggggtattcgtgggggcgtcagaagatgtggattggctacggcgttcgctgtcacagattggcggcgatgcgctgaatcgttttgagtctggcatctatggtcataaggatctgctcgcacatctcattgcctacagtctcaatctcaacggtccggtgtatagtctctacaccagttgttcgacttctctgagtgcaacgcatatcgcttgccgcagcttgttgtttggcgaatgtgatctggcgttggcgggtggaattactatcgatttaccgcagaagtcaggctacttctgtcaacagggcatgatccactccaccgatggccactgccgtccttttgacagtcaggcttctggcaccctgtttggcgatggcgcgggtgtggttgtgcttcggcgtttggaagatgctctggcagcgggcgatcgcatctatgcggtgatccggggtagcgcggtcaacaatgacggtaaacaaaaaatcggttttgtcgcgcctggtcatgagggacagaaggcggtcatttgtgcggcctgtcatctggcagaagtaagcccagaaagcatcggctacgttgaaacccacggtaccggcacccgtattggcgatcctatcgagttcgctgcgttgacggaggcgttcgatacttcacaccgccaatactgtgcactaggggctgtaaaggcgaatattggccacacccacgcggcggcgggcgtggctgggttaatcaagacggcgctggtacttcatcaccggaccattccgccgctcgccaactatcaaatgcctaactcgaagctggatctggcgcattcaccgttttatatcccgatacagccgcaggagtggcccgcatcgcggatgccgccccgcgctggcgtcagttcttttggcattggcggtactaatgtgcatatgattttggaagggctgaatcctgcggtgcgcgatgaccatgaccaagtgcgagcaccggtgtttatccctctctccgcgccgtctttcgagcagttggatgagctgacgcaacagcttaccccgttgctggctacccttgatgcgtcaacgctagcctacacacaacaagtggcgcgccccgtgtttgattgccgccgagtgatacaagtggaaaacgatggtacgcaagcgatgctggcatcgctggataacctaatgcccgacgctccttggggcctacactgtccagatctacgtactacgaacgattgtacttacgcacagtggctggcgcattcggcacattatcaacgcgaagcgactgctctgacggcattactcgacggcatgaatattccacccgcttattgccacgctgaaacgtgggcggcacaagcgaacagcagcctgctaatcagaggctgccagactatcgccgcgctaaaaacctggatgaacctgctaccgacattaacactgctgtcgggtgctggaacaggccttttgcctgccgctgcagccagtggcatgattgcgacgcaagacgtgttgcatttgctgtgggaaatggagcaaaaggcgcttcatctctggcttcctgagcgccatgaacctatccccggctacgtgctggcttggcaaggaaatcccatcaccgatgcacagcgtaacgacagagggttttggagtgaagcgctgttggcagatacccgagaactgggggagggcgttcacagtatcaactgggttaggctgccgccggaaataagagaagacgttgatgtattgcgctacgtggcgcaactgtggtgtgcaggtatcaatgtggattgggccgtgtggtacggcactccgctgccgcaacgcgggagcgcatcagcatatcccttcgcacacaaccactacccattgcctggacgggtaatgggtagtgtggagacccaacccgaagcaggacccgaaacgcaccacccttatcaggcacgtcccgtgttaagcgtacctttcgtagccgcacactcgcggggtatgcagtatatcacaggtctgatggaactgttgctggaaatatcgccggtcggggtggacgacgacttttttgaattaggcgggcattcgctgctggtgacgcaattgacctcccgtttagaacgcgatttcaacgtacatatcgatcttttgaccctgatggaaaaccccaatccgcgcaatatctacgcgcatatcgcggcgcaactggggggcgaagacaacctcgaaatagcctgtcagtaa

SEQ ID NO: 1068 - DNA, 870 bp, Escherichia coli
atgatgaacgtggcggtaataggtgcaggagtaatgggaactggcgtcgctcataacatggcgcaatacggcatatcaactaacgttgtggatatttctcaatctcagttggataaatgccgacagatgatcgaagcgaacttacgcttatataattttcacccgcagcataaaaaaaagacgcatagcacagcggagataatggaaaatatccgtttcacaactgagttggacgacattgtagaatgtgatttggtgattgagaatattactgaggatatagagaaaaaaaatgcactatacacacggatgaatacgatctgcggcgcgtcgacagtgtttggtgttaatacgtccgccatatctatcactgcgttgtcaaaattaatgcgacatccggagaatgtagtcggcgtccattttatgaacccggtaccgctgatgcacaccgtcgaattaatccgtggtgtacatactgcagagcgtacactgaacatatttcaccacttatttgctcagttgaataaaaccggcatcgtggtcaatgattctccgggctttgtcactaatcgtgccatgatgatttttgttaacgaagccatatttatggtgcaggagcagatcgcccgtgctgaggatatcgatacgttgtttaaaacctgcttcgggcacaagatggggccactacaaactgccgatttgattggcttggatacgattttgcaatcgttgcaggtgctatatgaaagttttaatgatgataagtatcgccccagctttttattaaagaaaatggtcgatgcagggtatttgggcgtgaaatcagggcaggggttttatcgctatcaacagacgtacgccgagcagtga

SEQ ID NO: 1069 - DNA, 249 bp, Escherichia coli
atgaaaaagcaagatatgaaagccgccattcgggaatttctttcacgctcattacgtgggcatacgttgaacgatgatgacgatattttttctctcgggcttgtccattcgttatttactgtgcaaatcatactgtttatagaaaaaaattttcaggttgagctggaagtgagtgagttgaaaacagaacagattgctaccgtcaataaaatagtggagctcattcagcgacaaacaggcctggagtaa

SEQ ID NO: 1070 - DNA, 1131 bp, Escherichia coli
atgtgcactgaaaattatgagctggctcagcaagaggcagtcctgtttgccaaacaacatctcgccctggctgcacagaatattgaacgtcagcagtttattgtgccagacattatttcttgcgtggcgcaggccgggtacttaggtgcgtcaatcccccaaaaatatggcggacgaggttacgattcttatcaactgtgcgctttgcatgaagtgatggctggggttcacggttcgttagaaaatctcataacagtgactggcatggtcagcacgctgctgcaacgcgtgggcagcgcagcgcaaaaagcccattatttgccaaagctggcaacgggggaattgatcggcgcgattgccctcacagagccgaatatcggcagtgacttagtgaacgtggaaacagaattacagcaggatggcgacggttggcggctgaacgggaaaaagaaatggatcacgctagggcagattgctgatttttttattgttttaatccactgtggtaatcaattagcgacagtgcttatcgatcgcaataccgacggctttactatcactccactcaacgacatgttggggttacgcggcaatatgttggcagagctgcattttaatgactgccgcctgaaggaggatgcattgctcggtccgctcacccctggggtaccgctggcggtgaattttgcgctcaatgaagggcggttcaccaccgcttgtggcagcctaggattatgtcgggccgctgtggatgtggcagcgcgctacatccgccagcgtaaacagttcaagcggcggttattttctcatggcattgttcagcacttgtttgccacaatgctgacccaaacgcgcagtgcgcaactaatgtgcttcagcgcggcggaataccgcgaaacgctgcacccggccatgataaaccaaatcctgatggcgaagtatgtcgcttccaaagctgcggtggacgtggccgggaaggcggtgcaactgctcggtgcgaatggttgccatgccgattatgccgtcgaacgttactaccgcgacgccaaaatcatggaaatcatcgaagggacatcgcaaattcatgaaatccaaattgcgatgaattacatgatggggagtgaagcatga

SEQ ID NO: 1071 - DNA, 1269 bp, Escherichia coli
atgacgaaggatgtcgcactgatgttccctggctccggttcgcaatatgtaggcatggcacggtggctgtatgagcgttatccgcaggtgcgtactctgtttgatgaagctagccagatcacggaacgggacatggcagcgctgtgcctgtcgggtactttggtacaacttgctgagccgacggcaatggcgctggctatctataccaccagcgtggcccactttgtcgcgtggcagcagtttttggcgcaaaccggtgtccatgtcaacctacgttatatgttgggtcatagtttgggcgaatatgctgcgctgacctgtagtggtgcgctgagcttttcccaagcactggcgctggtggcgatgcgtagccgtttagccagtgagatcgcccgcgaaatggacgcgagtaccactatcatcaagcaagggaatcaggcactggtggcagcggcgtgcgaggtggcggagcgtgaaacccgacagcaggtcggtattgcctgcttcaattcgccgcaacagtttatgttatccggacaaaactcggccattatcgccgccgagcaatacctgctggatcatgatcgccaagtcgaagtagtgccgctgattggtggtgttccttaccacagcccactattgaaaccttgcggacagcaattgcgcaaggcgctggatcgctgcgaatggcgacgtccgtgctgcccagtgatctccaatgtcaacgctcaaccctatcccgatacggtcacgccagtccagtggctagaacaacagctctctcagccagtgcagtggcagcgctctctcacctacctgacaggacacttgtcaccgatcgcgatagagatcggtccgcaaagtgtgctgaaaaacctgctgctggagaatcgctatccagcaccagtttacgcatttgacaatcgtcatgatcgtgcgcaactagcattggtgctgggggataacatggcggtgaagaccgatccggaagccgtgcgccgccaacgcatcacgctgctcactaacgccctgacagccacacgccatcaccgcgccgccgatgtggctgccagtgcgcaattgaaagaattgctcagtcgtttttttgagcgcatccagcagattgagcagcgcggcacatccagcgaagaggacattgcttttttgcatgagcttcttgaacagggatttcaactcaaaggcagtagtcaggcagaaatcgacgcctgccacgcgcggttggcttccgacaacggcggacaggcgtaa

SEQ ID NO: 1072 - DNA, 4797 bp, Escherichia coli
atggaacagcaagggattatgagacagttgcctaccgacgaccaaacgatagtcgactatttgtatcgtatcgccggagaatatggggaaaaagccgctgtattgatgggggacgcggcgctgagctatcacgatcttaatgcacgctctaaccaactggcgcattatctgcgtgggctggggatcggcgaggatcgtgtggtagctatccgcctgccgcgcggcatggcaatgctgattgccattttcgctattgtaaaagctggtggtgcctatttaccgctggcgtacaatgcaccgcgcagccgcattgaaaatatactgagcaacagcggcgctgtttgtctgatcggtactgacgatggtgatcgctggccgattcctcgcgtcgaaatcgacagcgcggcggtcagtgccatgccaacgacggatctgcgttaccgaccacatgcgcggcaactggcgtatattatttacacctccggttccaccggtgtccctaaaggggtggcgacggaacatgcagcgctgctaaaccgaattgtctggatgcagaacgcttatcctatcagttctcaggacgtgctgtttcagaagacggtgtacacctttgacgtctctgtctgggagatgttctggtgggcgatgtacggcgcatctgtagtgctgttaccgtccggactggagagcgatccgcgaaccttggctcgactgattcagcgtcaccgcgtgtcggttgtgcactttgtcccttcgatgctgaacctgtttgtcgagtatctggagatgaaacaggatcctcgtttgaccgcctcattgcgattagtgttttccagcggtgagaaactcacggtccacagtgtggctcgcttttatcaatcggtggcgcagggtgatcttataaatctctatggcccgaccgaagcagcaatcgatgtcagtcatcaccgctgcctgcgcgggtacgactacgacgatatccctatcggtcaagcgattgacggttgccgactctatgtgctggatgaccatggtaatccggtagcagacggcgaagagggcgaactgtatctcgctggtatcgggctggcacgtggctatctcaacaacgtggcgttaactgatcgctgttttactatacatccaactttgcgccatttagggaaaccggagcggctgtataaaactggagatctggtgtggcgcgacggggaaagccaacaaattcattacattggccgtaatgattttcagattaaaattagagggttgcgcgttgaattgggagaaatcgaagcccatgcgatgcgtttcccgggggtacagcaggcagtcgtggtggcggatcaggatgatcccgacaatcagttgatttacgcttttgtcgtcagtagcgtgccgctcaatttggcggccttaatggacgcactgtccaaaaacttgcctgcctacatgctgccgaaccgtttgttggcaatgtcagagttaccactctccgacaatggcaagtgctgtcgtaaaacgttgctcgacttggcgcgggcgtactcagccagtcgcgtcgatttacgtgaaactcccgccgtgcgctacctaccgttgtcatcggctcaatcgtcgatgtggtttatgcaacaattggcgccgcatactgcactatacaataaccccaccgccttgctgcttgaaggagaactggatcgcacgcggatggacggcgcgattcgtcaattgatgagtcggcatactctgttacgtgctatggcggaaacccacaatggacaaccagtattggcggtgcctcagtgcgtatcgtcgcaggcgttgctgaccatagtgccactcccctcggtgagtgatgataacgcgctacaggcgatgatcaaccagcgtgcggcacaccccatgcccttaacgtcaggcacaccgctgtgccggtttgaactgttgacgctcgatgacgatcgtagcgtattgttgattcatctgcaccacatcatcagtgatggctggtcgaaaggcgttttgttacgcgaattgcaggccgcatataacggtgaatcgctgacgccagaaccgttgttggagtatgccgactacatggagtaccaagaagagtggcgtcagagtgacgcctatcaggacgcgatgcgttactggcaaaataccctggcggggacgttgccgattcttgatatccccaccgatcaaccgcgtcagaaggtggcacgttaccaaggcgcgtttgttgccttcgcattgtctgccaacacatgcgagcgagtgttggcggcggcgcgtgcgcagcgcgtgtcgttgtataactatctgctgaccgccttcgtcctcctgctgcatcgtaatgcgcgtcaacaggaatacatcgtcggtatgccgattgctgcgcggctgacgaaagaacaggagcatatgatcgcgccgctggtcaacgtactaccgctgcgcttacctcttgacgaagcggcatcgttttccgagttagtacagacgatcagaggtattctgtttgccgctttcaggcaccagcgtctcgaatttaccgacatagtgcgcgcagtgaatgtggatcgcagcgccggacatttcccaatatatcagtgcatgttccaactcgacaatatgcctctggcgagcccgacactaaacggtgtcaacgttacaccattattgttggataccagtgcatctcaggtggatatttcgctaagtatgcagcatatcgatgggcgcatcaccggcactttcgaatacgacgctgggttatacagtgcggatcgtattcagcacctggtggcgcagtggagagaactgctagatgaggccagcagccaacccacgcagttagtccgcgatttgattcgtttcacccctcgagagcacgcttggctggcgcggcacaacgccactgaagttgctctgccgcccgtagataacctgctggcgctagtgttaccgcactgccagcagcgccctacacaggtggctttgcgccacgctgacgacgccatgacctatggcgaattgcaacaggccacgatgcagatgtgtacttggctgcgtgcgcaaggcgtaaaacgcggagaatctgtcgcactgcaactgcctttttgtttcgaattgattatcgcacaattggcgatcctctctttgggggcgagctatgtgccgctggatggtaacgcgccagcggcccgcaatgccctgatcttggcgcaggcaacgccgtgcatgctgctggtggcgcaaccgctggaatcccctcatgggctgacgataccttgggtgctagtgcctgactggcgtagcctgctgacagaaataccgaacctgccagttagtgttgcgccagatgctttagattgcgatgcggtggtgatctttacctccggtactaccgggcagcccaaaggcgtccgactcagccaacgtaatctggtcaacttaaccgcatcctttatatccagctatcaagtgacgcatcaggatgttttgctacccattacctcagtcgcgtctgctagctttgtcggggaggtactgccgctgctcgccgctggcggtaccttagtattggcgcaaaaagcgcagagtttggatagtgatgcgctcattgcgctgctggcatctcagcgggtgactatcctgagcaccaccccatcgctttctgccagcctctctgtgctggctcagtcgatgggatcgctgcgcttgtttctgtgcggtggtgaagcgttagagtatgagcagatagcgccgcttctgccgcacatggcagtggttaacggttatggcctgaccgaaagcggtatctgctcgacctactttcctgtcgcaaagcgtagggagcaagaaacgggagcattgcccattggccgcccgattcagaacacccaagcttatgtggtggatgcatataatcgtttggtaccgccaggtgcctgtggagaactctgtttctctggtctgggcatttcgcccggctaccttgatgcacgacaggatcccgagcgctttgtcgagttgccggaataccctggcgttcgggtgctgaaaactggcgatcgtgctcgttgggcaaccgacgggatgttgttctatctcggcagacaagatcgccaagtgcagatccgtggataccgagttgaactgggcgacatcgaaagcctactgaaacagcatccggatattgctgatgcgtgggtcgatgtgcgacgcaatgcggcggcaacgccgctactagtggccttctattgcagcgtcaacggcgttgcgctggatgctcagcaattgcgcgtatggctcagcctgcggcttccgttgcatatgctgccgttgctctacgtgccgctgagcgccatgccgctaggtgtaaacggcaaaatcgatccccagtgcctgccgctggtcgatctgcggcagttggaagggccgggcgagtatgtccctccggcaaccgaactggaacagcgtctggcggagatctggcagcagttgcttggcctggagcgtgtcggcaccaccacaaatttcttcgatctcggtggacactcacttctgctagtacagatgcagcaatacatcgggcagcagtgcggtcagcacgtggcgttggttgacctgctgcgcttcactaccatcaaacgcttggcggaatttctgctggccccggacgcagcgcaagggaccacaggagatcagacacaactgcgtgcggcgaagcagcgtttggcctttggtcacacgcgctgggcagccacaaccgacagtcatcactga

SEQ ID NO: 1073 - DNA, 3033 bp, Escherichia coli
atggcagagaatgattttggtatagctatcattgggatggcggggcgtttccctcaagccgatacggtacaggcgttttgggaaaatcttctggcaagccgagagtgcatttccttttacagcgatgaggaactgctggcgatgggaatcagtcctgaatttgttcagcacccagattacgtcaaggccaaaggggaggtagctgacatcgataaatttgatgctgctttcttcggtatcgcaccccgagaggcggagctgatggatcctcagcaccgtgtcctgttggaaaccgcgtgggcagccttcgaagatgcgggctatgtggctgccgattatccgggggatgtgggtatttttgctggcaaaagcatggattcctatttgatgctgaacttgatgccgcactttaaacgcgttttctcttctggcagtttgcaggcggccattggtaacgacaaagacagtatcactactaccatcgcctatcacctcaacctgcgcggcccggcgatcacggtgcagacttcctcatcgacatccttggtggcggtgtgcgtggcatgccaaagtctgttgacttggcagtgtgacatggcgattgctggcggagtaacgctggggccaccagcaaaaaccggctacttgtcgcaggaaggtgggatcaccgccgccgatggccactgtcgggcgttttccgataacagcagcggatttgtgcccggcaccggcgcggggctggtggtgttaaagcgtgttgatgaagcgctgcgtgatggcgataacatctatgcggtcatcaaaggatttgccgtcaacaacgacggttcggagaagatcagttataccgcgccgagcgtggatgcccaagcgcgcgccattgctcaggcacagcggttggcaggtttgacaccgcaggatatcacttatgtggaagcccacggtaccggtacgcgtttgggcgatccggttgagttttctgcgctaagccaagcctttgcgggcgcgtcacaaaagcagtactgtgcgctggggtcggtaaaaaccaatatcggccatttggatacggcagcgggcgtcgcagggttgataaaaaccgcgctggcagtgcagcagggaataattcccgcaacattgcactttgaacggcccaatgcacagatcgatctgaccaatagtccgttttacatcaacactacctgtcaaccgtggcaaccggaaagcggcatccgtcgcgcgggcgtcacctcgcttggcatgggcggcaccaatgcccatgttgtgctggaacaagcgccagccgttgacttgcaagcgcgagcgcctgtacctgcctacagtattttgccgttttctgccaagaccgacagtgccttatcctcggggctggcgcgttttgccgattttctgcaacatgaatcgctgccggataggcgggatctggcgtggacactctcacagggacgtaaagccttcgcacatcgcgccgcgctcgtaaccagagatctacatgctgccgggacgctgttgcagcaggccgcgacagcgccgtttgctcgtggtgtagcgcagacacagctcgggctggggctgttgttttccgggcagggtagccaatatcagcgcatgggccatcagctctatcaggtttggccggcctacgccgatgccttcgaccgttgcgcgacgttactcgagcgtgaataccaactggatatccgccatgagttgttcagagcagaagtctcgttagcccaaggcgaacgccttgcgcaaacctgcctcacacaaccgctgctgttcagcgtcgagtatgccttagcccaattgtggctcagttggggaatcacgccaacggtaatgattggtcattcgctgggcgaatgggtggcagcgacgttggcaggcgtgttctctttagaggatgcgttgcgcttggtggcgcgtcgagcagagctgatgcatcaggcaccaagcggtgccatgttgatggtggcgctgcccgaagcacagattcgcgcactgattaccgccccgctggcgattgccgctgttaacgcccctgactattcggttatcgccgggcctacgtcggagatcctcgccgtcagccagcgtctgacggagcagaacatcatcaacaaacgattgcatacctctcatgccttccactctagcatgatgcaagatgcggcgcaggcgctacgtcaggcatttgagaatgtacgactgaacccgccgacgctcaccatcatctctaccgtaaccggcgcgcacgtcagtgccgacactctcaccacaccggactactggattgaacagatgctgatgcctgtgcagttctccgctgcactgcaagaggcgcaagctacatttgatgtcgattttctggaaatcgggccaggtgccaccctgacccagctgaccaacggtcatgcattaggtgatcgtctcgcgttcagctcgctgccagcaggtgcccgcagcagcgatgagcacaaacatatcttagataccgtggcagcgctttgggtgcgagggcataacatcgatttgtctgcgtttgcaggtgagcaaccgcgccgtgtctctctaccgacctacgcctttgacaaaatccgttactgggtcgacagtccagaagaacagaggagcgccgtaacgccggtggcggacgcgggaagtgtcatccctagcgaaccgtcggtgcgccgtcagccgcgtccggcgttttcggtgccttacgcggctccagagagcaaaacgcagcgcggcttagtggcgatttgtgaagcattgctgggaattgacggcttgggcattgacgacaacttcttcgaagctggcggacattcattgatgctgggcatgttgctggcgcaggtacaggaacggttcgccgtcactctttcctttttcgacgtgatggaggatgccagcgtgcgcgcgttagcgcagttggtcgagcaggagcagcaggatgatggagggtccgcacttgccgtgctagttaacgacatgattaatgagtga

SEQ ID NO: 1074 - DNA, 6501 bp, Escherichia coli
atgacgatacatcatgccgcattggcgcgaatgttaccggcggaaaaaaaggaaaagctattacgacagttggcgcaatcaggcgtgtcgcccagccgcattcccatcatcaaagcggatccggcgcaggcaatcccgctgtcgtttaatcaggagcggttgtggtttttgcaaaaatacgacagtaccgccactaactacaacctttatgtggtgtatcgtttgcatggcgtggtggatatgccgatgctcacggaggcattgcgccatgtgcaagcacgccacgccatcttgcgtacccgtatcatagtacgtaacgatcggccttgccaggtgatagatgatgcgtcttcgttagttctcgatacggtcacgctggcagcgcaggccccaacttcagcgctagatgcggtaattcagcaggtgataaacacccggttcgatctggcacgcggtcctctgtggggggtaactcagattattcaacccgatcaaggttgtcatttggtgttttgcgcgcaccatatcatcatcgacggtatctcgttacggctattgttcgatgagttacagcagcagtatgctcgattgcatgctggtaatgaaacgtcgctgcctccgccgccgctgcaatatgccgattatgccttctggcagcgcgaatggttccaggataccttgctggcgaacgaacttgcctactggcgtgcgcgtttgcaagacgcgcctctgctgtcgacatttccatcgctgcacccgcgcccggcacaaccctctacacatggttcccggttcagcatcaccctggatgaaacattaagcctcgcgttgaaacacgtagcccgcacgcaggaaacaacgccgttcgtgctgatgctgactgccttccaactggtgctgatgcgttacgctcagcaacagcgattggtgataggcatgccagtatcggggcgcattcgcccggaattgcagagtagcattggttattatgccagcactgcggttatctacaccgattttaatggggttgaggtagggcgcgaggcgctccagcgtgtgaaggccagcgtgaaagagacgcaggggcgccagcaactgccgtttgaaaacttggtgaatatgctggatctccctcgcagcctttcccattcaccgttgttccagatcctctatatctaccataaccacgtgaccccacgcgcttttaccttggctggtgcgtattgggagcaggtgacgtatcacaatcagaccgtcaaatacgacatgacagtcgaggtgttccaaaacgacgccacgttcgacgtctcctttgagtacgatttggggttatacgatgctgatgtggtgaagcagattgccgaggcgctgcgtcagcactgcttatcgttgacatcatcactggagaccccgataggggcgatccctctgcatgcaccggagaccgcaacgccgcggcgtgatccgctcaacgccactaacgtcccgtggctcgggccgcaggatgtgctgcgtatcattgaacagcgctgtgtgcagcacccaaagcaactggcaatacagcagcatgacggcacactgacctacgctgagctctgggcgcgtgtgcagttcatcgcgatgcgttttcgagcgcatggcatacaaccgggcgatcgtattggcgtgctgttgccacgtcacagggatgtgattgcaaccatgttggcgacgtggtttgtgggggcgtgctacgtgccgttcgatattcatcagcctgccgcgcgtttgcaacgccttatgcaacgcgcgcgtttggtctgtctggtggtccgtcagccgggagagtggggtgaaattgtacaactgtcgttgccggaattgatgcaggacatgtcgaatgcgatccggtattctacaccttgcgcgctgttgccggatatgcaggcctacctgttgtttacctccggcagtaccggtgaacctaaaggcgtgtgcgttgttcaccgcgggttgctgaacttgttgttggatatgcagcgtacctttgcggttggctcgcaagaccggctgctctcggtgacgacgccaacatttgatatctcattcttggagtatctgttgccgctgatctccggtgcgagtctctatctgacagaggcggaacgcgccgcagacagcttccgtatgattccgctgattgccgactatcgaccaacgctgatgcaggcgacgccctcgttctggcacgggctgttgatggcgggttggcgtggcgacccggaactatgtgtgctggcaggcggtgaagcgctgccaacgaaagtggcggaagaactgttgcgctgttgtggttcattgtggaacctctacggtcccaccgaaacaaccatttggtcgctgaaatcgcagataacccaagcggaaaacatcaccctcggcgctcccattgccaatacccgtatatacattctggacaatgagggccatccagtgccgcaaggcgttgacggcgagctttacatcgccggggatggtgtggcgcaagggtatgatgggcagcctgagttgaacgcacagttcttcttgtcagaaccaggggtccccggtggcaggatgttccgcactggcgatctggtcaggagtgatgcgcaggggcagttgttttttgttgggcgcaaagatagccagatcaaactgcgcggttatcgtattgagttaggcgaaattgaacggacgttggcacggcatccgcatgtggacgccgcggtggtggcctgtattgaaagagcaccgttacacaaagcactggcagcattcatcatcaccagtgagcctccctcgctattcgagcaactgaaaaacgagcttcggcagcaactgccagactatatggtgccaacgctgtggcagagggtggccgacttcccgaacactgacaacggtaaaatcgatcgtaagcggttagcggaaaatttcgttgccgatagctcccttgtgtcgccgcagacgcaagcgctgagcgacacggaacagatgttgctggcgctgtggatgcgctatttgccgataaaaaacgtcgatcctgagtgcgatttctttcgcttggggggacattcgctgttggcggtaacgttggtagcagagatcaaccgcacttttcactgcgccttgaccctgaaggacattttccactactccacactacgggcactgagtgcgcgtattgcacagcaatctatcacggacgctgccgcgtctcaggatgactgggtgatagtgcacgaccctgagcatcgtcatcaaccgttcccattgacggatgttcagcgcgcctactggcttggacgacagacgggtgctacctcgatcgcgacccacatctaccatgaatttgacgtagaacactttaatgttacgcgttttacccatgcggtgaatgcgctgatcgctcgccatgaaatgctacgtgcgcgggtactccccgacggtactcagcagattctggcgcaagtgccggcgtatcagttagagcagcgcgatctgagtgctttgtcccctaacgcacgaaacgatgccttgatggcgatccgcgatcggctgtcgcatcatgtgcatcccgcagatcgttggccgctgtttgatttcagttattcggcttgcacggcgcaacatggccgcttgcatttcagtctcgatctgctgattgccgatgctctgagtatgcgcacgctacagcaggagttgatgatgctgtaccgtgagccccatgtgtcactgccgttgctaccgttctcttttcgtgactacgtgcaggcgctgttggtagagcaggcgagtgaagcctatgcacgcgatcaggcctattggcaacgggcgctgccgcagctgtatggcccaccaacgctgcccgtacagggcgatttggcgcaactgtctgcgatcaggttcgtacgtcgccgtcatcggctgtcagcccacaactggggagtgctgagcgcgctggcccaacgcacacgtatcaccaagacggcattgttgttgacagtctttagccaagtgctggcacgttggagccttagcccgacgtttacgctcaatctgacgttgttcaaccgcccgcagggttaccccaacgcagaggcagttattggtgattttaccgctgtcagcttgctgaatgtttgttacgacagccagcactcttatgcccacaacgctcagcgtattcaggtgcaactgtgggaagatctcgaacatcgtcgtttcagtgggatccgcgccagcgaggcgctgatccatagcggtcgtttccatgcgccgatgccggtggtattcactagtatgttggatatcgacggggagacgactgcgcaagaccctcgggacacaacccgttttactctgtgtccggacgccaatattacccaaacaccgcaggtgtggctcgatcaccaggtgatcgagttggctggggagttgcatttcaactgggacgcggtcgagcaactgtttgataccacgctgctggatcagatgtttggtgcttattgtcatgcgctgcaggcgctggttgccatgccgcaaagttggtggggggtaaatagttctctggcgctgcccaccgttagtgcaccggtcacgcaggctcctgcacctacggccttgttgcaccatggattactgcgtcaggcagcactgacgccacaggaaactgcgctgatcagtcctatccgtgaattgacctatcgccaactgtcgacggcggcggatcatgtggcccgcgccctgttagcgctgggcgtgcagcatggcgaccgcgtggcggtggtgatggaaaaaggctggcagcagattgccgccgtacacggcattttacgactgggtgcggtctatctgccagtggatccggtgctaccgccacagcgtcgccagcttttgctgacggtgggcgaggtgcgggtacaagtaacgcagccgggtctcacgcaattggagccgtcgctgcccgtgctgatcatcgacgacggaatgctggacacgcctgctgcgccgttgcctgaagtggctggggatgtcacggatctggcctatatcattttcacttccggctccaccggtaccccgaaaggagtgatgatcgaccaccgtgcggccatgaacacgctggaagacatcaacgaacgctttggcctcaatgcgcaggatagggtgttcgggctgtcatcattgagctttgacctgtcggtttacgatgcctttgcgccttttatggtgggtgcagcgctggtactgccggaagcaggacgggaaaaagatccgcgtcattggcagacagttatggcacacggtcatgtaagcgtctggaatgcagtgcccgcactgatgcagatgctgtgcgaataccacagcggcgatcggatgagttatccgacgttgcgtctggcactgttgagcggcgactggatcccgctaacgttaccggagcagatgcgcgagcggctcaatgaaacgatggacatcatcagtctgggtggagcgaccgagtgcgccatctggtcggtctactacccgataggtgaggtggaatcgacgtggaccagtattccctacggtcggggcctgcgcaaccagccagtatacgtgctaaatgcgcaactggaggaatgtccggtcggggtggaaggagagatttgcattggcgggatggggctggcacaaggctacctgaacgacgcagagaaaacggcggcgagctttgtctggcgcgaagcgagtggtgagcgaatttaccgcactggggatcgcgggcgctactttgctgacgggcaagtcgcctttttggggcgcaacgatacccaagtgaaggtgaatggttaccgtatcgaactgggggaagtcaaaagccaccttgaacagctcgacagcgtagggagtgccgccgtggtgtgccaccagggacagctgtatgcctttatcactgccgcagaaaacctgcatcctgacgatactgacgccctgttagcgcgtgttcgtgctcagttagccgtgcagttgccgtattacctgctgccccaacatttctttcttctcaaggtgctgccgatgacaggcaacggcaaaattgatcaggcggcaatggttcaagaggttatccaacgtatgtcgcaatctacatcacagaagtcgagggccctcgcgcacgcctcgccctatgaacagcaggtggccgctctctggtgcgaggtactacaacgagaacagatcggactgaatgacaacttttttgaagcggggggcggctcaatccagatcgtgctgttgcatcgccgtattgaggagatttttaaggttacggtacctattgctgagctatttcgcttaaccaccgtgaaaagaattgccggttatttgcaggctatgcaggacaatgcacgggcggtgaatcaaacacagcagcgtgatgcttcccgatcccgcgcccagcaacgacttgtacgtcgtcaccagcgtcagcgttaa

SEQ ID NO: 1075 - DNA, 6465 bp, Escherichia coli
atgacttacagtgaaagcgatattgccattgttggcatgaactgccggtaccccggcgtgcacagcgttgcggcattcgaaacggtgcttcgcaccggatgcaacattttggaccccaaagtgaccccgagcaacggccacaatcacattacgctgaacaacgtctatgagcacatggcggagttcgatgccaacttttttggctacagccgtgcagaagcagaaatcatggatccacagcagcgcgtatttctgacctgtgcttgggagatgtttgaacaaagcggttacaacccgaagcagcacgatgcgcgcgtcggcctatatgcgggcgtgagtaccagcttctatttgcttactcacctgatgaacaacccagacaagcttgcacaactcggtggtctccaaatcatggtcggcaatgacaaagatcatttgacgtcccaactggcttaccgcctgaatattaccggcccttgcgtgacggttcaggccagttgtgccacctcgctggtggcagtgcatctggcctgcgaagggctgctgagcggtcagtgcgatatggcgctagcgggtggcgtcacgtttcgcatggaggagcagcgcagctatgaatcgcacggtgatgggctgcaagcagaggatggcctgattcatacttttgatgcgcaggcgagcggtacggtctacagcagtggtttgggcatggtgctgctcaaacgggctacagacgcgcaggtgcaaggggataacatccttgccgtcatcaagggaagcgccatcaacaatgatggcggtgcgcgcagtggttataccgtgccgggtgttgatggtcaggaagctgtgatgattgaggcgcatagcttggcggaggtgacgccgcagcaaattcagtatctagaactgcatggcagcggcacgccgttgggtgacgctatcgaattcgcggccatcaaacgtgtgtttgggacgccagcgccgaatgcgacaccgtggcgtctgggggctgtgaaacctaacgtgggtcacgtagaaatggcatctggcatcaccagtctcataaaaacggtgctgagtcttactaaccgggtgttttaccctacgctgaattttcaacgggctaatccgcaactggggttggaggacagcccgtttgaagtggtgtcccgtttaacgccttggccggagggcaccacgccacgcaccgctggtgtgagtgcgttcggattgggcggcaccaacgcacatctggtggtacaagcgccgttatcgacgccgcaggcgagggcgcagcagatggggccttgtgtcgttgtactctcggcaaaaaaccacaatgccctggaacagatgcaaaacgccctgctggcgaaactggccgcacatccagagatacgcttacaggacgtggcttacaccctgcgtcacgggcgtttctccgccccggtacgtaagtgtgtcatcgcagagaattgcacccagctagcccgacaactccgcgacgcaccgatggtggaggcaaccacgggttgcacgatctactggagattggggcaccgtttcgttgttgcactcgagacgcttagtgactggctggcgtgctctgaggtgttgtcacaggcggtgggacaactgctcgagcattttccgctcgaaccagcctgtctgcaagatctctcccctgcgcaacggacgtttatcagccagtatgcgctgatcgcgttgattgacgaacgggaaacgttgaatgtggtgctatgcggcgacggtgatggtggctatgctgccgccgtactacgaggagactgcacactagagcaagcatggcacaggttgaatgcgggccaaccgtttgatgatgtgccgacgaatcccctcttgcagcccgatgtctgctctttgatgctggacgacgcggcgagtgatgccaatcgcaccgcgctagaggcgttagggcagttatggttggctggcgtcagcctagattggcgctgggtggatgcggcggagcgtatgttgggcagtcaacgcatcgccctgccggggaccgtttttacaccgcagcgctattgggtcgaggccgtacggcctgccacgttctcccacgaatcgtcgaataatctcttatcccgcgcaacaaaatccgatattattgcagtggtgacggagatatgggaacgcacgcttggtgtcagcattgatgatcaccacgccagcttcttcgaactgggtgggcattcgctgttagcgtcaaccattttatatgatattcagcaacgttacggcatcacctgtacgctaagcgcattctttgccgatcccaccatcgagggattaagttgctatctgctcgaacaaggcggcagtgagacagcggtttcagcattgcctgatacggtctttgcccctgaccagcagcacctaccttttccgctgactgacgtgcaacaggcctattgggttgggcggcgaaaatccttgggattgggcaatatttccacccatatctatgtggaatatgaactacaggggctagatgaaacagcgttcaaccgcgcgctcaatgcggtgattgcgcggcacagcatgttgcgtgcaattgtcaacgatgacggtatgcagcagatattgccaaacgtgccggaataccacgttgccttctataccacccagtgcgaggatgcgtttcaacagcgttgccgtgagctgcgtgacaccctctcacatcagatgattgattgtagtcgctggcctctgtttcagatggaggtcgtggttgatccgcagcaaaaagcccggctacatgtatctatcgacctgctgatagccgatgcatggagtctggagttgttcattcgggaactagcttatcactaccgccatccgcaggcggcattgcctacattgacgtacagtttccgcgactatgtgctgaccttaaaatcctacgagaaaacgccgcagtttgaacgggcacgtgactactggcgcgcccgcatcgaaactctgccgccggggccacgattgccgctgcgtaccgaccccacgaagctggaaaaccccacattcgtgcgccgtagctattgtctatctcgtgctatctggcagcgcttgaaaacgcaggcgggtcagatgagtatcacgcctactacactgctgctcacggggtttgctcaggtgttggcgcgctttagttcctcgccgcactttagccttaacctgacgctgtttaatcgtctgccgttgcacgcagatatcaatcacttaattggcgattttactgcactgacgttgctggagattgatatgtcgcagggagaaaccttgcaggcgcgggcaaacgtgatccactctcaactgtggcgcgatttggataaccgcttgtttggcggtatccaggtctctcgactgctggtacaaactcaccgcgatccggcgaaatcggtgattcctatcgtgttcaccagcttgctcaatcaatatgaggcgagctgggaaacggatgatacgctgttcaaccaaccgcaggatgatctgtacagtatttcacagacaccgcaggtgtggctcgatcatcaggtaatggagcgtaacggggaattgcactttaattgggacgtggtggaacaattgtttgaaccggcgctaatggatcagatgttccagtgttattgccaactcctgcacgccctagcccagcgaccacagctctggcatgagactcaagatgtgctggcgctgcccaccgttagtgcaccggtcacgcaggctcctgcacctacggccttgttgcaccatgggttactgcgtcaggcagcactgacgccacaggaaactgcgctgatcagtcctatccgtgaattgacctatcgccaactgtcgacggcggcggatcatgtggcccgcgccctgttagcgctgggcgtgcagcatggcgaccgcgtggcggtggtgatggaaaaaggctggcagcagattgccgccgtacacggcattttacgactgggtgcggtctatctgccagtggatccggtgctaccgccacagcgtcgccagcttttgctgacggtgggcgaggtgcgggtacaagtaacgcagccgggtctcacgcaattggagccgtcgctgcccgtgctgatcatcgacgacggaatgctggacacgcctgctgcgccgttgcctgaagtggctggggatgtcacggatctggcctatatcattttcacttccggctccaccggtaccccgaaaggagtgatgatcgaccaccgtgcggccatgaacacactggaagacatcaacgaacgctttggcctcaatgcgcaggatagggtgttcgggctgtcatcattgagctttgacctgtcggtttacgatgcctttgcgccttttatggtgggtgcagcgctggtactgccggaagcaggacgggaaaaagatccgcgtcattggcagacagttatggcacacggtcatgtaagcgtctggaatgcagtgcccgcactgatgcagatgctgtgcgaataccacagcggcgatcggatgagttatccgacgttgcgtctggcactgttgagcggcgactggatcccgctaacgttaccggagcagatgcgcgagcggctcaatgaaacgatggacatcatcagtctgggtggagcgaccgagtgcgccatctggtcggtctactacccgataggtgaggtggaatcgacgtggaccagtattccctacggtcggggcctgcgcaaccagccagtatacgtgctaaatgcgcaactggaggaatgtccggtcggggtggaaggagagatttgcattggcgggatggggctggcacaaggctacctgaacgacgcagagaaaacggcggcgagctttgtctggcgcgaagcgagtggtgagcgaatttaccgcactggggatcgcgggcgctactttgctgacgggcaagtcgcctttttggggcgcaacgatacccaagtgaaggtgaatggttaccgtatcgaactgggggaaatcgagcgctgcattgcgcgacatcccgatgtggagcagtcagtggtggtggcagtgggtaattctcaacatcgtcggctggtcgcttttgccaaactgcacgatcgccaccaggcgcaggcattgcaagctaaggaagcggaggcggcggcactggcgcagggtattattgtgaatccggcacagcgtctagcgttcaaactcaaggagccacatattcgcgcgctggatggtctgggcattgcactgacggcaccggcggatagcacacgttacatcaaacgccgcagctatcgtcatttcagcgcgcaaaaaaccacgctggcacagttggggcaattgctgtcgggcttggggcagatgcgtctacccggtctaccttttgccaagtatgcctatgcgtccgccggggggctatacccggtgcaaacctacgtgtacctgcatccagacaagatcgaagagggagtatccggtatttactacttcgacccgcgacagagctgtcttatgccggtagcaccagaagtcgagctgaacagtggttttcatgccggacctaatcagtctattgccgatcgggcggcattcacgctgtttatggtggctgatatggcggtgatctcgccattctatgggcaagaggcagcttggcacttctcggtgatggaagcaggtactctctgccatttactggaagaagatgcgccgcgctacggattggggctgtgccaacttgggatggcagacttttccgctgtggcatcgcattttcaattgtcgccacatcatcgctatgtccattgcaccgtggggggcgcgatagggcaagaggcggcaagtgctgcagcattgctgcgcgatttctccacctatgagaaaccgaaggaaaccgctgcgccgctggacatgcagagctacaaagatgccatgctgcgcggcctgcgtcagcaactgcctgactatatggtgccgagtgatctgatgttagcgaccgattttccgttaaccgctaacggcaagctagatcggcaaaaattacagctgcagggcgaacaaattgcccaccagcgtgacggcgtgggtccaatccaggtggacagtgcgttacaacagcggctggtggcgctctggcaggaggtactcggcgtgagccacgtgtcggccgaagacgatttcttctcgctggggggcagttctatagaattggtgcgtattcagcaggcacttgaggcgattatcgggcaggagattcccattgtcgatctgttccgtctgccaaccatcgcggatgtggcgcgttaccttgatgagcaactgcacaatctacccgccgcccacgatatcgtattagcgcaggctgaggtttcgcaggtcagcgctgcgcgcgagaatctggcgttgcggcgtaaacgtgcccaacagggagaaaagggcgatgagtga

SEQ ID NO: 1076 - DNA, 1464 bp, Escherichia coli
atgagtgagcagagctatcgtagcgcagggacactgttggcacagttggcgtccggagaaaccacctcagtggcgttggtgaatcactatttttcacgtatggcgcagtttaacaagccgttgaatgcggtggtgcagcagcactatgcgttggcgttggaggccgccgcgcgcgccgatcgtgagcgactggaggggcgcgcaaggggcgtgttgcacggattaccttgtactgttaaggagtcgtttgacgtgcaagggtggttaaccacgtccggtgcccattatctgaaagataaccgggcgacgcaggatgctccttccatcgcacgcttacgcgctgccggggcgatcctgatgggaaaaaccaatgtgccgatgatgactgccgattggcaaacctataacgatctgtatggcaccacccataacctgtgggataggcaacgttcacccggcggatcgtccggaggagcggcggtggcggtggcggcggatttcacccccgtggaatttggcagcgatttgtttggctcactgcgcattcccgcacactacacaggtgtctatgcccatcgttgtagcttggggttgatgtcggtcagaggacatgtgcccggtggtggtccgcaagcgactgatgagccggatctctcgacggcgggccccatggcgcgcagcgcggcggatctgcgtctgatgatgcgcgcattgagcacattctgggtggaaccgccgcgtattcccgattttagccgttatcaggccaaagcaaactaccgcgtgtgcacgtggtttagtgcgccccaccatgagatagatcaacagatcgcacagcgttttcaatcgtttatcgacaagttacgcgcacagcctggcgtggaggttgatgatgctatgcccgccgatatcgatcccgacgcactgtttgatatagcagtcaaactcagtggtcggctggtaagtacggcgctcaatggacgccagcgtcttacagccgggctggctgcgctgggcttcaggcttgtgggcaagctggcggatgtgcctgaggggataacgagctattaccagggcatgctgaaagacagtggcgaacagcggaatacagacaaactccgccacgaatacagccgggtgatagaaacccttttcgcgcgttatgacgtgttgttgacacctgtcagcccggtgctggcgtttgcgcacatgcagcagccggtgcgtaagcgcaagcttatcgtcaatggcgaaccgcaagattacaacgaacatttgttctggaacatgttagcaacggtttttggtttgcctgccaccgtttacccattggcgaaaacgatggatgagcttccgtgcggcatacagatcatttccgggcattttcacgatgatgtgacgattaactttgctgagttctgcgaaagcatcagtggcggattcacggtaccggaagggtactag

SEQ ID NO: 1077 - DNA, 1440 bp, Escherichia coli
atgacggtgaataagcttgagccagacaacggcacgccgaacgatgagctgttcacggtggcgggtatgtttgacggctcgctgtataaattactcctgcgcatggcgttgccgatgtttgtcggtatgttaacgcaggtgacctatgccatcgccgatattttttggctgagccacattgatgtcaccaacagcggcatcattgccggtgtggggctggttttccccgttggaatggggttatttgccatcgccaacggcattcaaatcggtatgggatctttgctgtcccgcgccatcggcatgcagcggttggatcgtgcacagcggattttgtcggtcggtatcatcatcgcgctattctttgcgatcgtgatcaccgtacttggctatgtttatgcccagccgctattgcgctcactgggggccacgaagagcatcatcggctacgcaacggagttctattattattcgctgctgaccgtgttcagcatcatgttgattggcgtcatgatggggctgtttcaaggcgcgggtaagatcatggtcatcatgaaggcctcattgttgggggcgttggtaaatattatgctcgatccaatcatgatctttgtattcgattttggtgtgaaaggggtggcattggcgtcgtttctggcgcaactgagtatggtcgcctatttcatctacacattgatgggattacatatcgggttgagtatacgcatcgcgttacgcccattctcctggaaaatctaccgagagtttctctccgtcggcatggcgcagatgctgatgcaactgatcattgcggtcggcatagtgatttataatttttttatcgtcaggctggacgtaaacgcgatggcagcgtttacgctcactggtcgcatcgactatttcatcatcacgccgatgctggctatcgcgaccgcgttgctgactgtggtggggcaaaactggggacacggaaatgtcacccgcacgctgaatgcctattgggccgccgtcgcgctggccttctctattgtgctggtgttagccgttatgcatattgtactggcaccctggatgtatcccctgtttactcgtgttgttgccgtgagtgactatgcggtactgcaaacccgtatcatggcactggcactaccctttgtcgctatcagcctgttagcaagtgaatattatcaggccattggtaagccttggtattcggtgttgttgacgctgatgcgtcacgtgtttatatctgtccctgtcgtatacctgctggcgattgtgttagagatgcgtattaccggcgtctattttggggcgatgagcggcacttttgtggctgcgttgttggcgtggcgtctgctgcgtctttcacctcgtctgctgcgatggaatcaggaggcggtgcgctcccaacacttggatatggaggtggcaccatga

SEQ ID NO: 1078 - DNA, 4368 bp, Escherichia coli
atgatgtcgggcaatccgttgtcgtggccacaggaacagtgccacatcattgatcaactgtatccttacagtgccgtaaacatcattggtggtgtggtcaccattgagggaattgtcgatctgccccgactacacgcggccatccagtccgccattcggcaatttgatgcgttgcgtatgtggtttgtgatgggagaggagagtgaagtggtcagccaagtgcagccttatcactggcgcgatatccgccatttgactttttccccagattatgacaaggagaacctacgcccagcggcgatcgaaacatttgttgatgagtggttccggcagccctttaccctgctggcgcacgatttgttcgagtttgtcacctttacgtgcggtgaacaatacagcggttatttatttaaagcccatcacggcattgctgatggttggtcaatggcgttgttgtcaaaccatgtcaagcgagcatacgaacagcaggacgtaccagatgatgcgtcaccagcctatagcgcatttctggcgcagcagcaatcttatcaggcgtcaacgcgctttgccgttgatcgtggctggtggcgtgactatatcgacgaataccgcgactgctttccagacagttcccccatcgttactactgagggcatcagttgttccacttggcttgagccagccatgattaaccgtctctacaggttgtgtaatcgttatggctgtacgcttaatacgctgtttattgcactcttcgctctctatcgcgccagagtatggggggaggaaaaaggcgtaataggcgtgccgttagccaatcggcatactcgcgaggcgcggcgttgctttggcatgtttactaatcaattgccgctggcctatcgactggtgcgcacggaacgattctgtgaacgggtggcctttttccagcgcgaactgaaacgcggttttaaacacagcaaatatcccattaccttattcaaccaggatttggcggaacaggggggagggaagttgcgggcgttcgattattgcgttaattattataactttacctatgaacgccacattgcaggtgccgcgcaacgcgtggagagttactacagcggcgagcagtcctataagctacagattgttttgcaaactgttaataatcataaagaaagccttaggttgagtctggaggcgctgcgtagcgctttcactccgcaccaactgacagcgatgaaaaatgggctgttggatttagtcactgcattggatcggcaacctgacgctcgtctaggcgatctcgaagtctatcctgcgccgcacgtcgcattggcatgtggctcactcaaaccctcatttacttcccgatttgcagcgcaagttgtggagcatggtgatcgcacagcactaattgataatgaacagtctttgacttatcggcagttggatgacgctgttgagcgggttgcgcgttatctgcgccaacaggggatcggacgcggtcaagtggttgggattatcgctgagcatagcgctcaaacggtgatggttatttatggtattttgcgctgcggcgcggcatttctgccgctaaatcccgcgttgccaacgacccgcctttacgccatgtgtcgtaaagcgcaggtagcgcatatcctttacgatccggcgatgcatgaattgacgcaggcgctggcgtttcccgcgtccagcttgctccaggccttagccacttcggcgcttgctagagaaccttggccagctattgaaccgcaggatctggcatatgtgctgtttacctccggatcgaccggcgagcctaagggcgtgcaagtctctcatggcaatctggctaactatctccatttcgccgccgaacgttactttacagcgcaggaccgggctgcgctctactcttcgctgtccttcgatttgaccatcactacgctgttcgcccccttatgtgtgggcgctagcatcagcgtttgtcggcacgccgagagcgaaacattgctgcgcatggccgtggttgatcagcccaacacagtgatcaaactgacgcctgcacacctgcggttgttgtgtgcggctggcataagcagtgagcagattcgtaccttggtggttggcggggaggatttcaagcgggatctggcgcgtaaggcggctgctctctttcctcaggccgtgatctataacgaatatgggccgacagaggccaccgttggttgcatgatctatcgctacaccgggcaagaaacgctgccttcattgcccatcggtatggcgatcgacggttgccaggtagcaatttgctccccttggggctgtccggtaccggaaggcgaaactggtgaattggtgatttatggcgcgtcagtgacgcagggctatatcgatgcgccgcaacaaacggccgccgcctaccttaaagataccaatggggtgatgattggctatcgcagcggtgatattggctacgccatcgctcctaatacgctggtttatcagggacgtaaagacgatcaggtcaaaattaatggctatcgtattgagctgtgcgaaatcgaacaagcgttgttgagcgcacctcaggtagaaagtgcggcggtggcggtgattgatgatgtgcaggggcagcacagcgggttgctagcctgcgtgacaccgtcatctgttgatgtagctaccgtaatgcaacatctgcgtcagcaattacccacctacatgcaacccaagcagtgctgtgctatcgcccaactgccgctatcgcacaatggcaaggtggacgtgcgtcagatggtcgcaacggtccgaaacaccgcgcctgcatcgggtagcgagcggctgggagatgcggcgataaggcattcagttcgggtatgtgtggaaggggcgctggagcaaactgagtttgatgataacgaaaatctttatgtcttggggttggactccatcaagagtatccagattgcagcgcagctcaggcaccacggctggacgatgtcagcggtgcaggtgatggagtgtggaacagtgaacgccatctgtgagtttctcgctagtcatacaacggtgtcacagctcgcgcagtatgctcataacacccgtatcgatctacccgcattgcgttggtttacgcagttggcgttgccagtgcccaatgtttataaccatgtcattgtgctgaaagtgttgccgggctgcccgctcgagcagttacacaacaggctgcatacattaatccagcagcagccagctttgcacagtgcgctggacgctgaggggcggttgctagtatgcgaccctaatgtgtgttatcccaacgaggtgctcacggaatattcgacggcgcagtggacgttggcagaggtgatcgctcagtgcaatagcatgcttgacgtgacaaatggtcgagtctttacggcagctctgttacacgccccgcagcctgcgtctagcacgctggtactctgtgcccatcacttgtgcgtcgatatgcactcttggtatttaatcctcagcacgctggacgccgtcagcactgtcaatggcaccagcaacagcggactgcatcgctggaatgactatctggcgagcaaaacggtagattccgccacacacgaaagctggcgcacagtatgtcaaacgctaccgctgcatttcccacccgtgtcattgccagatgactcattacctcgtacgcgcgcttggcgtgaagactttcgccatccttgcgtcaggaggttgtttgaatccagtggtaataccgcgtacagcgccgaaacctatgtgctaacggcattggcgctggtgctgcgttactacagcgaagagccttggtgtcgcatcgagatggaaggcatgggacgcggctgctggcccgatgagcctgacgttgcggatacggtcgggtggtttactctattttatccttgggcgatccctctgcatggcgatatggcgacactgttatctgcgatcgcttccgatttggcaaaacgcacgcatggtgggggagattacggtctgctgcaaatgcggcatgcaccggaagactctcttgcgcaagggatccgaatgaattacatcggtgtgcaggcgcaaccttcactccgctattttcatattgatcactttaatagtgatatctataccgcgcctgaaaacgcgctcggctgcgtgctggagtttaatattgcgcgctcggcagctgatggtctctccttccactgccgtttcgatcccacgcgcattgcgctgaatgatgtgcaactcctgctggcgcgctacaagaacagcctcactgatctggatgcctggctgtgccaacacagcgccacgctgacgggcgcgccgactttgtggacactataa

SEQ ID NO: 1079 - DNA, 2460 bp, Escherichia coli
atggcaaaggatgattttacctgtggctcactggatattgccattattggcatgagcgggcgtttttccggtgcggaatcggtgcccgagtggtgggataagttgctggcgggtgaggagttcactcaaccgacatgcacagaagacgataatgggaacccttggattaggctgcgcaatataatcaccgctccctatgacttcgatgctgcatttttcaatattccgcctggcgaagccctgttgatggatccgcaacaacgaatatttcttgaatgctgctacaacgctcttgagcatgcgggctatattcccacgcaacttaagcgagtcggcgtctatggggcgacctacgccaataactattttatcgatcgcgtctatccatacctgaagatgagcggcgatcaccattatcttcaggcgcagattggtaacgaaaaggattacttgtgtgctcaggttgcctacaagttggggtttaccggacccgctgtgagtgtacagaccgcctgttccagttcgttggtggcagcatatttagcatgcgaggggttattgacttttcaagccgatgttgcgttggcaggtggggtcaccttgggttttttacaggcgcacggctacagtccgcaaggtgataagctggtatcgcaagacggacactgcgccccgtttagcgctgaggcgaccggcacggtatacagcagcggtgcgggagttgtggtgctaaaacgtcttgaggatgcactacgcgaccaagacagagtgtatgcggtgattaaaggaggtgcggtaaataacgacggcgggcgtcggctggggtttgtcgcccccagcgtagaaggacaggtagaagcgatcaatacggcgcttgccgccgcagaagtcgtaccgactgatatcgcactgatcgaaacgcacggtaccggtaccccgttgggtgatgagattgagcttgaagctctgcaccgggttttcgcaccggcatgtgcgccgcacagcattcaattgggtgcggtaaaagccaacttgggtcacctgggagtcgcttccggtatcgtcagcttaatgaaaaccgcactgacgctatatactggtttagttcccccgcagatcaacctagtaaataaacataaaaaactgttgcagccagcctcgccattttatctcagtgatgtagtgacttctgtcccacaaacgaaaaggatccatgccaccgttagttcgtttgggctgggagggaccaatgctcatttggttctgcaaaactggtgcgagacgcctgcgcaagcggtgcaagaaaatgagcgccgcctgtttttcttcagcgccaaaacgccattagcccttagacaacagttggatgctcattatcacgctctggcgacctatgcggaagcagataaagatcgtatcgcctatactctcgcgcaacgccgggcacatttcccgtatcgctgtgcgctggcggcagacagtgtggtagcgttgcgtgcaagtttggcgaaactgcgggatgctgacatgtcttttacccctatcaatatggaaactaccttggtgttcctatatccggaccgggacgataagctggagagtgccctgacgcatttgctggcttgtcaacctaacttgcgccagcggcaccagcgcctgtctcaggatgttgcacaaatctgcgaaccagccgattggacgccagcattgcgtcagtttatccagcaggtgagtcttagtgaatggctgatcgaacaaagtatctcgcctgtgcagcacatcggttacctgacgggggctgcagcagcgcagtatgttgcgcgcatcatctcactggaaaacgctgttcagcaggtgattgtggctgaaacgacaccggagcagacgctggccggcaacagtgaactgagcgaaatattggctaatctggcggtgacggagggaacgctgatgctggaaatcggtcgcgcggggacgttttccatattgtatcaccagcatgcgcagtgggttgggcaaacagtattttcccccatgctgaatacggatacgcctgaggacatcctgccgctgttgggcactctttggcaacgaggagtgactatttgtttaccggaaatgccagccgtgcagactataggcttacccggttattcgtttgatcgcgtccggtatgaaattcagtccagtgatgcgagagagaacgctatgttaccagtgagttatttgtcggtcagcgactttgtggagaaaacctggcgttcattgttatgcatcgatcattatgacgaacacgcggttattttcgaatacggagcgacctcgatgcacgttatctctttcgtcgacagctgtaaccacatttataaaatcggactgactgccgccgatatttatgccagacccgctatccgtgagcacagtgaatttatctccgaatgtgttgatggtattttatga

SEQ ID NO: 1080 - DNA, 1515 bp, Escherichia coli
atgacaataatggaacacgttagcattaaaacattatatcatctcctgtgctgtatgctgctctttatttccgctatgtgcgctttggcgcaagaacatgagcctatcggggcgcaagatgagcgcctgtcgacattaattcaccaacggatgcaggaggccaaggtcccagccctttccgtaagtgtgaccattaagggggtacgtcagcgatttgtctacggtgttgccgatgtggctagtcagaaagcgaatactctagacacagtttacgagctgggatcgatgagtaaggcgtttaccggacttgtggtgcaaatactgattcaggaaggcagactccggcaaggggatgatatcattacctatctgccggaaatgcgcttgaattatcagggaaaacctgcttccctgaccgtggctgatttcctttatcatacatcaggattgcctttttcaacactggctcggctggaaaaccctatgcctgggagcgctgtggcacagcaactgcgcaacgagaatctgctgtttgcgccgggtgcgaagtttagctatgcctccgccaattatgatgtgttgggcgcggtgattgaaaatgtgacgggaaaaacctttacagaggtcattgcggaacgactcacgcagccgctgggcatgtcggcgactgtggcagttaagggggatgagattattgtcaacaaggcaagcggctataaactgggattcggcaaacccgttctgtttcatgcgcctctggcccggaaccatgttcctgccgcctatatccatagcactctgcctgatatggaaatatggatagacgcctggttgcacagaaaggctttgccggcaacgctgcgtgaggcgatgagtaacagttggcgtggtaatagtgatgttccgcttgccgcagacaatcgtatcctctatgccagcggttggtttatcgaccagaatcaaggcccttacatcagtcacggtgggcagaatccaaacttttcttcttgcattgcgttgcgaccggatcagcagattggcattgttgcgctggcaaatatgaattcgaatctgatactacagctttgcgcggatatcgataattatctgcgcattggcaaatatgctgacggcgctggtgatgcaattacagccaccgatacccttttcgtctacctcacgttgttgctgtgtttttggggggcggtggttgtagtgcgcggtgctttccgtgtttatcgcgcaacggcgcatggccctggaaaacagcagaggttacgtttacgcgtacgtgactatatcatcgccttggcggttcctgggctcgtggccgccatgctctatgtcgcaccgggtatactatctccaggacttgactggcgttttatcttggtatggggtccatcgagcgtgttggcgataccgttcggaattatcctgttagctttcgttctgacattaaatcatcaaattaaacgaattctattacacaacaaggagtgggacgatgagtaa

SEQ ID NO: 1081 - DNA, 723 bp, Escherichia coli
atgagtaatatcagtttgtattgtttgccatattcaggtggttctgccgccatgtattataaatggcgtagcgtgctgtcggacaatattactttgcggcctttagaacctgcggggaggggaactagaatacgccagccgctgtgtcttacgatggtggatgccgtcgctgacctttatcaacaatttgtgaaacactacacaggtggagactacgccatttttgggcatagtctcggagggatcatggccttcgaactggtgcattatattctcgatcatggacatgacatgccatgcgcgctgtttttttccggctgtcgcccacccgatcgggcctctcatgaagtaatactgcataccttgcccgatcaggcgtttatggaagagatcgtcaagctgggcggaactccggttgatgtctttcgtaataaagagttaatgacaattttcacccccatcattaaaaacgattatcggctctatgagcagtatgtatttcaggccaaggcgcgcacattaacctgtccgatcgtgctatttcatggcgatgctgacaatctggtaatgcaggatgaattacttgcatgggaaaaattcaccacacgaaagacgcggactattatatttcctgctgccgatcatttttttgtcgataagcattttgaacaggtggtaggttatgtgaaccagacgattgaatcactcgaaatagtagggtag

SEQ ID NO: 1082 - DNA, 213 bp, Escherichia coli
atggataagttcaaagaaaaaaacccgttatctctgcgtgaaagacaagtattgcgcatgctggcacaaggtgatgagtactctcaaatatcacataatcttaacatatcaataaacacagtaaagtttcatgtgaaaaacatcaaacataaaatacaagctcggaatacgaatcacgctatacacattgctaacaggaatgagattatctaa

SEQ ID NO: 1083 - DNA, 513 bp, Escherichia coli
atggctgttccatcatcaaaagaagagttaattaaagctattaatagtaatttttctttattaaataagaagctagaatctattacgccccaactcgcctttgaacctctattggaagggcacgcgaaggggactacgattagcgtagcgaatctggtttcctatctgattggctggggagagctggtgttacactggcatgaccaagaggcaaaaggaaaaactattatttttcctgaggaaggatttaaatggaatgaattggggcgtttagcacagaaattctaccgtgactatgaggatattacagagtacgaagttttattggcacggttaaaggaaaataagcagcaactcgtggctttgattgaacgattcagtaacgacgagctttacggtaaaccttggtataataaatggacccgaggtcgtatgattcaatttaataccgcctcgccttataaaaatgcttcggggaggttaaataaactgcagaaatgtcttgcagaatag

SEQ ID NO: 1084 - protein, 244 aa, Escherichia coli
MRIDILIGHTSFFHQTSRDNFLHYLNEEEIKRYDQFHFVSDKELYILSRILLKTALKRYQPDVSLQSWQFSTCKYGKPFIVFPQLAKKIFFNLSHTIDTVAVAISSHCELGVDIEQIRDLDNSYLNISQHFFTPQEATNIVSLPRYEGQLLFWKMWTLKEAYIKYRGKGLSLGLDCIEFHLTNKKLTSKYRGSPVYFSQWKICNSFLALASPLITPKITIELFPMQSQLYHHDYQLIHSSNGQN

SEQ ID NO: 1085 - protein, 3206 aa, Escherichia coli
MDNTSGDFPCNKMDTRKQLPLTPSQQGFLFHSLKDKKRSNYHEHFTCIFSQHVDSAHFKWALETLFRKHECFRTDYNWEIDERPCQVVKTDVLPDIYVLDCEQEEIRFLLANDDIIIPVPQDDGIDAIIPQLLQADLKYPFSLKTIPVRAYLIQSTKESAFILSYHHIVMDGWSLSLFIKQLLQLYGAAVVSGVRDDSAIIPSSLKPLVDTLSARRHTFQHDYWAAYLREGTPTCIVPLSQYHTDTEAENNSYVNQTNHVEINLSPDVCQKIQTLCSDYRITPAVIFYVAWGILLQRWCYADDVLFGATISGRNIPIDGIEETLGLFINTLPLRLRDDGATLLQHLQRMHQTLIAHYSNEHDALASIQRLVHKEGHAGDLFNTLVVLENYPVDMTLLSCASPVAIRHLSVHEQTHYPLTLTITQQKGFRFSIAYALNYLTNNMAQALLMHLSYLLEQLVDNPQRPIAALVNLSPCQQAQVLQPYLERMACRDWDSQSNVIEQFHQVAATSPAQVAVVDELCALTYSELAAQAEQLAAYLVQQGVMVGDTVGIISERRVNTVVAIIAIMLIGAAYVPISPDYPVGRMQEIIDDSGLALLLVHGKPLDALNVAQSDLCAFPVAPSVVFPVITPDSRAYVIYSSGSTGKPKGIAVAHRGLLRLIQGDSPLKVESGETTLLTCPFEFDVSVFEMWSTLLNHGKLVLLSKQALLDINHIRRTIADEQVARAWFTSSLFNSYVAEGADFFGMLQHITVGGEAVSAWHVNDVMQKYPHLVVTNGYGPTENTIFTTAYRFNGLQPARVPIGYAVPGTSLYITDLHGHLLPIGATGELVAGGVGVAIGYQNNPALSATVFVPDPFIPGGMMYKTGDYARLLDDGCVDCFGRKDGQIKINGQRIETGEIEQRLLECSGIIEAVVVPYRVRETLHIAAVVCVNDSYDEVEVRGQLADRLPPFAIPESLVVVTEIAKSHSGKADLAQLRYLLPATQCNAVSTTISEVHSDMEHALHAIWQRVLDRQDIDSNASFFALGGTSLDTIRVKGDIKRQLGLEIDITDLFKYPTLTALAHFLDTAVSPEDAIPTRAVVYSDMPVAIVGMAGRFPGAANIAALWTLVVGGESGLTLFSDEELRAHGVTPDTLKQANYIKTKGIVDDHEWFDADFFGYTPNEAECMDPQIRLLHQCCWQTLEHAGCDPATFTGAIGIYAGLLTSPHWLNAVMQDTTDSTALYKASILNIHSVTALIAHALNLTGPAVTLDTACSTSAVAIHQACIALRNRDCDAALAGGVSIEMPAYRGYEYHEGMINARDGVCRPFDSQASGTVTGDGLGMLLLKRLDDALADRDCIYGVIKGSAVNNDGNNKIGYTAPSVIGQSTVIRTSLRRAGFDSDSIGLVEAHGTGTVLGDPIELRALNEVFGPTPVPFCVVSALKSNIGHLNSAAGVAGVIKTTLALHHQVLPPTAHFRQLNPAIDLSRSALYVNQQVQPWPSTRPRRALVSSFGIGGTNASIALEAHQHEDDPSATGVRDSYLLLFSAKTPAALELRVASTLEYVKHGVGVRLPDVAYTLQTGRTAFDHRRAYLVSRGSKIDLSCATILQAEIFNGQRTTAEICFMFPGQGSQYHGMASALYAHQPMFRQHMDRCFAAFQRYSTVDLKALLFDDEDTRDIDQTQFTQPALFCVEYSLARTLIDLGITPDSMIGHSLGEYVAACIAGVFTLEDALHVIEARGRLMQSMRPGSMMAVYLSREQLTPWLAAERGIELAANNSAHFCVVAGEQAAISRLSTRLVEGGIQHRRLKTSHAFHSAMMTPMLHDFAQLLGQIPMHAPHKRFISNVSGTWITEEQATSPDYWVQQVRNAVLFSEGAAQLLVQPTLFIECGPGNTLSTFIQGHNQYSDQPTLLTLRKANAAIDDEHMLHRTLAALWVRGENIDWRRFNQTALGKHIPLPDYPFEQTYYYRYGAALSGYRQYPNPLRRPQDEWLQRVLWRMHDTSLREAFYAPGELIIIISADGDKLQQTLMSSGVDSITMPLPISSEDDVWDNDRILTHFHDICALLAHKTYRQLHCLYAPGAEAGSSLTQSLSGLYRVARWCMHSTTPLASLTVLTHGAFRVQEEDNPEPTLAALSGAVNVFAQELHPTEVRLIDIDAQSSDENLNLLTQRLAPKQETVMALRQGMLYLRRFIPTRLLAHLPPQTGCIPGNVLWIIGGEKGIGRMIGEALAQREGVRVVLSSRTGYHHEAVQQDALDVIHCDVTQAEAVRACLATLLERYGRLDGVIFAADATTTLTLHQLSESALRDTLTVKERGTANVLHALAQRNLLDERLLLLFCNSLAAVNAEIGQTGYATASAYLDALAQQLRTRYKVNALSIGLDALREQGMLLDAINGSEYDVLRGLRPLMTGTLLQAYKQQGADTSYYARLSPESDWLLDEHRISGIATLPGTGYLALAYEALRHYFVQDQICIDELVFLAPLTVMDNCSVDVFVDISPNGQGVSVEVKSMTERFSGTLTTHARGRATRLMVDDNVVCDLTGLMREMHTITPPTKELSSTHFHYGPRWHSVQQLYGNTAQTQVFATLALPTVAANDTIALHPALLDIASSVVEQLPGFHTDSVPFLYQDLRLYRPLPNTLHVALTVNRHDEEGDSYAFTLYDMAGEMVARCAAMVKRKVQLHIQDVDDDTRLRVPSADNYQLRLAAEGEGAGKLALCPTPRLALGDSQVEIEVLATGLNFKDVLFTTGLLRQQPGEAPLQLGLECAGRITRVGKNVTEFAPGEDVMAVLNGGFVQYARVESDCVVRKPAHCRIEQAAALPIAYLTAYYALVVRANLQPGERVLIHSAAGGVGLAALHIAKRCGAQIFATAGSEQKRDYLLSLGVHAVADSHDEQFAATLLTASDGQGMDVILNSLTGRLLDASLALLAPLGRFLELGSKDIVEDKALPMRFFAQGGTFIPINFHAAHGAFSRYLQQIVAWIDDNTLPLLPCKSVPLPEVARAFATLTTPQHIGKVVVTHRTAAGMDRLNAMIAERRLGGYALSMSNAEVMRQLWPILNTRSPWAQLLLSPRAIDRLARGNRVDRGVPSAANDTITQQTVKKRPRPEIGVPYSPATREVERVLCQILEEYLGLDRVGIDDNYAELGATSLDMVQLSGQMARHYPQVSVVSLYNHATVRQLATFCQPPEGESNAPSPQPAVQTNTRANQIAKRALQIAKNTARSHTSLH

SEQ ID NO: 1086 - protein, 866 aa, Escherichia coli
MEYASEMNGMEIAIIGMAVRFPQSRTLHEFWHNIVQGKECVTFFSEEELLAEGVEQSTLDNPAYVRAKPYIEGICDFDAAFFGYSHKEAQTLDPKSRVLHEVAYHALEDAGYAQRTSDLITGVFVGASEDVDWLRRSLSQIGGDALNRFESGIYGHKDLLAHLIAYSLNLNGPVYSLYTSCSTSLSATHIACRSLLFGECDLALAGGITIDLPQKSGYFCQQGMIHSTDGHCRPFDSQASGTLFGDGAGVVVLRRLEDALAAGDRIYAVIRGSAVNNDGKQKIGFVAPGHEGQKAVICAACHLAEVSPESIGYVETHGTGTRIGDPIEFAALTEAFDTSHRQYCALGAVKANIGHTHAAAGVAGLIKTALVLHHRTIPPLANYQMPNSKLDLAHSPFYIPIQPQEWPASRMPPRAGVSSFGIGGTNVHMILEGLNPAVRDDHDQVRAPVFIPLSAPSFEQLDELTQQLTPLLATLDASTLAYTQQVARPVFDCRRVIQVENDGTQAMLASLDNLMPDAPWGLHCPDLRTTNDCTYAQWLAHSAHYQREATALTALLDGMNIPPAYCHAETWAAQANSSLLIRGCQTIAALKTWMNLLPTLTLLSGAGTGLLPAAAASGMIATQDVLHLLWEMEQKALHLWLPERHEPIPGYVLAWQGNPITDAQRNDRGFWSEALLADTRELGEGVHSINWVRLPPEIREDVDVLRYVAQLWCAGINVDWAVWYGTPLPQRGSASAYPFAHNHYPLPGRVMGSVETQPEAGPETHHPYQARPVLSVPFVAAHSRGMQYITGLMELLLEISPVGVDDDFFELGGHSLLVTQLTSRLERDFNVHIDLLTLMENPNPRNIYAHIAAQLGGEDNLEIACQ

SEQ ID NO: 1087 - protein, 289 aa, Escherichia coli
MMNVAVIGAGVMGTGVAHNMAQYGISTNVVDISQSQLDKCRQMIEANLRLYNFHPQHKKKTHSTAEIMENIRFTTELDDIVECDLVIENITEDIEKKNALYTRMNTICGASTVFGVNTSAISITALSKLMRHPENVVGVHFMNPVPLMHTVELIRGVHTAERTLNIFHHLFAQLNKTGIVVNDSPGFVTNRAMMIFVNEAIFMVQEQIARAEDIDTLFKTCFGHKMGPLQTADLIGLDTILQSLQVLYESFNDDKYRPSFLLKKMVDAGYLGVKSGQGFYRYQQTYAEQ

SEQ ID NO: 1088 - protein, 82 aa, Escherichia coli
MKKQDMKAAIREFLSRSLRGHTLNDDDDIFSLGLVHSLFTVQIILFIEKNFQVELEVSELKTEQIATVNKIVELIQRQTGLE

SEQ ID NO: 1089 - protein, 376 aa, Escherichia coli
MCTENYELAQQEAVLFAKQHLALAAQNIERQQFIVPDIISCVAQAGYLGASIPQKYGGRGYDSYQLCALHEVMAGVHGSLENLITVTGMVSTLLQRVGSAAQKAHYLPKLATGELIGAIALTEPNIGSDLVNVETELQQDGDGWRLNGKKKWITLGQIADFFIVLIHCGNQLATVLIDRNTDGFTITPLNDMLGLRGNMLAELHFNDCRLKEDALLGPLTPGVPLAVNFALNEGRFTTACGSLGLCRAAVDVAARYIRQRKQFKRRLFSHGIVQHLFATMLTQTRSAQLMCFSAAEYRETLHPAMINQILMAKYVASKAAVDVAGKAVQLLGANGCHADYAVERYYRDAKIMEIIEGTSQIHEIQIAMNYMMGSEA

SEQ ID NO: 1090 - protein, 422 aa, Escherichia coli
MTKDVALMFPGSGSQYVGMARWLYERYPQVRTLFDEASQITERDMAALCLSGTLVQLAEPTAMALAIYTTSVAHFVAWQQFLAQTGVHVNLRYMLGHSLGEYAALTCSGALSFSQALALVAMRSRLASEIAREMDASTTIIKQGNQALVAAACEVAERETRQQVGIACFNSPQQFMLSGQNSAIIAAEQYLLDHDRQVEVVPLIGGVPYHSPLLKPCGQQLRKALDRCEWRRPCCPVISNVNAQPYPDTVTPVQWLEQQLSQPVQWQRSLTYLTGHLSPIAIEIGPQSVLKNLLLENRYPAPVYAFDNRHDRAQLALVLGDNMAVKTDPEAVRRQRITLLTNALTATRHHRAADVAASAQLKELLSRFFERIQQIEQRGTSSEEDIAFLHELLEQGFQLKGSSQAEIDACHARLASDNGGQA

SEQ ID NO: 1091 - protein, 2310 aa, Escherichia coli
MEQQGIMRQLPTDDQTIVDYLYRIAGEYGEKAAVLMGDAALSYHDLNARSNQLAHYLRGLGIGEDRVVAIRLPRGMAMLIAIFAIVKAGGAYLPLAYNAPRSRIENILSNSGAVCLIGTDDGDRWPIPRVEIDSAAVSAMPTTDLRYRPHARQLAYIIYTSGSTGVPKGVATEHAALLNRIVWMQNAYPISSQDVLFQKTVYTFDVSVWEMFWWAMYGASVVLLPSGLESDPRTLARLIQRHRVSVVHFVPSMLNLFVEYLEMKQDPRLTASLRLVFSSGEKLTVHSVARFYQSVAQGDLINLYGPTEAAIDVSHHRCLRGYDYDDIPIGQAIDGCRLYVLDDHGNPVADGEEGELYLAGIGLARGYLNNVALTDRCFTIHPTLRHLGKPERLYKTGDLVWRDGESQQIHYIGRNDFQIKIRGLRVELGEIEAHAMRFPGVQQAVVVADQDDPDNQLIYAFVVSSVPLNLAALMDALSKNLPAYMLPNRLLAMSELPLSDNGKCCRKTLLDLARAYSASRVDLRETPAVRYLPLSSAQSSMWFMQQLAPHTALYNNPTALLLEGELDRTRMDGAIRQLMSRHTLLRAMAETHNGQPVLAVPQCVSSQALLTIVPLPSVSDDNALQAMINQRAAHPMPLTSGTPLCRFELLTLDDDRSVLLIHLHHIISDGWSKGVLLRELQAAYNGESLTPEPLLEYADYMEYQEEWRQSDAYQDAMRYWQNTLAGTLPILDIPTDQPRQKVARYQGAFVAFALSANTCERVLAAARAQRVSLYNYLLTAFVLLLHRNARQQEYIVGMPIAARLTKEQEHMIAPLVNVLPLRLPLDEAASFSELVQTIRGILFAAFRHQRLEFTDIVRAVNVDRSAGTPCALLPDMQAYLLFTSGSTGEPKGVCVVHRGLLNLLLDMQRTFAVGSQDRLLSVTTPTFDISFLEYLLPLISGASLYLTEAERAADSFRMIPLIADYRPTLMQATPSFWHGLLMAGWRGDPELCVLAGGEALPTKVAEELLRCCGSLWNLYGPTETTIWSLKSQITQAENITLGAPIANTRIYILDNEGHPVPQGVDGELYIAGDGVAQGYDGQPELNAQFFLSEPGVPGGRMFRTGDLVRSDAQGQLFFVGRKDSQIKLRGYRIELGEIERTLARHPHVDAAVVACIERAPLHKALAAFIITSEPPSLFEQLKNELRQQLPDYMVPTLWQRVADFPNTDNGKIDRKRLAENFVADSSLVSPQTQALSDTEQMLLALWMRYLPIKNVDPECDFFRLGGHSLLAVTLVAEINRTFHCALTLKDIFHYSTLRALSARIAQQSITDAAASQDDWVIVHDPEHRHQPFPLTDVQRAYWLGRQTGATSIATHIYHEFDVEHFNVTRFTHAVNALIARHEMLRARVLPDGTQQILAQVPAYQLEQRDLSALSPNARNDALMAIRDRLSHHVHPADRWPLFDFSYSACTAQHGRLHFSLDLLIADALSMRTLQQELMMLYREPHVSLPLLPFSFRDYVQALLVEQASEAYARDQAYWQRALPQLYGPPTLPVQGDLAQLSAIRFVRRRHRLSAHNWGVLSALAQRTRITKTALLLTVFHFPIYQCMFQLDNMPLASPTLNGVNVTPLLLDTSASQVDISLSMQHIDGRITGTFEYDAGLYSADRIQHLVAQWRELLDEASSQPTQLVRDLIRFTPREHAWLARHNATEVALPPVDNLLALVLPHCQQRPTQVALRHADDAMTYGELQQATMQMCTWLRAQGVKRGESVALQLPFCFELIIAQLAILSLGASYVPLDGNAPAARNALILAQATPCMLLVAQPLESPHGLTIPWVLVPDWRSLLTEIPNLPVSVAPDALDCDAVVIFTSGTTGQPKGVRLSQRNLVNLTASFISSYQVTHQDVLLPITSVASASFVGEVLPLLAAGGTLVLAQKAQSLDSDALIALLASQRVTILSTTPSLSASLSVLAQSMGSLRLFLCGGEALEYEQIAPLLPHMAVVNGYGLTESGICSTYFPVAKRREQETGALPIGRPIQNTQAYVVDAYNRLVPPGACGELCFSGLGISPGYLDARQDPERFVELPEYPGVRVLKTGDRARWATDGMLFYLGRQDRQVQIRGYRVELGDIESLLKQHPDIADAWVDVRRNAAATPLLVAFYCSVNGVALDAQQLRVWLSLRLPLHMLPLLYVPLSAMPLGVNGKIDPQCLPLVDLRQLEGPGEYVPPATELEQRLAEIWQQLLGLERVGTTTNFFDLGGHSLLLVQMQQYIGQQCGQHVALVDLLRFTTIKRLAEFLLAPDAAQGTTGDQTQLRAAKQRLAFGHTRWAATTDSHH

SEQ ID NO: 1092 - protein, 1010 aa, Escherichia coli
MAENDFGIAIIGMAGRFPQADTVQAFWENLLASRECISFYSDEELLAMGISPEFVQHPDYVKAKGEVADIDKFDAAFFGIAPREAELMDPQHRVLLETAWAAFEDAGYVAADYPGDVGIFAGKSMDSYLMLNLMPHFKRVFSSGSLQAAIGNDKDSITTTIAYHLNLRGPAITVQTSSSTSLVAVCVACQSLLTWQCDMAIAGGVTLGPPAKTGYLSQEGGITAADGHCRAFSDNSSGFVPGTGAGLVVLKRVDEALRDGDNIYAVIKGFAVNNDGSEKISYTAPSVDAQARAIAQAQRLAGLTPQDITYVEAHGTGTRLGDPVEFSALSQAFAGASQKQYCALGSVKTNIGHLDTAAGVAGLIKTALAVQQGIIPATLHFERPNAQIDLTNSPFYINTTCQPWQPESGIRRAGVTSLGMGGTNAHVVLEQAPAVDLQARAPVPAYSILPFSAKTDSALSSGLARFADFLQHESLPDRRDLAWTLSQGRKAFAHRAALVTRDLHAAGTLLQQAATAPFARGVAQTQLGLGLLFSGQGSQYQRMGHQLYQVWPAYADAFDRCATLLEREYQLDIRHELFRAEVSLAQGERLAQTCLTQPLLFSVEYALAQLWLSWGITPTVMIGHSLGEWVAATLAGVFSLEDALRLVARRAELMHQAPSGAMLMVALPEAQIRALITAPLAIAAVNAPDYSVIAGPTSEILAVSQRLTEQNIINKRLHTSHAFHSSMMQDAAQALRQAFENVRLNPPTLTIISTVTGAHVSADTLTTPDYWIEQMLMPVQFSAALQEAQATFDVDFLEIGPGATLTQLTNGHALGDRLAFSSLPAGARSSDEHKHILDTVAALWVRGHNIDLSAFAGEQPRRVSLPTYAFDKIRYWVDSPEEQRSAVTPVADAGSVIPSEPSVRRQPRPAFSVPYAAPESKTQRGLVAICEALLGIDGLGIDDNFFEAGGHSLMLGMLLAQVQERFAVTLSFFDVMEDASVRALAQLVEQEQQDDGGSALAVLVNDMINE

SEQ ID NO: 1093 - protein, 1454 aa, Escherichia coli
MTIHHAALARMLPAEKKEKLLRQLAQSGVSPSRIPIIKADPAQAIPLSFNQERLWFLQKYDSTATNYNLYVVYRLHGVVDMPMLTEALRHVQARHAILRTRIIVRNDRPCQVIDDASSLVLDTVTLAAQAPTSALDAVIQQVINTRFDLARGPLWGVTQIIQPDQGCHLVFCAHHIIIDGISLRLLFDELQQQYARLHAGNETSLPPPPLQYADYAFWQREWFQDTLLANELAYWRARLQDAPLLSTFPSLHPRPAQPSTHGSRFSITLDETLSLALKHVARTQETTPFVLMLTAFQLVLMRYAQQQRLVIGMPVSGRIRPELQSSIGYYASTAVIYTDFNGVEVGREALQRVKASVKETQGRQQLPFENLVNMLDLPRSLSHSPLFQILYIYHNHVTPRAFTLAGAYWEQVTYHNQTVKYDMTVEVFQNDATFDVSFEYDLGLYDADVVKQIAEALRQHCLSLTSSLETPIGAIPLHAPETATPRRDPLNATNVPWLGPQDVLRIIEQRCVQHPKQLAIQQHDGTLTYAELWARVQFIAMRFRAHGIQPGDRIGVLLPRHRDVIATMLATWFVGACYVPFDIHQPAARLQRLMQRARLVCLVVRQPGEWGEIVQLSLPELMQDMSNAIRYSSQVLARWSLSPTFTLNLTLFNRPQGYPNAEAVIGDFTAVSLLNVCYDSQHSYAHNAQRIQVQLWEDLEHRRFSGIRASEALIHSGRFHAPMPVVFTSMLDIDGETTAQDPRDTTRFTLCPDANITQTPQVWLDHQVIELAGELHFNWDAVEQLFDTTLLDQMFGAYCHALQALVAMPQSWWGVNSSLALPTVSAPVTQAPAPTALLHHGLLRQAALTPQETALISPIRELTYRQLSTAADHVARALLALGVQHGDRVAVVMEKGWQQIAAVHGILRLGAVYLPVDPVLPPQRRQLLLTVGEVRVQVTQPGLTQLEPSLPVLIIDDGMLDTPAAPLPEVAGDVTDLAYIIFTSGSTGTPKGVMIDHRAAMNTLEDINERFGLNAQDRVFGLSSLSFDLSVYDAFAPFMVGAALVLPEAGREKDPRHWQTVMAHGHVSVWNAVPALMQMLCEYHSGDRMSYPTLRLALLSGDWIPLTLPEQMRERLNETMDIISLGGATECAIWSVYYPIGEVESTWTSIPYGRGLRNQPVYVLNAQLEECPVGVEGEICIGGMGLAQGYLNDAEKTAASFVWREASGERIYRTGDRGRYFADGQVAFLGRNDTQVKVNGYRIELGEVKSHLEQLDSVGSAAVVCHQGQLYAFITAAENLHPDDTDALLARVRAQLAVQLPYYLLPQHFFLLKVLPMTGNGKIDQAAMVQEVIQRMSQSTSQKSRALAHASPYEQQVAALWCEVLQREQIGLNDNFFEAGGGSIQIVLLHRRIEEIFKVTVPIAELFRLTTVKRIAGYLQAMQDNARAVNQTQQRDASRSRAQQRLVRRHQRQR

SEQ ID NO: 1094 - protein, 2154 aa, Escherichia coli
MTYSESDIAIVGMNCRYPGVHSVAAFETVLRTGCNILDPKVTPSNGHNHITLNNVYEHMAEFDANFFGYSRAEAEIMDPQQRVFLTCAWEMFEQSGYNPKQHDARVGLYAGVSTSFYLLTHLMNNPDKLAQLGGLQIMVGNDKDHLTSQLAYRLNITGPCVTVQASCATSLVAVHLACEGLLSGQCDMALAGGVTFRMEEQRSYESHGDGLQAEDGLIHTFDAQASGTVYSSGLGMVLLKRATDAQVQGDNILAVIKGSAINNDGGARSGYTVPGVDGQEAVMIEAHSLAEVTPQQIQYLELHGSGTPLGDAIEFAAIKRVFGTPAPNATPWRLGAVKPNVGHVEMASGITSLIKTVLSLTNRVFYPTLNFQRANPQLGLEDSPFEVVSRLTPWPEGTTPRTAGVSAFGLGGTNAHLVVQAPLSTPQARAQQMGPCVVVLSAKNHNALEQMQNALLAKLAAHPEIRLQDVAYTLRHGRFSAPVRKCVIAENCTQLARQLRDAPMVEATTGCTIYWRLGHRFVVALETLSDWLACSEVLSQAVGQLLEHFPLEPACLQDLSPAQRTFISQYALIALIDERETLNVVLCGDGDGGYAAAVLRGDCTLEQAWHRLNAGQPFDDVPTNPLLQPDVCSLMLDDAASDANRTALEALGQLWLAGVSLDWRWVDAAERMLGSQRIALPGTVFTPQRYWVEAVRPATFSHESSNNLLSRATKSDIIAVVTEIWERTLGVSIDDHHASFFELGGHSLLASTILYDIQQRYGITCTLSAFFADPTIEGLSCYLLEQGGSETAVSALPDTVFAPDQQHLPFPLTDVQQAYWVGRRKSLGLGNISTHIYVEYELQGLDETAFNRALNAVIARHSMLRAIVNDDGMQQILPNVPEYHVAFYTTQCEDAFQQRCRELRDTLSHQMIDCSRWPLFQMEVVVDPQQKARLHVSIDLLIADAWSLELFIRELAYHYRHPQAALPTLTYSFRDYVLTLKSYEKTPQFERARDYWRARIETLPPGPRLPLRTDPTKLENPTFVRRSYCLSRAIWQRLKTQAGQMSITPTTLLLTGFAQVLARFSSSPHFSLNLTLFNRLPLHADINHLIGDFTALTLLEIDMSQGETLQARANVIHSQLWRDLDNRLFGGIQVSRLLVQTHRDPAKSVIPIVFTSLLNQYEASWETDDTLFNQPQDDLYSISQTPQVWLDHQVMERNGELHFNWDVVEQLFEPALMDQMFQCYCQLLHALAQRPQLWHETQDVLALPTVSAPVTQAPAPTALLHHGLLRQAALTPQETALISPIRELTYRQLSTAADHVARALLALGVQHGDRVAVVMEKGWQQIAAVHGILRLGAVYLPVDPVLPPQRRQLLLTVGEVRVQVTQPGLTQLEPSLPVLIIDDGMLDTPAAPLPEVAGDVTDLAYIIFTSGSTGTPKGVMIDHRAAMNTLEDINERFGLNAQDRVFGLSSLSFDLSVYDAFAPFMVGAALVLPEAGREKDPRHWQTVMAHGHVSVWNAVPALMQMLCEYHSGDRMSYPTLRLALLSGDWIPLTLPEQMRERLNETMDIISLGGATECAIWSVYYPIGEVESTWTSIPYGRGLRNQPVYVLNAQLEECPVGVEGEICIGGMGLAQGYLNDAEKTAASFVWREASGERIYRTGDRGRYFADGQVAFLGRNDTQVKVNGYRIELGEIERCIARHPDVEQSVVVAVGNSQHRRLVAFAKLHDRHQAQALQAKEAEAAALAQGIIVNPAQRLAFKLKEPHIRALDGLGIALTAPADSTRYIKRRSYRHFSAQKTTLAQLGQLLSGLGQMRLPGLPFAKYAYASAGGLYPVQTYVYLHPDKIEEGVSGIYYFDPRQSCLMPVAPEVELNSGFHAGPNQSIADRAAFTLFMVADMAVISPFYGQEAAWHFSVMEAGTLCHLLEEDAPRYGLGLCQLGMADFSAVASHFQLSPHHRYVHCTVGGAIGQEAASAAALLRDFSTYEKPKETAAPLDMQSYKDAMLRGLRQQLPDYMVPSDLMLATDFPLTANGKLDRQKLQLQGEQIAHQRDGVGPIQVDSALQQRLVALWQEVLGVSHVSAEDDFFSLGGSSIELVRIQQALEAIIGQEIPIVDLFRLPTIADVARYLDEQLHNLPAAHDIVLAQAEVSQVSAARENLALRRKRAQQGEKGDE

SEQ ID NO: 1095 - protein, 487 aa, Escherichia coli
MSEQSYRSAGTLLAQLASGETTSVALVNHYFSRMAQFNKPLNAVVQQHYALALEAAARADRERLEGRARGVLHGLPCTVKESFDVQGWLTTSGAHYLKDNRATQDAPSIARLRAAGAILMGKTNVPMMTADWQTYNDLYGTTHNLWDRQRSPGGSSGGAAVAVAADFTPVEFGSDLFGSLRIPAHYTGVYAHRCSLGLMSVRGHVPGGGPQATDEPDLSTAGPMARSAADLRLMMRALSTFWVEPPRIPDFSRYQAKANYRVCTWFSAPHHEIDQQIAQRFQSFIDKLRAQPGVEVDDAMPADIDPDALFDIAVKLSGRLVSTALNGRQRLTAGLAALGFRLVGKLADVPEGITSYYQGMLKDSGEQRNTDKLRHEYSRVIETLFARYDVLLTPVSPVLAFAHMQQPVRKRKLIVNGEPQDYNEHLFWNMLATVFGLPATVYPLAKTMDELPCGIQIISGHFHDDVTINFAEFCESISGGFTVPEGY

SEQ ID NO: 1096 - protein, 479 aa, Escherichia coli
MTVNKLEPDNGTPNDELFTVAGMFDGSLYKLLLRMALPMFVGMLTQVTYAIADIFWLSHIDVTNSGIIAGVGLVFPVGMGLFAIANGIQIGMGSLLSRAIGMQRLDRAQRILSVGIIIALFFAIVITVLGYVYAQPLLRSLGATKSIIGYATEFYYYSLLTVFSIMLIGVMMGLFQGAGKIMVIMKASLLGALVNIMLDPIMIFVFDFGVKGVALASFLAQLSMVAYFIYTLMGLHIGLSIRIALRPFSWKIYREFLSVGMAQMLMQLIIAVGIVIYNFFIVRLDVNAMAAFTLTGRIDYFIITPMLAIATALLTVVGQNWGHGNVTRTLNAYWAAVALAFSIVLVLAVMHIVLAPWMYPLFTRVVAVSDYAVLQTRIMALALPFVAISLLASEYYQAIGKPWYSVLLTLMRHVFISVPVVYLLAIVLEMRITGVYFGAMSGTFVAALLAWRLLRLSPRLLRWNQEAVRSQHLDMEVAP

SEQ ID NO: 1097 - protein, 1455 aa, Escherichia coli
MMSGNPLSWPQEQCHIIDQLYPYSAVNIIGGVVTIEGIVDLPRLHAAIQSAIRQFDALRMWFVMGEESEVVSQVQPYHWRDIRHLTFSPDYDKENLRPAAIETFVDEWFRQPFTLLAHDLFEFVTFTCGEQYSGYLFKAHHGIADGWSMALLSNHVKRAYEQQDVPDDASPAYSAFLAQQQSYQASTRFAVDRGWWRDYIDEYRDCFPDSSPIVTTEGISCSTWLEPAMINRLYRLCNRYGCTLNTLFIALFALYRARVWGEEKGVIGVPLANRHTREARRCFGMFTNQLPLAYRLVRTERFCERVAFFQRELKRGFKHSKYPITLFNQDLAEQGGGKLRAFDYCVNYYNFTYERHIAGAAQRVESYYSGEQSYKLQIVLQTVNNHKESLRLSLEALRSAFTPHQLTAMKNGLLDLVTALDRQPDARLGDLEVYPAPHVALACGSLKPSFTSRFAAQVVEHGDRTALIDNEQSLTYRQLDDAVERVARYLRQQGIGRGQVVGIIAEHSAQTVMVIYGILRCGAAFLPLNPALPTTRLYAMCRKAQVAHILYDPAMHELTQALAFPASSLLQALATSALAREPWPAIEPQDLAYVLFTSGSTGEPKGVQVSHGNLANYLHFAAERYFTAQDRAALYSSLSFDLTITTLFAPLCVGASISVCRHAESETLLRMAVVDQPNTVIKLTPAHLRLLCAAGISSEQIRTLVVGGEDFKRDLARKAAALFPQAVIYNEYGPTEATVGCMIYRYTGQETLPSLPIGMAIDGCQVAICSPWGCPVPEGETGELVIYGASVTQGYIDAPQQTAAAYLKDTNGVMIGYRSGDIGYAIAPNTLVYQGRKDDQVKINGYRIELCEIEQALLSAPQVESAAVAVIDDVQGQHSGLLACVTPSSVDVATVMQHLRQQLPTYMQPKQCCAIAQLPLSHNGKVDVRQMVATVRNTAPASGSERLGDAAIRHSVRVCVEGALEQTEFDDNENLYVLGLDSIKSIQIAAQLRHHGWTMSAVQVMECGTVNAICEFLASHTTVSQLAQYAHNTRIDLPALRWFTQLALPVPNVYNHVIVLKVLPGCPLEQLHNRLHTLIQQQPALHSALDAEGRLLVCDPNVCYPNEVLTEYSTAQWTLAEVIAQCNSMLDVTNGRVFTAALLHAPQPASSTLVLCAHHLCVDMHSWYLILSTLDAVSTVNGTSNSGLHRWNDYLASKTVDSATHESWRTVCQTLPLHFPPVSLPDDSLPRTRAWREDFRHPCVRRLFESSGNTAYSAETYVLTALALVLRYYSEEPWCRIEMEGMGRGCWPDEPDVADTVGWFTLFYPWAIPLHGDMATLLSAIASDLAKRTHGGGDYGLLQMRHAPEDSLAQGIRMNYIGVQAQPSLRYFHIDHFNSDIYTAPENALGCVLEFNIARSAADGLSFHCRFDPTRIALNDVQLLLARYKNSLTDLDAWLCQHSATLTGAPTLWTL

SEQ ID NO: 1098 - protein, 819 aa, Escherichia coli
MAKDDFTCGSLDIAIIGMSGRFSGAESVPEWWDKLLAGEEFTQPTCTEDDNGNPWIRLRNIITAPYDFDAAFFNIPPGEALLMDPQQRIFLECCYNALEHAGYIPTQLKRVGVYGATYANNYFIDRVYPYLKMSGDHHYLQAQIGNEKDYLCAQVAYKLGFTGPAVSVQTACSSSLVAAYLACEGLLTFQADVALAGGVTLGFLQAHGYSPQGDKLVSQDGHCAPFSAEATGTVYSSGAGVVVLKRLEDALRDQDRVYAVIKGGAVNNDGGRRLGFVAPSVEGQVEAINTALAAAEVVPTDIALIETHGTGTPLGDEIELEALHRVFAPACAPHSIQLGAVKANLGHLGVASGIVSLMKTALTLYTGLVPPQINLVNKHKKLLQPASPFYLSDVVTSVPQTKRIHATVSSFGLGGTNAHLVLQNWCETPAQAVQENERRLFFFSAKTPLALRQQLDAHYHALATYAEADKDRIAYTLAQRRAHFPYRCALAADSVVALRASLAKLRDADMSFTPINMETTLVFLYPDRDDKLESALTHLLACQPNLRQRHQRLSQDVAQICEPADWTPALRQFIQQVSLSEWLIEQSISPVQHIGYLTGAAAAQYVARIISLENAVQQVIVAETTPEQTLAGNSELSEILANLAVTEGTLMLEIGRAGTFSILYHQHAQWVGQTVFSPMLNTDTPEDILPLLGTLWQRGVTICLPEMPAVQTIGLPGYSFDRVRYEIQSSDARENAMLPVSYLSVSDFVEKTWRSLLCIDHYDEHAVIFEYGATSMHVISFVDSCNHIYKIGLTAADIYARPAIREHSEFISECVDGIL

SEQ ID NO: 1099 - protein, 504 aa, Escherichia coli
MTIMEHVSIKTLYHLLCCMLLFISAMCALAQEHEPIGAQDERLSTLIHQRMQEAKVPALSVSVTIKGVRQRFVYGVADVASQKANTLDTVYELGSMSKAFTGLVVQILIQEGRLRQGDDIITYLPEMRLNYQGKPASLTVADFLYHTSGLPFSTLARLENPMPGSAVAQQLRNENLLFAPGAKFSYASANYDVLGAVIENVTGKTFTEVIAERLTQPLGMSATVAVKGDEIIVNKASGYKLGFGKPVLFHAPLARNHVPAAYIHSTLPDMEIWIDAWLHRKALPATLREAMSNSWRGNSDVPLAADNRILYASGWFIDQNQGPYISHGGQNPNFSSCIALRPDQQIGIVALANMNSNLILQLCADIDNYLRIGKYADGAGDAITATDTLFVYLTLLLCFWGAVVVVRGAFRVYRATAHGPGKQQRLRLRVRDYIIALAVPGLVAAMLYVAPGILSPGLDWRFILVWGPSSVLAIPFGIILLAFVLTLNHQIKRILLHNKEWDDE

SEQ ID NO: 1100 - protein, 240 aa, Escherichia coli
MSNISLYCLPYSGGSAAMYYKWRSVLSDNITLRPLEPAGRGTRIRQPLCLTMVDAVADLYQQFVKHYTGGDYAIFGHSLGGIMAFELVHYILDHGHDMPCALFFSGCRPPDRASHEVILHTLPDQAFMEEIVKLGGTPVDVFRNKELMTIFTPIIKNDYRLYEQYVFQAKARTLTCPIVLFHGDADNLVMQDELLAWEKFTTRKTRTIIFPAADHFFVDKHFEQVVGYVNQTIESLEIVG

SEQ ID NO: 1101 - protein, 70 aa, Escherichia coli
MDKFKEKNPLSLRERQVLRMLAQGDEYSQISHNLNISINTVKFHVKNIKHKIQARNTNHAIHIANRNEII

SEQ ID NO: 1102 - protein, 170 aa, Escherichia coli
MAVPSSKEELIKAINSNFSLLNKKLESITPQLAFEPLLEGHAKGTTISVANLVSYLIGWGELVLHWHDQEAKGKTIIFPEEGFKWNELGRLAQKFYRDYEDITEYEVLLARLKENKQQLVALIERFSNDELYGKPWYNKWTRGRMIQFNTASPYKNASGRLNKLQKCLAE

SEQ ID NO: 1103 - DNA, 4039 bp, Artificial Sequence
  Description of Artificial Sequence: Synthetic polynucleotide
ccgcccagttgtccgagggcttcctgcagaccttcagccagagcagagaagctcttaccacccagcacaacccgcatccagctccagtggctccattccagacggaagtgatacaacttaccgacgatggtgacaactacacctttcagttcagtaaagtccgacaggcctcgcagaccgggctgatgtcgctggcggaacatgacctcctgctctgcactatactgtagcttccattcgcgaacccgccgttgcattgttcttcgaaggctgttgggatactggccgggatatttatcctgtagcatctccagcagagttgttggtgtcagagccggcctctctttcaacagaggaacaagcatgctgtcccacatagcttccagaggaagctttgcgtgtgcgccagtgccgaacactgttgttttcccactctcctttttcgatccgacgaccagaatggactgagataccagccttcatggccgagatatgcggtcggtcaggtggtatttgaaaccagactgaagcccacggtcgtcatcgggacgcaggccgccgccgtgcatggtatcagtgagcaggcactctgcggagcaccatcatggactgatgtggtgcggcaactgcgtcatgcaatcggggaccgaccagtaattatctttaatgcccggttcgacatccgcattctgaaaaagactgctgccgcacatagcgatccggctgactggctggaagaactgacggtatattgtgtgatggagctggctgcaggatattatggagcctccaaccgctatggcactatttcactggcctgtgctgccagccagaccggactgaactgggaagggcaggcacactcagcgatcgctgacgcacggatgacggcaggggtggtaaacgctattgctgcatatcatctggaactgctgcaggaacaggcacggctgaaaacctgactgcctggcctgtataccgcaatcatttacgttatccagtcgacgcatcatcatgacgtcccatggctcgagcatgcgagagtagggaactgccaggcatcaaataaaatgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcagattgcagcattacacgtcttgagcgattgtgtaggctggagctgcttcgaagttcctatactttctagagaataggaacttcggaataggaactaaggaggatattcatatggaccatggcgtgcaccctgcagggctagctggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatatgaataatccacaaacaggacaatcaacaggcctcttgggcaatcgttggttctacttggtattagcagttttgctgatgtgtatgatctcgggtgtccaatattcctggacactgtacgctaacccggttaaagacaaccttggcgtttctttggctgcggttcagacggctttcacactctctcaggtcattcaagctggttctcagcctggtggtggttacttcgttgataaattcggtccaagaattccattgatgttcggtggtgcgatggttctcgctggctggaccttcatgggtatggttgacagtgttcctgctctgtatgctctttatactctggccggtgcaggtgttggtatcgtttacggtatcgcgatgaacacggctaacagatggttcccggacaaacgcggtctggcttccggtttcaccgctgccggttacggtctgggtgttctgccgttcctgccactgatcagctccgttctgaaagttgaaggtgttggcgcagcattcatgtacaccggtttgatcatgggtatcctgattatcctgatcgctttcgttatccgtttccctggccagcaaggcgccaaaaaacaaatcgttgttaccgacaaggatttcaattctggcgaaatgctgagaacaccacaattctgggttctgtggaccgcattcttttccgttaactttggtggtttgctgctggttgccaacagcgtcccttacggtcgcagcctcggtcttgccgcaggtgtgctgacgatcggtgtttcgatccagaacctgttcaatggtggttgccgtcctttctggggtttcgtttccgataaaatcggccgttacaaaaccatgtccgtcgttttcggtatcaatgctgttgttctcgcacttttcccgacgattgctgccttgggcgatgtagcctttatcgccatgttggcaatcgcattcttcacatggggtggtagctacgctctgttcccatcgaccaacagcgatattttcggtacggcatactctgccagaaactatggtttcttctgggctgcaaaagcaactgcctcgatcttcggtggtggtctgggtgctgcaattgcaaccaacttcggatggaataccgctttcctgattactgcgattacttctttcatcgcatttgctctggctaccttcgttattccaagaatgggccgtccagtcaagaaaatggtcaaattgtctccagaagaaaaagctgtacattaatgcaattattaatgatcaacttcctctgccagaaatcatgctggaagaataataaggtaccctgcagactagtgacgtaaatatatcaataacctttggtcgaaaaaaaaagcccgcactgtcaggtgcgggcttttttctgtgtttccatagcaatcccccagatacctaatgtagttccagcaagcacggccgggcagcttgttctgcctgcgttttcttcaattgagcagtagaccatttagctgtggcatgaatggctgcagaactttcactgttgctacctccagttccaccaccgctgccagagccactcccgtctggattatcattcaaaagagtaatgattacctgccccttatcatcataaggaacaccatctttatagtacgctacagctgtttccattataaaatcctctttgacattaaaaacaatcagttaaaaataagtactgcatatataattactggttttatatacagcataaaaattacgccgctgcgttttccctgtcaaccctgtggattttcatttttgtgaaaacgatcaaaaaacaactactcacaattcgacagtcccgccagataccgcaaaaccggccagacgttaccgttttccgggaccatgatatgagccccgttggggagggtatggagttgctgaaaatgacggtcagattgagttcaccgttttattgttacaggaggccagggcttgtctgctaccggtccggacgagagggataccgggaatttgaatccggttaactgagccggacatacggtaaataaggaatgcaggcagaacgggagtcactgcaggatgagaccggcactacagcagaaaaacaggtgattctggtacggctcagccactaaacacagttggaaactgatgataagatagtcagcggttatataactcactgagtaaaaagatataaaggtaaggagacactcatgagtcgttccggtagcacactgtatcttatcgccctgctgacagcggcaacggtcctgacagcctgcacgccaaagggcagtatggaacaacatacccggcattacgtttatgcatcagatgacggttttgatcctaacttttccacccagaaggccgacacaaca

SEQ ID NO: 1104 - DNA, 7543 bp, Artificial Sequence
  Description of Artificial Sequence: Synthetic polynucleotide
taacgtggtgacgtcgtttgccatcgacagtacccacagcgtcgcactgcaacaggatttcttcgataaagtcttcccgaatattttaccgatggcctacaccctgctgatgtattacttcctgcgggtgaaaaaagcgcatccggtgctgttaatcggcgtgacgtttgttctttctattgtttgttctgcattcggcattttgtaaatggaacgaggcaccgcgtctggcggtgcctctttacttaaggaatttcatcctgtgcaaccccttcaacaagttgaaaactatacggcattaagtgaacgtgccagcgaatatttattggccgtgatccgtcgcaaaccggatgccgtgatttgcctggcgaccggagccacgccattactgacgtatcattatctggtagaaaaaatccaccagcagcaggttgatgtcagccatctcaccttcgtgaagctcgacgaatgggtggatctgccattaacgatgcctggcacctgcgaaactttcctacaacagcatatcgtgcagccgctggggctgcgtgaagaccagctcatcagctttcgctccgaagagataaatgagacagagtgcgaacgggtaacgaacctgattgcgcgcaaaggcggtctggatttatgcgttctcggattggggaaaaacggtcatcttgggctgaacgaaccgggagaaagcctgcaaccagcctgccatatcagtcaacttgatgccagaacacagcaacatgagatgttaaaaaccgcggaccgtcccgtgactcgtgggatcaccttaggcctgaaggatattctcaatgcccgcgaagttttgttactggtgactggcgaaggaaaacaggatgcgacagaacgttttctcacggctaaagtctccaccgctatcccggcttcatttttatggctgcataaccatttcacatgcgtgattgatgaaatgtgtcgtcgttaactcgccaggtaaaggcgttgtagcgtcgacgcatgcatcgatgacgtcccatggctcgagacgtcccatggctcgagacgtcccatggctcgagacgtcccatggctcgagtactgccagaaatcatgctggaagaataagacgtcccatggctcgagcatgcgagagtagggaactgccaggcatcaaataaaatgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgcagattgcagcattacacgtcttgagcgattgtgtaggctggagctgcttcgaagttcctatactttctagagaataggaacttcggaataggaactaaggaggatattcatatggaccatggcgtgcaccctgcagggctagctggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatatgaccagtgcagctacggtgaccgcgagctttaatgacactttttctgtgagcgataatgtcgcggtaatcgtaccggaaaccgatacgcaggtcacctaccgtgatctttcccacatggtaggacactttcaaacaatgttcacgaacccgaatagtcctctgtacggggcggtctttcgtcaagacacggtagcgattagcatgcgtaacggccttgaatttattgtggctttccttggagccacgatggatgcgaaaattggtgcgccactgaatcccaattataaagagaaggagtttaatttttacctgaatgacttaaagtccaaagccatctgcgtgccgaaaggcaccaccaaactgcaaagttcagaaattcttaagagtgcgtccacgttcgggtgctttattgtggaactggcgtttgacgccacccgttttcgtgttgaatatgacatttactccccggaggacaattataaacgtgtgatctaccgcagccttaacaatgctaagtttgtcaacacaaaccctgtcaagttcccgggtttcgcccgcagctcggatgttgcacttattttgcatacctcaggcaccactagtaccccaaagaccgtacccctcttgcatctgaatattgtccgttcaaccctgaatatcgccaacacttacaaacttaccccgctggatcgctcctatgttgtaatgccgctgtttcatgtacatggattaatcggcgtcttactgagtacgttccgcacccagggcagtgtagtcgtcccggacggctttcatccgaagctcttctgggatcagtttgttaaatataactgcaattggtttagttgcgtcccaacgatctctatgattatgttgaatatgcccaaaccgaatccgtttccgcacattcgctttatccgctcatgtagcagcgcgctggcgccagcaacgtttcacaagctggaaaaagaatttaatgccccagttctggaagcgtacgcgatgacagaagcatctcatcagatgaccagtaacaatctgcctcccggtaaacgtaaaccggggaccgtgggccaacctcaaggtgtaaccgtagtaatcctggatgacaacgataacgttctgccgcccggcaaagttggcgaggtgtcgatccgtggggagaacgtcaccctgggctacgctaataacccgaaagctaacaaagaaaacttcactaaacgtgaaaactatttccgtaccggggatcagggctacttcgacccggagggctttctcgtgctgaccggccgcattaaagaattgatcaatcgcggtggtgaaaaaattagtcctattgaactggacggaatcatgctctcgcatcctaaaatcgacgaggcggtggcgttcggcgttccagatgatatgtatggccaagtcgttcaggcggcaatcgtgttgaaaaagggggaaaagatgacctatgaagaattagtgaatttcctgaaaaagcatttagcaagctttaaaatcccaaccaaagtctactttgtggataagctgcctaaaacggccaccgggaagattcaacgtcgcgtaatcgccgaaaccttcgcgaaatctagtcgcaacaaaagcaaactttaaaagaaggagatatacatatgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataataagaaggagatatacatatgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttgatgcaattattaatgatcaacttcctctgccagaaatcatgctggaagaataataaggtaccctgcagactagtgctattcctgccctttggtcgaaaaaaaaagcccgcactgtcaggtgcgggcttttttctgtgtttcctgcgacgcctgtaatcactctccctgctgctccagcgcatacttatacagcgcatttttcttcacgccgtgaatttctgcggccagcgccgccgcttttttcagcggcagttctgcctgtagcagcgccagcgtgcgcagggcatcagcaggcaagtcattttcctgcgctttatggccttcgacaatcagcaccatttcgcctttgcgacggttttcatcttcttttacccacgccagcagctcgccaacgggcgcgccgtgaatggtttcccaggttttggttagctcacgcgccagaaccacgtaacgggattcgcctaataccgcaacgatatcttccaggctatctaacagacggtgggtagattcataaaaaatcagcgtgcgcggctccgcttcaatagcttttagcgcatcacggcggccttttgatttggcaggtaaaaagccttcataacaaaaacgatcggatggtaaacccgcggcgcttaacgcagtgatagcagcacacggccctggtagcggcaccacgcgaatccccgcttcacgacaggtacgcaccagatggtagccaggatcgttaattagcggcgttccggcatcggaaaccagcgcaatgttttgcccctcttgcagcttcgccagcagcgtttcggctttttgttgttcattatggtcgtgcagcgcaaacaaccgggcattaatcccaaaatgttgcagcaataaaccggtgtgacgcgtatcctcggcggcaatcagatcaacggcctgtaatacctccaacgcacgctgggtgatatccgccagattgccgattggcgtcggtacaatgtaaagctgcccctgagaattatccgccgattggtgttgtttcattgtgtcgtccgtattgccgatttaatattgagcattgcgtaaaaaaaaatatcactggatacattatggtaccctcaacattttctcgtttgaaagccgcgcgttgtctgc